SEQUENCE LISTING (3 sequences)

SEQ ID NO: 1 - protein, 595 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 2 - protein, 597 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 3 - protein, 593 aa, Homo sapiens [sequence omitted]

(19) United States
(12) Patent Application Publication
(10) Pub. No.: US 2018/0121597 A1
(43) Pub. Date: May 3, 2018

(54) Methods to Prepare and Employ Binding Site Models for Modulation of Phosphatase Activity and Selectivity Determination

(71) Applicant: Allosta Pharmaceuticals, Winchester, MA (US)

(72) Inventors: Thomas Chan, Winchester, MA (US); Mark A Ashwell, Carlisle, MA (US); Jerome F Baker, Buffalo, NY (US); Rocio Palmer, North Andover, MA (US); Xincai Luo, Bear, DE (US)

(21) Appl. No.: 15/575,802

(22) PCT Filed: May 22, 2016
(86) PCT No.: PCT/US2016/033681

§ 371 Date: Nov. 20, 2017

Related U.S. Application Data
(60) Provisional application No. 62/165,524, filed on May 22, 2015.
(60) Provisional application No. 62/165,503, filed on May 22, 2015.
(60) Provisional application No. 62/165,453, filed on May 22, 2015.

(51) Int. Cl. G06F 19/16; C12N 9/16

(52) U.S. Cl. CPC G06F 19/16; C12Y 301/03048; C12N 9/16

(57) ABSTRACT

The present invention provides SHP2, PTP-PEST (PTPN12, PTPG1), LYP (PTPN22, PEP, PTPN8), PIP1B and STEP Enrichment models, and methods of deriving enrichment models for other tyrosine phosphatases, which function depends on movements of the WPD-loop. Also provided are methods to compare phosphatase Enrichment Models. This provides an implementable process to identify selective modulators of phosphatase activity. Furthermore, it provides methods to select modulators expected to a pre-determined modulators activity across a pre-selected subset of phosphatases. The phosphatase Enrichment Models of the present invention can be used to screen for or design modulators of tyrosine phosphatase function.

### BACKGROUND OF THE INVENTION

Protein phosphatases are classified according to their substrate specificity and are generally divided into two major categories—protein serine/threonine phosphatases (PSTPs) and protein tyrosine phosphatases (PTPs), with dual-specificity phosphatases (DSPs) existing as a subclass of the tyrosine phosphatases. PTPs catalyze dephosphorylation reactions on phospho-tyrosine residues while PSTPs on phospho-serine and phospho-threonine residues, and DSPs on phospho-tyrosine, phospho-serine, and phospho-threonine residues.

Tyrosine phosphorylation and dephosphorylation of proteins are key regulatory events in many cellular signal transduction pathways leading to proliferation, migration, differentiation, and cell death. The level of tyrosine phosphorylation on a protein is determined by the relative contributions of protein tyrosine kinases (PTKs) and protein tyrosine phosphatases (PTPs). While modulation of PTKs by small molecule drugs has been shown to be a clinically relevant strategy for disease control in for example oncology, this has not been the case for PTPs. Protein phosphatases are classified according to their substrate specificity and are generally divided into two major categories—protein serine/threonine phosphatases (PSTPs) and protein tyrosine phosphatases (PTPs), with dual-specificity phosphatases (DSPs) existing as a subclass of the tyrosine phosphatases. PTPs catalyze dephosphorylation reactions on phospho-tyrosine residues, PSTPs on phospho-serine and phospho-threonine residues, and DSPs on phospho-tyrosine, phospho-serine, and phospho-threonine residues.

It is likely therefore that modulators of PTP activity will offer therapeutic benefit in disease treatments or in disease control.

Two such PTPs are Src homology protein phosphatase 1 (SHP-1) and 2 (SHP-2). They have become targets for developing novel therapeutic agents. It is known that SHP-1 plays a negative regulatory role in immune cells and cytokine signaling indicating that small molecule inhibitors of SHP-1 may increase the anti-cancer efficacy of immunotherapy or cytokine therapy. SHP-2, on the other hand, is an oncogenic molecule in human malignancies and a mitogenic signal transducer. Small molecule inhibitors of SHP-2 may be expected to inhibit tumor cell growth. However, due to the biological complexity in these systems it is not possible to say with certainty what full effect inhibitors of SHP-1 and/or SHP-2 will have. In the absence of this knowledge there is a need for the identification of small molecule modulators of SHP function and methods for their identification as tool molecules, and their eventual optimization as drug candidates.

Among the approximately one hundred PTPs encoded in the human genome, many of them can be considered as targets for developing novel therapeutic agents. One such PTP is PTB1B. This PTP is an attractive target for the treatment of diabetes and obesity and has been shown to be a negative regulator of insulin signaling by directly interacting with the insulin receptor.

A further PTP of interest is PTP-PEST (sometimes referred to as PTPN12, PTPG1). It is ubiquitously expressed and plays a role in cell motility, cytokinesis and apoptosis. It is implicated also as a negative regulator of B and T cell signaling. Furthermore, PTP-PEST has been shown to regulate mitogen and cell-adhesion-inducted signaling events in cancer cells.

An even further PTP of interest is LYP (also known as PTPN22, PEP, PTPN8), which is primarily expressed in lymphoid tissue and is involved directly in controlling several immune response pathways. The Arg620Trp mutation in LYP is associated with autoimmune disorders including an increased risk of rheumatoid arthritis, systemic lupus erythematosus, vitiligo and Graves disease.

An additional PTP of interest is the striatal-enriched phosphatase (STEP). Up-regulation of STEP and/or increased activity of the protein contribute to the pathology of diseases such as Alzheimer's disease, schizophrenia, fragile X syndrome, epileptogenesis, and alcohol-induced memory loss.

In order to further understand the biological roles of these and other PTPs, there is a need for the identification of small molecule modulators of functions of these PTPs and development of new methods for their identification and optimization as drug candidates.

Elucidation of their functions and development of methods for their identification will enable the eventual optimization enzyme ligands as drug candidates.

To date the majority of modulators of tyrosine phosphatases described bind at the phosphate binding site. The disadvantages with that being: 1. The phosphate binding sites are lined with positive charges; 2. The generally poor drug-like properties of these inhibitors limit their oral absorption, cell penetration, resulting in high metabolic clearance; 3. Their highly charged nature makes them difficult to make and to purify. There is thus a need for new approaches for the identification of modulators of tyrosine phosphatases.

In part the failure to identify phosphatase inhibitors with good drug-like properties has been the result of the approaches used traditionally to identify these modulators. These have predominantly focused on the use of the active or closed conformation of the phosphatase as the drug-target. In silico methods have almost entirely utilized the active form in which the catalytically essential general acid/base aspartic acid residues are orientated for catalysis and assays have been established which focus on inhibitors that bind at this site.

An additional challenge for the identification of suitable PTP modulators for the treatment of human disease is that methods to demonstrate selective modulation of a particular single or sub-set of PTPs have not been identified.

Therefore, methods are described herein which address this limitation and are shown to have utility for categorization and ranking of the Enrichment Models of PTPs such that determination of the selectivity for a particular PTP can be estimated.

Recently it has become recognized that the conformation of the WPD loop (which contains the catalytically essential residues) can also be in an inactive “open” conformation. In this orientation the WPD loop is found distal to the catalytic pocket. It has become recognized that binding of the substrate or an inhibitor to the bottom of the catalytic site causes the WPD loop to shift to the closed active conformation. Other states involving intermediate and atypically open conformations of the WPD-loop have also been observed. Additionally water molecules play important roles in the WPD-loop closure mechanism.

To perform their biological functions, proteins in solution are in constant motion which can result in large conformational changes. Conformational flexibility defines the binding site location, binding modes and interactions with small molecule modulators, as well as cofactors and substrates. Molecular dynamics (MD) simulations are widely used to explore protein flexibility but MD usually explores the system's global minimum. Other methods such as Normal Mode Analysis operate on vibrational modes found to be relevant for biological function. In general these methods are applied to the full macromolecule target making their application slow and computationally expensive. Since not all of the regions are important for a target's catalytic function, exploring the plasticity of only those regions important for function will make the process more efficient.

There is thus a need for methods to be developed which allow for the identification of modulators of phosphatase function which take into account the plasticity of the phosphatase target. These methods (both in silico and physical screening), if applied to the identification of modulators of phosphatase function, should provide access to new drugs which target phosphatases as their mode of action.

Conformational change is frequently associated with protein function. Structural flexibility and protein movement allow appropriate responses to take place to external changes. Increasingly protein dynamics are being utilized to assess the impact of small molecules on protein structure and function.

Structure-based drug design is severely limited in cases where large conformational changes of the protein take place on binding of a small molecule. Accurate receptor models in the ligand bound state are essential and creating these can be challenging without additional information to guide the receptor model construction.

Studies have shown that poor enrichment factors are typically found when only an unbound protein is available as compared to a pre-existing small molecule bound structure.

Some small degree of receptor flexibility can be accommodated in docking studies by using an ensemble of structures or by modeling flexibility of the side-chains, or small pre-defined sections of a protein, or in some cases by small backbone variations. However, progress is limited since the degree of flexibility is limited. Despite the identification of agents which have been described to affect phosphatase function, there remains a need for additional, novel and selective agents which offer the benefits of increased potency, specificity, and reduced side effects.

Despite the identification of agents which have been described to affect phosphatase function, there remains a need for additional, novel and selective agents which offer the benefits of increased potency, better specificity, and reduced side effects.

The references cited herein are not admitted to be prior art to the claimed invention.

### SUMMARY OF THE INVENTION

Applicant describes herein a method for making an enrichment model for a phosphatase enzyme. The phosphatase is preferably a tyrosine phosphatase, such as SHP1 or, more preferably, SHP2. Methods are provided for the identification of modulators of SHP function. Methods are also provided to enrich a chemical library for binding to the SHP2 protein, or to enrich a chemical library for modulators of the SHP2 protein function.

Described herein are processes for constructing 3-dimensional enrichment models of the SHP2 protein and applying the data generated from this analysis to a computer algorithm, and generating from the computer algorithm binding models suitable for screening or designing SHP2 modulators. Further described is a process for screening or designing SHP2 modulators including using the SHP2 enrichment models to screen or design SHP2 inhibitors. SHP2 enrichment models can be used for the identification of modulators of SHP2 function.

In one aspect of the invention, methods for making Enrichment Models for phosphorylation enzymes are described. In certain embodiments of the invention, the phosphorylation enzyme is a phosphatase. Alternatively, or in addition, the phosphatase is a tyrosine phosphatase. Exemplary tyrosine phosphatases are selected from the group consisting of PTP-PEST, LYP, PTP1B and STEP.

In another aspect of the invention methods are described for assessing phosphatase Enrichment Models by comparison with a further phosphatase. In certain embodiments of the invention this phosphatase is SHP-2. In additional embodiments the phosphatase is selected from PTP-PEST, LYP, PTP1B and STEP.

The invention provides methods for the identification of modulators of PTP-PEST, LYP, PTP1B and STEP function.

In certain embodiments of the invention, methods are provided to enrich a chemical library for binding to the PTP-PEST, LYP, PTP1B and STEP.

In certain embodiments of the invention, methods are provided to enrich a chemical library for modulators of the PTP-PEST, LYP, PTP1B and STEP functions.

The invention provides processes for constructing 3-dimensional Enrichment Models of the PTP-PEST, LYP, PTP1B and STEP proteins and applying the data generated from this analysis to a computer algorithm, and generating from the computer algorithm binding models suitable for screening or designing PTP-PEST, LYP, PTP1B and STEP modulators. The invention further provides a process for screening or designing PTP-PEST, LYP, PTP1B and STEP modulators including using the PEST, PTP1B and STEP Enrichment Models to screen or design PTP-PEST, LYP, PTP1B and STEP inhibitors.

The invention provides PTP-PEST, LYP, PTP1B and STEP Enrichment Models for use in the identification of modulators of PTP-PEST, LYP, PTP1B and STEP function.

Further the invention provides a multi-stage process for the identification of selective modulators of the PTP-PEST, LYP, PTP1B and STEP proteins by comparison of the respective Enrichment Models.

Furthermore, the invention provides a multi-stage process for the application of methods described herein for the identification of modulators of any phosphatase, especially protein tyrosine phosphatases.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 is a two dimensional rendering of the three dimensional back-bone residues of the Enrichment Model 4.1 (SHP-2 EM4.1) (black spheres), compared to those corresponding residue of STEP (white spheres). Numerical values beside the residue number correspond to the acceptor hydrogen bond values reported in Table 31.

FIG. 1a: Two-dimensional rendering of enrichment model 1

FIG. 1b: Representative Hit structures for Enrichment Model 1

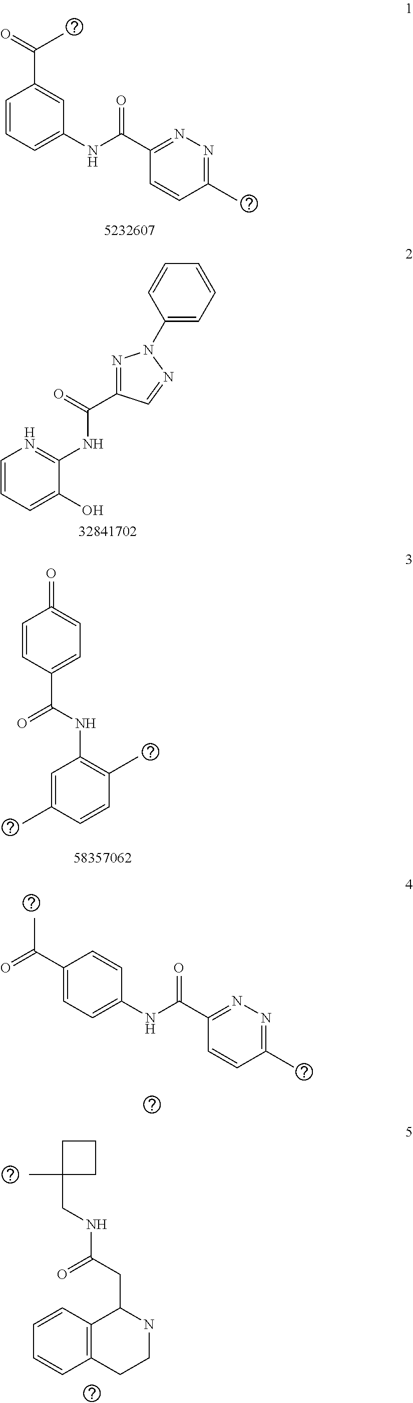

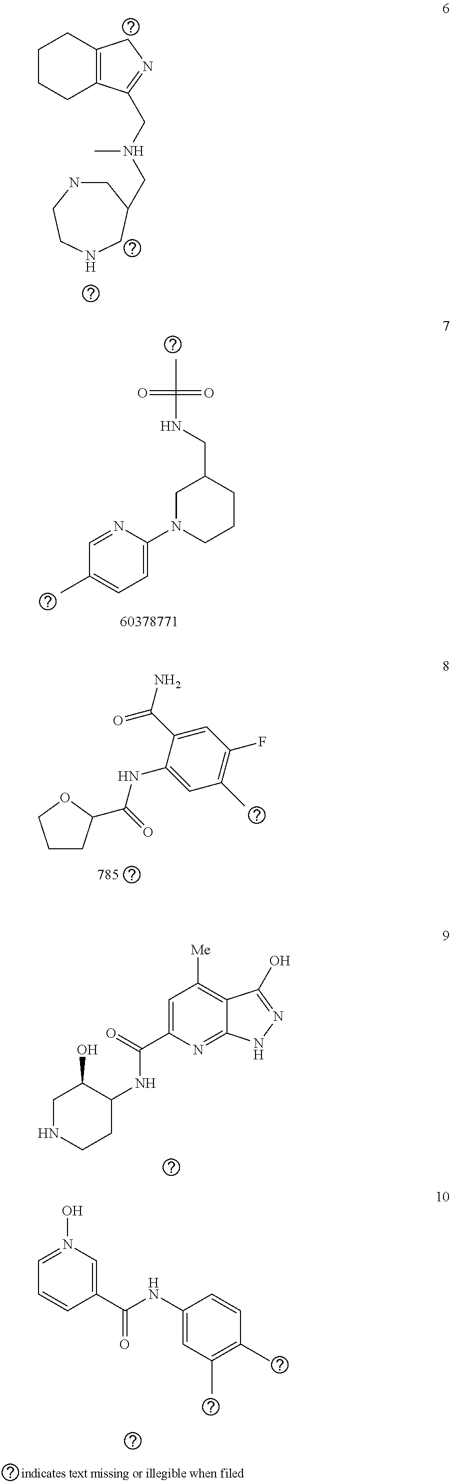

FIG. 2a: Two-dimensional rendering of Enrichment Model 2

FIG. 2b: Representative Hit structures for Enrichment Model 2

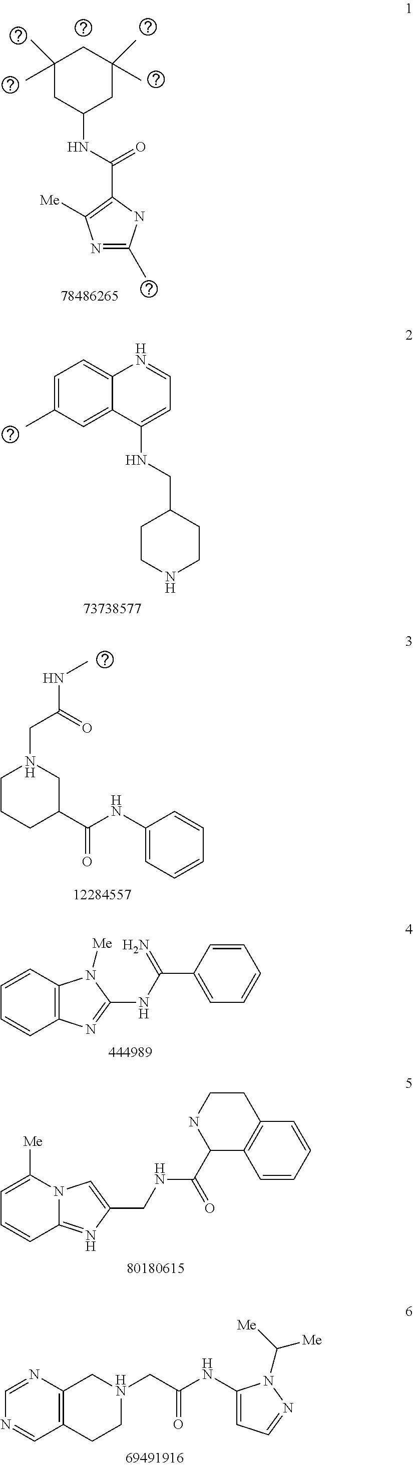

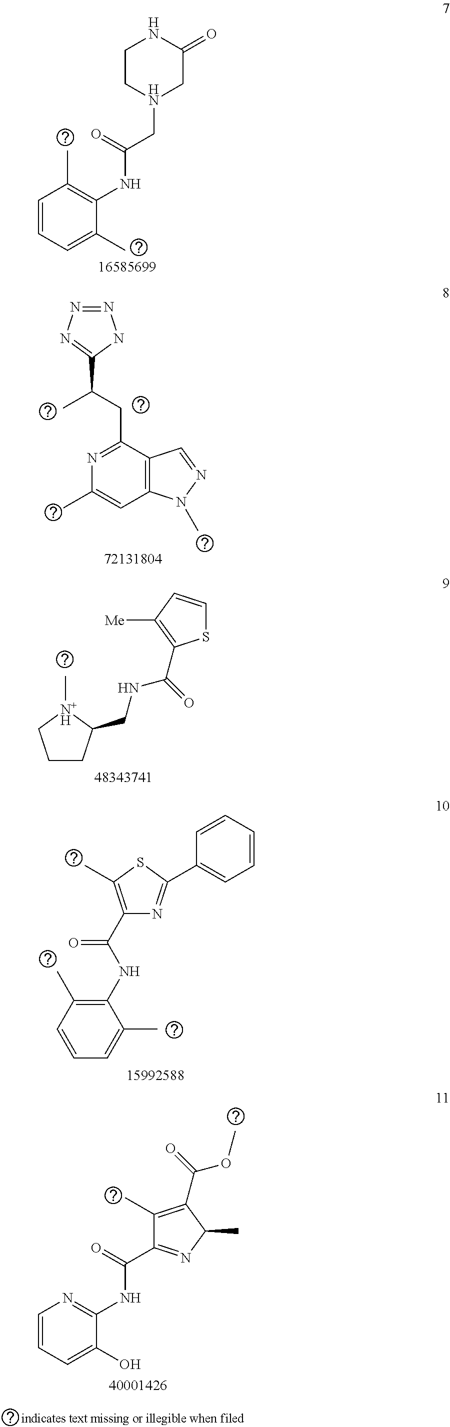

FIG. 3a: Two-dimensional rendering of Enrichment Model 3

FIG. 3b: Representative Hit structures for Enrichment Model 3

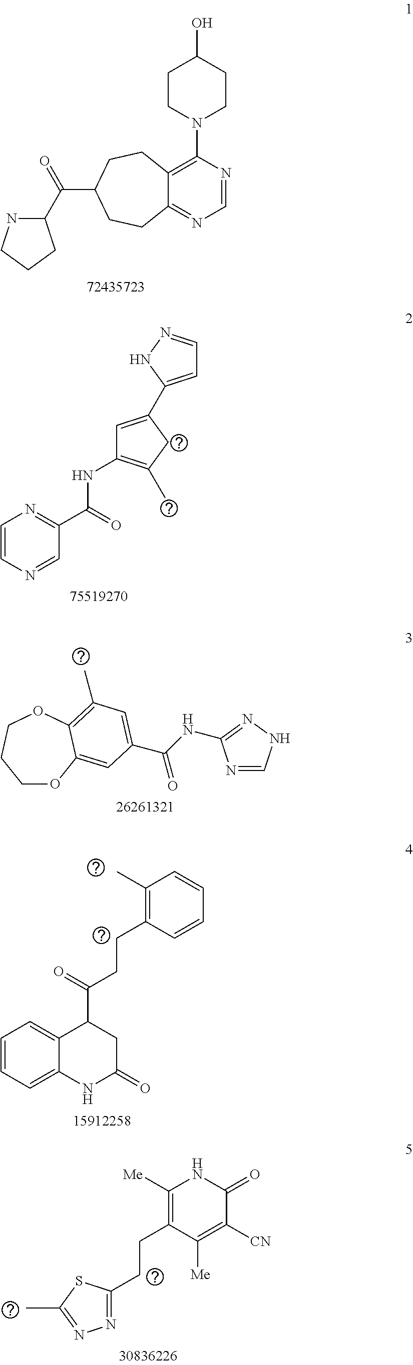 [SMILES: O=C(C1CCc2ncnc(N3CCC(O)CC3)c2CC1)C1CCCN1 ; CC1=C(NC(=O)c2cnccn2)C=C(c2ccn[nH]2)C1 ; Cc1cc(C(=O)Nc2nc[nH]n2)cc2c1OCCCO2 ; Cc1ccccc1CCC(=O)C1CC(=O)Nc2ccccc21 ; Cc1nnc(CCc2c(C)[nH]c(=O)c(C#N)c2C)s1]

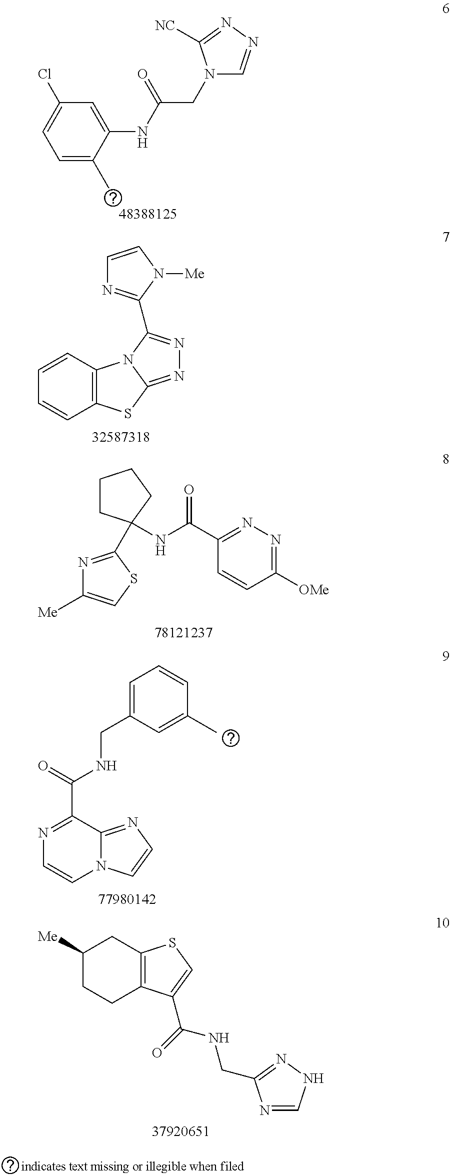 [SMILES: Cc1ccc(Cl)cc1NC(=O)Cn1cnnc1C#N ; Cn1ccnc1-c1nnc2sc3ccccc3n12 ; COc1ccc(C(=O)NC2(c3nc(C)cs3)CCCC2)nn1 ; Cc1cccc(CNC(=O)c2nccn3ccnc23)c1 ; C[C@@H]1CCc2c(C(=O)NCc3nc[nH]n3)csc2C1]

FIG. 4a: Two-dimensional rendering of Enrichment Model 4 Collection Example 1

FIG. 4b: Representative Hit structure for Enrichment Model 4 Collection Example 1

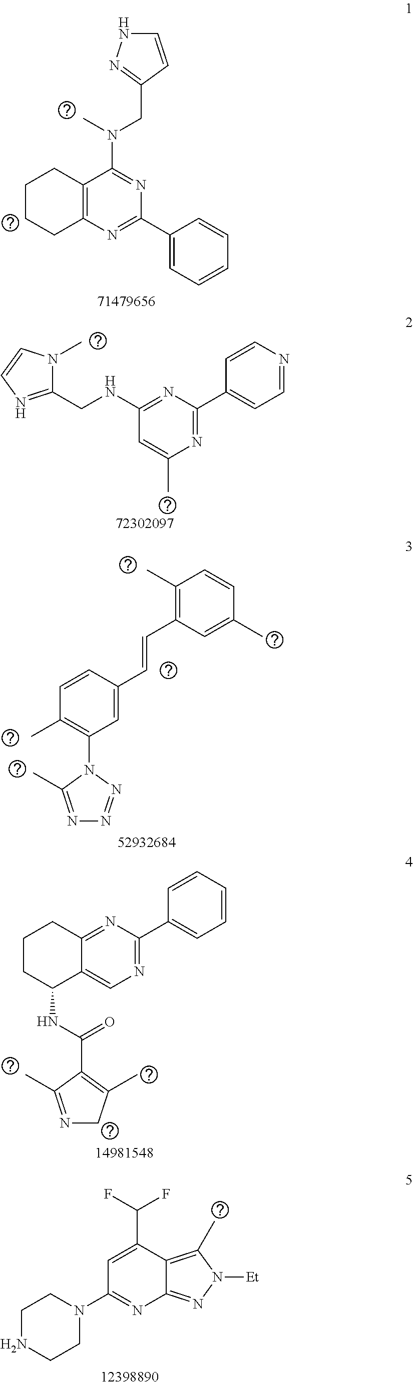

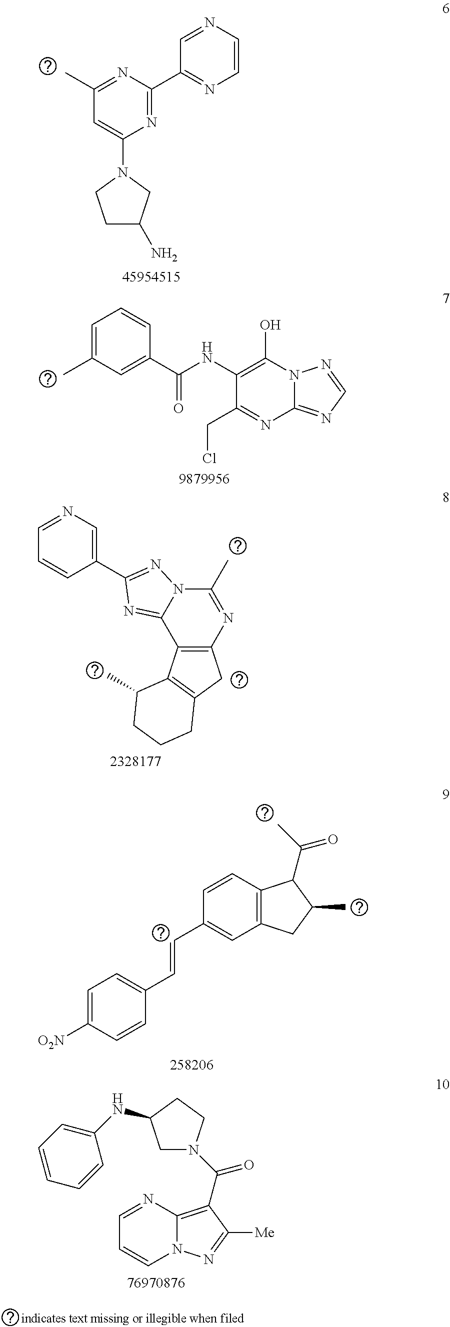 [SMILES: Cc1cc(N2CCC(N)C2)nc(-c2cnccn2)n1 ; Cc1cccc(C(=O)Nc2c(CCl)nc3ncnn3c2O)c1 ; Cc1nc2c(c3nc(-c4cccnc4)nn13)C1=C(CCC[C@@H]1C)C2 ; CC(=O)C1c2ccc(/C=C/c3ccc([N+](=O)[O-])cc3)cc2C[C@@H]1C ; Cc1nn2cccnc2c1C(=O)N1CC[C@H](Nc2ccccc2)C1]

FIG. 5a: Two-dimensional rendering of Enrichment Model 4 Collection Example 2

FIG. 5b: Representative Hit structure for Enrichment Model 4 Collection Example 2

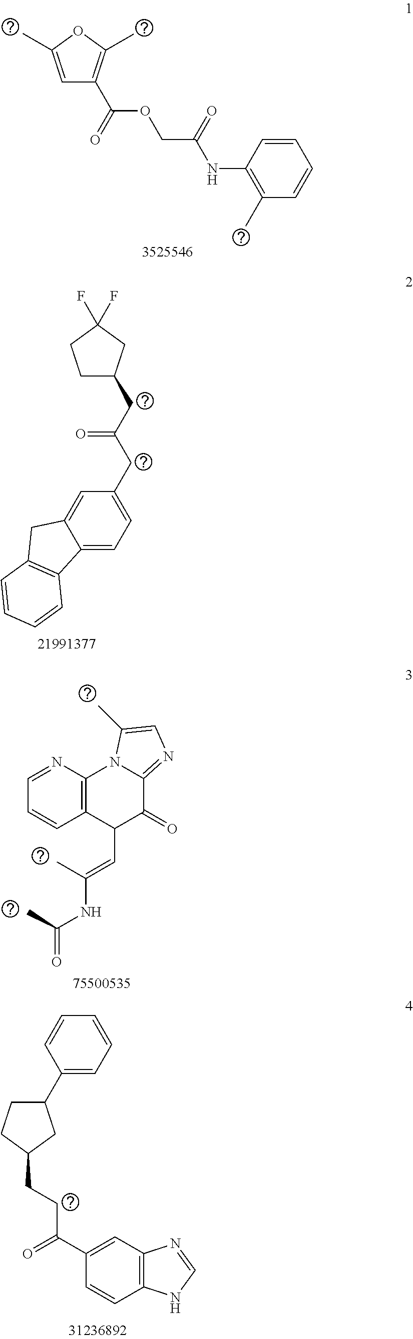

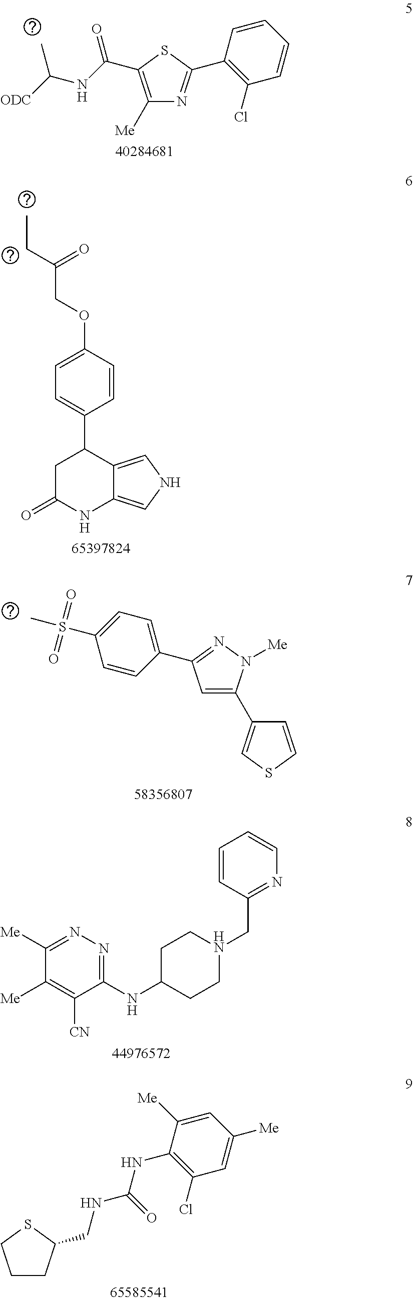

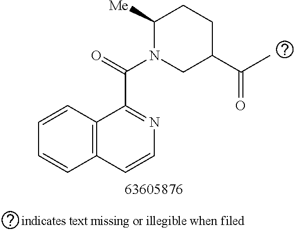 [SMILES: CC(=O)C1CC[C@H](C)N(C(=O)c2nccc3ccccc23)C1]

### DETAILED DESCRIPTION OF THE INVENTION

Described herein are SHP2 three-dimensional computational models, methods of chemical library enrichment for binding to SHP2, and methods for the design of SHP2 modulators.

The present invention provides PTP-PEST, LYP, PTP1B and STEP 3-dimensional computational models.

The present invention provides methods for the design of PTP-PEST, LYP, PTP1B and STEP modulators.

The present invention provides multi-stage methodology for comparing three dimensional Enrichment Models for selective enrichment of chemical libraries for binding to PTP-PEST, LYP, PTP1B and STEP. As described more fully below, an Enrichment Model is comprised of a set of amino acid residues within a region of a protein. This collection of residues may be used to devise putative binding site models which may, with further transformation and process, provide pharmacophore models for the identification of modulators of the protein's function. One use for the Enrichment Model is to identify chemical modulators from a library of small chemical entities. In order for an Enrichment Model to provide the basis for the identification of modulators a number of steps are required. These include, but are not limited to, the generation of 3-dimensional representations of putative interaction sites within the Enrichment Model. Such processes may include visualization and computational analysis, or creation of prospective binding sites with molecular complementarity for modulator interaction, which may themselves form the basis for further process such as molecular dynamic simulations, conformational analysis, molecular docking, pharmacophore generation, and construction of database queries.

Another use for a first Enrichment Model is to determine the degree of similarity between additional Enrichment Models derived from different proteins. In this way comparison of the amino acid residues and their properties within the respective Enrichment Models will indicate the likelihood of identifying modulators with either similar or dissimilar structural features. Two methods are described herein. Method 1 relies upon comparisons of the amino acids within the Enrichment Models and Method 2 provides calculations of certain properties of the amino acid residues within the Enrichment Models being compared. Thus the two methods provide information which may be translated to the modulators of the protein's function.

In this way, methods are provided for chemical library enrichment for binding to PTP-PEST, LYP, PTP1B and STEP, and for multi-stage methodology for selective enrichment of chemical libraries for binding to any phosphatase.

### Computers, Computer Software, Computer Modeling and Methods

Computers are known in the art and may include a central processing unit (CPU), a working memory, which can be random-access memory, core memory, mass-storage memory, or combinations of all of the aforementioned. Computers may also include display, and input and output devices, such as one or more cathode-ray tube or other video display terminals, keyboards, modems, input lines and output lines. Further, said computers may be networked to computer servers (the machine on which large calculations can be run in batches), and file servers (the main machine for all the centralized databases).

Machine-readable media containing data, such as the crystal structure coordinates of the polypeptides of the invention may be inputted using various hardware, including modems, CD-ROM drives, disk drives, or keyboards.

Output hardware, such as a CRT display or other video display terminals, may be used for displaying a graphical representation of the SHP-2, PTP-PEST, LYP, PTP1B and STEP polypeptides of the invention or the SHP-2, PTP-PEST, LYP, PTP1B and STEP Enrichment Models of these polypeptides. Output hardware may also include a printer, and disk drives.

The CPU may encode one or more programs. The CPU coordinates the use of the various input and output devices, coordinates data accesses from storage and accesses to and from working memory, and determines the sequence of data processing steps. A number of programs may be used to process the machine-readable data of this invention. Such programs are discussed in reference to the computational methods of drug discovery as described herein.

X-ray coordinate data can be modified according to the methods described herein, and then processed into a three dimensional graphical display of a molecule or molecular complex that comprises a SHP-2-, PTP-PEST-, LYP-, PTP1B- or STEP-like substrate binding pocket stored in a machines-readable storage medium. The three-dimensional structure of a molecule or molecular complex comprising a SHP-2-, PTP-PEST-, LYP-, PTP1B- and STEP-like substrate-binding pocket may be used for a variety of purposes, including, but not limited to, library enrichment and drug discovery. By a process of electronic representation, lists of structure coordinates is converted into a structural models, which can be a graphical representation in three-dimensional space.

The three dimensional structure may be rendered in two-dimensions by 3D rendering or alternative display may serve as the source for computer simulations.

Using the three-dimensional structure derived from the structure coordinate data, Applicants designed an Enrichment Model of the region or regions of the protein that Applicants predict can be used to design associations with another chemical entity or compound. These regions are formed by amino acid residues which Applicants interpret to be key for ligand binding, or the regions may be amino acid residues that are spatially related and define a three-dimensional shape which can be used to model a binding pocket. The amino acid residues may be contiguous or non-contiguous in primary sequence. The region or regions may be embodied as a dataset (e.g., an array) recorded on computer readable media.

This virtual 3-dimensional computer generated representation of what is suitable for a small molecule chemical entity to bind is useful as a library enrichment model. Such a process, referred to here as an enrichment method, requires that an Enrichment Model be converted to a putative binding site model in order to generate 3-dimensional pharmacophores. The pharmacophores are then utilized to identify modulators through the use of computer methods such as docking experiments. The Enrichment method can be used to design potential drug candidates and to evaluate the ability of prospective drug candidates to inhibit or otherwise modulate the activity of SHP-2, PTP-PEST, LYP, PTP1B and STEP.

An Enrichment Model can contain, but is not synonymous with, the concept of a motif, a group of amino acid residues in a protein that defines a structural compartment or carries out a function in the protein, for example, catalysis, structural stabilization, or phosphorylation. A motif may be conserved in sequence, structure and function. A motif is generally contiguous in primary sequence. Examples of a motif include, but are not limited to, a binding pocket for ligands or substrates; WPD-loop, C(X)5R, or more explicitly (I/V)HCXAGXGR(S/T)G sequence motif. Andersen et al., ‘Structural and Evolutionary Relationships among Protein Tyrosine Phosphatase Domains,’ Mol. Cell. Biol., 2001, 21(21):7117-7136.

A chemical entity which is associated with an Enrichment Model can be a chemical compound, a complex of at least two chemical compounds, or a fragment of such compounds or complexes. A chemical entity can be an analog, e.g., a functional analog, a structural analog, a transitional state analog, or a substrate analog. A chemical entity can also be, depending on context, a scaffold, which is a chemical skeleton somewhere between a fragment and a ligand—it can be present in several ligands—or a ligand which binds to a binding site, or target or target site, of interest. Such chemical entities have a chemical structure, which includes an atom or group of atoms that constitute a part of a molecule. Normally, chemical structures of a scaffold or ligand have a role in binding to a target molecule.

A chemical entity or compound, or portion thereof, may bind to or have binding affinity for a protein when in a condition of proximity to the library Enrichment Model, or binding pocket or binding site on a protein. The association may be non-covalent, for example, wherein the juxtaposition is energetically favored by hydrogen bonding, van der Waals forces, and/or electrostatic interactions. Some, albeit not all, such chemical entities can serve as modulators, a modulator being a small molecule which is capable of interacting with the target protein in a way that is sufficient to alter the normal function of the protein. A modulator can be, e.g., an activator or an inhibitor, or an up-regulator or a down-regulator, or an agonist, an inverse agonist, or an antagonist. In another aspect, a modulator can act in an allosteric manner. In yet another aspect, a modulator can act by enhancing the activity of another chemical entity.

Interactions between a chemical entity and a binding pocket, domain, molecule or molecular complex or portion thereof, include but are not limited to one or more of covalent interactions, non-covalent interactions such as hydrogen bond, electrostatic, hydrophobic, aromatic, van der Waals interactions, and non-complementary electrostatic interactions such as repulsive charge-charge, dipole-dipole and charge-dipole interactions. Such interactions generate and are characterized by a certain level of interaction energy. As interaction energies are measured in negative values, the lower the value the more favorable the interaction.

The crystal structure of a composition can be represented in a computer readable medium in which is stored a representation of three-dimensional positional information for atoms of the composition.

An Enrichment Model is not to be confused with a homology model, which refers to a set of coordinates derived from known three-dimensional structure used as a template. Generation of the homology model involves sequence alignment, residue replacement, and residue conformation adjustment through energy minimization. Homology modeling is based on the primary assumption that if proteins share a degree of similarity then their fold and three dimensional structures could be similar as well. The general procedure to build a homology model requires the following steps: sequence alignment, identification of structurally conserved regions, coordinate generation where all heavy-atom coordinates are copied when residue identity is conserved between the target sequence and its template; otherwise, only backbone coordinates are copied. Next coordinates for loops are generated and search for possible side-chain conformations is carried out. Finally the new structure is refined and evaluated. For sequence alignment a commonly used benchmark is CLUSTALW (Higgins et al., Nucleic Acids Res., 1994, 22:4673-4680; Chenna et al., Nucleic Acids Res., 2003, 31:3497-3500) and for model building studies is SWISS-MODEL (Schwede et al., Nucleic Acids Res., 2003, 31:3381-3385). Both of these programs are accessible through their Web sites. Homology modeling can also be performed using commercial software packages; non-limiting examples of such programs are MOE (CCG, Montreal, Canada), ICM (Molsoft, La Jolla, Calif.), and Insight II/Discover (Accelrys, Inc., San Diego, Calif.).

By a process of structure preparation, protein structures are computationally checked for errors to produce high quality models. Common problems include missing hydrogen atoms, incomplete side chains and loops, ambiguous protonation states, and flipped residues. CONECT records are ignored and bonds are assigned based on geometry. Standard residues, such as amino acids, are bonded according to their atom names, hydrogen atoms are included and partial charges are calculated. To remove bad crystallographic contacts and other geometry issues the models are energy minimized in the presence of solvent using standard force fields provided by programs and methods such as MMFF94x within MOE (i.e., “Molecular Operating Environment”) (CCG, Montreal, Canada), QUANTA/CHARMM (Accelrys, Inc., San Diego, Calif.); Gaussian (M. J. Frisch, Gaussian, Inc., Carnegie, Pa.); AMBER (P. A. Kollman, University of California at San Francisco); Jaguar (Schrödinger, Portland, Oreg.); SPARTAN (Wavefunction, Inc., Irvine, Calif.); Impact (Schrödinger, Portland, Oreg.); Insight II/Discover (Accelrys, Inc., San Diego, Calif.); MacroModel (Schrödinger, Portland, Oreg.); Maestro (Schrödinger, Portland, Oreg.); and DelPhi (Accelrys, Inc., San Diego, Calif.). Softwares such as MOE (CCG, Montreal, Canada), ICM (Molsoft, La Jolla, Calif.), and Insight II/Discover (Accelrys, Inc., San Diego, Calif.), Protein Preparation Wizard (Schrödinger, Portland, Oreg.) allow for an automated protein structure preparation.

Binding sites are identified by computational methods used to find such sites which include geometric analyses, energy calculations, evolutionary considerations, machine learning and others. A number of applications are available. These include, but are not limited to the SiteFinder algorithm (Prot. Pept. Lett., 2011, 10:997-1001), which considers the relative positions and accessibility of the receptor atoms and their chemical type. The methodology is based on the concept of Alpha Spheres, a generalization of convex hulls. This procedure classifies the Alpha Spheres as hydrophobic or hydrophilic, depending on whether the sphere provides a hydrogen bonding spot (Edelsbrunner et al., Proceedings of the 28th Hawaii International Conference on Systems Science, 1995, 1:256-264). (MOE, CCG, Montreal, Canada), pocket cavity detection algorithm based on Voronoi tesellation, LIGSITE automatic detection of pockets using Connolly surfaces, Cavitator, which detects pockets or cavities in a protein structure, using a grid-based geometric analysis (Center for the Study of Systems Biology, Atlanta, Ga.). ICM-PocketFinder is a binding site predictor based on calculating the drug-binding density field and contouring it at a certain level (Molsoft, La Jolla, Calif.). SiteMap is a software program for binding site identification (Schrodinger Portland, Oreg.); POCASA (POcket-CAvity Search Application) can predict binding sites by detecting pockets and cavities of proteins of known 3D structure (Hokkaido University, Japan; http://altair.sci.hokudai.ac.jp/g6/service/pocasa/). FTSite method is based on experimental evidence that ligand binding sites also bind small organic molecules of various shapes and polarity (Boston University, Boston, Mass.: ftsite.bu.edu).

By using molecular docking methods, chemical entities are positioned in different orientations and conformations within the identified binding sites. For each chemical entity, a number of configurations, so-called poses, are generated and scored. A set of conformations is generated from a single 3D conformer by selecting preferred torsion angles of rotatable bonds. Bond lengths and bond angles are not altered. Rings are not flexed. The results of the fitting operation are then analyzed to quantify the association between the chemical entity and the binding site. The quality of fitting of these entities to the model is evaluated either by using a scoring function, shape complementarity, or estimating the interaction energy. Methods for evaluating the association of a chemical entity with the binding site include energy minimization with standard molecular mechanics force fields. Examples of such programs include: MOE (CCG, Montreal, Canada), QUANTA/CHARMM (Accelrys, Inc., San Diego, Calif.); Gaussian: (M. J. Frisch, Gaussian, Inc., Carnegie, Pa.); AMBER (P. A. Kollman, University of California at San Francisco); Jaguar (Schrödinger, Portland, Oreg.); SPARTAN (Wavefunction, Inc., Irvine, Calif.); Impact (Schrödinger, Portland, Oreg.); Insight II/Discover (Accelrys, Inc., San Diego, Calif.); MacroModel (Schrödinger, Portland, Oreg.); Maestro (Schrödinger, Portland, Oreg.); and DelPhi (Accelrys, Inc., San Diego, Calif.). Potential hits are identified based on favorable geometric fit and energetically favorable complementary interactions. Energetically favorable electrostatic interactions include attractive charge-charge, dipole-dipole and charge-dipole interactions between the target enzyme, and the small molecule. Available docking programs, for example, are MOE (CCG, Montreal, Canada), ICM (Molsoft, La Jolla, Calif.), FelxiDock (Tripos, St. Louis, Mo.), GRAM (Medical Univ. of South Carolina), DOCK3.5 and 4.0 (Univ. Calif. San Francisco), Glide (Schrödinger, Portland, Oreg.), Gold (Cambridge Crystallographic Data Centre, UK), FLEX-X (BioSolveIT, GmbH, Germany), or AUTODOCK (Scripps Research Institute).

To further understand a drug's biological activity, a pharmacophore model is defined. A pharmacophore model is a set of steric and electronic features necessary for a strong ligand interaction with the biological target responsible for its biological activity. The: pharmacophore model shows the location and type of important atoms and groups like aromatic centers, hydrophobic, hydrogen bond donor and acceptor features. A variety of automated and manual tools are available to assist with building a pharmacophore model from ligands, receptor structures, or protein-ligand complexes. These include, but are not limited to, commercially available software such as Pharmacophore Query Editor, Query Generator and PLIF Protein Ligand Interaction Fingerprints, and MOE (CCG, Montreal, Canada); Catalyst, HipHop, and HypoGen (Accelrys, Inc., San Diego, Calif.); and DISCO, GASP, and GALAHAD (Tripos, St. Louis, Mo.); and PHASE (Schrödinger, Portland, Oreg.).

A protein editor allows one to modify a protein by mutating, inserting or deleting residues or segments at specific location in the chain. The newly created residues may make energetically unfavorable interactions with their neighbors. To accommodate the change the system has to be energy minimized. Protein editors include but are not limited to Copy/Paste, where the insertion point or region to replace is chosen first, then the fragment to be grafted onto the target chain is specified and copied to the clipboard, and finally Paste joins the objects together. Program suites such as MOE (CCG, Montreal, Canada), and QUANTA Modeling Environment (Accelrys, Inc., San Diego, Calif.) provide protein editors, and energy minimization is carried out with standard molecular mechanics force fields. Examples of such programs and program suites include: MOE (CCG, Montreal, Canada), QUANTA/CHARMM (Accelrys, Inc., San Diego, Calif.); Gaussian (M. J. Frisch, Gaussian, Inc., Carnegie, Pa.); AMBER (P. A. Kollman, University of California at San Francisco); Jaguar (Schrödinger, Portland, Oreg.); SPARTAN (Wavefunction, Inc., Irvine, Calif.); Impact (Schrödinger, Portland, Oreg.); Insight II/Discover (Accelrys, Inc., San Diego, Calif.); MacroModel (Schrödinger, Portland, Oreg.); Maestro (Schrödinger, Portland, Oreg.); and DelPhi (Accelrys, Inc., San Diego, Calif.).

Another useful tool is a conformational search, which is applied preferably to a protein loops. Protein loops often play a vital role in protein functions, mainly because they usually interact with the solvent and other molecules. In some cases experimentally determined structures show loops corresponding to ‘open’ and ‘closed’ states. In some cases other important intermediate states may exist since the motions of protein loops depend on secondary structure or large domain motions but these may not be experimentally determined. Several methods have been implemented for conformational searching of molecular systems. Examples include but are not limited to LowModeMD Conformational Search method [Labute, J. Chem. Inf. Model., 2010, 50:792-800] which generates conformations using a short (˜1 ps) Molecular Dynamics (MD) run at constant temperature. MD Velocities are randomly applied mainly to the low-frequency vibrational modes of the system resulting in rapid and more realistic conformational transitions. LowModeMD Search takes into account detailed information about possibly complex non-bonded interaction network, force-field restraints, macrocyclic structure and concerted motions MOE (CCG, Montreal, Canada). LOOPER (Prot. Engineer., Des. Select., 2008, 21:91-100), in contrast to many ab initio algorithms that use Monte-Carlo schemes or exhaustive sampling, adopts a systematic search strategy with minimal sampling of the backbone torsion angles (Accelrys, Inc., San Diego, Calif.).

Methods to prepare the small molecule database from which Candidate Modulators are identified. A source of Candidate Modulators was prepared from a large collection of small molecules in the ZINC database. The ZINC data base is located at the zinc.docking.org website. This data base contains commercially available compounds originally designed for target based virtual screening. The service is provided by the Shoichet Laboratory (UCSF)—Irwin and Shoichet, J. Chem. Inf. Model., 2005, 45(1):177-182.

A 3D conformation database of Candidate Modulators of SHP2 modulators was prepared as follows:
a. Forty five compressed files of Lead Like compounds were downloaded from the ZINC data base. Each raw data file contains a subset of approximately 150K compounds providing a total of 6.3 million compounds. Each subset was prepared to clean errors, missing annotations, and other omissions. Illegal or unrecognized molecules were eliminated using structure preparation tools.
b. Abbreviations were translated and molecules with unrecognized atoms or formats were rejected. Transition metals or atoms with too many bonds were eliminated. Undesirable molecules were filtered using a coded SMARTS pattern language.
c. The enumeration of tautomers and protonation states, stereochemical states, and standardization of molecular structure (e.g. with respect to bonding patterns) was performed.
d. The resulting data file was filtered using Oprea's test for leadlikeness. To pass the filter a candidate modulator can have at most one violation of the following conditions: a) the number N or O that are hydrogen bond donors must be 5 or less; b) the number of N and O atoms must be 8 or less; c) the molecular weight must be 450 or less; d) the logP must be in the range [−3.5, 4.5], inclusive; e) the number of rings of size three through eight must be 4 or less; and, f) the number of rotatable bonds (as defined by Oprea) must be 10 or less. To provide Candidate Modulators the number of rotatable bonds to was further reduced to less than 4 and the number of chiral centers to no more than one. About two thirds of each set was rejected providing about 45-50 K molecules in each set.
e. To prepare the 3D Candidate Modulator database a conformational analysis was performed. Low-energy conformations of Candidate Modulators were calculated by decomposing each molecule into constituent overlapping fragments, then performing a stochastic conformational search on each fragment, followed by the assembly of fragments into unique conformers.
f. To speed the docking process a Diverse Subset of 500 Candidate Modulators was selected from each set using the following process. 2D descriptors were calculated: a_acc, a_acid, a_aro, a₁₃base, a_count, a_don, a_hyd, b_count, b_double, PEOE_PC+, PEOE_PC−, PEOE_VSA_HYD, PEOE_VSA_POL, vdw_vol. MOE's Diverse Subset application used to select diverse subsets of compounds ranks entries based on their distance from a reference set and from each other. The distance between two entries is calculated as Euclidean distance between their corresponding points in n-dimensional descriptor space.

### Construction of SHP2 Library Enrichment Models

Models for the modulation of SHP2 are constructed by the preparation of the 3-dimensional representation of the SHP2 protein based on but not limited to the crystallographic structure of the SHP2 protein and the application of computer algorithms to modify regions important for phosphatase function as explained in methods.

The electronic representation of the SHP2 structures are then displayed on a computer screen for visual inspection and analysis. All important motifs involved in SHP2 ligand recognition and binding were identified, including those described above.

Three dimensional graphical representation of the SHP2 modulation sites were then generated as part of an electronic representation of the ligand bound binding site. In an embodiment, the electronic representation of the binding site contains the coordinates of SHP2 residues, up to 4.5 Å from the center of every Alpha Sphere in each selected site

The structure coordinates of amino acid residues that constitute the binding site define the chemical environment important for ligand binding, and thereby are useful in designing compounds that may interact with those residues.

The binding site amino acid residues are key residues for ligand binding. Alternatively, the binding site amino acid residues may be residues that are spatially related in the definition of the three-dimensional shape of the binding site. The amino acid residues may be contiguous or non-contiguous in the primary sequence.

The SHP2 binding sites are formed by three-dimensional coordinates of amino acid residues selected after modifying the X-ray crystallographic structure of the SHP2 protein as explained in methods. These models are mostly hydrophobic in nature but also contain polar moieties, which correspond to backbone atoms.

Computer programs are also employed to estimate the attraction, repulsion, and steric hindrance of the ligand to the SHP2 Enrichment Model. Generally the tighter the fit between the inhibitor and SHP2 at the molecular level and atomic level (e.g., the lower the steric hindrance, and/or the greater the attractive force), the more potent the potential drug will be because these properties are consistent with a tighter-binding constant.

A ligand selected in the manner described above is expected to overcome the known randomness of screening all chemical matter for the identification of hit molecules. Once the enrichment methods have identified SHP2 modulators they can be systematically modified by computer-modeling programs until one or more promising potential ligands are identified. Such computer modeling allows the selection of a finite number of rational chemical modifications, as opposed to the countless number of essentially random chemical modifications that could be made, any of which any one might lead to a useful drug. Each chemical modification requires additional chemical steps, which while being reasonable for the synthesis of a finite number of compounds, quickly becomes overwhelming if all possible modifications needed to be synthesized. Thus, through the use of the structure coordinates disclosed herein and computer modeling, a large number of these compounds are rapidly screened on the computer monitor screen, and a few likely candidates are determined or identified without the laborious synthesis of untold numbers of compounds.

Once a potential ligand (agonist or antagonist) is identified, it is either selected from commercial libraries of compounds or synthesized de novo. As mentioned above, the de novo synthesis of one or even a relatively small group of specific compounds is reasonable in the art of drug design.

For the drug design strategies described herein further refinement(s) of the structure of the drug are generally necessary and are made by the successive iterations of any and/or all of the steps provided by the aforementioned strategies.

The structure coordinates generated from the SHP2 complex can be used to generate a three-dimensional shape. This is achieved through the use of commercially available software that is capable of generating three-dimensional graphical representations of molecules or portions thereof from a set of structure coordinates.

Various computational analyses can be performed to analyze SHP2 or other phosphatases. Such analyses may be carried out through the use of known software applications, such as ProMod, SWISS-MODEL (Swiss Institute of Bioinformatics), and the Molecular Similarity application of QUANTA (Accelrys, Inc., San Diego, Calif.). Programs such as QUANTA permit comparisons between different structures, different conformations of the same structure, and different parts of the same structure. Comparison of structures using such computer software may involve the following steps: 1) loading the structures to be compared; 2) defining the atom equivalencies in the structures; 3) performing a fitting operation; and 4) analyzing the results. Each structure is identified by a name. One structure is identified as the target (i.e., the fixed structure) and all remaining structures are working structures (i.e., moving structures). Since atom equivalency with QUANTA is defined by user input, equivalent atoms can be defined as protein backbone atoms (N, Cα, C, and O) for all conserved residues between the two structures being compared. Rigid fitting operations are also considered. When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atoms is an absolute minimum. This number, given in angstroms (Å), is reported by software applications, such as QUANTA.

### Use of the Enrichment Models for Ligand Screening (Enrichment), Fitting and Selection

The SHP2 Enrichment models are used for ligand screening (enrichment), fitting, and selection.

The electronic representation of compounds and/or fragments is generated as described above, Electronic representations of compounds and/or fragments are assembled into electronic databases. These databases include chemical entities' coordinates in any SMILES, mol, sdf, or mol2 formats.

Selected chemical entities or fragments may be positioned in a variety of orientations inside the Enrichment model. Chemical entities come from different sources including, but not limited to, proprietary compound repositories, commercial data bases, or virtual data bases. Non-limiting exemplary sources of fragments include reagent data bases, de-novo design, etc.

The selected chemical entities or fragments are used to perform a fitting of the electronic representation of compounds and/or fragments and the Enrichment model. The fitting is done manually or is computer assisted (docking).

The results of the fitting operation are then analyzed to quantify the association between the chemical entity and the Enrichment model. The quality of fitting of these entities to the Enrichment model is evaluated either by using a scoring function, shape complementarity, or estimating the interaction energy.

Methods for evaluating the association of a chemical entity with the Enrichment model include energy minimization and molecular dynamics with standard molecular mechanics force fields, such as CHARMM (Accelrys, Inc., San Diego, Calif.) and AMBER (P. A. Kollman, University of California at San Francisco).

Additional data is obtained using Free Energy Perturbations (FEP), to account for other energetic effects such as desolvation penalties. Information about the chemical interactions with the Enrichment model are used to elucidate chemical modifications that can enhance selectivity of binding of the modulator.

Potential binding compounds are identified based on favorable geometric fit and energetically favorable complementary interactions. Energetically favorable electrostatic interactions include attractive charge-charge, dipole-dipole and charge-dipole interactions between the target enzyme, and the small molecule.

The association with the Enrichment Model is further assessed by means of visual inspection followed by energy minimization and molecular dynamics. Examples of such programs include: MOE (CCG, Montreal, Canada), QUANTA/CHARMM (Accelrys, Inc., San Diego, Calif.); Gaussian (M. J. Frisch, Gaussian, Inc., Carnegie, Pa.); AMBER (P. A. Kollman, University of California at San Francisco); Jaguar (Schrödinger, Portland, Oreg.); SPARTAN (Wavefunction, Inc., Irvine, Calif.); Impact (Schrödinger, Portland, Oreg.); Insight II/Discover (Accelrys, Inc., San Diego, Calif.); MacroModel (Schrödinger; Portland, Oreg.); Maestro (Schrödinger, Portland, Oreg.); and DelPhi (Accelrys, Inc., San Diego, Calif.).

Once suitable fragments have been identified, they are connected into a single compound or complex on the three-dimensional image displayed on a computer screen in relation to all or a portion of the Enrichment Model.

### Use of the Enrichment Models for Ligand Design

The design of compounds using the Enrichment Models includes calculation of non-covalent molecular interactions important in the compound's binding association including hydrogen bonding, van der Waals interactions, hydrophobic interactions and electrostatic interactions.

The compound's binding affinity to the Enrichment Model is further optimized by computational evaluation of the deformation energy of binding, i.e. the energy difference between bound and free states of the chemical entity.

Computer calculations may suggest more than one conformation similar in overall binding energy for a chemical entity. In these cases the deformation energy of binding is defined as the difference between the energy of the free entity and the average energy of the conformations observed when the inhibitor binds to the protein.

### Enrichment Model Examples

Enrichment Model 1 takes advantage of the presence of water molecules in the autoinhibited structure of SHP2. Including water molecules in the model reduces the polarity of the site and allows for the identification of neutral molecules during virtual screening. Water molecules have been proposed to play a role in tyrosine phosphatase function. A crystallographic water molecule tightly bound to two conserved glutamine residues Gln262 and 266 in PTP1B has been proposed to play a role in the WPD-loop closure mechanism. In structures with open WPD-loop the ‘catalytic water’ is not present or it is displaced.

### EXAMPLE 1

### Enrichment Model 1 and Its Use

General Description of Enrichment Model 1

This method describes the use of autoinhibited conformations of SHP2 for the identification of Candidate Modulators which are expected to bind to SHP2 and affect its function. The human triple mutant SHP2 structure was used for the Enrichment Model construction. This 2 Å resolution structure includes the PTP, N- and C-SH2 domains and corresponds to the autoinhibited phosphatase. The PDB access code is 2SHP.

General Method Description: The Construction of Enrichment Model 1

To prepare the SHP2 Enrichment Model 1 missing loops and side-chains were constructed for the SHP2 structure (PDB access code: 2SHP) using homology modeling with the available full sequence (UnitProtKB entry Q06124) from the SWISPROT data base. Once these were added to the SHP2 structure it was fully relaxed in the presence of solvent to relieve bad crystallographic contacts or other geometry issues.

Construction of the Enrichment Model 1
1—The SHP2 structure contains residues 1-527, the following mutations are present T2K, F41L F513S.
2—The full sequence of human SHP2 was downloaded from the SWISPROT data base UnitProtKB entry Q06124;
3—Missing data was replaced and corrected before using the structure for Enrichment Model construction. Missing side chain residues were placed into the Enrichment Model 1 using Homology Modeling techniques.
4—Once constructed the Enrichment Model 1 was checked for errors and energy minimized in the presence of solvent with a standard Molecular Mechanics force field using Structure Preparation tools.

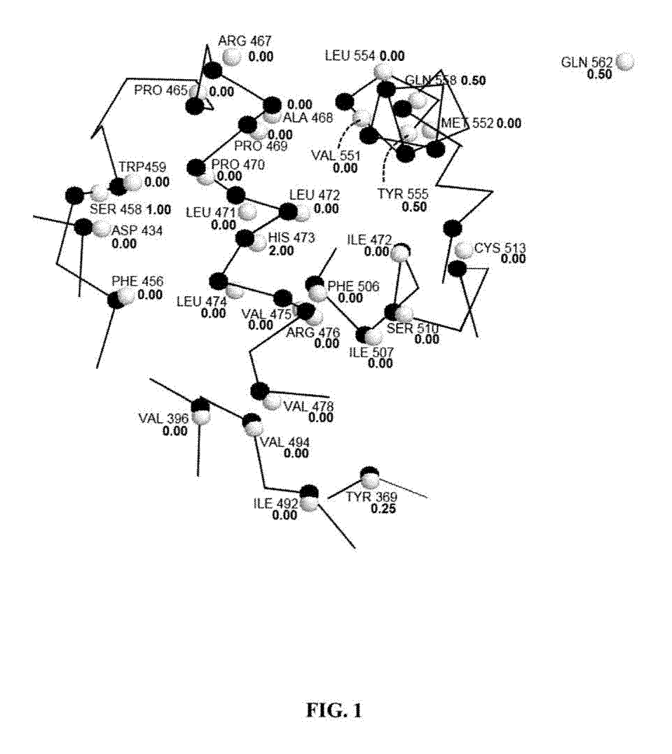

Preparation of the Enrichment Model 1
1—The Enrichment Model 1 was searched for the presence of molecular features suitable for the binding of SHP2 modulators using a Binding Site Identification technique.
2—Sites were checked for size and polarity giving preference to more hydrophobic rather than hydrophilic sites.
3—Visual inspection of the SHP2 residues occupying the Enrichment Model 1 was performed.
4—Enrichment Model 1 contains two aromatic hydrophobic residues: Tyr 62 and Trp 423, several water molecules and polar side chains. See FIG. 1a for a 2-dimensional rendering. Table 1 contains the Enrichment Model 1 three dimensional coordinates

Identification of Candidate Modulators of SHP2 Using the Enrichment Model 1
1—Molecular Docking of the Diverse Subset with Enrichment Model 1 identified Candidate Modulators.
2—The Enrichment Model 1 included the catalytic site of SHP2 water molecules present in the original structure to increase the number of neutral Candidate Modulators present in the results.
3—Candidate Modulators from the use of the Enrichment Model 1 were accepted if they contained at least two rings.
4—Candidate Modulators were energy minimized and their interactions with the Enrichment Model 1 were analyzed looking for complementarity with compound's features.
5—The analysis allowed for the creation of a Pharmacophore Model with excluded volumes representing the binding site protein atoms.
6—A further filtering of the Diverse Subset hits employing the pharmacophore query provided the final Candidate Modulators.
7—A set of analogs was selected from those hits showing an excellent match with the pharmacophore query. The analogs were identified by searching the previously prepared ZINC database. Representative examples of small molecule hits are in FIG. 1b

TABLE 1
Coordinates of the sites used for docking within the Enrichment Model

| | | | | | | | | | |
|---|---|---|---|---|---|---|---|---|---|
| ATOM | 1 | N | THR | A | 59 | 10.325 | −29.084 | 41.232 | N |
| ATOM | 2 | CA | THR | A | 59 | 10.423 | −27.868 | 40.432 | C |
| ATOM | 3 | C | THR | A | 59 | 11.77 | −27.833 | 39.701 | C |
| ATOM | 4 | O | THR | A | 59 | 11.974 | −27.075 | 38.746 | O |
| ATOM | 5 | CB | THR | A | 59 | 10.315 | −26.599 | 41.304 | C |
| ATOM | 6 | OG1 | THR | A | 59 | 11.392 | −26.542 | 42.262 | O |
| ATOM | 7 | CG2 | THR | A | 59 | 9.004 | −26.532 | 42.078 | C |
| ATOM | 8 | N | GLY | A | 60 | 12.772 | −28.573 | 40.308 | N |
| ATOM | 9 | CA | GLY | A | 60 | 14.132 | −28.47 | 39.879 | C |
| ATOM | 10 | C | GLY | A | 60 | 15.184 | −28.824 | 40.899 | C |
| ATOM | 11 | O | GLY | A | 60 | 16.243 | −29.337 | 40.527 | O |
| ATOM | 12 | N | ASP | A | 61 | 14.973 | −28.371 | 42.182 | N |
| ATOM | 13 | CA | ASP | A | 61 | 16.082 | −28.287 | 43.11 | C |
| ATOM | 14 | C | ASP | A | 61 | 16.212 | −29.551 | 43.97 | C |
| ATOM | 15 | O | ASP | A | 61 | 17.292 | −29.754 | 44.539 | O |
| ATOM | 16 | CB | ASP | A | 61 | 16.006 | −27.037 | 43.983 | C |
| ATOM | 17 | CG | ASP | A | 61 | 16.68 | −25.804 | 43.403 | C |
| ATOM | 18 | OD1 | ASP | A | 61 | 17.433 | −25.968 | 42.39 | O |
| ATOM | 19 | OD2 | ASP | A | 61 | 16.446 | −24.709 | 44.03 | O1− |
| ATOM | 20 | N | TYR | A | 62 | 15.04 | −30.269 | 44.153 | N |
| ATOM | 21 | CA | TYR | A | 62 | 14.891 | −31.403 | 45.067 | C |
| ATOM | 22 | C | TYR | A | 62 | 13.501 | −32.063 | 44.971 | C |
| ATOM | 23 | O | TYR | A | 62 | 12.468 | −31.423 | 44.764 | O |
| ATOM | 24 | CB | TYR | A | 62 | 15.172 | −31.02 | 46.537 | C |
| ATOM | 25 | CG | TYR | A | 62 | 14.9 | −29.582 | 46.941 | C |
| ATOM | 26 | CD1 | TYR | A | 62 | 13.649 | −28.974 | 46.779 | C |
| ATOM | 27 | CD2 | TYR | A | 62 | 15.959 | −28.803 | 47.425 | C |
| ATOM | 28 | CE1 | TYR | A | 62 | 13.487 | −27.596 | 46.983 | C |
| ATOM | 29 | CE2 | TYR | A | 62 | 15.802 | −27.435 | 47.65 | C |
| ATOM | 30 | CZ | TYR | A | 62 | 14.573 | −26.835 | 47.397 | C |
| ATOM | 31 | OH | TYR | A | 62 | 14.441 | −25.476 | 47.521 | O |
| ATOM | 32 | N | GLU | A | 361 | 14.358 | −17.887 | 47.009 | N |
| ATOM | 33 | CA | GLU | A | 361 | 13.504 | −18.179 | 45.846 | C |
| ATOM | 34 | C | GLU | A | 361 | 12.198 | −18.664 | 46.549 | C |
| ATOM | 35 | O | GLU | A | 361 | 12.142 | −19.753 | 47.123 | O |
| ATOM | 36 | CB | GLU | A | 361 | 14.191 | −19.227 | 44.946 | C |
| ATOM | 37 | CG | GLU | A | 361 | 15.266 | −18.645 | 44.008 | C |
| ATOM | 38 | CD | GLU | A | 361 | 16.2 | −19.635 | 43.308 | C |
| ATOM | 39 | OE1 | GLU | A | 361 | 15.702 | −20.579 | 42.618 | O |
| ATOM | 40 | OE2 | GLU | A | 361 | 17.465 | −19.457 | 43.464 | O1− |
| ATOM | 41 | N | ARG | A | 362 | 11.193 | −17.708 | 46.661 | N |
| ATOM | 42 | CA | ARG | A | 362 | 9.909 | −17.917 | 47.357 | C |
| ATOM | 43 | C | ARG | A | 362 | 9.991 | −17.943 | 48.898 | C |
| ATOM | 44 | O | ARG | A | 362 | 9.115 | −18.455 | 49.597 | O |
| ATOM | 45 | CB | ARG | A | 362 | 9.126 | −19.154 | 46.872 | C |
| ATOM | 46 | CG | ARG | A | 362 | 8.772 | −19.083 | 45.395 | C |
| ATOM | 47 | CD | ARG | A | 362 | 8.219 | −20.405 | 44.872 | C |
| ATOM | 48 | NE | ARG | A | 362 | 8.074 | −20.317 | 43.413 | N |
| ATOM | 49 | CZ | ARG | A | 362 | 8.125 | −21.369 | 42.559 | C |
| ATOM | 50 | NH1 | ARG | A | 362 | 8.132 | −22.659 | 42.961 | N |
| ATOM | 51 | NH2 | ARG | A | 362 | 8.147 | −21.103 | 41.237 | N1+ |
| ATOM | 52 | N | LYS | A | 364 | 12.198 | −19.385 | 50.645 | N |
| ATOM | 53 | CA | LYS | A | 364 | 12.894 | −20.598 | 51.041 | C |
| ATOM | 54 | C | LYS | A | 364 | 14.377 | −20.238 | 51.059 | C |
| ATOM | 55 | O | LYS | A | 364 | 14.945 | −19.758 | 50.076 | O |
| ATOM | 56 | CB | LYS | A | 364 | 12.681 | −21.741 | 50.04 | C |
| ATOM | 57 | CG | LYS | A | 364 | 11.373 | −22.498 | 50.285 | C |
| ATOM | 58 | CD | LYS | A | 364 | 11.578 | −23.846 | 50.982 | C |
| ATOM | 59 | CE | LYS | A | 364 | 12.225 | −23.737 | 52.357 | C |
| ATOM | 60 | NZ | LYS | A | 364 | 12.383 | −25.097 | 52.901 | N1+ |
| ATOM | 61 | N | LYS | A | 366 | 17.384 | −20.943 | 50.344 | N |
| ATOM | 62 | CA | LYS | A | 366 | 18.177 | −21.697 | 49.395 | C |
| ATOM | 63 | C | LYS | A | 366 | 19.634 | −21.599 | 49.881 | C |
| ATOM | 64 | O | LYS | A | 366 | 20.469 | −22.469 | 49.651 | O |
| ATOM | 65 | CB | LYS | A | 366 | 18.073 | −21.055 | 48.009 | C |
| ATOM | 66 | CG | LYS | A | 366 | 16.7 | −21.117 | 47.331 | C |
| ATOM | 67 | CD | LYS | A | 366 | 16.243 | −22.519 | 46.917 | C |
| ATOM | 68 | CE | LYS | A | 366 | 15.153 | −22.435 | 45.85 | C |
| ATOM | 69 | NZ | LYS | A | 366 | 14.798 | −23.764 | 45.346 | N1+ |
| ATOM | 70 | N | TRP | A | 423 | 18.069 | −16.644 | 37.148 | N |
| ATOM | 71 | CA | TRP | A | 423 | 17.57 | −18.017 | 37.057 | C |
| ATOM | 72 | C | TRP | A | 423 | 16.21 | −17.947 | 37.777 | C |
| ATOM | 73 | O | TRP | A | 423 | 16.16 | −17.639 | 38.979 | O |
| ATOM | 74 | CB | TRP | A | 423 | 18.511 | −18.973 | 37.812 | C |
| ATOM | 75 | CG | TRP | A | 423 | 18.061 | −20.402 | 37.801 | C |
| ATOM | 76 | CD1 | TRP | A | 423 | 17.032 | −20.934 | 38.551 | C |
| ATOM | 77 | CD2 | TRP | A | 423 | 18.606 | −21.485 | 37.045 | C |
| ATOM | 78 | NE1 | TRP | A | 423 | 16.901 | −22.257 | 38.234 | N |
| ATOM | 79 | CE2 | TRP | A | 423 | 17.842 | −22.62 | 37.306 | C |
| ATOM | 80 | CE3 | TRP | A | 423 | 19.684 | −21.608 | 36.152 | C |
| ATOM | 81 | CZ2 | TRP | A | 423 | 18.08 | −23.854 | 36.693 | C |
| ATOM | 82 | CZ3 | TRP | A | 423 | 19.967 | −22.849 | 35.565 | C |
| ATOM | 83 | CH2 | TRP | A | 423 | 19.167 | −23.953 | 35.825 | C |
| ATOM | 84 | N | PRO | A | 424 | 15.076 | −18.16 | 37.022 | N |
| ATOM | 85 | CA | PRO | A | 424 | 13.758 | −18.089 | 37.621 | C |
| ATOM | 86 | C | PRO | A | 424 | 13.396 | −19.408 | 38.315 | C |
| ATOM | 87 | O | PRO | A | 424 | 13.694 | −20.529 | 37.895 | O |
| ATOM | 88 | CB | PRO | A | 424 | 12.831 | −17.865 | 36.426 | C |
| ATOM | 89 | CG | PRO | A | 424 | 13.526 | −18.624 | 35.295 | C |
| ATOM | 90 | CD | PRO | A | 424 | 15.008 | −18.431 | 35.59 | C |
| ATOM | 91 | N | ASP | A | 425 | 12.525 | −19.268 | 39.357 | N |
| ATOM | 92 | CA | ASP | A | 425 | 11.855 | −20.363 | 40.043 | C |
| ATOM | 93 | C | ASP | A | 425 | 10.809 | −21.116 | 39.153 | C |
| ATOM | 94 | O | ASP | A | 425 | 9.862 | −21.745 | 39.641 | O |
| ATOM | 95 | CB | ASP | A | 425 | 11.149 | −19.824 | 41.299 | C |
| ATOM | 96 | CG | ASP | A | 425 | 11.724 | −18.602 | 42.023 | C |
| ATOM | 97 | OD1 | ASP | A | 425 | 12.245 | −17.722 | 41.256 | O |
| ATOM | 98 | OD2 | ASP | A | 425 | 11.517 | −18.562 | 43.266 | O1− |
| ATOM | 99 | N | HIS | A | 426 | 11.052 | −21.152 | 37.795 | N |
| ATOM | 100 | CA | HIS | A | 426 | 10.098 | −21.61 | 36.782 | C |
| ATOM | 101 | C | HIS | A | 426 | 10.916 | −22.21 | 35.624 | C |
| ATOM | 102 | O | HIS | A | 426 | 10.857 | −21.774 | 34.474 | O |
| ATOM | 103 | CB | HIS | A | 426 | 9.217 | −20.47 | 36.265 | C |
| ATOM | 104 | CG | HIS | A | 426 | 8.298 | −19.93 | 37.29 | C |
| ATOM | 105 | ND1 | HIS | A | 426 | 7.135 | −20.579 | 37.684 | N1+ |
| ATOM | 106 | CD2 | HIS | A | 426 | 8.319 | −18.76 | 38.013 | C |
| ATOM | 107 | CE1 | HIS | A | 426 | 6.443 | −19.824 | 38.542 | C |
| ATOM | 108 | NE2 | HIS | A | 426 | 7.182 | −18.738 | 38.791 | N |
| ATOM | 109 | N | GLY | A | 427 | 11.666 | −23.308 | 36.004 | N |
| ATOM | 110 | CA | GLY | A | 427 | 12.442 | −24.115 | 35.074 | C |
| ATOM | 111 | C | GLY | A | 427 | 13.865 | −23.569 | 35.012 | C |
| ATOM | 112 | O | GLY | A | 427 | 14.6 | −23.564 | 36.004 | O |
| ATOM | 113 | N | VAL | A | 428 | 14.198 | −23.008 | 33.801 | N |
| ATOM | 114 | CA | VAL | A | 428 | 15.539 | −22.548 | 33.422 | C |
| ATOM | 115 | C | VAL | A | 428 | 15.441 | −21.111 | 32.829 | C |
| ATOM | 116 | O | VAL | A | 428 | 14.341 | −20.61 | 32.562 | O |
| ATOM | 117 | CB | VAL | A | 428 | 16.171 | −23.567 | 32.443 | C |
| ATOM | 118 | CG1 | VAL | A | 428 | 16.377 | −24.923 | 33.126 | C |
| ATOM | 119 | CG2 | VAL | A | 428 | 15.351 | −23.744 | 31.159 | C |
| ATOM | 120 | N | GLY | A | 464 | 20.105 | −29.895 | 40.733 | N |
| ATOM | 121 | CA | GLY | A | 464 | 19.488 | −29.57 | 39.462 | C |
| ATOM | 122 | C | GLY | A | 464 | 20.4 | −28.66 | 38.66 | C |
| ATOM | 123 | O | GLY | A | 464 | 20.84 | −28.936 | 37.539 | O |
| ATOM | 124 | N | ARG | A | 465 | 20.641 | −27.455 | 39.305 | N |
| ATOM | 125 | CA | ARG | A | 465 | 21.393 | −26.395 | 38.651 | C |
| ATOM | 126 | C | ARG | A | 465 | 22.83 | −26.879 | 38.397 | C |
| ATOM | 127 | O | ARG | A | 465 | 23.46 | −26.546 | 37.389 | O |
| ATOM | 128 | CB | ARG | A | 465 | 21.418 | −25.105 | 39.5 | C |
| ATOM | 129 | CG | ARG | A | 465 | 20.082 | −24.345 | 39.482 | C |
| ATOM | 130 | CD | ARG | A | 465 | 19.502 | −23.974 | 40.85 | C |
| ATOM | 131 | NE | ARG | A | 465 | 19.601 | −22.542 | 41.166 | N |
| ATOM | 132 | CZ | ARG | A | 465 | 18.53 | −21.73 | 41.46 | C |
| ATOM | 133 | NH1 | ARG | A | 465 | 17.252 | −22.154 | 41.621 | N |
| ATOM | 134 | NH2 | ARG | A | 465 | 18.803 | −20.414 | 41.631 | N1+ |
| ATOM | 135 | N | GLN | A | 510 | 15.305 | −29.294 | 32.964 | N |
| ATOM | 136 | CA | GLN | A | 510 | 16.739 | −29.047 | 32.999 | C |
| ATOM | 137 | C | GLN | A | 510 | 17.375 | −29.851 | 31.873 | C |
| ATOM | 138 | O | GLN | A | 510 | 18.294 | −29.371 | 31.204 | O |
| ATOM | 139 | CB | GLN | A | 510 | 17.408 | −29.504 | 34.293 | C |
| ATOM | 140 | CG | GLN | A | 510 | 17.145 | −28.58 | 35.468 | C |
| ATOM | 141 | CD | GLN | A | 510 | 17.206 | −29.335 | 36.78 | C |
| ATOM | 142 | OE1 | GLN | A | 510 | 17.586 | −30.497 | 36.916 | O |
| ATOM | 143 | NE2 | GLN | A | 510 | 16.739 | −28.591 | 37.83 | N |
| TER | 144 | GLN | A | 510 | | | | | |
| HETATM | 145 | O | HOH | A3038 | | 15.539 | −26.12 | 37.846 | |
| HETATM | 146 | O | HOH | A3069 | | 12.584 | −28.356 | 43.573 | |
| HETATM | 147 | O | HOH | A3192 | | 14.131 | −22.997 | 38.4 | |
| HETATM | 148 | O | HOH | A3222 | | 16.513 | −25.804 | 40.146 | |
| HETATM | 149 | O | HOH | A3369 | | 10.669 | −29.304 | 44.91 | |
| HETATM | 150 | O | HOH | A3449 | | 9.087 | −28.495 | 46.926 | |
| HETATM | 151 | O | HOH | A3623 | | 14.897 | −23.995 | 40.584 | |
| END | | | | | | | | | |

Enrichment Models 2-4 result from exploration of conformational flexibility of the tyrosine phosphatase WPD-loop, the αF-helix and adjacent regions. These regions have been shown to play an important role on stabilization of the catalytic conformation of tyrosine phosphatases. In PTP1B an additional helix α7 stabilizes the closure of the WPD-loop by interacting with helices α3 and α6. In the structure of PTPL1 the α0 helix is located at a topological equivalent position to helix α7 in PTP1B suggesting a similar role in the stabilization of the WPD-loop. A small molecule interacting with those regions could destabilize the WPD-loop and therefore inhibit the tyrosine phosphatase catalytic activity

### EXAMPLE 2

### Enrichment Models 2 and 3 and Their Use

General Description of Enrichment-Model 2 and 3

The SHP2 structure (PDB access code: 4DGP) last resolved residue is Glu528 out of 533 residues in the construct, while the full sequence has 597 residues. The last 67 residues correspond to the C-terminus region which has been implicated in the SHP2 phosphatase function. This region undergoes phosphorylation by PDGFR at residues 546 and 584 and then interacts with the N-SH2 domain removing it from the PTP domain and activating SHP2. This Enrichment Method describes the use of C-terminus of SHP2 which is further expected to be located close to the αF helix (residues 437-451) which is connected to the WPD loop. Modulators of SHP2 identified in this enrichment method are expected to bind and modulate the movement of the WPD-loop which is essential for activation of SHP2.

General Method Description: The Construction of Enrichment Model 2 and 3

To prepare the SHP2 Enrichment Models 2 and 3 missing loops and side-chains were constructed using the SHP2 structure (PDB access code: 4DGP) using homology modeling with the available full sequence (UnitProtKB entry Q06124) from the SWISPROT data base, excluding the C-terminus. The backbone and sidechains were completed and errors corrected. Hydrogen atoms were included and partial charges calculated. Once these were added to the SHP2 structures the protein models were fully relaxed in the presence of solvent to avoid clashes using the standard Molecular Mechanics force field to relieve bad crystallographic contacts or other geometry issues. A C-terminus short peptide was further included in the Enrichment Models 2 and 3. To construct Enrichment Model 2 a homology model of the catalytic domain of SHP2 was built employing the structure of PTP1B phosphatase (PDB access code 2NT7) which includes the C-terminus α7 helix (S285-D298). Then the short C-terminus peptide was saved as a chain and then connected to the SHP2 structure. To construct Enrichment Model 3 the C-terminus α7 helix (S285-D298) of PTP1B phosphatase (PDB access code 2NT7) was employed as the short peptide with direct grafting of the α7 helix from the homology model on to the SHP2 structure using a Protein Editor.

Construction of the Enrichment Model 2
1—A homology model of the catalytic domain of SHP2 was built employing the structure of PTP1B phosphatase (PDB access code 2NT7) which includes the C-terminus α7 helix (S285-D298). Then the short C-terminus peptide was manually grafted onto the SHP2 structure.
2—The last 14 residues (S285-D298) of the α7 helix of the catalytic domain of PTP1B (PDB access code 2NT7) were grafted to the prepared SHP2 structure of the General Method.
3—To avoid clashes with residues from the SHP2 beta strands βJ-βK only the last eight SHP2 residues I⁵³³EEEQKSK⁵⁴⁰ were retained.
4—Enrichment Model 2 residues are G⁴³⁷ L⁴⁴⁰ D⁴⁴¹ E⁴⁴⁴ E⁴⁴⁵ H⁴⁴⁸ H⁵²⁴ Y⁵²⁵ E⁵²⁷ T⁵²⁸ R⁵³¹ R⁵³² I⁵³³ E⁵³⁴ E⁵³⁵ E⁵³⁶ K⁵⁴⁰.

Construction of the Enrichment Model 3
1. The PTP1B (S285-D298) α7 helix was grafted directly to the full length of SHP2 prepared in the general method using the Protein Editor. The Helix did not overlay with the PTP1B template structure. In this case the application placed the short peptide avoiding clashes with SHP2 beta strands βJ-βK which are placed differently in the PTP1B structure.
2. Enrichment Model 3 residues are P³¹² E³¹³ F³¹⁴ E³¹⁵ K³²² P³²³ K³²⁴ K³²⁵ S³²⁶ Y³²⁷ H⁴⁴⁷ Q⁴⁵⁰ E⁴⁵¹ I⁴⁵³ M⁴⁵⁴ A⁴⁵⁶ G⁴⁵⁷ P⁴⁵⁸ V⁴⁵⁹ D⁴⁷⁷ I⁴⁷⁸ D⁴⁸¹ I⁴⁸² R⁴⁸⁴ E⁴⁸⁵ K⁴⁸⁶ E⁵³⁴ E⁵³⁵ E⁵³⁶ Q⁵³⁷ K⁵³⁸ S⁵³⁹ K⁵⁴⁰ R⁵⁴¹ K⁵⁴² G⁵⁴³ H⁵⁴⁴ E⁵⁴⁵ Y⁵⁴⁶ T⁵⁴⁷.

Construction of Enrichment Model 2 and 3
1. Missing data was replaced and corrected before Enrichment Model construction. Homology Modeling was used to place missing side chains and residues into the Enrichment Models 2 and 3.
2. The Enrichment Models 2 and 3 were checked for errors and energy minimized in the presence of solvent by using a standard Molecular Mechanics force field using Structure Preparation tools.

Preparation of the Enrichment Models 2 and 3
1. The Enrichment Models were obtained after Conformational Searching of the grafted segment.
2. Coordinates were saved and searched for molecular features sufficient to provide binding of the SHP2 modulators using Binding Site Identification tools.
3. Sites were checked for size and polarity giving preference to more hydrophobic rather than hydrophilic sites.
4. Enrichment Models with at least two aromatic hydrophobic residues and several polar side chains were selected. Enrichment Model 2 has only one aromatic hydrophobic residue Tyr 525 but in this case Leu 440 is providing the required hydrophobic nature as well as the carbon chains of polar residues (FIG. 2a). The 3-dimensional coordinates of Enrichment Model 2 are in Table 2. Enrichment Model 3 includes the hydrophobic aromatic Tyr 327 and Tyr 547. Size wise Enrichment Model 2 is smaller than Enrichment Model 3 (FIG. 3a); 3-dimensional coordinates are in Table 3.
5. No solvent molecules were included in the Enrichments Models 2 & 3 for docking.

Utilization of the Enrichment Model 2 and 3
6. Molecular Docking of the Diverse Subset with Enrichment Models 2 and 3 identified Candidate Modulators.
7. Candidate Modulators were energy minimized and their interactions with the Enrichment Model analyzed for complementarity with the Candidate Modulator features.
8. The analysis allowed for the creation of a Pharmacophore Model with excluded volumes representing the binding site protein atoms.
9. A further filtering of the Diverse Subset hits employing the pharmacophore query provided final Candidate Modulators.
A set of analogs was selected from those hits showing an excellent match with the pharmacophore query. The analogs were identified by searching the previously prepared ZINC database. Representative examples of small molecule hits for Enrichment Model 2 are in FIG. 2b and those from Enrichment Model 3 are in FIG. 3b.

TABLE 2
Enrichment Model 2 Coordinates

| | | | | | | | | |
|---|---|---|---|---|---|---|---|---|
| ATOM | 1 | N | GLY | 437 | 12.305 | 46.964 | −1.369 | N |
| ATOM | 2 | CA | GLY | 437 | 12.82 | 47.783 | −0.298 | C |
| ATOM | 3 | C | GLY | 437 | 11.802 | 48.528 | 0.546 | C |
| ATOM | 4 | O | GLY | 437 | 12.019 | 48.83 | 1.723 | O |
| ATOM | 5 | N | LEU | 440 | 10.665 | 46.636 | 3.25 | N |
| ATOM | 6 | CA | LEU | 440 | 11.71 | 46.404 | 4.235 | C |
| ATOM | 7 | C | LEU | 440 | 11.831 | 47.618 | 5.135 | C |
| ATOM | 8 | O | LEU | 440 | 11.907 | 47.47 | 6.357 | O |
| ATOM | 9 | CB | LEU | 440 | 13.072 | 46.127 | 3.603 | C |
| ATOM | 10 | CG | LEU | 440 | 13.19 | 44.718 | 3.015 | C |
| ATOM | 11 | CD1 | LEU | 440 | 14.456 | 44.648 | 2.172 | C |
| ATOM | 12 | CD2 | LEU | 440 | 13.236 | 43.652 | 4.107 | C |
| ATOM | 13 | N | ASP | 441 | 11.927 | 48.842 | 4.511 | N |
| ATOM | 14 | CA | ASP | 441 | 12.149 | 50.067 | 5.285 | C |
| ATOM | 15 | C | ASP | 441 | 10.917 | 50.324 | 6.163 | C |
| ATOM | 16 | O | ASP | 441 | 10.985 | 50.915 | 7.24 | O |
| ATOM | 17 | CB | ASP | 441 | 12.371 | 51.278 | 4.395 | C |
| ATOM | 18 | CG | ASP | 441 | 13.755 | 51.311 | 3.766 | C |
| ATOM | 19 | OD1 | ASP | 441 | 14.538 | 50.371 | 4.039 | O |
| ATOM | 20 | OD2 | ASP | 441 | 13.908 | 52.288 | 2.958 | O1− |
| ATOM | 21 | N | GLU | 444 | 11.219 | 47.706 | 8.829 | N |
| ATOM | 22 | CA | GLU | 444 | 12.255 | 48.189 | 9.737 | C |
| ATOM | 23 | C | GLU | 444 | 11.641 | 49.301 | 10.611 | C |
| ATOM | 24 | O | GLU | 444 | 11.7 | 49.228 | 11.842 | O |
| ATOM | 25 | CB | GLU | 444 | 13.488 | 48.618 | 8.937 | C |
| ATOM | 26 | CG | GLU | 444 | 14.738 | 48.861 | 9.765 | C |
| ATOM | 27 | CD | GLU | 444 | 15.309 | 47.7 | 10.563 | C |
| ATOM | 28 | OE1 | GLU | 444 | 14.719 | 46.575 | 10.505 | O |
| ATOM | 29 | OE2 | GLU | 444 | 16.289 | 47.987 | 11.318 | O1− |
| ATOM | 30 | N | GLU | 445 | 10.951 | 50.31 | 9.964 | N |
| ATOM | 31 | CA | GLU | 445 | 10.189 | 51.312 | 10.734 | C |
| ATOM | 32 | C | GLU | 445 | 9.204 | 50.643 | 11.743 | C |
| ATOM | 33 | O | GLU | 445 | 9.165 | 50.953 | 12.939 | O |
| ATOM | 34 | CB | GLU | 445 | 9.49 | 52.272 | 9.749 | C |
| ATOM | 35 | CG | GLU | 445 | 8.173 | 52.882 | 10.199 | C |
| ATOM | 36 | CD | GLU | 445 | 8.211 | 53.663 | 11.497 | C |
| ATOM | 37 | OE1 | GLU | 445 | 9.259 | 54.314 | 11.742 | O |
| ATOM | 38 | OE2 | GLU | 445 | 7.144 | 53.571 | 12.179 | O1− |
| ATOM | 39 | N | HIS | 448 | 11.067 | 48.917 | 14.352 | N |
| ATOM | 40 | CA | HIS | 448 | 11.73 | 49.868 | 15.25 | C |
| ATOM | 41 | C | HIS | 448 | 10.709 | 50.536 | 16.193 | C |
| ATOM | 42 | O | HIS | 448 | 11.037 | 50.934 | 17.312 | O |
| ATOM | 43 | CB | HIS | 448 | 12.473 | 50.989 | 14.52 | C |
| ATOM | 44 | CG | HIS | 448 | 13.835 | 50.625 | 14.029 | C |
| ATOM | 45 | ND1 | HIS | 448 | 14.827 | 51.58 | 14 | N |
| ATOM | 46 | CD2 | HIS | 448 | 14.301 | 49.441 | 13.508 | C |
| ATOM | 47 | CE1 | HIS | 448 | 15.855 | 50.999 | 13.396 | C |
| ATOM | 48 | NE2 | HIS | 448 | 15.558 | 49.713 | 13.041 | N |
| ATOM | 49 | N | HIS | 524 | 18.381 | 42.143 | −0.292 | N |
| ATOM | 50 | CA | HIS | 524 | 18.368 | 43.619 | −0.263 | C |
| ATOM | 51 | C | HIS | 524 | 18.802 | 44.14 | 1.126 | C |
| ATOM | 52 | O | HIS | 524 | 19.508 | 45.139 | 1.271 | O |
| ATOM | 53 | CB | HIS | 524 | 16.948 | 44.124 | −0.523 | C |
| ATOM | 54 | CG | HIS | 524 | 16.78 | 45.249 | −1.479 | C |
| ATOM | 55 | ND1 | HIS | 524 | 16.361 | 46.48 | −1.029 | N |
| ATOM | 56 | CD2 | HIS | 524 | 16.755 | 45.204 | −2.86 | C |
| ATOM | 57 | CE1 | HIS | 524 | 15.951 | 47.101 | −2.11 | C |
| ATOM | 58 | NE2 | HIS | 524 | 16.11 | 46.346 | −3.235 | N |
| ATOM | 59 | N | TYR | 525 | 18.278 | 43.443 | 2.196 | N |
| ATOM | 60 | CA | TYR | 525 | 18.501 | 43.849 | 3.58 | C |
| ATOM | 61 | C | TYR | 525 | 19.964 | 43.708 | 4.023 | C |
| ATOM | 62 | O | TYR | 525 | 20.403 | 44.253 | 5.039 | O |
| ATOM | 63 | CB | TYR | 525 | 17.593 | 43.032 | 4.495 | C |
| ATOM | 64 | CG | TYR | 525 | 17.442 | 43.611 | 5.878 | C |
| ATOM | 65 | CD1 | TYR | 525 | 16.744 | 44.808 | 6.085 | C |
| ATOM | 66 | CD2 | TYR | 525 | 17.951 | 42.911 | 6.976 | C |
| ATOM | 67 | CE1 | TYR | 525 | 16.543 | 45.292 | 7.373 | C |
| ATOM | 68 | CE2 | TYR | 525 | 17.759 | 43.394 | 8.266 | C |
| ATOM | 69 | CZ | TYR | 525 | 17.065 | 44.58 | 8.448 | C |
| ATOM | 70 | OH | TYR | 525 | 16.854 | 45.073 | 9.694 | O |
| ATOM | 71 | N | GLU | 527 | 22.357 | 44.934 | 2.292 | N |
| ATOM | 72 | CA | GLU | 527 | 23.078 | 46.158 | 2.035 | C |
| ATOM | 73 | C | GLU | 527 | 22.922 | 47.116 | 3.238 | C |
| ATOM | 74 | O | GLU | 527 | 23.374 | 47.962 | 3.521 | O |
| ATOM | 75 | CB | GLU | 527 | 22.609 | 46.897 | 0.802 | C |
| ATOM | 76 | CG | GLU | 527 | 22.631 | 46.068 | −0.499 | C |
| ATOM | 77 | CD | GLU | 527 | 22.344 | 46.979 | −1.592 | C |
| ATOM | 78 | OE1 | GLU | 527 | 23.075 | 48.128 | −1.328 | O |
| ATOM | 79 | OE2 | GLU | 527 | 21.633 | 46.567 | −2.5 | O1− |
| ATOM | 80 | N | THR | 528 | 21.722 | 47 | 3.906 | N |
| ATOM | 81 | CA | THR | 528 | 21.435 | 47.793 | 5.095 | C |
| ATOM | 82 | C | THR | 528 | 22.376 | 47.303 | 6.206 | C |
| ATOM | 83 | O | THR | 528 | 23.012 | 48.095 | 6.899 | O |
| ATOM | 84 | CB | THR | 528 | 19.951 | 47.7 | 5.463 | C |
| ATOM | 85 | OG1 | THR | 528 | 19.183 | 48.363 | 4.447 | O |
| ATOM | 86 | CG2 | THR | 528 | 19.63 | 48.3 | 6.823 | C |
| ATOM | 87 | N | ARG | 531 | 25.26 | 49.003 | 5.594 | N |
| ATOM | 88 | CA | ARE | 531 | 24.67 | 50.328 | 5.298 | C |
| ATOM | 89 | C | ARG | 531 | 25.07 | 51.054 | 3.996 | C |
| ATOM | 90 | O | ARG | 531 | 25.926 | 51.935 | 4.027 | O |
| ATOM | 91 | CB | ARG | 531 | 24.56 | 51.227 | 6.554 | C |
| ATOM | 92 | CG | ARG | 531 | 25.69 | 51.249 | 7.609 | C |
| ATOM | 93 | CD | ARG | 531 | 26.834 | 52.151 | 7.17 | C |
| ATOM | 94 | NE | ARG | 531 | 27.636 | 51.371 | 6.243 | N |
| ATOM | 95 | CZ | ARG | 531 | 28.831 | 51.695 | 5.783 | C |
| ATOM | 96 | NH1 | ARG | 531 | 29.472 | 52.771 | 6.168 | N |
| ATOM | 97 | NH2 | ARG | 531 | 29.405 | 50.911 | 4.907 | N1+ |
| ATOM | 98 | N | ARG | 532 | 24.371 | 50.78 | 2.878 | N |
| ATOM | 99 | CA | ARG | 532 | 24.481 | 51.584 | 1.633 | C |
| ATOM | 100 | C | ARG | 532 | 24.081 | 53.062 | 1.83 | C |
| ATOM | 101 | O | ARG | 532 | 24.673 | 53.939 | 1.211 | O |
| ATOM | 102 | CB | ARG | 532 | 23.663 | 50.944 | 0.491 | C |
| ATOM | 103 | CG | ARG | 532 | 24.037 | 51.505 | −0.907 | C |
| ATOM | 104 | CD | ARG | 532 | 22.786 | 51.76 | −1.74 | C |
| ATOM | 105 | NE | ARG | 532 | 22.247 | 50.468 | −2.154 | N |
| ATOM | 106 | CZ | ARG | 532 | 21.051 | 50.295 | −2.702 | C |
| ATOM | 107 | NH1 | ARG | 532 | 20.291 | 51.325 | −2.998 | N |
| ATOM | 108 | NH2 | ARG | 532 | 20.565 | 49.098 | −2.97 | N1+ |
| ATOM | 109 | N | ILE | 533 | 23.091 | 53.336 | 2.694 | N |
| ATOM | 110 | CA | ILE | 533 | 22.67 | 54.676 | 3.17 | C |
| ATOM | 111 | C | ILE | 533 | 22.1 | 55.649 | 2.101 | C |
| ATOM | 112 | O | ILE | 533 | 21.89 | 56.83 | 2.374 | O |
| ATOM | 113 | CB | ILE | 533 | 23.755 | 55.25 | 4.132 | C |
| ATOM | 114 | CG1 | ILE | 533 | 23.191 | 55.571 | 5.534 | C |
| ATOM | 115 | CG2 | ILE | 533 | 24.638 | 56.38 | 3.572 | C |
| ATOM | 116 | CD1 | ILE | 533 | 22.158 | 56.705 | 5.591 | C |
| ATOM | 117 | N | GLU | 534 | 21.803 | 55.15 | 0.892 | N |
| ATOM | 118 | CA | GLU | 534 | 21.429 | 55.969 | −0.277 | C |
| ATOM | 119 | C | GLU | 534 | 19.921 | 55.973 | −0.622 | C |
| ATOM | 120 | O | GLU | 534 | 19.464 | 56.872 | −1.326 | O |
| ATOM | 121 | CB | GLU | 534 | 22.371 | 55.588 | −1.442 | C |
| ATOM | 122 | CG | GLU | 534 | 21.978 | 56.052 | −2.859 | C |
| ATOM | 123 | CD | GLU | 534 | 20.946 | 55.149 | −3.556 | C |
| ATOM | 124 | OE1 | GLU | 534 | 20.868 | 53.939 | −3.227 | O |
| ATOM | 125 | OE2 | GLU | 534 | 20.212 | 55.649 | −4.447 | O1− |
| ATOM | 126 | N | GLU | 535 | 19.109 | 55.046 | −0.09 | N |
| ATOM | 127 | CA | GLU | 535 | 17.676 | 54.857 | −0.425 | C |
| ATOM | 128 | C | GLU | 535 | 16.725 | 55.967 | 0.12 | C |
| ATOM | 129 | O | GLU | 535 | 15.538 | 55.747 | 0.382 | O |
| ATOM | 130 | CB | GLU | 535 | 17.25 | 53.43 | −0.014 | C |
| ATOM | 131 | CG | GLU | 535 | 16.215 | 52.814 | −0.972 | C |
| ATOM | 132 | CD | GLU | 535 | 16.813 | 52.508 | −2.354 | C |
| ATOM | 133 | OE1 | GLU | 535 | 17.75 | 51.686 | −2.461 | O |
| ATOM | 134 | OE2 | GLU | 535 | 16.37 | 53.125 | −3.357 | O1− |
| ATOM | 135 | N | GLU | 536 | 17.273 | 57.177 | 0.274 | N |
| ATOM | 136 | CA | GLU | 536 | 16.64 | 58.44 | 0.678 | C |
| ATOM | 137 | C | GLU | 536 | 16.489 | 59.421 | −0.504 | C |
| ATOM | 138 | O | GLU | 536 | 15.588 | 60.259 | −0.492 | O |
| ATOM | 139 | CB | GLU | 536 | 17.492 | 59.093 | 1.786 | C |
| ATOM | 140 | CG | GLU | 536 | 17.772 | 58.19 | 3.002 | C |
| ATOM | 141 | CD | GLU | 536 | 16.49 | 57.627 | 3.622 | C |
| ATOM | 142 | OE1 | GLU | 536 | 16.355 | 56.38 | 3.667 | O |
| ATOM | 143 | OE2 | GLU | 536 | 15.626 | 58.438 | 4.016 | O1− |
| ATOM | 144 | N | LYS | 540 | 12.221 | 55.475 | −1.332 | N |
| ATOM | 145 | CA | LYS | 540 | 11.837 | 55.677 | 0.073 | C |
| ATOM | 146 | C | LYS | 540 | 10.406 | 55.248 | 0.28 | C |
| ATOM | 147 | O | LYS | 540 | 9.851 | 55.344 | 1.344 | O |
| ATOM | 148 | CB | LYS | 540 | 12.016 | 57.175 | 0.341 | C |
| ATOM | 149 | CG | LYS | 540 | 11.951 | 57.529 | 1.849 | C |
| ATOM | 150 | CD | LYS | 540 | 13.13 | 57.046 | 2.713 | C |
| ATOM | 151 | CE | LYS | 540 | 13.125 | 55.57 | 3.131 | C |
| ATOM | 152 | NZ | LYS | 540 | 14.374 | 54.909 | 2.725 | N1+ |
| TER | 153 | LYS | 540 | | | | | |
| END | | | | | | | | |

TABLE 3
Enrichment Model 3 Coordinates

| | | | | | | | | |
|---|---|---|---|---|---|---|---|---|
| ATOM | 1 | N | PRO | 312 | 5.782 | 31.906 | 15.968 | N |
| ATOM | 2 | CA | PRO | 312 | 6.856 | 31.304 | 15.192 | C |
| ATOM | 3 | C | PRO | 312 | 8.064 | 30.797 | 16.011 | C |
| ATOM | 4 | O | PRO | 312 | 8.128 | 29.658 | 16.476 | O |
| ATOM | 5 | CB | PRO | 312 | 7.185 | 32.391 | 14.173 | C |
| ATOM | 6 | CG | PRO | 312 | 6.82 | 33.707 | 14.859 | C |
| ATOM | 7 | CD | PRO | 312 | 5.973 | 33.341 | 16.07 | C |
| ATOM | 8 | N | GLU | 313 | 9.068 | 31.718 | 16.207 | N |
| ATOM | 9 | CA | GLU | 313 | 10.403 | 31.355 | 16.655 | C |
| ATOM | 10 | C | GLU | 313 | 10.546 | 31.502 | 18.169 | C |
| ATOM | 11 | O | GLU | 313 | 9.701 | 32.015 | 18.902 | O |
| ATOM | 12 | CB | GLU | 313 | 11.462 | 32.151 | 15.886 | C |
| ATOM | 13 | CG | GLU | 313 | 11.492 | 33.641 | 16.24 | C |
| ATOM | 14 | CD | GLU | 313 | 12.403 | 34.301 | 15.213 | C |
| ATOM | 15 | OE1 | GLU | 313 | 13.503 | 34.734 | 15.659 | O |
| ATOM | 16 | OE2 | GLU | 313 | 11.94 | 34.301 | 14.037 | O1− |
| ATOM | 17 | N | LYS | 324 | 9.21 | 35.132 | 21.411 | N |
| ATOM | 18 | CA | LYS | 324 | 9.21 | 36.59 | 21.35 | C |
| ATOM | 19 | C | LYS | 324 | 8.068 | 37.19 | 20.533 | C |
| ATOM | 20 | O | LYS | 324 | 6.93 | 37.265 | 21.004 | O |
| ATOM | 21 | CB | LYS | 324 | 10.614 | 37.13 | 21.058 | C |
| ATOM | 22 | CG | LYS | 324 | 11.458 | 37.191 | 22.337 | C |
| ATOM | 23 | CD | LYS | 324 | 11.074 | 38.37 | 23.242 | C |
| ATOM | 24 | CE | LYS | 324 | 10.826 | 37.963 | 24.686 | C |
| ATOM | 25 | NZ | LYS | 324 | 9.494 | 37.313 | 24.807 | N1+ |
| ATOM | 26 | N | LYS | 325 | 8.379 | 37.609 | 19.262 | N |
| ATOM | 27 | CA | LYS | 325 | 7.416 | 38.339 | 18.449 | C |
| ATOM | 28 | C | LYS | 325 | 6.393 | 37.317 | 17.948 | C |
| ATOM | 29 | O | LYS | 325 | 6.704 | 36.174 | 17.617 | O |
| ATOM | 30 | CB | LYS | 325 | 8.127 | 39.041 | 17.293 | C |
| ATOM | 31 | CG | LYS | 325 | 7.242 | 40.063 | 16.574 | C |
| ATOM | 32 | CD | LYS | 325 | 7.87 | 40.505 | 15.258 | C |
| ATOM | 33 | CE | LYS | 325 | 8.973 | 41.539 | 15.388 | C |
| ATOM | 34 | NZ | LYS | 325 | 9.655 | 41.646 | 14.098 | N1+ |
| ATOM | 35 | N | SER | 326 | 5.101 | 37.777 | 17.927 | N |
| ATOM | 36 | CA | SER | 326 | 3.974 | 36.922 | 17.559 | C |
| ATOM | 37 | C | SER | 326 | 3.151 | 37.723 | 16.558 | C |
| ATOM | 38 | O | SER | 326 | 3.083 | 38.957 | 16.603 | O |
| ATOM | 39 | CB | SER | 326 | 3.167 | 36.529 | 18.792 | C |
| ATOM | 40 | OG | SER | 326 | 3.112 | 37.596 | 19.745 | O |
| ATOM | 41 | N | TYR | 327 | 2.492 | 36.96 | 15.611 | N |
| ATOM | 42 | CA | TYR | 327 | 1.756 | 37.626 | 14.547 | C |
| ATOM | 43 | C | TYR | 327 | 0.316 | 37.159 | 14.632 | C |
| ATOM | 44 | O | TYR | 327 | −0.001 | 35.99 | 14.859 | O |
| ATOM | 45 | CB | TYR | 327 | 2.287 | 37.385 | 13.132 | C |
| ATOM | 46 | CG | TYR | 327 | 3.75 | 37.709 | 12.984 | C |
| ATOM | 47 | CD1 | TYR | 327 | 4.704 | 36.728 | 13.272 | C |
| ATOM | 48 | CD2 | TYR | 327 | 4.177 | 38.984 | 12.591 | C |
| ATOM | 49 | CE1 | TYR | 327 | 6.058 | 37.018 | 13.174 | C |
| ATOM | 50 | CE2 | 'TYR | 327 | 5.54 | 39.267 | 12.474 | C |
| ATOM | 51 | CZ | TYR | 327 | 6.468 | 38.28 | 12.773 | C |
| ATOM | 52 | OH | TYR | 327 | 7.814 | 38.477 | 12.705 | O |
| ATOM | 53 | N | HIS | 447 | 9.256 | 47.227 | 12.924 | N |
| ATOM | 54 | CA | HIS | 447 | 9.983 | 46.533 | 13.973 | C |
| ATOM | 55 | C | HIS | 447 | 10.586 | 47.521 | 14.993 | C |
| ATOM | 56 | O | HIS | 447 | 10.53 | 47.279 | 16.204 | O |
| ATOM | 57 | CB | HIS | 447 | 11.074 | 45.616 | 13.416 | C |
| ATOM | 58 | CG | HIS | 447 | 11.723 | 44.87 | 14.523 | C |
| ATOM | 59 | ND1 | HIS | 447 | 11.125 | 43.762 | 15.08 | N |
| ATOM | 60 | CD2 | HIS | 447 | 12.855 | 45.223 | 15.225 | C |
| ATOM | 61 | CE1 | HIS | 447 | 11.894 | 43.451 | 16.105 | C |
| ATOM | 62 | NE2 | HIS | 447 | 12.908 | 44.35 | 16.273 | N |
| ATOM | 63 | N | GLU | 451 | 9.695 | 47.001 | 18.724 | N |
| ATOM | 64 | CA | GLU | 451 | 10.681 | 46.682 | 19.767 | C |
| ATOM | 65 | C | GLU | 451 | 10.668 | 47.716 | 20.92 | C |
| ATOM | 66 | O | GLU | 451 | 11.275 | 47.523 | 21.973 | O |
| ATOM | 67 | CB | GLU | 451 | 12.085 | 46.54 | 19.154 | C |
| ATOM | 68 | CG | GLU | 451 | 13.095 | 45.855 | 20.081 | C |
| ATOM | 69 | CD | GLU | 451 | 14.379 | 45.353 | 19.41 | C |
| ATOM | 70 | OE1 | GLU | 451 | 14.244 | 44.828 | 18.264 | O |
| ATOM | 71 | OE2 | GLU | 451 | 15.434 | 45.465 | 20.095 | O1− |
| ATOM | 72 | N | ASP | 481 | 14.371 | 38.892 | 11.338 | N |
| ATOM | 73 | CA | ASP | 481 | 14.639 | 39.061 | 12.762 | C |
| ATOM | 74 | C | ASP | 481 | 15.673 | 38.053 | 13.274 | C |
| ATOM | 75 | O | ASP | 481 | 16.416 | 38.322 | 14.222 | O |
| ATOM | 76 | CB | ASP | 481 | 13.392 | 39.023 | 13.616 | C |
| ATOM | 77 | CG | ASP | 481 | 12.362 | 40.393 | 13.768 | C |
| ATOM | 78 | OD1 | ASP | 481 | 13.486 | 41.433 | 13.667 | O |
| ATOM | 79 | OD2 | ASP | 481 | 11.517 | 40.376 | 14.023 | O1 |
| ATOM | 80 | N | ARG | 484 | 18.594 | 39.855 | 12.32 | N |
| ATOM | 81 | CA | ARG | 484 | 18.782 | 41.002 | 13.209 | C |
| ATOM | 82 | C | ARG | 484 | 19.45 | 40.552 | 14.53 | C |
| ATOM | 83 | O | ARG | 484 | 20.201 | 41.304 | 15.159 | O |
| ATOM | 84 | CB | ARG | 484 | 17.419 | 41.626 | 13.532 | C |
| ATOM | 85 | CG | ARG | 484 | 17.486 | 42.98 | 14.246 | C |
| ATOM | 86 | CD | ARG | 484 | 16.154 | 43.329 | 14.9 | C |
| ATOM | 87 | NE | ARG | 484 | 15.023 | 43.123 | 14.006 | N |
| ATOM | 88 | CZ | ARG | 484 | 14.706 | 43.811 | 12.894 | C |
| ATOM | 89 | NH1 | ARG | 484 | 15.383 | 44.889 | 12.483 | N |
| ATOM | 90 | NH2 | ARG | 484 | 13.672 | 43.36 | 12.165 | N1+ |
| ATOM | 91 | N | GLU | 485 | 18.962 | 39.367 | 15.053 | N |
| ATOM | 92 | CA | GLU | 485 | 19.342 | 38.872 | 16.382 | C |
| ATOM | 93 | C | GLU | 485 | 20.697 | 38.142 | 16.314 | C |
| ATOM | 94 | O | GLU | 485 | 21.699 | 38.516 | 16.926 | O |
| ATOM | 95 | CB | GLU | 485 | 18.199 | 38.007 | 16.944 | C |
| ATOM | 96 | CG | GLU | 485 | 18.141 | 37.962 | 18.469 | C |
| ATOM | 97 | CD | GLU | 485 | 18.554 | 36.613 | 19.013 | C |
| ATOM | 98 | OE1 | GLU | 485 | 17.652 | 35.835 | 19.439 | O |
| ATOM | 99 | OE2 | GLU | 485 | 19.817 | 36.405 | 19.047 | O1− |
| ATOM | 100 | N | LYS | 538 | 26.979 | 44.241 | 16.156 | N |
| ATOM | 101 | CA | LYS | 538 | 26.332 | 44.35 | 17.447 | C |
| ATOM | 102 | C | LYS | 538 | 26.082 | 42.957 | 18.078 | C |
| ATOM | 103 | O | LYS | 538 | 26.178 | 42.813 | 19.301 | O |
| ATOM | 104 | CB | LYS | 538 | 25.104 | 45.272 | 17.464 | C |
| ATOM | 105 | CG | LYS | 538 | 23.97 | 44.971 | 16.469 | C |
| ATOM | 106 | CD | LYS | 538 | 22.883 | 46.065 | 16.559 | C |
| ATOM | 107 | CE | LYS | 538 | 21.631 | 45.811 | 15.72 | C |
| ATOM | 108 | NZ | LYS | 538 | 21.81 | 46.229 | 14.322 | N1+ |
| ATOM | 109 | N | SER | 539 | 25.797 | 41.909 | 17.22 | N |
| ATOM | 110 | CA | SER | 539 | 25.588 | 40.549 | 17.731 | C |
| ATOM | 111 | C | SER | 539 | 26.882 | 39.887 | 18.264 | C |
| ATOM | 112 | O | SER | 539 | 26.834 | 38.835 | 18.909 | O |
| ATOM | 113 | CB | SER | 539 | 24.952 | 39.61 | 16.695 | C |
| ATOM | 114 | OG | SER | 539 | 23.658 | 40.062 | 16.318 | O |
| ATOM | 115 | N | LYS | 542 | 27.932 | 43.382 | 21.381 | N |
| ATOM | 116 | CA | LYS | 542 | 27.046 | 43.46 | 22.544 | C |
| ATOM | 117 | C | LYS | 542 | 25.581 | 43.233 | 22.108 | C |
| ATOM | 118 | O | LYS | 542 | 24.876 | 44.16 | 21.688 | O |
| ATOM | 119 | CB | LYS | 542 | 27.116 | 44.834 | 23.248 | C |
| ATOM | 120 | CG | LYS | 542 | 26.323 | 44.824 | 24.563 | C |
| ATOM | 121 | CD | LYS | 542 | 25.704 | 46.171 | 24.938 | C |
| ATOM | 122 | CE | LYS | 542 | 24.765 | 46.8 | 23.913 | C |
| ATOM | 123 | NZ | LYS | 542 | 23.835 | 45.826 | 23.329 | N1+ |
| ATOM | 124 | N | GLY | 543 | 25.137 | 41.935 | 22.253 | N |
| ATOM | 125 | CA | GLY | 543 | 23.796 | 41.537 | 21.865 | C |
| ATOM | 126 | C | GLY | 543 | 23.374 | 40.282 | 22.623 | C |
| ATOM | 127 | O | GLY | 543 | 24.056 | 39.793 | 23.525 | O |
| ATOM | 128 | N | HIS | 544 | 22.173 | 39.757 | 22.17 | N |
| ATOM | 129 | CA | HIS | 544 | 21.564 | 38.545 | 22.715 | C |
| ATOM | 130 | C | HIS | 544 | 21.034 | 38.771 | 24.149 | C |
| ATOM | 131 | O | HIS | 544 | 20.577 | 37.829 | 24.804 | O |
| ATOM | 132 | CB | HIS | 544 | 22.468 | 37.284 | 22.716 | C |
| ATOM | 133 | CG | HIS | 544 | 23.303 | 37.06 | 21.493 | C |
| ATOM | 134 | ND1 | HIS | 544 | 22.941 | 36.169 | 20.495 | N |
| ATOM | 135 | CD2 | HIS | 544 | 24.537 | 37.611 | 21.207 | C |
| ATOM | 136 | CE1 | HIS | 544 | 23.88 | 36.307 | 19.577 | C |
| ATOM | 137 | NE2 | HIS | 544 | 24.86 | 37.179 | 19.957 | N |
| ATOM | 138 | N | GLU | 545 | 21.153 | 40.052 | 24.663 | N |
| ATOM | 139 | CA | GLU | 545 | 21.136 | 40.326 | 26.093 | C |
| ATOM | 140 | C | GLU | 545 | 19.84 | 40.947 | 26.624 | C |
| ATOM | 141 | O | GLU | 545 | 19.434 | 40.668 | 27.754 | O |
| ATOM | 142 | CB | GLU | 545 | 22.373 | 41.144 | 26.512 | C |
| ATOM | 143 | CG | GLU | 545 | 22.358 | 42.655 | 26.271 | C |
| ATOM | 144 | CD | GLU | 545 | 22.185 | 43.178 | 24.851 | C |
| ATOM | 145 | OE1 | GLU | 545 | 21.432 | 42.509 | 24.086 | O |
| ATOM | 146 | OE2 | GLU | 545 | 22.801 | 44.266 | 24.603 | O1− |
| ATOM | 147 | N | TYR | 546 | 19.248 | 41.926 | 25.847 | N |
| ATOM | 148 | CA | TYR | 546 | 18.027 | 42.622 | 26.29 | C |
| ATOM | 149 | C | TYR | 546 | 16.746 | 41.983 | 25.719 | C |
| ATOM | 150 | O | TYR | 546 | 15.666 | 42.572 | 25.666 | O |
| ATOM | 151 | CB | TYR | 546 | 18.089 | 44.152 | 26.135 | C |
| ATOM | 152 | CG | TYR | 546 | 17.94 | 44.686 | 24.733 | C |
| ATOM | 153 | CD1 | TYR | 546 | 16.672 | 45.021 | 24.237 | C |
| ATOM | 154 | CD2 | TYR | 546 | 19.055 | 44.857 | 23.905 | C |
| ATOM | 155 | CE1 | TYR | 546 | 16.521 | 45.454 | 22.924 | C |
| ATOM | 156 | CE2 | TYR | 546 | 18.908 | 45.294 | 22.59 | C |
| ATOM | 157 | CZ | TYR | 546 | 17.639 | 45.566 | 22.104 | C |
| ATOM | 158 | OH | TYR | 546 | 17.522 | 45.936 | 20.795 | O |
| ATOM | 159 | N | THR | 547 | 16.874 | 40.641 | 25.428 | N |
| ATOM | 160 | CA | THR | 547 | 15.785 | 39.828 | 24.867 | C |
| ATOM | 161 | C | THR | 547 | 15.898 | 38.373 | 25.37 | C |
| ATOM | 162 | O | THR | 547 | 16.131 | 37.44 | 24.534 | O |
| ATOM | 163 | CB | THR | 547 | 15.653 | 40.152 | 23.365 | C |
| ATOM | 164 | OG1 | THR | 547 | 14.296 | 39.983 | 22.953 | O |
| ATOM | 165 | CG2 | THR | 547 | 16.583 | 39.395 | 22.426 | C |
| ATOM | 166 | OXT | THR | 547 | 15.78 | 38.207 | 26.622 | O1− |
| TER | 167 | THR | 547 | | | | | |
| END | | | | | | | | |

### EXAMPLE 3

### Enrichment Model 4 Collection and Their Use

General Description of Enrichment Model 4 Collection

This method describes the use of a process to identify SHP2 modulators by utilization of the movement of the WPD-loop and the connecting αF helix (residues 437-451). Multiple conformations of the WPD are expected to provide Enrichment Models, which change in electrostatic and steric properties as the WPD-loop changes its orientation. The process employed provides multiple Enrichment Models which are hereto collected and described as the Enrichment Model Collection 4. Collectively or singularly the use of these models will identify Candidate Modulators of SHP2. The SHP2 structure (PDB access code: 4DGP) was employed for the construction of the Enrichment Model 4 Collection.

General Method Description: The Construction of Enrichment Model 4 Collection

To construct the Enrichment Model 4 Collection different conformations of the WPD-loop and the αF helix were generated by Conformational Search. In order to provided the SHP2 structures for construction of the Enrichment Model 4 Collection two approaches were used to select residues for the conformational search. In the first case residues within 4.5 Å sphere from Leu440 in the αF-helix were selected and in the second case WPD-loop residues Phe424 to Gly433 were selected.

Enrichment Model 4 Example 1 contains residues: Y³²⁷V³⁵⁴D³⁹⁵F⁴²⁴T⁴²⁶W⁴²⁷P⁴³³D⁴³⁵P⁴³⁶G⁴³⁷G⁴³⁸V⁴³⁹L⁴⁴⁰D⁴⁴¹F⁴⁴²L⁴⁴³E⁴⁴⁴ V⁴⁴⁶ V⁴⁵⁹ V⁴⁶¹ F⁴⁷³ I⁴⁷⁴ I⁴⁷⁶ D⁴⁷⁷ I⁴⁸⁰ F⁵¹⁷ A⁵²¹ V⁵²² H⁵²⁴ Y⁵²⁵ T⁵²⁸ R⁵³².

Enrichment Model 4 Example 2 contains residues: H³⁹⁴ D³⁹⁵ F⁴²⁴ T⁴²⁶ W⁴²⁷ P⁴²⁸ V⁴³² P⁴³³ S⁴³⁴ D⁴³⁵ P⁴³⁶ G⁴³⁷ G⁴³⁸ V⁴³⁹ R⁴⁶⁹ T⁴⁷² F⁴⁷³ Q⁵¹⁴ F⁵¹⁷.

Construction of the Enrichment Model 4 Collection
1. Enrichment Model 4 Example 1 contains selected residues within 4.5 Å sphere from L440 in the αF-helix.
2. For Enrichment Model 4 example 2 the WPD loop residues Phe424 to Gly433 were selected.
3. Conformational Search to generate the Enrichment Model 4 collection employed Force field calculations disregarding atoms distant from center of the Enrichment Model 4.
4. Molecular Dynamic calculations were accelerated by fixing the coordinates of atoms hear the active zone used for conformational search.
5. Enrichment Model coordinates were saved in a data base and checked for the ability of SHP2 Modulators to bind using Binding Site Identification tools.
6. Sites were checked for size and polarity giving preference to more hydrophobic rather than hydrophilic sites.
7. Enrichment Models with at least two aromatic hydrophobic residues and several polar side chains were selected. Enrichment Model 4 example 1 contains seven aromatic hydrophobic residues: Phe 424, Phe 442, Phe 473, Phe 517, Tyr 525 and the WPD-loop's Trp 427. This model corresponds to a super-open conformation of the WPD-loop where Trp 427 is out of its binding pocket (Fig. 4a). The 3-dimensional coordinates for this model are in Table 4. Enrichment Model 4 example 2 is located along the αF-helix and shares with example 1 Phe 424, Phe 473, Phe 517 and Trp 427, but those residues are in differed rotamer conformations. For example Trp 427 is occupying its own pocket (Fig. 5a). 3D coordinates for this model are in Table 5.

Utilization of the Enrichment Model 4 Collection

A conformational database of small molecules was prepared as described in the Enrichment Model 1
1. Molecular Docking of the Diverse Subset with Enrichment Model 4 Collection identified Candidate Modulators.
2. Candidate Modulators were energy minimized and their interactions with the Enrichment Model analyzed for complementarity with the Candidate Modulator features.
3. The analysis allowed for the creation of a Pharmacophore Model with excluded volumes representing the binding site protein atoms.
4. A further filtering of the Diverse Subset hits employing the pharmacophore query provided final Candidate Modulators.
5. A set of analogs was selected from those hits showing an excellent match with the pharmacophore query. The analogs were identified by searching the previously prepared ZINC database. Representative small molecule hits for Enrichment Model 4 example 1 are in FIG. 4b, and for example 2, in FIG. 5b.
6. A set of analogs was selected from those hits showing an excellent match with the pharmacophore query. The analogs were identified by searching the previously prepared ZINC database.

TABLE 4
Enrichment Model 4—Example 1 Coordinates

| | | | | | | | | |
|---|---|---|---|---|---|---|---|---|
| ATOM | 1 | N | TYR | 327 | 10.782 | 8.085 | 60.247 | N |
| ATOM | 2 | CA | TYR | 327 | 11.958 | 8.495 | 61.005 | C |
| ATOM | 3 | C | TYR | 327 | 12.344 | 9.9 | 60.569 | C |
| ATOM | 4 | O | TYR | 327 | 12.306 | 10.265 | 59.391 | O |
| ATOM | 5 | CB | TYR | 327 | 13.155 | 7.551 | 60.845 | C |
| ATOM | 6 | CG | TYR | 327 | 12.789 | 6.116 | 61.128 | C |
| ATOM | 7 | CD1 | TYR | 327 | 12.239 | 5.33 | 60.109 | C |
| ATOM | 8 | CD2 | TYR | 327 | 12.915 | 5.575 | 62.412 | C |
| ATOM | 9 | CE1 | TYR | 327 | 11.791 | 4.044 | 60.375 | C |
| ATOM | 10 | CE2 | TYR | 327 | 12.488 | 4.272 | 62.671 | C |
| ATOM | 11 | CZ | TYR | 327 | 11.914 | 3.524 | 61.651 | C |
| ATOM | 12 | OH | TYR | 327 | 11.423 | 2.268 | 61.846 | O |
| ATOM | 13 | N | VAL | 354 | 15.798 | 9.521 | 69.864 | N |
| ATOM | 14 | CA | VAL | 354 | 16.777 | 8.429 | 69.907 | C |
| ATOM | 15 | C | VAL | 354 | 18.182 | 9.044 | 69.653 | C |
| ATOM | 16 | O | VAL | 354 | 18.587 | 9.38 | 68.534 | O |
| ATOM | 17 | CB | VAL | 354 | 16.449 | 7.338 | 68.862 | C |
| ATOM | 18 | CG1 | VAL | 354 | 17.393 | 6.139 | 69.001 | C |
| ATOM | 19 | CG2 | VAL | 354 | 15.006 | 6.845 | 68.989 | C |
| ATOM | 20 | N | ASP | 395 | 23.437 | 0.237 | 79.318 | N |
| ATOM | 21 | CA | ASP | 395 | 23.584 | 0.607 | 77.922 | C |
| ATOM | 22 | C | ASP | 395 | 22.947 | 1.969 | 77.643 | C |
| ATOM | 23 | O | ASP | 395 | 23.159 | 2.55 | 76.571 | O |
| ATOM | 24 | CB | ASP | 395 | 22.96 | −0.388 | 76.95 | C |
| ATOM | 25 | CG | ASP | 395 | 23.574 | −1.775 | 77.072 | C |
| ATOM | 26 | OD1 | ASP | 395 | 24.654 | −1.968 | 76.429 | O |
| ATOM | 27 | OD2 | ASP | 395 | 22.892 | −2.557 | 77.82 | O1− |
| ATOM | 28 | N | PHE | 424 | 21.701 | 6.454 | 74.27 | N |
| ATOM | 29 | CA | PHE | 424 | 22.472 | 5.196 | 74.251 | C |
| ATOM | 30 | C | PHE | 424 | 23.911 | 5.459 | 74.74 | C |
| ATOM | 31 | O | PHE | 424 | 24.568 | 6.437 | 74.371 | O |
| ATOM | 32 | CB | PHE | 424 | 22.537 | 4.615 | 72.829 | C |
| ATOM | 33 | CG | PHE | 424 | 23.011 | 3.181 | 72.758 | C |
| ATOM | 34 | CD1 | PHE | 424 | 22.312 | 2.164 | 73.418 | C |
| ATOM | 35 | CD2 | PHE | 424 | 24.133 | 2.83 | 72 | C |
| ATOM | 36 | CE1 | PHE | 424 | 22.71 | 0.831 | 73.309 | C |
| ATOM | 37 | CE2 | PHE | 424 | 24.545 | 1.497 | 71.907 | C |
| ATOM | 38 | CZ | PHE | 424 | 23.834 | 0.499 | 72.563 | C |
| ATOM | 39 | N | THR | 426 | 26.523 | 2.428 | 75.264 | N |
| ATOM | 40 | CA | THR | 426 | 27.515 | 1.406 | 74.889 | C |
| ATOM | 41 | C | THR | 426 | 27.792 | 1.337 | 73.37 | C |
| ATOM | 42 | O | THR | 426 | 27.717 | 0.296 | 72.721 | O |
| ATOM | 43 | CB | THR | 426 | 27.09 | 0.039 | 75.471 | C |
| ATOM | 44 | OG1 | THR | 426 | 25.662 | −0.029 | 75.44 | O |
| ATOM | 45 | CG2 | THR | 426 | 27.548 | −0.109 | 76.917 | C |
| ATOM | 46 | N | TRP | 427 | 28.29 | 2.508 | 72.827 | N |
| ATOM | 47 | CA | TRP | 427 | 28.771 | 2.63 | 71.439 | C |
| ATOM | 48 | C | TRP | 427 | 30.102 | 3.404 | 71.549 | C |
| ATOM | 49 | O | TRP | 427 | 30.166 | 4.464 | 72.19 | O |
| ATOM | 50 | CB | TRP | 427 | 27.743 | 3.375 | 70.581 | C |
| ATOM | 51 | CG | TRP | 427 | 28.027 | 3.37 | 69.115 | C |
| ATOM | 52 | CD1 | TRP | 427 | 28.953 | 4.175 | 68.488 | C |
| ATOM | 53 | CD2 | TRP | 427 | 27.395 | 2.594 | 68.086 | C |
| ATOM | 54 | NE1 | TRP | 427 | 29.014 | 3.811 | 67.174 | N |
| ATOM | 55 | CE2 | TRP | 427 | 28.044 | 2.885 | 66.886 | C |
| ATOM | 56 | CE3 | TRP | 427 | 26.339 | 1.66 | 68.051 | C |
| ATOM | 57 | CZ2 | TRP | 427 | 27.705 | 2.28 | 65.669 | C |
| ATOM | 58 | CZ3 | TRP | 427 | 25.968 | 1.069 | 66.834 | C |
| ATOM | 59 | CH2 | TRP | 427 | 26.644 | 1.378 | 65.659 | C |
| ATOM | 60 | N | PRO | 433 | 30.224 | −1.414 | 67.792 | N |
| ATOM | 61 | CA | PRO | 433 | 30.304 | −2.31 | 68.944 | C |
| ATOM | 62 | C | PRO | 433 | 30.683 | −3.753 | 68.575 | C |
| ATOM | 63 | O | PRO | 433 | 30.549 | −4.254 | 67.458 | O |
| ATOM | 64 | CB | PRO | 433 | 28.894 | −2.324 | 69.551 | C |
| ATOM | 65 | CG | PRO | 433 | 28.207 | −1.119 | 68.934 | C |
| ATOM | 66 | CD | PRO | 433 | 28.845 | −1.022 | 67.561 | C |
| ATOM | 67 | N | ASP | 435 | 29.352 | −6.515 | 70.067 | N |
| ATOM | 68 | CA | ASP | 435 | 28.226 | −7.444 | 70.21 | C |
| ATOM | 69 | C | ASP | 435 | 27.015 | −6.726 | 69.581 | C |
| ATOM | 70 | O | ASP | 435 | 26.789 | −5.536 | 69.833 | O |
| ATOM | 71 | CB | ASP | 435 | 27.958 | −7.753 | 71.68 | C |
| ATOM | 72 | CG | ASP | 435 | 26.816 | −8.757 | 71.686 | C |
| ATOM | 73 | OD1 | ASP | 435 | 27.152 | −9.973 | 71.66 | O |
| ATOM | 74 | OD2 | ASP | 435 | 25.652 | −8.255 | 71.635 | O1− |
| ATOM | 75 | N | PRO | 436 | 26.297 | −7.377 | 68.618 | N |
| ATOM | 76 | CA | PRO | 436 | 25.147 | −6.724 | 68.01 | C |
| ATOM | 77 | C | PRO | 436 | 23.85 | −6.925 | 68.809 | C |
| ATOM | 78 | O | PRO | 436 | 22.762 | −6.49 | 68.43 | O |
| ATOM | 79 | CB | PRO | 436 | 25.056 | −7.424 | 66.655 | C |
| ATOM | 80 | CG | PRO | 436 | 25.469 | −8.857 | 66.975 | C |
| ATOM | 81 | CD | PRO | 436 | 26.544 | −8.692 | 68.037 | C |
| ATOM | 82 | N | GLY | 437 | 23.976 | −7.841 | 69.827 | N |
| ATOM | 83 | CA | GLY | 437 | 23.132 | −8.985 | 69.87 | C |
| ATOM | 84 | C | GLY | 437 | 22.104 | −9.168 | 70.964 | C |
| ATOM | 85 | O | GLY | 437 | 21.826 | −8.387 | 71.869 | O |
| ATOM | 86 | N | GLY | 438 | 21.476 | −10.39 | 70.788 | N |
| ATOM | 87 | CA | GLY | 438 | 20.202 | −10.753 | 71.341 | C |
| ATOM | 88 | C | GLY | 438 | 20.207 | −11.145 | 72.809 | C |
| ATOM | 89 | O | GLY | 438 | 19.646 | −12.17 | 73.202 | O |
| ATOM | 90 | N | VAL | 439 | 20.761 | −10.168 | 73.6 | N |
| ATOM | 91 | CA | VAL | 439 | 20.686 | −10.08 | 75.053 | C |
| ATOM | 92 | C | VAL | 439 | 20.597 | −8.602 | 75.52 | C |
| ATOM | 93 | O | VAL | 439 | 20.08 | −8.306 | 76.602 | O |
| ATOM | 94 | CB | VAL | 439 | 21.826 | −10.883 | 75.716 | C |
| ATOM | 95 | CG1 | VAL | 439 | 23.216 | −10.309 | 75.44 | C |
| ATOM | 96 | CG2 | VAL | 439 | 21.614 | −11.038 | 77.224 | C |
| ATOM | 97 | N | LEU | 440 | 21.22 | −7.646 | 74.733 | N |
| ATOM | 98 | CA | LEU | 440 | 21.271 | −6.233 | 75.118 | C |
| ATOM | 99 | C | LEU | 440 | 20.05 | −5.557 | 74.463 | C |
| ATOM | 100 | O | LEU | 440 | 20.076 | −4.945 | 73.397 | O |
| ATOM | 101 | CB | LEU | 440 | 22.585 | −5.56 | 74.695 | C |
| ATOM | 102 | CG | LEU | 440 | 23.829 | −6.231 | 75.323 | C |
| ATOM | 103 | CD1 | LEU | 440 | 24.583 | −7.056 | 74.282 | C |
| ATOM | 104 | CD2 | LEU | 440 | 24.782 | −5.197 | 75.918 | C |
| ATOM | 105 | N | ASP | 441 | 18.884 | −5.749 | 75.175 | N |
| ATOM | 106 | CA | ASP | 441 | 17.518 | −5.53 | 74.653 | C |
| ATOM | 107 | C | ASP | 441 | 17.103 | −4.042 | 74.393 | C |
| ATOM | 108 | O | ASP | 441 | 15.936 | −3.649 | 74.483 | O |
| ATOM | 109 | CB | ASP | 441 | 16.549 | −6.177 | 75.651 | C |
| ATOM | 110 | CG | ASP | 441 | 15.294 | −6.817 | 75.077 | C |
| ATOM | 111 | OD1 | ASP | 441 | 14.357 | −6.967 | 75.915 | O |
| ATOM | 112 | OD2 | ASP | 441 | 15.365 | −7.208 | 73.878 | O1− |
| ATOM | 113 | N | PHE | 442 | 18.08 | −3.203 | 73.869 | N |
| ATOM | 114 | CA | PHE | 442 | 17.88 | −1.739 | 73.776 | C |
| ATOM | 115 | C | PHE | 442 | 16.864 | −1.364 | 72.69 | C |
| ATOM | 116 | O | PHE | 442 | 16.137 | −0.372 | 72.778 | O |
| ATOM | 117 | CB | PHE | 442 | 19.2 | −0.996 | 73.485 | C |
| ATOM | 118 | CG | PHE | 442 | 19.036 | 0.495 | 73.247 | C |
| ATOM | 119 | CD1 | PHE | 442 | 18.618 | 1.339 | 74.283 | C |
| ATOM | 120 | CD2 | PHE | 442 | 19.233 | 1.047 | 71.971 | C |
| ATOM | 121 | CE1 | PHE | 442 | 18.425 | 2.705 | 74.056 | C |
| ATOM | 122 | CE2 | PHE | 442 | 19.025 | 2.414 | 71.746 | C |
| ATOM | 123 | CZ | PHE | 442 | 18.634 | 3.247 | 72.791 | C |
| ATOM | 124 | N | LEU | 443 | 16.918 | −2.158 | 71.561 | N |
| ATOM | 125 | CA | LEU | 443 | 16.096 | −1.859 | 70.393 | C |
| ATOM | 126 | C | LEU | 443 | 14.642 | −1.868 | 70.868 | C |
| ATOM | 127 | O | LEU | 443 | 13.775 | −1.14 | 70.382 | O |
| ATOM | 128 | CB | LEU | 443 | 16.39 | −2.917 | 69.32 | C |
| ATOM | 129 | CG | LEU | 443 | 15.632 | −2.836 | 67.985 | C |
| ATOM | 130 | CD1 | LEU | 443 | 14.231 | −3.438 | 68.074 | C |
| ATOM | 131 | CD2 | LEU | 443 | 15.603 | −1.431 | 67.393 | C |
| ATOM | 132 | N | GLU | 444 | 14.378 | −2.851 | 71.803 | N |
| ATOM | 133 | CA | GLU | 444 | 13.028 | −3.185 | 72.162 | C |
| ATOM | 134 | C | GLU | 444 | 12.452 | −2.115 | 73.09 | C |
| ATOM | 135 | O | GLU | 444 | 11.257 | −1.813 | 72.999 | O |
| ATOM | 135 | CB | GLU | 444 | 12.957 | −4.593 | 72.748 | C |
| ATOM | 137 | CG | GLU | 444 | 11.718 | −5.314 | 72.243 | C |
| ATOM | 138 | CD | GLU | 444 | 11.905 | −5.917 | 70.863 | C |
| ATOM | 139 | OE1 | GLU | 444 | 11.803 | −5.165 | 69.844 | O |
| ATOM | 140 | OE2 | GLU | 444 | 12.106 | −7.172 | 70.833 | O1− |
| ATOM | 141 | N | VAL | 446 | 13.049 | 1.102 | 72.95 | N |
| ATOM | 142 | CA | VAL | 446 | 12.607 | 2.272 | 72.184 | C |
| ATOM | 143 | C | VAL | 446 | 11.266 | 1.887 | 71.539 | C |
| ATOM | 144 | O | VAL | 446 | 10.26 | 2.597 | 71.625 | O |
| ATOM | 145 | CB | VAL | 446 | 13.668 | 2.664 | 71.126 | C |
| ATOM | 146 | CG1 | VAL | 446 | 13.146 | 3.6 | 70.036 | C |
| ATOM | 147 | CG2 | VAL | 446 | 14.89 | 3.318 | 71.779 | C |
| ATOM | 148 | N | VAL | 459 | 9.831 | 9.657 | 64.503 | N |
| ATOM | 149 | CA | VAL | 459 | 10.842 | 9.332 | 65.52 | C |
| ATOM | 150 | C | VAL | 459 | 12.102 | 10.141 | 65.182 | C |
| ATOM | 151 | O | VAL | 459 | 12.557 | 10.152 | 64.037 | O |
| ATOM | 152 | CB | VAL | 459 | 11.131 | 7.815 | 65.541 | C |
| ATOM | 153 | CG1 | VAL | 459 | 12.173 | 7.446 | 66.6 | C |
| ATOM | 154 | CG2 | VAL | 459 | 9.853 | 7.007 | 65.805 | C |
| ATOM | 155 | N | VAL | 461 | 15.87 | 10.677 | 65.283 | N |
| ATOM | 156 | CA | VAL | 461 | 17.041 | 9.822 | 65.417 | C |
| ATOM | 157 | C | VAL | 461 | 18.249 | 10.68 | 65.068 | C |
| ATOM | 158 | O | VAL | 461 | 18.395 | 11.228 | 63.972 | O |
| ATOM | 159 | CB | VAL | 461 | 16.977 | 8.608 | 64.471 | C |
| ATOM | 160 | CG1 | VAL | 461 | 18.105 | 7.621 | 64.798 | C |
| ATOM | 161 | CG2 | VAL | 461 | 15.626 | 7.895 | 64.539 | C |
| ATOM | 162 | N | PHE | 473 | 20.988 | 2.569 | 62.679 | N |
| ATOM | 163 | CA | PHE | 473 | 19.93 | 2.276 | 63.639 | C |
| ATOM | 164 | C | PHE | 473 | 18.584 | 2.267 | 62.899 | C |
| ATOM | 165 | O | PHE | 473 | 17.685 | 1.489 | 63.227 | O |
| ATOM | 166 | CB | PHE | 473 | 19.861 | 3.288 | 64.794 | C |
| ATOM | 167 | CG | PHE | 473 | 20.815 | 3.038 | 65.947 | C |
| ATOM | 168 | CD1 | PHE | 473 | 20.315 | 2.72 | 67.217 | C |
| ATOM | 169 | CD2 | PHE | 473 | 22.196 | 3.202 | 65.806 | C |
| ATOM | 170 | CE1 | PHE | 473 | 21.174 | 2.598 | 68.313 | C |
| ATOM | 171 | CE2 | PHE | 473 | 23.052 | 3.108 | 66.905 | C |
| ATOM | 172 | CZ | PHE | 473 | 22.541 | 2.801 | 68.161 | C |
| ATOM | 173 | N | ILE | 474 | 18.405 | 3.238 | 61.93 | N |
| ATOM | 174 | CA | ILE | 474 | 17.116 | 3.336 | 61.234 | C |
| ATOM | 175 | C | ILE | 474 | 16.907 | 2.064 | 60.398 | C |
| ATOM | 176 | O | ILE | 474 | 15.821 | 1.48 | 60.385 | O |
| ATOM | 177 | CB | ILE | 474 | 17.024 | 4.629 | 60.386 | C |
| ATOM | 178 | CG1 | ILE | 474 | 16.713 | 5.813 | 61.325 | C |
| ATOM | 179 | CG2 | ILE | 474 | 15.977 | 4.535 | 59.27 | C |
| ATOM | 180 | CD1 | ILE | 474 | 16.812 | 7.171 | 60.656 | C |
| ATOM | 181 | N | ILE | 476 | 18.354 | −0.814 | 60.761 | N |
| ATOM | 182 | CA | ILE | 476 | 18.106 | −1.919 | 61.69 | C |
| ATOM | 183 | C | ILE | 476 | 16.63 | −1.865 | 62.106 | C |
| ATOM | 184 | O | ILE | 476 | 15.923 | −2.872 | 61.994 | O |
| ATOM | 185 | CB | ILE | 476 | 19.04 | −1.919 | 62.927 | C |
| ATOM | 186 | CG1 | ILE | 476 | 20.436 | −2.415 | 62.507 | C |
| ATOM | 187 | CG2 | ILE | 476 | 18.502 | −2.803 | 64.067 | C |
| ATOM | 188 | CD1 | ILE | 476 | 21.505 | −2.207 | 63.566 | C |
| ATOM | 189 | N | ASP | 477 | 16.175 | −0.673 | 62.653 | N |
| ATOM | 190 | CA | ASP | 477 | 14.805 | −0.583 | 63.167 | C |
| ATOM | 191 | C | ASP | 477 | 13.81 | −0.972 | 62.061 | C |
| ATOM | 192 | O | ASP | 477 | 12.825 | −1.674 | 62.309 | O |
| ATOM | 193 | CB | ASP | 477 | 14.5 | 0.804 | 63.712 | C |
| ATOM | 194 | CG | ASP | 477 | 13.134 | 0.757 | 64.374 | C |
| ATOM | 195 | OD1 | ASP | 477 | 13.092 | 0.26 | 65.552 | O |
| ATOM | 196 | OD2 | ASP | 477 | 12.169 | 1.249 | 63.713 | O1− |
| ATOM | 197 | N | ILE | 480 | 14.126 | −4.85 | 61.295 | N |
| ATOM | 198 | CA | ILE | 480 | 13.541 | −5.614 | 62.388 | C |
| ATOM | 199 | C | ILE | 480 | 12.02 | −5.422 | 62.402 | C |
| ATOM | 200 | O | ILE | 480 | 11.285 | −6.396 | 62.61 | O |
| ATOM | 201 | CB | ILE | 480 | 14.244 | −5.405 | 63.739 | C |
| ATOM | 202 | CG1 | ILE | 480 | 13.931 | −6.529 | 64.746 | C |
| ATOM | 203 | CG2 | ILE | 480 | 14.014 | −4.031 | 64.34 | C |
| ATOM | 204 | CD1 | ILE | 480 | 12.667 | −6.353 | 65.576 | C |
| ATOM | 205 | N | PHE | 517 | 27.131 | −4.748 | 60.449 | N |
| ATOM | 206 | CA | PHE | 517 | 26.455 | −4.608 | 61.741 | C |
| ATOM | 207 | C | PHE | 517 | 24.933 | −4.771 | 61.562 | C |
| ATOM | 208 | O | PHE | 517 | 24.254 | −5.345 | 62.419 | O |
| ATOM | 209 | CB | PHE | 517 | 26.786 | −3.279 | 62.429 | C |
| ATOM | 210 | CG | PHE | 517 | 26.366 | −3.246 | 63.88 | C |
| ATOM | 211 | CD1 | PHE | 517 | 27.06 | −3.988 | 64.843 | C |
| ATOM | 212 | CD2 | PHE | 517 | 25.253 | −2.497 | 64.279 | C |
| ATOM | 213 | CE1 | PHE | 517 | 26.641 | −3.983 | 66.175 | C |
| ATOM | 214 | CE2 | PHE | 517 | 24.832 | −2.497 | 65.609 | C |
| ATOM | 215 | CZ | PHE | 517 | 25.524 | −3.241 | 66.558 | C |
| ATOM | 216 | N | ALA | 521 | 22.986 | −7.746 | 63.399 | N |
| ATOM | 217 | CA | ALA | 521 | 21.893 | −7.369 | 64.286 | C |
| ATOM | 218 | C | ALA | 521 | 20.609 | −8.097 | 63.876 | C |
| ATOM | 219 | O | ALA | 521 | 19.859 | −8.604 | 64.716 | O |
| ATOM | 220 | CB | ALA | 521 | 21.669 | −5.867 | 64.295 | C |
| ATOM | 221 | N | VAL | 522 | 20.319 | −8.104 | 62.526 | N |
| ATOM | 222 | CA | VAL | 522 | 19.105 | −8.765 | 62.047 | C |
| ATOM | 223 | C | VAL | 522 | 19.247 | −10.283 | 62.286 | C |
| ATOM | 224 | O | VAL | 522 | 18.299 | −10.952 | 62.706 | O |
| ATOM | 225 | CB | VAL | 522 | 18.786 | −8.401 | 60.587 | C |
| ATOM | 226 | CG1 | VAL | 522 | 17.619 | −9.229 | 60.043 | C |
| ATOM | 227 | CG2 | VAL | 522 | 18.405 | −6.918 | 60.471 | C |
| ATOM | 228 | N | HIS | 524 | 21.134 | −11.671 | 64.713 | N |
| ATOM | 229 | CA | HIS | 524 | 20.95 | −11.925 | 66.131 | C |
| ATOM | 230 | C | HIS | 524 | 19.466 | −11.837 | 66.508 | C |
| ATOM | 231 | O | HIS | 524 | 18.967 | −12.615 | 67.329 | O |
| ATOM | 232 | CB | HIS | 524 | 21.781 | −11.033 | 67.055 | C |
| ATOM | 233 | CG | HIS | 524 | 22.801 | −11.838 | 67.797 | C |
| ATOM | 234 | ND1 | HIS | 524 | 22.619 | −12.171 | 69.127 | N |
| ATOM | 235 | CD2 | HIS | 524 | 23.987 | −12.364 | 67.329 | C |
| ATOM | 236 | CE1 | HIS | 524 | 23.675 | −12.892 | 69.445 | C |
| ATOM | 237 | NE2 | HIS | 524 | 24.522 | −13.046 | 68.384 | N |
| ATOM | 238 | N | TYR | 525 | 18.731 | −10.807 | 65.939 | N |
| ATOM | 239 | CA | TYR | 525 | 17.322 | −10.673 | 66.307 | C |
| ATOM | 240 | C | TYR | 525 | 16.52 | −11.911 | 65.854 | C |
| ATOM | 241 | O | TYR | 525 | 15.623 | −12.373 | 66.569 | O |
| ATOM | 242 | CB | TYR | 525 | 16.64 | −9.377 | 65.842 | C |
| ATOM | 243 | CG | TYR | 525 | 15.412 | −9.093 | 66.696 | C |
| ATOM | 244 | CD1 | TYR | 525 | 15.486 | −8.242 | 67.812 | C |
| ATOM | 245 | CD2 | TYR | 525 | 14.201 | −9.751 | 66.436 | C |
| ATOM | 246 | CE1 | TYR | 525 | 14.392 | −8.096 | 68.676 | C |
| ATOM | 247 | CE2 | TYR | 525 | 13.128 | −9.641 | 67.322 | C |
| ATOM | 248 | CZ | TYR | 525 | 13.233 | −8.824 | 68.44 | C |
| ATOM | 249 | OH | TYR | 525 | 12.17 | −8.81 | 69.298 | O |
| ATOM | 250 | N | THR | 528 | 16.802 | −14.389 | 68.261 | N |
| ATOM | 251 | CA | THR | 528 | 15.978 | −14.093 | 69.44 | C |
| ATOM | 252 | C | THR | 528 | 14.48 | −14.296 | 69.211 | C |
| ATOM | 253 | O | THR | 528 | 13.712 | −14.439 | 70.169 | O |
| ATOM | 254 | CB | THR | 528 | 16.066 | −12.674 | 70.051 | C |
| ATOM | 255 | OG1 | THR | 528 | 15.221 | −11.712 | 69.387 | O |
| ATOM | 256 | CG2 | THR | 528 | 17.454 | −12.117 | 70.186 | C |
| ATOM | 257 | N | ARG | 532 | 12.876 | −17.639 | 71.592 | N |
| ATOM | 258 | CA | ARG | 532 | 12.027 | −17.254 | 72.73 | C |
| ATOM | 259 | C | ARG | 532 | 10.569 | −17.794 | 72.636 | C |
| ATOM | 260 | O | ARG | 532 | 10.46 | −19.055 | 72.733 | O |
| ATOM | 261 | CB | ARG | 532 | 12.032 | −15.729 | 72.899 | C |
| ATOM | 262 | CG | ARG | 532 | 13.387 | −15.184 | 73.353 | C |
| ATOM | 263 | CD | ARG | 532 | 13.33 | −13.666 | 73.491 | C |
| ATOM | 264 | NE | ARG | 532 | 13.295 | −13.004 | 72.183 | N |
| ATOM | 265 | CZ | ARG | 532 | 12.904 | −11.716 | 72 | C |
| ATOM | 266 | NH1 | ARG | 532 | 13.315 | −11.003 | 70.923 | N |
| ATOM | 267 | NH2 | ARG | 532 | 12.113 | −11.07 | 72.877 | N1+ |
| ATOM | 268 | OXT | ARG | 532 | 9.657 | −16.927 | 72.508 | O1− |
| TER | 269 | ARG | 532 | | | | | |
| END | | | | | | | | |

TABLE 5
Enrichment Model 4—Example 2 Coordinates
(the WPD loop was selected as active zone)

| | | | | | | | | |
|---|---|---|---|---|---|---|---|---|
| ATOM | 1 | N | HIS | 394 | 7.657 | 58.464 | 1.629 | N |
| ATOM | 2 | CA | HIS | 394 | 7.432 | 58.077 | 0.228 | C |
| ATOM | 3 | C | HIS | 394 | 6.628 | 56.765 | 0.167 | C |
| ATOM | 4 | O | HIS | 394 | 5.701 | 56.612 | −0.628 | O |
| ATOM | 5 | CB | HIS | 394 | 8.78 | 57.957 | −0.508 | C |
| ATOM | 6 | CG | HIS | 394 | 8.716 | 57.197 | −1.785 | C |
| ATOM | 7 | CD2 | HIS | 394 | 8.322 | 57.515 | −3.067 | C |
| ATOM | 8 | ND1 | HIS | 394 | 9.057 | 55.867 | −1.849 | N |
| ATOM | 9 | NE2 | HIS | 394 | 8.414 | 56.424 | −3.895 | N |
| ATOM | 10 | CE1 | HIS | 394 | 8.858 | 55.451 | −3.134 | C |
| ATOM | 11 | N | ASP | 395 | 7.116 | 55.747 | 0.966 | N |
| ATOM | 12 | CA | ASP | 395 | 6.575 | 54.389 | 0.884 | C |
| ATOM | 13 | C | ASP | 395 | 5.336 | 54.13 | 1.765 | C |
| ATOM | 14 | O | ASP | 395 | 4.648 | 53.114 | 1.608 | O |
| ATOM | 15 | CB | ASP | 395 | 7.636 | 53.335 | 1.229 | C |
| ATOM | 16 | CG | ASP | 395 | 8.612 | 53.248 | 0.069 | C |
| ATOM | 17 | OD1 | ASP | 395 | 8.481 | 52.258 | −0.717 | O |
| ATOM | 18 | OD2 | ASP | 395 | 9.437 | 54.221 | 0.027 | O1− |
| ATOM | 19 | N | PHE | 424 | 0.636 | 50.732 | 4.402 | N |
| ATOM | 20 | CA | PHE | 424 | 1.571 | 50.572 | 3.277 | C |
| ATOM | 21 | C | PHE | 424 | 0.729 | 50.798 | 2.01 | C |
| ATOM | 22 | O | PHE | 424 | −0.416 | 50.354 | 1.895 | O |
| ATOM | 23 | CB | PHE | 424 | 2.164 | 49.16 | 3.235 | C |
| ATOM | 24 | CG | PHE | 424 | 3.35 | 49.008 | 2.312 | C |
| ATOM | 25 | CD1 | PHE | 424 | 4.577 | 49.599 | 2.642 | C |
| ATOM | 26 | CD2 | PHE | 424 | 3.256 | 48.258 | 1.13 | C |
| ATOM | 27 | CE1 | PHE | 474 | 5.7 | 49.414 | 1.834 | C |
| ATOM | 28 | CE2 | PHE | 424 | 4.377 | 48.09 | 0.309 | C |
| ATOM | 29 | CZ | PHE | 424 | 5.598 | 48.656 | 0.67 | C |
| ATOM | 30 | N | THR | 426 | 2.683 | 51.108 | −1.424 | N |
| ATOM | 31 | CA | THR | 426 | 3.509 | 50.855 | −2.611 | C |
| ATOM | 32 | C | THR | 426 | 3.532 | 49.353 | −2.962 | C |
| ATOM | 33 | O | THR | 426 | 4.561 | 48.711 | −3.179 | O |
| ATOM | 34 | CB | THR | 426 | 4.902 | 51.469 | −2.375 | C |
| ATOM | 35 | OG1 | THR | 426 | 5.203 | 51.36 | −0.977 | O |
| ATOM | 36 | CG2 | THR | 426 | 4.915 | 52.953 | −2.756 | C |
| ATOM | 37 | N | TRP | 427 | 2.257 | 48.808 | −3.191 | N |
| ATOM | 38 | CA | TRP | 427 | 1.962 | 47.444 | −3.707 | C |
| ATOM | 39 | C | TRP | 427 | 1.346 | 47.676 | −5.11 | C |
| ATOM | 40 | O | TRP | 427 | 0.275 | 48.286 | −5.245 | O |
| ATOM | 41 | CB | TRP | 427 | 0.971 | 46.651 | −2.824 | C |
| ATOM | 42 | CG | TRP | 427 | 0.322 | 45.475 | −3.524 | C |
| ATOM | 43 | CD1 | TRP | 427 | −0.949 | 45.479 | −4.072 | C |
| ATOM | 44 | CD2 | TRP | 427 | 0.853 | 44.164 | −3.783 | C |
| ATOM | 45 | NE1 | TRP | 427 | −1.16 | 44.303 | −4.742 | N |
| ATOM | 46 | CE2 | TRP | 427 | −0.084 | 43.472 | −4.557 | C |
| ATOM | 47 | CE3 | TRP | 427 | 2.05 | 43.495 | −3.454 | C |
| ATOM | 48 | CZ2 | TRP | 427 | 0.12 | 42.17 | −5.032 | C |
| ATOM | 49 | CZ3 | TRP | 427 | 2.267 | 42.186 | −3.911 | C |
| ATOM | 50 | CH2 | TRP | 427 | 1.316 | 41.537 | −4.692 | C |
| ATOM | 51 | N | PRO | 428 | 2.077 | 47.224 | −6.195 | N |
| ATOM | 52 | CA | PRO | 428 | 1.875 | 47.718 | −7.563 | C |
| ATOM | 53 | C | PRO | 428 | 0.648 | 47.272 | −8.386 | C |
| ATOM | 54 | O | PRO | 428 | 0.62 | 47.399 | −9.614 | O |
| ATOM | 55 | CB | PRO | 428 | 3.169 | 47.313 | −8.278 | C |
| ATOM | 56 | CG | PRO | 428 | 3.533 | 46.003 | −7.593 | C |
| ATOM | 57 | CD | PRO | 428 | 3.198 | 46.29 | −6.141 | C |
| ATOM | 58 | N | VAL | 432 | 4.119 | 51.345 | −10.539 | N |
| ATOM | 59 | CA | VAL | 432 | 5.313 | 50.995 | −11.329 | C |
| ATOM | 60 | C | VAL | 432 | 6.46 | 50.96 | −10.295 | C |
| ATOM | 61 | O | VAL | 432 | 6.954 | 52.009 | −9.869 | O |
| ATOM | 62 | CB | VAL | 432 | 5.573 | 52.043 | −12.435 | C |
| ATOM | 63 | CG1 | VAL | 432 | 6.829 | 51.689 | −13.239 | C |
| ATOM | 64 | CG2 | VAL | 432 | 4.381 | 52.154 | −13.393 | C |
| ATOM | 65 | N | PRO | 433 | 6.8 | 49.727 | −9.773 | N |
| ATOM | 66 | CA | PRO | 433 | 7.675 | 49.605 | −8.606 | C |
| ATOM | 67 | C | PRO | 433 | 9.175 | 49.662 | −8.96 | C |
| ATOM | 68 | O | PRO | 433 | 9.601 | 49.498 | −10.103 | O |
| ATOM | 69 | CB | PRO | 433 | 7.333 | 48.215 | −8.064 | C |
| ATOM | 70 | CG | PRO | 433 | 7.054 | 47.416 | −9.336 | C |
| ATOM | 71 | CD | PRO | 433 | 6.355 | 48.421 | −10.242 | C |
| ATOM | 72 | N | SER | 434 | 10.019 | 49.808 | −7.871 | N |
| ATOM | 73 | CA | SER | 434 | 11.466 | 49.684 | −8.036 | C |
| ATOM | 74 | C | SER | 434 | 12.049 | 49.366 | −6.653 | C |
| ATOM | 75 | O | SER | 434 | 11.7 | 49.998 | −5.657 | O |
| ATOM | 76 | CB | SER | 434 | 12.101 | 50.965 | −8.585 | C |
| ATOM | 77 | OG | SER | 434 | 13.318 | 50.653 | −9.267 | O |
| ATOM | 78 | N | ASP | 435 | 12.957 | 48.323 | −6.66 | N |
| ATOM | 79 | CA | ASP | 435 | 13.719 | 47.852 | −5.496 | C |
| ATOM | 80 | C | ASP | 435 | 12.809 | 47.117 | −4.468 | C |
| ATOM | 81 | O | ASP | 435 | 11.831 | 47.661 | −3.943 | O |
| ATOM | 82 | CB | ASP | 435 | 14.543 | 48.979 | −4.878 | C |
| ATOM | 83 | CG | ASP | 435 | 15.532 | 48.349 | −3.924 | C |
| ATOM | 84 | OD1 | ASP | 435 | 15.016 | 47.766 | −2.926 | O |
| ATOM | 85 | OD2 | ASP | 435 | 16.759 | 48.451 | −4.21 | O1− |
| ATOM | 86 | N | PRO | 436 | 13.135 | 45.805 | −4.141 | N |
| ATOM | 87 | CA | PRO | 436 | 12.377 | 45.085 | −3.111 | C |
| ATOM | 88 | C | PRO | 436 | 12.705 | 45.459 | −1.645 | C |
| ATOM | 89 | O | PRO | 436 | 12.088 | 44.978 | −0.69 | O |
| ATOM | 90 | CB | PRO | 436 | 12.711 | 43.616 | −3.377 | C |
| ATOM | 91 | CG | PRO | 436 | 14.137 | 43.673 | −3.91 | C |
| ATOM | 92 | CD | PRO | 436 | 14.155 | 44.951 | −4.737 | C |
| ATOM | 93 | N | GLY | 437 | 13.689 | 46.398 | −1.442 | N |
| ATOM | 94 | CA | GLY | 437 | 14.095 | 46.867 | −0.132 | C |
| ATOM | 95 | C | GLY | 437 | 13.211 | 47.974 | 0.431 | C |
| ATOM | 96 | O | GLY | 437 | 13.406 | 48.451 | 1.553 | O |
| ATOM | 97 | N | GLY | 438 | 12.127 | 48.324 | −0.353 | N |
| ATOM | 98 | CA | GLY | 438 | 11.092 | 49.223 | 0.143 | C |
| ATOM | 99 | C | GLY | 438 | 10.33 | 48.566 | 1.3 | C |
| ATOM | 100 | O | GLY | 438 | 9.884 | 49.206 | 2.253 | O |
| ATOM | 101 | N | VAL | 439 | 10.17 | 47.198 | 1.172 | N |
| ATOM | 102 | CA | VAL | 439 | 9.492 | 46.383 | 2.183 | C |
| ATOM | 103 | C | VAL | 439 | 10.391 | 46.311 | 3.428 | C |
| ATOM | 104 | O | VAL | 439 | 9.934 | 46.229 | 4.571 | O |
| ATOM | 105 | CB | VAL | 439 | 9.15 | 44.993 | 1.602 | C |
| ATOM | 106 | CG1 | VAL | 439 | 8.968 | 43.908 | 2.664 | C |
| ATOM | 107 | CG2 | VAL | 439 | 7.891 | 45.109 | 0.734 | C |
| ATOM | 108 | N | ARG | 469 | 0.256 | 38.237 | 0.716 | N |
| ATOM | 109 | CA | ARG | 469 | 1.055 | 39.414 | 1.04 | C |
| ATOM | 110 | C | ARG | 469 | 1.501 | 39.246 | 2.508 | C |
| ATOM | 111 | O | ARG | 469 | 2.633 | 39.534 | 2.893 | O |
| ATOM | 112 | CB | ARG | 469 | 0.296 | 40.751 | 0.904 | C |
| ATOM | 113 | CG | ARG | 469 | −0.572 | 40.906 | −0.35 | C |
| ATOM | 114 | CD | ARG | 469 | −1.215 | 42.298 | −0.428 | C |
| ATOM | 115 | NE | ARG | 469 | −2.422 | 42.281 | −1.269 | N |
| ATOM | 116 | CZ | ARG | 469 | −3.08 | 43.382 | −1.737 | C |
| ATOM | 117 | NH1 | ARG | 469 | −2.685 | 44.632 | −1.399 | N |
| ATOM | 118 | NH2 | ARG | 469 | −4.143 | 43.244 | −2.57 | N1+ |
| ATOM | 119 | N | THR | 472 | 4.118 | 36.781 | 2.976 | N |
| ATOM | 120 | CA | THR | 472 | 5.363 | 37.334 | 2.434 | C |
| ATOM | 121 | C | THR | 472 | 5.993 | 38.213 | 3.519 | C |
| ATOM | 122 | O | THR | 472 | 7.176 | 38.089 | 3.839 | O |
| ATOM | 123 | CB | THR | 472 | 5.117 | 38.106 | 1.127 | C |
| ATOM | 124 | OG1 | THR | 472 | 4.769 | 37.181 | 0.088 | O |
| ATOM | 125 | CG2 | THR | 472 | 6.334 | 38.876 | 0.637 | C |
| ATOM | 126 | N | PHE | 473 | 5.154 | 39.156 | 4.086 | N |
| ATOM | 127 | CA | PHE | 473 | 5.661 | 40.086 | 5.096 | C |
| ATOM | 128 | C | PHE | 473 | 6.113 | 39.331 | 6.364 | C |
| ATOM | 129 | O | PHE | 473 | 7.14 | 39.656 | 6.969 | O |
| ATOM | 130 | CB | PHE | 473 | 4.628 | 41.161 | 5.485 | C |
| ATOM | 131 | CG | PHE | 473 | 4.52 | 42.29 | 4.481 | C |
| ATOM | 132 | CD1 | PHE | 473 | 5.517 | 43.27 | 4.408 | C |
| ATOM | 133 | CD2 | PHE | 473 | 3.422 | 42.396 | 3.62 | C |
| ATOM | 134 | CE1 | PHE | 473 | 5.414 | 44.326 | 3.501 | C |
| ATOM | 135 | CE2 | PHE | 473 | 3.346 | 43.425 | 2.677 | C |
| ATOM | 136 | CZ | PHE | 473 | 4.335 | 44.4 | 2.626 | C |
| ATOM | 137 | N | GLN | 514 | 6.216 | 35.626 | −5.095 | N |
| ATOM | 138 | CA | GLN | 514 | 6.389 | 35.998 | −3.691 | C |
| ATOM | 139 | C | GLN | 514 | 7.604 | 35.274 | −3.091 | C |
| ATOM | 140 | O | GLN | 514 | 8.285 | 35.761 | −2.189 | O |
| ATOM | 141 | CB | GLN | 514 | 5.164 | 35.633 | −2.854 | C |
| ATOM | 142 | CG | GLN | 514 | 4.061 | 36.668 | −3.003 | C |
| ATOM | 143 | CD | GLN | 514 | 2.787 | 36.245 | −2.307 | C |
| ATOM | 144 | OE1 | GLN | 514 | 2.632 | 35.202 | −1.678 | O |
| ATOM | 145 | NE2 | GLN | 514 | 1.792 | 37.173 | −2.436 | N |
| ATOM | 146 | N | PHE | 517 | 10.639 | 36.858 | −4.387 | N |
| ATOM | 147 | CA | PHE | 517 | 10.645 | 38.135 | −3.658 | C |
| ATOM | 148 | C | PHE | 517 | 11.189 | 37.949 | −2.219 | C |
| ATOM | 149 | O | PHE | 517 | 11.981 | 38.764 | −1.736 | O |
| ATOM | 150 | CB | PHE | 517 | 9.238 | 38.744 | −3.647 | C |
| ATOM | 151 | CG | PHE | 517 | 9.132 | 40.147 | −3.11 | C |
| ATOM | 152 | CD1 | PHE | 517 | 9.321 | 41.249 | −3.956 | C |
| ATOM | 153 | CD2 | PHE | 517 | 8.807 | 40.363 | −1.762 | C |
| ATOM | 154 | CE1 | PHE | 517 | 9.172 | 42.542 | −3.46 | C |
| ATOM | 155 | CE2 | PHE | 517 | 8.652 | 41.658 | −1.27 | C |
| ATOM | 156 | CZ | PHE | 517 | 8.833 | 42.745 | −2.121 | C |
| TER | 157 | PHE | 517 | | | | | |
| END | | | | | | | | |

Construction of PTP-PEST, LYP, PTP1B and STEP

Models for the modulation of PTP-PEST, LYP, PTP1B and STEP are constructed by the preparation of the 3-dimensional representation of the PTP-PEST, LYP, PTP1B and STEP protein based on but not limited to the crystallographic structure of the PTP-PEST, LYP, PTP1B and STEP proteins and the application of computer algorithms to modify regions important for phosphatase function as explained in methods.

The electronic representation of the PTP-PEST, LYP, PTP1B and STEP structures are then displayed on a computer screen for visual inspection and analysis. All important motifs involved in PTP-PEST, LYP, PTP1B and STEP ligand recognition and binding were identified, including those described above.

Three dimensional graphical representation of the PTP-PEST, LYP, PTP1B and STEP modulation sites were then generated as part of an electronic representation of the ligand bound binding site. In an embodiment, the electronic representation of the binding site contains the coordinates of PTP-PEST, LYP, PTP1B and STEP residues.

The structure coordinates of amino acid residues that constitute the binding site define the chemical environment important for ligand binding, and thereby are useful in designing compounds that may interact with those residues.

The binding site amino acid residues are key residues for ligand binding. Alternatively, the binding site amino acid residues may be residues that are spatially related in the definition of the three-dimensional shape of the binding site. The amino acid residues may be contiguous or non-contiguous in the primary sequence.

The PTP-PEST, LYP, PTP1B and STEP binding sites are formed by three-dimensional coordinates of amino acid residues selected after modifying this X-ray crystallographic structure of the PTP-PEST, LYP, PTP1B and STEP protein as explained in methods. These models are mostly hydrophobic in nature but also contain polar moieties, which correspond to backbone atoms.

Computer programs are also employed to estimate the attraction, repulsion, and steric hindrance of the ligand to the PTP-PEST, LYP, PTP1B and STEP Enrichment Model. Generally the tighter the fit between the inhibitor and PTP-PEST, LYP, PTP1B and STEP at the molecular level and atomic level (e.g., the lower the steric hindrance, and/or the greater the attractive force), the more potent the potential drug will be because these properties are consistent with a tighter-binding constant.

A ligand selected in the manner described above is expected to overcome the known randomness of screening all chemical matter for the identification of hit molecules. Once the enrichment methods have identified PTP-PEST, LYP, PTP1B and STEP modulators they can be systematically modified by computer-modeling programs until one or more promising potential ligands are identified. Such computer modeling allows the selection of a finite number of rational modifications, as opposed to the countless number of essentially random chemical modifications that could be made, any of which any one might lead to a useful drug. Each chemical modification requires additional chemical steps, which while being reasonable for the synthesis of a finite number of compounds, quickly becomes overwhelming if all possible modifications needed to be synthesized. Thus, through the use of the structure coordinates disclosed herein and computer modeling, a large number of these compounds are rapidly screened on the computer monitor screen, and a few likely candidates are determined or identified without the laborious synthesis of untold numbers of compounds.

Once a potential ligand (agonist or antagonist) is identified, it is either selected from commercial libraries of compounds or synthesized de novo. As mentioned above, the de nova synthesis of one or even a relatively small group of specific compounds is reasonable in the art of drug design.

For the drug design strategies described herein further refinement(s) of the structure of the drug are generally necessary and are made by the successive iterations of any and/or all of the steps provided by the aforementioned strategies.

Another aspect of the invention involves using the structure coordinates generated from the PTP-PEST, LYP, PTP1B and STEP complexes to generate a three-dimensional shape. This is achieved through the use of commercially available software that is capable of generating three-dimensional graphical representations of molecules or portions thereof from a set of structure coordinates.

Various computational analyses can be performed to analyze PTP-PEST, LYP, PTP1B, STEP or other phosphatases. Such analyses may be carried out through the use of known software applications, such as ProMod, SWISS-MODEL (Swiss Institute of Bioinformatics), and the Molecular Similarity application of QUANTA (Accelrys, Inc., San Diego, Calif.). Programs such as QUANTA permit comparisons between different structures, different conformations of the same structure, and different parts of the same structure. Comparison of structures using such computer software may involve the following steps: 1) loading the structures to be compared; 2) defining the atom equivalencies in the structures; 3) performing a fitting operation; and 4) analyzing the results. Each structure is identified by a name. One structure is identified as the target (i.e., the fixed structure) and all remaining structures are working structures (i.e., moving structures). Since atom equivalency with QUANTA is defined by user input, for the purpose of this invention, applicants define equivalent atoms as protein backbone atoms (N, Cα, C, and O) for all conserved residues between the two structures being compared. Rigid fitting operations are also considered. When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atoms is an absolute minimum. This number, given in angstroms (Å), is reported by software applications, such as QUANTA.

Use of the Enrichment Models for Ligand Screening (Enrichment), Fitting and Selection

The PTP-PEST, LYP, PTP1B and STEP Enrichment Models are used for ligand screening (enrichment), fitting, and selection.

The electronic representation of compounds and/or fragments is generated as described above. In one embodiment of the invention, electronic representations of compounds and/or fragments are assembled into electronic databases. In another embodiment of the invention, these databases include chemical entities coordinates in any SMILES, mol, sdf, or mol2 formats.

Selected chemical entities or fragments may be positioned in a variety of orientations inside the Enrichment Model. Chemical entities come from different sources including, but not limited to, proprietary compound repositories, commercial data bases, or virtual data bases. Non-limiting exemplary sources of fragments include reagent data bases, de-novo design, etc.

The selected chemical entities or fragments are used to perform a fitting of the electronic representation of compounds and/or fragments and the Enrichment Model. The fitting is done manually or is computer assisted (docking).

The results of the fitting operation are then analyzed to quantify the association between the chemical entity and the Enrichment Model. The quality of fitting of these entities to the Enrichment Model is evaluated either by using a scoring function, shape complementarity, or estimating the interaction energy.

Methods for evaluating the association of a chemical entity with the Enrichment Model include energy minimization and molecular dynamics with standard molecular mechanics force fields, such as CHARMM (Accelrys, Inc., San Diego, Calif.) and AMBER (P. A. Kollman, University of California at San Francisco).

Additional data is obtained using Free Energy Perturbations (FEP), to account for other energetic effects such as desolvation penalties. Information about the chemical interactions with the Enrichment Model is used to elucidate chemical modifications that can enhance selectivity of binding of the modulator.

Potential binding compounds are identified based on favorable geometric fit and energetically favorable complementary interactions. Energetically favorable electrostatic interactions include attractive charge-charge, dipole-dipole and charge-dipole interactions between the target enzyme, and the small molecule.

The association with the Enrichment Model is further assessed by means of visual inspection followed by energy minimization and molecular dynamics. Examples of such programs include: MOE (CCG, Montreal, Canada), QUANTA/CHARMM (Accelrys, Inc., San Diego, Calif.); Gaussian (M. J. Frisch, Gaussian, Inc., Carnegie, Pa.); AMBER (P. A. Kollman, University of California at San Francisco); Jaguar (Schrödinger, Portland, Oreg.); SPARTAN (Wavefunction, Inc., Irvine, Calif.); Impact (Schrödinger, Portland, Oreg.); Insight II/Discover (Accelrys, Inc., San Diego, Calif.); MacroModel (Schrödinger, Portland, Oreg.); Maestro (Schrödinger, Portland, Oreg.); and DelPhi (Accelrys, Inc., San Diego, Calif.).

Once suitable fragments have been identified, they are connected into a single compound or complex on the three-dimensional image displayed on a computer screen in relation to all or a portion of the Enrichment Model.

### Use of the Enrichment Models for Ligand Design

The design of compounds using the Enrichment Models includes calculation of non-covalent molecular interactions important in the compound's binding association including hydrogen bonding, van der Waals interactions, hydrophobic interactions and electrostatic interactions.

The compound's binding affinity to the Enrichment Model is further optimized by computational evaluation of the deformation energy of binding, i.e. the energy difference between bound and free states of the chemical entity.

Computer calculations may suggest more than one conformation similar in overall binding energy for a chemical entity. In these cases the deformation energy of binding is defined as the difference between the energy of the free entity and the average energy of the conformations observed when the inhibitor binds to the protein.

### Enrichment Models for PTP-PEST (PTPN12, PTPG1), LYP (PTPN22, PEP, PTPN8), PTP1B and STEP

Examples are provided below to further illustrate different features of the present invention. The examples also illustrate useful methodology for practicing the invention. These examples do not limit the claimed invention.

The Enrichment Models for PTP-PEST (PTPN12, PTPG1), LYP (PTPN22, PEP, PTPN8), PTP1B and STEP result from exploration of conformational flexibility of the tyrosine phosphatase WPD-loop, the αF-helix and adjacent regions. These regions have been shown to play an important role on stabilization of the catalytic conformation of tyrosine phosphatases. A small molecule interacting with those regions could destabilize the WPD-loop and therefore inhibit the tyrosine phosphatase catalytic activity.

### Enrichment Models for PTP-PEST (PTPN12, PTPG1), LYP (PTPN22, PEP, PTPN8), PTP1B and STEP and the Use Thereof

### General Description of Enrichment Models

The method describes the use of a process to identify PTP-PEST, LYP, PTP1B and STEP modulators by utilization of the movement of the WPD-loop. Multiple conformations of the WPD arc expected to provide Enrichment Models, which change in electrostatic and steric properties as the WPD-loop changes its orientation. The process employed provides multiple Enrichment Models which are hereto collected and described as the Enrichment Model Collection 4. Collectively or singularly the use of these models will identify Candidate Modulators of PTP-PEST, LYP, PTP1B and STEP. The PTP-PEST, LYP, PTP1B and STEP structure employed for the construction of the Enrichment Models for PTP-PEST, LYP, PTP1B and STEP.

### General Method Description: The Construction of Enrichment Models for PTP-PEST, LYP, PTP1B and STEP

To construct the Enrichment Models for PTP-PEST, LYP, PTP1B and STEP different conformations of the WPD-loop and were generated by Conformational Search. In order to provide the PTP-PEST, LYP, PTP1B and STEP structures for construction of the Enrichment Model, a single approach was used to select residues for the conformational search. The following WPD-loop residues were used:

Example 1: PTP-PEST-TYR194 to PRO203

Example 2: PTP1B-THR177 to PRO188

Example 3: STEP-THR433 to ASP422

Example 4: LYP-TYP190 to PRO199

Enrichment Model 1: PTP-PEST (PTPN12, PTPG1)

The PTP-PEST Enrichment model contains the residues: A₁₃₂, Y₁₉₄, N₁₉₆, W₁₉₇, H₂₀₀, D₂₀₁, V₂₀₂, S₂₀₅, F₂₀₆, S₂₀₈, I₂₀₉, G₂₃₆, R₂₃₇, A₂₄₀, I₂₄₁, E₂₈₁, Q₂₈₂, E₂₈₄, L₂₈₅, R₂₈₈.

Construction of the Enrichment for PTP-PEST (PTPN12, PTPG1)
1. The WPD-loop residues TYR194 to PRO203 were selected.
2. A conformational search of the Enrichment Model was employed.
3. Force field calculations were set to disregard atoms distant from center of the Enrichment Model.
4. Molecular Dynamic calculations were accelerated by fixing the coordinates of atoms near the active zone used for the conformational search.
5. The Enrichment Model coordinates were saved in a data base and checked for the ability of Modulators to bind using Binding Site Identification tools.
6. The binding sites were checked for size and polarity giving preference to more hydrophobic rather than hydrophilic sites.
7. Enrichment Models with at least two aromatic hydrophobic residues and several polar side chains were selected.
8. The Enrichment Model contains three aromatic hydrophobic residues: TYR194 TRP197 and PHE206 (this includes Trp197 of the WPD-loop).
9. This model corresponds to a super-open conformation of the WPD-loop. The 3-dimensional coordinates for this model are in Table 4.

Enrichment Model: Example 2: PTP1B

The PTP1B Enrichment module contains the residues: L₁₁₀, Y₁₇₆, T₁₇₈, W₁₇₉, V₁₈₄, P₁₈₅, E₁₈₆, S₁₈₇, S₁₉₀, F₁₉₁, R₂₂₁, T₂₂₄, D₂₆₅, Q₂₆₆, R₂₆₈, F₂₆₉, L₂₇₂.

Construction of the Enrichment Model for PTP1B.
1. The WPD loop residues THR177 to PRO188 were selected.
2. A conformational search of the Enrichment Model was employed.
3. Force field calculations were set to disregard atoms distant from center of the Enrichment Model.
4. Molecular Dynamic calculations were accelerated by fixing the coordinates of atoms near the active zone used for conformational search.
5. Enrichment Model coordinates were saved in a data base and checked for the ability of PTP1B Modulators to bind using Binding Site Identification tools.
6. Sites were checked for size and polarity giving preference to more hydrophobic rather than hydrophilic sites.
7. Enrichment Models with at least two aromatic hydrophobic residues and several polar side chains were selected.
8. The Enrichment Model contains four aromatic hydrophobic residues: TYR176 TRP179 PHE191 and PHE269.
9. This model corresponds to a super-open conformation of the WPD-loop. The 3-dimensional coordinates for this model are in Table 5.

Enrichment Model: Example 3: STEP

The STEP Enrichment model contains the residues: I₃₇₄, N₃₇₆, F₄₃₂, S₄₃₄, W₄₃₅, P₄₃₆, D₄₃₇, Q₄₃₈, K₄₃₉, P₄₄₁, D₄₄₂, R₄₄₃, P₄₄₅, P₄₄₆, L₄₄₇, R₄₇₈, C₄₈₁, F₄₈₂, T₅₁₇, E₅₁₉, Q₅₂₀, Q₅₂₂, F₅₂₃, H₅₂₆.

Construction of the Enrichment Model for STEP
1. The Enrichment Model for STEP
2. The WPD loop residues THR433 to ASP422 were selected.
3. A conformational search of the Enrichment Model was employed.
4. Force field calculations were set to disregard atoms distant from center of the Enrichment Model were utilized.
5. Molecular Dynamic calculations were accelerated by fixing the coordinates of atoms near the active zone used for the conformational search.
6. The Enrichment Model coordinates were saved in a data base and checked for the ability of Modulators to bind using Binding Site Identification tools.
7. The Sites were checked for size and polarity giving preference to more hydrophobic rather than hydrophilic sites.
8. The Enrichment Models with at least two aromatic hydrophobic residues and several polar side chains were selected.
9. The Enrichment Model contains four aromatic hydrophobic residues: PHE432, TRP435, PHE482 and PHE423.
10. This model corresponds to a super-open conformation of the WPD-loop. The 3-dimensional coordinates for this model are in Table 6.

Enrichment Model 4: LYP

The LYP (PTPN22, PEP, PTPN8) Enrichment model contains the residues: Y₁₉₀, K₁₉₁, W₁₉₃, D₁₉₇, V₁₉₈, P₁₉₉, S₂₀₁, I₂₀₂, I₂₀₅, G₂₃₂, R₂₃₃, V₂₃₆, I₂₃₇, T₂₇₅, E₂₇₇, Q₂₇₈, E₂₈₀, L₂₈₁, N₂₈₄

Construction of the Enrichment Model for LYP
1. The TYR190 to PRO199 WPD loop residues were selected.
2. A conformational search to generate the Enrichment Model was employed.
3. Force field calculations were set to disregard atoms distant from center of the Enrichment Model.
4. Molecular Dynamic calculations were accelerated by fixing the coordinates of atoms near the active zone used for the conformational search.
5. Enrichment Model coordinates were saved in a data base and checked for the ability of PTP1B Modulators to bind using Binding Site Identification tools.
6. Sites were checked for size and polarity giving preference to more hydrophobic rather than hydrophilic sites.
7. Enrichment Models with at least two aromatic hydrophobic residues and several polar side chains were selected.
8. The Enrichment Model contains two aromatic hydrophobic residues: TYR190 and TRP193. This model corresponds to a super-open conformation of the WPD-loop. The 3-dimensional coordinates for this model are in Table 7.

The data in each of Tables 4-7 is set forth in columns 1-11, where: Column 1: each line or record begins with the record type ATOM; column 2: atom serial number; column 3: atom name, which consists of the chemical symbol for the atom type. All the atom names beginning with C are carbon atoms; N indicates a nitrogen and O indicates oxygen. In amino acid residues, the next character is the remoteness indicator code, which is transliterated according to:
α A
β B
γ G
δ D
ϵ E
ζ Z
η H
   Column 4: amino acid residue type; column 5: residue sequence number; columns 6-8: X, Y, and Z coordinate values, respectively; column 9: occupancy; column 10: temperature factor; and column 11: Element symbol. Further details are available at the wwpdb.org/documentation/format33/sect9.html#ATOM.

TABLE 4
Coordinates of the Enrichment Model Example 1: PTP-PEST (PTPN12, PTPG1)

| 1 | 2 | 3 | 4 | 5 | 6 | 7 | 8 | 9 | 10 | 11 |
|---|---|---|---|---|---|---|---|---|---|---|
| ATOM | 1 | N | ALA | 132 | 3.193 | 22.380 | 75.140 | 1.00 | 0.00 | N |
| ATOM | 2 | CA | ALA | 132 | 3.514 | 23.736 | 74.720 | 1.00 | 0.00 | C |
| ATOM | 3 | C | ALA | 132 | 3.198 | 24.713 | 75.860 | 1.00 | 0.00 | C |
| ATOM | 4 | O | ALA | 132 | 2.390 | 25.637 | 75.753 | 1.00 | 0.00 | O |
| ATOM | 5 | CB | ALA | 132 | 2.754 | 24.082 | 73.450 | 1.00 | 0.00 | C |
| ATOM | 6 | N | TYR | 194 | 8.088 | 21.845 | 74.739 | 1.00 | 0.00 | N |
| ATOM | 7 | CA | TYR | 194 | 8.432 | 23.013 | 73.957 | 1.00 | 0.00 | C |
| ATOM | 8 | C | TYR | 194 | 8.177 | 24.268 | 74.781 | 1.00 | 0.00 | C |
| ATOM | 9 | O | TYR | 194 | 7.081 | 24.459 | 75.323 | 1.00 | 0.00 | O |
| ATOM | 10 | CB | TYR | 194 | 7.772 | 23.001 | 72.571 | 1.00 | 0.00 | C |
| ATOM | 11 | CG | TYR | 194 | 8.065 | 24.275 | 71.805 | 1.00 | 0.00 | C |
| ATOM | 12 | CD1 | TYR | 194 | 7.286 | 25.430 | 72.025 | 1.00 | 0.00 | C |
| ATOM | 13 | CD2 | TYR | 194 | 9.223 | 24.343 | 71.006 | 1.00 | 0.00 | C |
| ATOM | 14 | CE1 | TYR | 194 | 7.712 | 26.668 | 71.508 | 1.00 | 0.00 | C |
| ATOM | 15 | CE2 | TYR | 194 | 9.648 | 25.577 | 70.485 | 1.00 | 0.00 | C |
| ATOM | 16 | CZ | TYR | 194 | 8.907 | 26.744 | 70.759 | 1.00 | 0.00 | C |
| ATOM | 17 | OH | TYR | 194 | 9.408 | 27.950 | 70.392 | 1.00 | 0.00 | O |
| ATOM | 18 | N | ASN | 196 | 10.350 | 27.553 | 75.103 | 1.00 | 0.00 | N |
| ATOM | 19 | CA | ASN | 196 | 11.213 | 28.720 | 74.970 | 1.00 | 0.00 | C |
| ATOM | 20 | C | ASN | 196 | 11.130 | 29.309 | 73.537 | 1.00 | 0.00 | C |
| ATOM | 21 | O | ASN | 196 | 10.519 | 28.722 | 72.634 | 1.00 | 0.00 | O |
| ATOM | 22 | CB | ASN | 196 | 12.622 | 28.223 | 75.369 | 1.00 | 0.00 | C |
| ATOM | 23 | CG | ASN | 196 | 13.556 | 29.332 | 75.801 | 1.00 | 0.00 | C |
| ATOM | 24 | OD1 | ASN | 196 | 13.578 | 29.713 | 76.965 | 1.00 | 0.00 | O |
| ATOM | 25 | ND2 | ASN | 196 | 14.361 | 29.815 | 74.882 | 1.00 | 0.00 | N |
| ATOM | 26 | N | TRP | 197 | 11.722 | 30.494 | 73.355 | 1.00 | 0.00 | N |
| ATOM | 27 | CA | TRP | 197 | 11.927 | 31.147 | 72.061 | 1.00 | 0.00 | C |
| ATOM | 28 | C | TRP | 197 | 13.202 | 32.017 | 72.166 | 1.00 | 0.00 | C |
| ATOM | 29 | O | TRP | 197 | 13.589 | 32.397 | 73.278 | 1.00 | 0.00 | O |
| ATOM | 30 | CB | TRP | 197 | 10.679 | 31.985 | 71.714 | 1.00 | 0.00 | C |
| ATOM | 31 | CG | TRP | 197 | 10.476 | 33.222 | 72.543 | 1.00 | 0.00 | C |
| ATOM | 32 | CD1 | TRP | 197 | 11.016 | 34.434 | 72.266 | 1.00 | 0.00 | C |
| ATOM | 33 | CD2 | TRP | 197 | 9.985 | 33.327 | 73.919 | 1.00 | 0.00 | C |
| ATOM | 34 | NE1 | TRP | 197 | 10.829 | 35.289 | 73.330 | 1.00 | 0.00 | N |
| ATOM | 35 | CE2 | TRP | 197 | 10.235 | 34.650 | 74.394 | 1.00 | 0.00 | C |
| ATOM | 36 | CE3 | TRP | 197 | 9.369 | 32.431 | 74.823 | 1.00 | 0.00 | C |
| ATOM | 37 | CZ2 | TRP | 197 | 9.914 | 35.058 | 75.697 | 1.00 | 0.00 | C |
| ATOM | 38 | CZ3 | TRP | 197 | 9.036 | 32.831 | 76.134 | 1.00 | 0.00 | C |
| ATOM | 39 | CH2 | TRP | 197 | 9.310 | 34.139 | 76.573 | 1.00 | 0.00 | C |
| ATOM | 40 | N | HIS | 200 | 13.589 | 37.855 | 71.449 | 1.00 | 0.00 | N |
| ATOM | 41 | CA | HIS | 200 | 12.728 | 38.776 | 70.696 | 1.00 | 0.00 | C |
| ATOM | 42 | C | HIS | 200 | 13.122 | 38.760 | 69.207 | 1.00 | 0.00 | C |
| ATOM | 43 | O | HIS | 200 | 14.293 | 38.560 | 68.886 | 1.00 | 0.00 | O |
| ATOM | 44 | CB | HIS | 200 | 12.832 | 40.199 | 71.269 | 1.00 | 0.00 | C |
| ATOM | 45 | CG | HIS | 200 | 12.094 | 41.195 | 70.410 | 1.00 | 0.00 | C |
| ATOM | 46 | ND1 | HIS | 200 | 10.719 | 41.173 | 70.174 | 1.00 | 0.00 | N |
| ATOM | 47 | CD2 | HIS | 200 | 12.658 | 42.204 | 69.683 | 1.00 | 0.00 | C |
| ATOM | 48 | CE1 | HIS | 200 | 10.491 | 42.146 | 69.279 | 1.00 | 0.00 | C |
| ATOM | 49 | NE2 | HIS | 200 | 11.634 | 42.782 | 68.967 | 1.00 | 0.00 | N |
| ATOM | 50 | N | ASP | 201 | 12.156 | 38.976 | 68.302 | 1.00 | 0.00 | N |
| ATOM | 51 | CA | ASP | 201 | 12.278 | 38.631 | 66.863 | 1.00 | 0.00 | C |
| ATOM | 52 | C | ASP | 201 | 11.283 | 39.369 | 65.947 | 1.00 | 0.00 | C |
| ATOM | 53 | O | ASP | 201 | 10.382 | 40.059 | 66.426 | 1.00 | 0.00 | O |
| ATOM | 54 | CB | ASP | 201 | 12.156 | 37.098 | 66.804 | 1.00 | 0.00 | C |
| ATOM | 55 | CG | ASP | 201 | 12.080 | 36.567 | 65.384 | 1.00 | 0.00 | C |
| ATOM | 56 | OD1 | ASP | 201 | 12.979 | 36.929 | 64.596 | 1.00 | 0.00 | O |
| ATOM | 57 | OD2 | ASP | 201 | 11.077 | 35.885 | 65.071 | 1.00 | 0.00 | O1− |
| ATOM | 58 | N | VAL | 202 | 11.397 | 39.172 | 64.619 | 1.00 | 0.00 | N |
| ATOM | 59 | CA | VAL | 202 | 10.304 | 39.383 | 63.648 | 1.00 | 0.00 | C |
| ATOM | 60 | C | VAL | 202 | 9.991 | 38.182 | 62.724 | 1.00 | 0.00 | C |
| ATOM | 61 | O | VAL | 202 | 8.805 | 37.984 | 62.441 | 1.00 | 0.00 | O |
| ATOM | 62 | CB | VAL | 202 | 10.362 | 40.740 | 62.899 | 1.00 | 0.00 | C |
| ATOM | 63 | CG1 | VAL | 202 | 8.949 | 41.259 | 62.593 | 1.00 | 0.00 | C |
| ATOM | 64 | CG2 | VAL | 202 | 11.128 | 41.850 | 63.635 | 1.00 | 0.00 | C |
| ATOM | 65 | N | SER | 205 | 12.463 | 33.502 | 64.861 | 1.00 | 0.00 | N |
| ATOM | 66 | CA | SER | 205 | 11.996 | 32.737 | 66.005 | 1.00 | 0.00 | C |
| ATOM | 67 | C | SER | 205 | 11.014 | 31.628 | 65.559 | 1.00 | 0.00 | C |
| ATOM | 68 | O | SER | 205 | 10.776 | 30.652 | 66.275 | 1.00 | 0.00 | O |
| ATOM | 69 | CB | SER | 205 | 11.366 | 33.643 | 67.065 | 1.00 | 0.00 | C |
| ATOM | 70 | OG | SER | 205 | 10.147 | 34.265 | 66.637 | 1.00 | 0.00 | O |
| ATOM | 71 | N | PHE | 206 | 10.368 | 31.825 | 64.345 | 1.00 | 0.00 | N |
| ATOM | 72 | CA | PHE | 206 | 9.278 | 30.938 | 63.912 | 1.00 | 0.00 | C |
| ATOM | 73 | C | PHE | 206 | 9.844 | 29.530 | 63.663 | 1.00 | 0.00 | C |
| ATOM | 74 | O | PHE | 206 | 9.198 | 28.506 | 63.882 | 1.00 | 0.00 | O |
| ATOM | 75 | CB | PHE | 206 | 8.605 | 31.402 | 62.608 | 1.00 | 0.00 | C |
| ATOM | 76 | CG | PHE | 206 | 7.999 | 32.787 | 62.564 | 1.00 | 0.00 | C |
| ATOM | 77 | CD1 | PHE | 206 | 7.666 | 33.521 | 63.706 | 1.00 | 0.00 | C |
| ATOM | 78 | CD2 | PHE | 206 | 7.793 | 33.379 | 61.305 | 1.00 | 0.00 | C |
| ATOM | 79 | CE1 | PHE | 206 | 7.152 | 34.816 | 63.596 | 1.00 | 0.00 | C |
| ATOM | 80 | CE2 | PHE | 206 | 7.307 | 34.680 | 61.196 | 1.00 | 0.00 | C |
| ATOM | 81 | CZ | PHE | 206 | 6.976 | 35.396 | 62.344 | 1.00 | 0.00 | C |
| ATOM | 82 | N | SER | 208 | 11.900 | 27.550 | 64.909 | 1.00 | 0.00 | N |
| ATOM | 83 | CA | SER | 208 | 12.038 | 26.513 | 65.911 | 1.00 | 0.00 | C |
| ATOM | 84 | C | SER | 208 | 10.725 | 25.748 | 66.091 | 1.00 | 0.00 | C |
| ATOM | 85 | O | SER | 208 | 10.734 | 24.650 | 66.662 | 1.00 | 0.00 | O |
| ATOM | 86 | CB | SER | 208 | 12.473 | 26.991 | 67.295 | 1.00 | 0.00 | C |
| ATOM | 87 | OG | SER | 208 | 12.812 | 25.860 | 68.114 | 1.00 | 0.00 | O |
| ATOM | 88 | N | ILE | 209 | 9.555 | 26.375 | 65.698 | 1.00 | 0.00 | N |
| ATOM | 89 | CA | ILE | 209 | 8.291 | 25.657 | 65.863 | 1.00 | 0.00 | C |
| ATOM | 90 | C | ILE | 209 | 8.253 | 24.554 | 64.785 | 1.00 | 0.00 | C |
| ATOM | 91 | O | ILE | 209 | 7.705 | 23.466 | 64.960 | 1.00 | 0.00 | O |
| ATOM | 92 | CB | ILE | 209 | 7.059 | 26.597 | 65.880 | 1.00 | 0.00 | C |
| ATOM | 93 | CG1 | ILE | 209 | 5.975 | 26.023 | 66.823 | 1.00 | 0.00 | C |
| ATOM | 94 | CG2 | ILE | 209 | 6.460 | 26.879 | 64.498 | 1.00 | 0.00 | C |
| ATOM | 95 | CD1 | ILE | 209 | 4.831 | 26.991 | 67.096 | 1.00 | 0.00 | C |
| ATOM | 96 | N | GLY | 236 | −5.238 | 28.553 | 72.228 | 1.00 | 0.00 | N |
| ATOM | 97 | CA | GLY | 236 | −4.289 | 29.550 | 71.781 | 1.00 | 0.00 | C |
| ATOM | 98 | C | GLY | 236 | −3.020 | 28.944 | 71.210 | 1.00 | 0.00 | C |
| ATOM | 99 | O | GLY | 236 | −2.775 | 28.860 | 70.007 | 1.00 | 0.00 | O |
| ATOM | 100 | N | ARG | 237 | −2.166 | 28.443 | 72.180 | 1.00 | 0.00 | N |
| ATOM | 101 | CA | ARG | 237 | −0.862 | 27.883 | 71.818 | 1.00 | 0.00 | C |
| ATOM | 102 | C | ARG | 237 | −1.088 | 26.517 | 71.129 | 1.00 | 0.00 | C |
| ATOM | 103 | O | ARG | 237 | −0.259 | 26.010 | 70.374 | 1.00 | 0.00 | O |
| ATOM | 104 | CB | ARG | 237 | 0.071 | 27.725 | 73.037 | 1.00 | 0.00 | C |
| ATOM | 105 | CG | ARG | 237 | 0.711 | 29.051 | 73.484 | 1.00 | 0.00 | C |
| ATOM | 106 | CD | ARG | 237 | 1.763 | 23.885 | 74.600 | 1.00 | 0.00 | C |
| ATOM | 107 | NE | ARG | 237 | 1.097 | 28.731 | 75.911 | 1.00 | 0.00 | N |
| ATOM | 108 | CZ | ARG | 237 | 1.704 | 28.770 | 77.139 | 1.00 | 0.00 | C |
| ATOM | 109 | NH1 | ARG | 237 | 3.044 | 28.794 | 77.298 | 1.00 | 0.00 | N |
| ATOM | 110 | NH2 | ARG | 237 | 0.900 | 28.751 | 78.226 | 1.00 | 0.00 | N1+ |
| ATOM | 111 | N | ALA | 240 | −2.491 | 26.786 | 67.450 | 1.00 | 0.00 | N |
| ATOM | 112 | CA | ALA | 240 | −1.460 | 27.386 | 66.627 | 1.00 | 0.00 | C |
| ATOM | 113 | C | ALA | 240 | −0.534 | 26.290 | 66.093 | 1.00 | 0.00 | C |
| ATOM | 114 | O | ALA | 240 | −0.024 | 26.357 | 64.978 | 1.00 | 0.00 | O |
| ATOM | 115 | CB | ALA | 240 | −0.662 | 28.426 | 67.393 | 1.00 | 0.00 | C |
| ATOM | 116 | N | ILE | 241 | −0.269 | 25.261 | 66.976 | 1.00 | 0.00 | N |
| ATOM | 117 | CA | ILE | 241 | 0.544 | 24.096 | 66.599 | 1.00 | 0.00 | C |
| ATOM | 118 | C | ILE | 241 | −0.317 | 23.140 | 65.748 | 1.00 | 0.00 | C |
| ATOM | 119 | O | ILE | 241 | 0.160 | 22.523 | 64.785 | 1.00 | 0.00 | O |
| ATOM | 120 | CB | ILE | 241 | 1.118 | 23.383 | 67.848 | 1.00 | 0.00 | C |
| ATOM | 121 | CG1 | ILE | 241 | 2.178 | 24.282 | 68.517 | 1.00 | 0.00 | C |
| ATOM | 122 | CG2 | ILE | 241 | 1.732 | 22.017 | 67.512 | 1.00 | 0.00 | C |
| ATOM | 123 | CD1 | ILE | 241 | 2.643 | 23.764 | 69.868 | 1.00 | 0.00 | C |
| ATOM | 124 | N | GLU | 281 | −2.258 | 37.792 | 67.828 | 1.00 | 0.00 | N |
| ATOM | 125 | CA | GLU | 281 | −0.821 | 37.520 | 67.697 | 1.00 | 0.00 | C |
| ATOM | 126 | C | GLU | 281 | −0.532 | 36.050 | 67.301 | 1.00 | 0.00 | C |
| ATOM | 127 | O | GLU | 281 | 0.430 | 35.738 | 66.597 | 1.00 | 0.00 | O |
| ATOM | 128 | CB | GLU | 281 | −0.067 | 37.923 | 68.979 | 1.00 | 0.00 | C |
| ATOM | 129 | CG | GLU | 281 | 0.267 | 39.422 | 69.039 | 1.00 | 0.00 | C |
| ATOM | 130 | CD | GLU | 281 | −0.911 | 40.368 | 68.811 | 1.00 | 0.00 | C |
| ATOM | 131 | OE1 | GLU | 281 | −2.049 | 39.980 | 69.200 | 1.00 | 0.00 | O |
| ATOM | 132 | OE2 | GLU | 281 | −0.615 | 41.389 | 68.127 | 1.00 | 0.00 | O1− |
| ATOM | 133 | N | GLN | 282 | −1.395 | 35.103 | 67.819 | 1.00 | 0.00 | N |
| ATOM | 134 | CA | GLN | 282 | −1.281 | 33.675 | 67.498 | 1.00 | 0.00 | C |
| ATOM | 135 | C | GLN | 282 | −1.691 | 33.428 | 66.027 | 1.00 | 0.00 | C |
| ATOM | 136 | O | GLN | 282 | −1.076 | 32.642 | 65.304 | 1.00 | 0.00 | O |
| ATOM | 137 | CB | GLN | 282 | −2.161 | 32.810 | 68.407 | 1.00 | 0.00 | C |
| ATOM | 138 | CG | GLN | 282 | −1.685 | 32.762 | 69.861 | 1.00 | 0.00 | C |
| ATOM | 139 | CD | GLN | 282 | −2.835 | 32.561 | 70.835 | 1.00 | 0.00 | C |
| ATOM | 140 | OE1 | GLN | 282 | −4.017 | 32.485 | 70.504 | 1.00 | 0.00 | O |
| ATOM | 141 | NE2 | GLN | 282 | −2.449 | 32.529 | 72.152 | 1.00 | 0.00 | N |
| ATOM | 142 | N | GLU | 284 | −1.621 | 35.757 | 63.756 | 1.00 | 0.00 | N |
| ATOM | 143 | CA | GLU | 284 | −0.573 | 36.492 | 63.057 | 1.00 | 0.00 | C |
| ATOM | 144 | C | GLU | 284 | 0.707 | 35.652 | 62.901 | 1.00 | 0.00 | C |
| ATOM | 145 | O | GLU | 284 | 1.485 | 35.844 | 61.959 | 1.00 | 0.00 | O |
| ATOM | 146 | CB | GLU | 284 | −0.212 | 37.766 | 63.835 | 1.00 | 0.00 | C |
| ATOM | 147 | CG | GLU | 284 | −0.924 | 39.010 | 63.304 | 1.00 | 0.00 | C |
| ATOM | 148 | CD | GLU | 284 | −0.027 | 40.237 | 63.475 | 1.00 | 0.00 | C |
| ATOM | 149 | OE1 | GLU | 284 | 1.081 | 40.201 | 62.823 | 1.00 | 0.00 | O |
| ATOM | 150 | OE2 | GLU | 284 | −0.455 | 41.122 | 64.282 | l.00 | 0.00 | C1− |
| ATOM | 151 | N | LEU | 285 | 0.962 | 34.803 | 63.955 | 1.00 | 0.00 | N |
| ATOM | 152 | CA | LEU | 285 | 2.056 | 33.838 | 63.952 | 1.00 | 0.00 | C |
| ATOM | 153 | C | LEU | 285 | 1.728 | 32.740 | 62.926 | 1.00 | 0.00 | C |
| ATOM | 154 | O | LEU | 285 | 2.530 | 32.457 | 62.031 | 1.00 | 0.00 | O |
| ATOM | 155 | CB | LEU | 285 | 2.293 | 33.262 | 65.354 | 1.00 | 0.00 | C |
| ATOM | 156 | CG | LEU | 285 | 3.280 | 32.082 | 65.433 | 1.00 | 0.00 | C |
| ATOM | 157 | CD1 | LEU | 285 | 4.663 | 32.454 | 64.911 | 1.00 | 0.00 | C |
| ATOM | 158 | CD2 | LEU | 285 | 3.377 | 31.592 | 66.879 | 1.00 | 0.00 | C |
| ATOM | 159 | N | ARG | 288 | 1.990 | 33.627 | 99.228 | 1.00 | 0.00 | N |
| ATOM | 160 | CA | ARG | 288 | 3.359 | 33.855 | 58.788 | 1.00 | 0.00 | C |
| ATOM | 161 | C | ARG | 288 | 4.168 | 32.543 | 58.826 | 1.00 | 0.00 | C |
| ATOM | 162 | O | ARG | 288 | 5.082 | 32.337 | 58.026 | 1.00 | 0.00 | O |
| ATOM | 163 | CB | ARG | 288 | 4.073 | 34.918 | 59.626 | 1.00 | 0.00 | C |
| ATOM | 164 | CG | ARG | 288 | 3.581 | 36.354 | 59.394 | 1.00 | 0.00 | C |
| ATOM | 165 | CD | ARG | 288 | 4.400 | 37.324 | 60.248 | 1.00 | 0.00 | C |
| ATOM | 166 | NE | ARG | 288 | 3.937 | 38.717 | 60.196 | 1.00 | 0.00 | N |
| ATOM | 167 | CZ | ARG | 238 | 2.939 | 39.184 | 60.968 | 1.00 | 0.00 | C |
| ATOM | 168 | NH1 | ARG | 288 | 2.749 | 40.513 | 61.144 | 1.00 | 0.00 | N |
| ATOM | 169 | NH2 | ARG | 288 | 2.098 | 38.372 | 61.620 | 1.00 | 0.00 | N1+ |
| TER | 170 | | ARG | 288 | | | | | | |
| END | | | | | | | | | | |

TABLE 5
Coordinates of the Enrichment Model Example 2: PTP1B

| | | | | | | | | | | | |
|---|---|---|---|---|---|---|---|---|---|---|---|
| ATOM | 28 | N | LEU | A | 110 | 42.277 | 21.934 | 11.431 | 1.00 | 0.00 | N |
| ATOM | 29 | CA | LEU | A | 110 | 43.546 | 22.457 | 10.935 | 1.00 | 0.00 | C |
| ATOM | 30 | C | LEU | A | 110 | 43.557 | 22.741 | 9.425 | 1.00 | 0.00 | C |
| ATOM | 31 | O | LEU | A | 110 | 44.617 | 22.861 | 8.809 | 1.00 | 0.00 | O |
| ATOM | 32 | CB | LEU | A | 110 | 44.696 | 21.522 | 11.327 | 1.00 | 0.00 | C |
| ATOM | 33 | CG | LEU | A | 110 | 44.886 | 21.287 | 12.831 | 1.00 | 0.00 | C |
| ATOM | 34 | CD1 | LEU | A | 110 | 45.780 | 20.081 | 13.083 | 1.00 | 0.00 | C |
| ATOM | 35 | CD2 | LEU | A | 110 | 45.451 | 22.528 | 13.515 | 1.00 | 0.00 | C |
| ATOM | 83 | CA | TYR | A | 176 | 43.454 | 27.109 | 12.561 | 1.00 | 0.00 | C |
| ATOM | 84 | C | TYR | A | 176 | 43.264 | 27.386 | 11.076 | 1.00 | 0.00 | C |
| ATOM | 85 | O | TYR | A | 176 | 43.211 | 26.464 | 10.267 | 1.00 | 0.00 | O |
| ATOM | 86 | CB | TYR | A | 176 | 44.637 | 26.173 | 12.775 | 1.00 | 0.00 | C |
| ATOM | 87 | CG | TYR | A | 176 | 45.989 | 26.811 | 12.569 | 1.00 | 0.00 | C |
| ATOM | 88 | CD1 | TYR | A | 176 | 46.708 | 26.592 | 11.399 | 1.00 | 0.00 | C |
| ATOM | 89 | CD2 | TYR | A | 176 | 46.562 | 27.614 | 13.557 | 1.00 | 0.00 | C |
| ATOM | 90 | CE1 | TYR | A | 176 | 47.961 | 27.163 | 11.210 | 1.00 | 0.00 | C |
| ATOM | 91 | CE2 | TYR | A | 176 | 47.810 | 28.192 | 13.377 | 1.00 | 0.00 | C |
| ATOM | 92 | CZ | TYR | A | 176 | 48.504 | 27.962 | 12.202 | 1.00 | 0.00 | C |
| ATOM | 93 | OH | TYR | A | 176 | 49.740 | 28.536 | 12.020 | 1.00 | 0.00 | O |
| ATOM | 101 | N | THR | A | 178 | 44.852 | 28.868 | 8.179 | 1.00 | 0.00 | N |
| ATOM | 102 | CA | THR | A | 178 | 45.879 | 29.229 | 7.174 | 1.00 | 0.00 | C |
| ATOM | 103 | C | THR | A | 178 | 46.313 | 28.037 | 6.305 | 1.00 | 0.00 | C |
| ATOM | 104 | O | THR | A | 178 | 45.429 | 27.354 | 5.793 | 1.00 | 0.00 | O |
| ATOM | 105 | CE | THR | A | 178 | 47.035 | 30.060 | 7.761 | 1.00 | 0.00 | C |
| ATOM | 106 | OG1 | THR | A | 178 | 47.897 | 29.247 | 8.513 | 1.00 | 0.00 | O |
| ATOM | 107 | CG2 | THR | A | 178 | 46.563 | 31.227 | 8.630 | 1.00 | 0.00 | C |
| ATOM | 108 | N | TRP | A | 179 | 47.613 | 27.879 | 6.002 | 1.00 | 0.00 | N |
| ATOM | 109 | CA | TRP | A | 179 | 48.178 | 27.317 | 4.753 | 1.00 | 0.00 | C |
| ATOM | 110 | C | TRP | A | 179 | 47.623 | 28.072 | 3.519 | 1.00 | 0.00 | C |
| ATOM | 111 | O | TRP | A | 179 | 46.810 | 27.528 | 2.773 | 1.00 | 0.00 | O |
| ATOM | 112 | CB | TRP | A | 179 | 47.995 | 25.784 | 4.693 | 1.00 | 0.00 | C |
| ATOM | 113 | CG | TRP | A | 179 | 48.710 | 25.068 | 3.573 | 1.00 | 0.00 | C |
| ATOM | 114 | CD1 | TRP | A | 179 | 48.266 | 24.940 | 2.297 | 1.00 | 0.00 | C |
| ATOM | 115 | CD2 | TRP | A | 179 | 50.103 | 24.614 | 3.533 | 1.00 | 0.00 | C |
| ATOM | 116 | NE1 | TRP | A | 179 | 49.241 | 24.378 | 1.503 | 1.00 | 0.00 | N |
| ATOM | 117 | CE2 | TRP | A | 179 | 50.398 | 24.147 | 2.214 | 1.00 | 0.00 | C |
| ATOM | 118 | CE3 | TRP | A | 179 | 51.144 | 24.534 | 4.485 | 1.00 | 0.00 | C |
| ATOM | 119 | CZ2 | TRP | A | 179 | 51.646 | 23.613 | 1.863 | 1.00 | 0.00 | C |
| ATOM | 120 | CZ3 | TRP | A | 179 | 52.405 | 24.011 | 4.142 | 1.00 | 0.00 | C |
| ATOM | 121 | CH2 | TRP | A | 179 | 52.655 | 23.541 | 2.840 | 1.00 | 0.00 | C |
| ATOM | 130 | N | VAL | A | 184 | 52.759 | 30.348 | 1.358 | 1.00 | 0.00 | N |
| ATOM | 131 | CA | VAL | A | 184 | 53.060 | 29.127 | 2.135 | 1.00 | 0.00 | C |
| ATOM | 132 | C | VAL | A | 184 | 53.825 | 29.362 | 3.448 | 1.00 | 0.00 | O |
| ATOM | 133 | O | VAL | A | 184 | 53.389 | 28.824 | 4.465 | 1.00 | 0.00 | O |
| ATOM | 134 | CB | VAL | A | 184 | 53.695 | 28.000 | 1.278 | 1.00 | 0.00 | C |
| ATOM | 135 | CG1 | VAL | A | 184 | 53.611 | 26.648 | 1.997 | 1.00 | 0.00 | C |
| ATOM | 136 | CG2 | VAL | A | 184 | 53.045 | 27.844 | −0.108 | 1.00 | 0.00 | C |
| ATOM | 137 | N | PRO | A | 185 | 54.905 | 30.172 | 3.600 | 1.00 | 0.00 | N |
| ATOM | 138 | CA | PRO | A | 185 | 55.710 | 30.295 | 4.716 | 1.00 | 0.00 | C |
| ATOM | 139 | C | PRO | A | 185 | 55.037 | 31.145 | 5.805 | 1.00 | 0.00 | C |
| ATOM | 140 | O | PRO | A | 185 | 54.792 | 32.335 | 5.616 | 1.00 | 0.00 | O |
| ATOM | 141 | CB | PRO | A | 185 | 57.065 | 30.877 | 4.282. | 1.00 | 0.00 | C |
| ATOM | 142 | CG | PRO | A | 185 | 57.112 | 30.613 | 2.779 | 1.00 | 0.00 | C |
| ATOM | 143 | CD | PRO | A | 185 | 55.644 | 30.752 | 2.387 | 1.00 | 0.00 | C |
| ATOM | 144 | N | GLU | A | 186 | 54.840 | 30.541 | 6.541 | 1.00 | 0.00 | N |
| ATOM | 145 | CA | GLU | A | 186 | 54.615 | 31.187 | 8.280 | 1.00 | 0.00 | C |
| ATOM | 146 | C | GLU | A | 186 | 54.929 | 30.168 | 9.395 | 1.00 | 0.00 | C |
| ATOM | 147 | O | GLU | A | 186 | 55.220 | 29.006 | 9.094 | 1.00 | 0.00 | O |
| ATOM | 148 | CB | GLU | A | 186 | 53.175 | 31.731 | 8.410 | 1.00 | 0.00 | C |
| ATOM | 149 | CG | GLU | A | 186 | 52.064 | 30.660 | 8.422 | 1.00 | 0.00 | C |
| ATOM | 150 | CD | GLU | A | 186 | 50.920 | 30.957 | 9.404 | 1.00 | 0.00 | C |
| ATOM | 151 | OE1 | GLU | A | 186 | 51.170 | 31.440 | 10.535 | 1.00 | 0.00 | O |
| ATOM | 152 | OE2 | GLU | A | 186 | 49.764 | 30.666 | 9.022 | 1.00 | 0.00 | O1− |
| ATOM | 153 | N | SER | A | 187 | 54.766 | 30.570 | 10.667 | 1.00 | 0.00 | N |
| ATOM | 154 | CA | SER | A | 187 | 54.903 | 29.695 | 11.846 | 1.00 | 0.00 | C |
| ATOM | 155 | C | SER | A | 187 | 56.337 | 29.138 | 12.055 | 1.00 | 0.00 | C |
| ATOM | 156 | O | SER | A | 187 | 57.211 | 29.474 | 11.258 | 1.00 | 0.00 | O |
| ATOM | 157 | CB | SER | A | 187 | 53.799 | 28.634 | 11.766 | 1.00 | 0.00 | C |
| ATOM | 158 | OG | SER | A | 187 | 53.030 | 28.727 | 12.938 | 1.00 | 0.00 | O |
| ATOM | 159 | N | SER | A | 190 | 52.858 | 30.797 | 15.326 | 1.00 | 0.00 | N |
| ATOM | 160 | CA | SER | A | 190 | 51.403 | 30.867 | 15.328 | 1.00 | 0.00 | C |
| ATOM | 161 | C | SER | A | 190 | 50.746 | 29.521 | 15.633 | 1.00 | 0.00 | C |
| ATOM | 162 | O | SER | A | 190 | 49.717 | 29.471 | 16.315 | 1.00 | 0.00 | O |
| ATOM | 163 | CB | SER | A | 190 | 50.891 | 31.442 | 14.006 | 1.00 | 0.00 | C |
| ATOM | 164 | OG | SER | A | 190 | 51.218 | 30.596 | 12.920 | 1.00 | 0.00 | O |
| ATOM | 165 | N | PHE | A | 191 | 51.344 | 28.442 | 15.130 | 1.00 | 0.00 | N |
| ATOM | 166 | CA | PHE | A | 191 | 50.875 | 27.088 | 15.424 | 1.00 | 0.00 | C |
| ATOM | 167 | C | PHE | A | 191 | 50.986 | 26.776 | 16.923 | 1.00 | 0.00 | C |
| ATOM | 168 | O | PHE | A | 191 | 50.040 | 26.269 | 17.534 | 1.00 | 0.00 | O |
| ATOM | 169 | CB | PHE | A | 191 | 51.645 | 26.059 | 14.590 | 1.00 | 0.00 | C |
| ATOM | 170 | CG | PHE | A | 191 | 51.018 | 24.692 | 14.575 | 1.00 | 0.00 | C |
| ATOM | 171 | CD1 | PHE | A | 191 | 49.799 | 24.472 | 13.933 | 1.00 | 0.00 | C |
| ATOM | 172 | CD2 | PHE | A | 191 | 51.651 | 23.621 | 15.195 | 1.00 | 0.00 | C |
| ATOM | 173 | CE1 | PHE | A | 191 | 49.214 | 23.199 | 13.915 | 1.00 | 0.00 | C |
| ATOM | 174 | CE2 | PHE | A | 191 | 51.077 | 22.349 | 15.184 | 1.00 | 0.00 | C |
| ATOM | 175 | CZ | PHE | A | 191 | 49.854 | 22.139 | 14.543 | 1.00 | 0.00 | C |
| ATOM | 209 | N | ARG | A | 221 | 48.632 | 17.447 | 9.204 | 1.00 | 0.00 | N |
| ATOM | 210 | CA | ARG | A | 221 | 48.648 | 18.557 | 10.154 | 1.00 | 0.00 | C |
| ATOM | 211 | C | ARG | A | 221 | 43.191 | 18.090 | 11.531 | 1.00 | 0.00 | C |
| ATOM | 212 | O | ARG | A | 221 | 48.718 | 18.539 | 12.545 | 1.00 | 0.00 | O |
| ATOM | 213 | CB | ARG | A | 221 | 47.777 | 19.710 | 9.656 | 1.00 | 0.00 | C |
| ATOM | 214 | CG | ARG | A | 221 | 48.020 | 20.077 | 8.194 | 1.00 | 0.00 | C |
| ATOM | 215 | CD | ARG | A | 221 | 47.131 | 21.184 | 7.698 | 1.00 | 0.00 | C |
| ATOM | 216 | NE | ARG | A | 221 | 47.229 | 21.394 | 6.256 | 1.00 | 0.00 | N |
| ATOM | 217 | CZ | ARG | A | 221 | 46.379 | 22.141 | 5.555 | 1.00 | 0.00 | C |
| ATOM | 218 | NH1 | ARG | A | 221 | 45.366 | 22.746 | 6.157 | 1.00 | 0.00 | N |
| ATOM | 219 | NH2 | ARG | A | 221 | 46.535 | 22.285 | 4.247 | 1.00 | 0.00 | N1− |
| ATOM | 220 | N | THR | A | 224 | 51.089 | 16.613 | 13.399 | 1.00 | 0.00 | N |
| ATOM | 221 | CA | THR | A | 224 | 51.935 | 17.688 | 13.902 | 1.00 | 0.00 | C |
| ATOM | 222 | C | THR | A | 224 | 51.421 | 18.255 | 15.219 | 1.00 | 0.00 | C |
| ATOM | 223 | O | THR | A | 224 | 52.196 | 18.448 | 16.147 | 1.00 | 0.00 | O |
| ATOM | 224 | CB | THR | A | 224 | 52.063 | 18.797 | 12.855 | 1.00 | 0.00 | C |
| ATOM | 225 | OG1 | THR | A | 224 | 52.699 | 18.270 | 11.681 | 1.00 | 0.00 | O |
| ATOM | 226 | CG2 | THR | A | 224 | 53.034 | 19.852 | 13.327 | 1.00 | 0.00 | C |
| ATOM | 243 | N | ASP | A | 265 | 58.894 | 17.486 | 5.045 | 1.00 | 0.00 | N |
| ATOM | 244 | CA | ASP | A | 265 | 58.678 | 18.884 | 5.405 | 1.00 | 0.00 | C |
| ATOM | 245 | C | ASP | A | 265 | 57.891 | 19.008 | 6.708 | 1.00 | 0.00 | C |
| ATOM | 246 | O | ASP | A | 265 | 58.180 | 19.875 | 7.534 | 1.00 | 0.00 | O |
| ATOM | 247 | CB | ASP | A | 265 | 57.945 | 19.621 | 4.280 | 1.00 | 0.00 | C |
| ATOM | 248 | CG | ASP | A | 265 | 58.893 | 20.301 | 3.304 | 1.00 | 0.00 | C |
| ATOM | 249 | OD1 | ASP | A | 265 | 58.685 | 21.501 | 3.016 | 1.00 | 0.00 | O |
| ATOM | 250 | OD2 | ASP | A | 265 | 55.862 | 13.717 | 2.767 | 1.00 | 0.00 | O1− |
| ATOM | 251 | N | GLN | A | 266 | 56.904 | 18.134 | 6.886 | 1.00 | 0.00 | N |
| ATOM | 252 | CA | GLN | A | 266 | 56.088 | 18.127 | 8.096 | 1.00 | 0.00 | C |
| ATOM | 253 | C | GLN | A | 266 | 56.877 | 17.635 | 9.303 | 1.00 | 0.00 | C |
| ATOM | 254 | O | GLN | A | 266 | 56.711 | 18.153 | 10.416 | 1.00 | 0.00 | O |
| ATOM | 255 | CB | GLN | A | 266 | 54.838 | 17.275 | 7.899 | 1.00 | 0.00 | C |
| ATOM | 256 | CG | GLN | A | 266 | 53.670 | 18.062 | 7.353 | 1.00 | 0.00 | C |
| ATOM | 257 | CD | GLN | A | 266 | 52.504 | 17.184 | 6.956 | 1.00 | 0.00 | C |
| ATOM | 258 | OE1 | GLN | A | 266 | 52.454 | 16.006 | 7.307 | 1.00 | 0.00 | O |
| ATOM | 259 | NE2 | GLN | A | 266 | 51.556 | 17.760 | 6.226 | 1.00 | 0.00 | N |
| ATOM | 260 | N | ARG | A | 268 | 60.176 | 17.896 | 9.676 | 1.00 | 0.00 | N |
| ATOM | 261 | CA | ARG | A | 268 | 61.074 | 18.998 | 9.998 | 1.00 | 0.00 | C |
| ATOM | 262 | C | ARG | A | 268 | 60.357 | 20.073 | 10.811 | 1.00 | 0.00 | C |
| ATOM | 263 | O | ARG | A | 268 | 60.904 | 20.593 | 11.784 | 1.00 | 0.00 | O |
| ATOM | 264 | CB | ARG | A | 268 | 61.668 | 19.598 | 8.719 | 1.00 | 0.00 | C |
| ATOM | 265 | CG | ARG | A | 268 | 62.688 | 20.691 | 8.975 | 1.00 | 0.00 | C |
| ATOM | 266 | CD | ARG | A | 268 | 63.197 | 21.407 | 7.733 | 1.00 | 0.00 | C |
| ATOM | 267 | NE | ARG | A | 268 | 64.285 | 22.315 | 8.087 | 1.00 | 0.00 | N |
| ATOM | 268 | CZ | ARG | A | 268 | 64.118 | 23.551 | 8.549 | 1.00 | 0.00 | C |
| ATOM | 269 | NH1 | ARG | A | 268 | 62.902 | 24.058 | 8.706 | 1.00 | 0.00 | N |
| ATOM | 270 | NH2 | ARG | A | 268 | 65.176 | 24.287 | 8.855 | 1.00 | 0.00 | N1+ |
| ATOM | 271 | N | PHE | A | 269 | 59.129 | 20.389 | 10.410 | 1.00 | 0.00 | N |
| ATOM | 272 | CA | PHE | A | 269 | 58.336 | 21.415 | 11.078 | 1.00 | 0.00 | C |
| ATOM | 273 | C | PHE | A | 269 | 57.952 | 20.995 | 12.497 | 1.00 | 0.00 | C |
| ATOM | 274 | O | PHE | A | 269 | 57.929 | 21.818 | 13.414 | 1.00 | 0.00 | O |
| ATOM | 275 | CB | PHE | A | 269 | 57.085 | 21.731 | 10.263 | 1.00 | 0.00 | C |
| ATOM | 276 | CG | PHE | A | 269 | 56.194 | 22.754 | 10.895 | 1.00 | 0.00 | C |
| ATOM | 277 | CD1 | PHE | A | 269 | 54.984 | 22.380 | 11.464 | 1.00 | 0.00 | C |
| ATOM | 278 | CD2 | PHE | A | 269 | 56.563 | 24.092 | 10.920 | 1.00 | 0.00 | C |
| ATOM | 279 | CE1 | PHE | A | 269 | 54.159 | 23.326 | 12.046 | 1.00 | 0.00 | C |
| ATOM | 280 | CE2 | PHE | A | 269 | 55.743 | 25.038 | 11.496 | 1.00 | 0.00 | C |
| ATOM | 281 | CZ | PHE | A | 269 | 54.542 | 24.657 | 12.059 | 1.00 | 0.00 | C |
| ATOM | 282 | N | LEU | A | 272 | 60.897 | 21.554 | 14.640 | 1.00 | 0.00 | N |
| ATOM | 283 | CA | LEU | A | 272 | 60.944 | 22.999 | 14.836 | 1.00 | 0.00 | C |
| ATOM | 284 | C | LEU | A | 272 | 59.957 | 23.437 | 15.917 | 1.00 | 0.00 | C |
| ATOM | 285 | O | LEU | A | 272 | 60.329 | 24.142 | 16.856 | 1.00 | 0.00 | O |
| ATOM | 286 | CB | LEU | A | 272 | 60.659 | 23.732 | 13.516 | 1.00 | 0.00 | C |
| ATOM | 287 | CG | LEU | A | 272 | 61.618 | 24.805 | 12.969 | 1.00 | 0.00 | C |
| ATOM | 288 | CD1 | LEU | A | 272 | 60.891 | 25.712 | 11.977 | 1.00 | 0.00 | C |
| ATOM | 289 | CD2 | LEU | A | 272 | 62.282 | 25.646 | 14.059 | 1.00 | 0.00 | C |
| TER | 290 | | LEU | A | 272 | | | | | | |
| END | | | | | | | | | | | |

TABLE 6
Coordinates of the Enrichment Model Example 3: STEP

| | | | | | | | | | | | |
|---|---|---|---|---|---|---|---|---|---|---|---|
| ATOM | 1 | N | ILE | A | 374 | −4.195 | −9.881 | −14.424 | 1.00 | 0.00 | N |
| ATOM | 2 | CA | ILE | A | 374 | −5.213 | −10.652 | −15.129 | 1.00 | 0.00 | C |
| ATOM | 3 | C | ILE | A | 374 | −5.531 | −11.901 | −14.282 | 1.00 | 0.00 | C |
| ATOM | 4 | O | ILE | A | 374 | −5.115 | −13.004 | −14.581 | 1.00 | 0.00 | O |
| ATOM | 5 | CB | ILE | A | 374 | −4.759 | −11.034 | −16.558 | 1.00 | 0.00 | C |
| ATOM | 6 | CG1 | ILE | A | 374 | −4.154 | −9.824 | −17.296 | 1.00 | 0.00 | C |
| ATOM | 7 | CG2 | ILE | A | 374 | −5.916 | −11.574 | −17.369 | 1.00 | 0.00 | C |
| ATOM | 8 | CD1 | ILE | A | 374 | −3.334 | −10.188 | −18.507 | 1.00 | 0.00 | C |
| ATOM | 9 | N | ASN | A | 376 | −8.817 | −13.605 | −11.835 | 1.00 | 0.00 | N |
| ATOM | 10 | CA | ASN | A | 376 | −10.218 | −13.466 | −11.456 | 1.00 | 0.00 | C |
| ATOM | 11 | C | ASN | A | 376 | −10.303 | −13.424 | −9.934 | 1.00 | 0.00 | C |
| ATOM | 12 | O | ASN | A | 376 | −9.386 | −13.863 | −9.245 | 1.00 | 0.00 | O |
| ATOM | 13 | CB | ASN | A | 376 | −11.078 | −14.600 | −12.047 | 1.00 | 0.00 | C |
| ATOM | 14 | CG | ASN | A | 376 | −11.145 | −14.556 | −13.585 | 1.00 | 0.00 | C |
| ATOM | 15 | OD1 | ASN | A | 376 | −10.934 | −13.501 | −14.211 | 1.00 | 0.00 | O |
| ATOM | 16 | ND2 | ASN | A | 376 | −11.454 | −15.702 | −14.194 | 1.00 | 0.00 | N |
| ATOM | 17 | N | PHE | A | 432 | −8.138 | −6.863 | −14.161 | 1.00 | 0.00 | N |
| ATOM | 18 | CA | PHE | A | 432 | −9.091 | −7.454 | −15.062 | 1.00 | 0.00 | C |
| ATOM | 19 | C | PHE | A | 432 | −9.589 | −8.734 | −14.420 | 1.00 | 0.00 | C |
| ATOM | 20 | O | PHE | A | 432 | −8.801 | −9.624 | −14.070 | 1.00 | 0.00 | O |
| ATOM | 21 | CE | PHE | A | 432 | −8.399 | −7.757 | −16.383 | 1.00 | 0.00 | C |
| ATOM | 22 | CG | PHE | A | 432 | −9.288 | −8.315 | −17.435 | 1.00 | 0.00 | C |
| ATOM | 23 | CD1 | PHE | A | 432 | −9.284 | −9.671 | −17.709 | 1.00 | 0.00 | C |
| ATOM | 24 | CD2 | PHE | A | 432 | −10.107 | −7.475 | −18.189 | 1.00 | 0.00 | C |
| ATOM | 25 | CE1 | PHE | A | 432 | −10.081 | −10.194 | −18.702 | 1.00 | 0.00 | C |
| ATOM | 26 | CE2 | PHE | A | 432 | −10.901 | −7.980 | −19.191 | 1.00 | 0.00 | C |
| ATOM | 27 | CZ | PHE | A | 432 | −10.866 | −9.349 | −19.458 | 1.00 | 0.00 | C |
| ATOM | 28 | N | SER | A | 434 | −13.043 | −11.655 | −14.177 | 1.00 | 0.00 | N |
| ATOM | 29 | CA | SER | A | 434 | −14.045 | −12.405 | −14.943 | 1.00 | 0.00 | C |
| ATOM | 30 | C | SER | A | 434 | −14.404 | −13.736 | −14.247 | 1.00 | 0.00 | C |
| ATOM | 31 | O | SER | A | 434 | −14.023 | −13.978 | −13.103 | 1.00 | 0.00 | O |
| ATOM | 32 | CB | SER | A | 434 | −13.458 | −12.643 | −16.348 | 1.00 | 0.00 | C |
| ATOM | 33 | CG | SER | A | 434 | −12.423 | −13.611 | −16.323 | 1.00 | 0.00 | O |
| ATOM | 34 | N | TRP | A | 435 | −15.098 | −14.631 | −14.959 | 1.00 | 0.00 | N |
| ATOM | 35 | CA | TRP | A | 435 | −15.315 | −16.037 | −14.596 | 1.00 | 0.00 | C |
| ATOM | 36 | C | TRP | A | 435 | −15.512 | −16.857 | −15.896 | 1.00 | 0.00 | C |
| ATOM | 37 | O | TRP | A | 435 | −16.049 | −16.296 | −16.854 | 1.00 | 0.00 | O |
| ATOM | 38 | CB | TRP | A | 435 | −16.535 | −16.133 | −13.667 | 1.00 | 0.00 | C |
| ATOM | 39 | CG | TRP | A | 435 | −16.789 | −17.478 | −13.055 | 1.00 | 0.00 | C |
| ATOM | 40 | CD1 | TRP | A | 435 | −17.541 | −18.462 | −13.600 | 1.00 | 0.00 | C |
| ATOM | 41 | CD2 | TRP | A | 435 | −16.089 | −18.105 | −11.933 | 1.00 | 0.00 | C |
| ATOM | 42 | CE1 | TRP | A | 435 | −17.445 | −19.608 | −12.837 | 1.00 | 0.00 | N |
| ATOM | 43 | CE2 | TRP | A | 435 | −16.542 | −19.454 | −11.808 | 1.00 | 0.00 | C |
| ATOM | 44 | CE3 | TRP | A | 435 | −15.110 | −17.667 | −11.015 | 1.00 | 0.00 | C |
| ATOM | 45 | CZ2 | TRP | A | 435 | −16.060 | −20.320 | −10.815 | 1.00 | 0.00 | C |
| ATOM | 46 | CZ3 | TRP | A | 435 | −14.616 | −18.529 | −10.015 | 1.00 | 0.00 | C |
| ATOM | 47 | CH2 | TRP | A | 435 | −15.093 | −19.849 | −9.910 | 1.00 | 0.00 | C |
| ATOM | 48 | N | PRO | A | 436 | −15.073 | −18.129 | −16.005 | 1.00 | 0.00 | N |
| ATOM | 49 | CA | PRO | A | 436 | −15.198 | −18.919 | −17.241 | 1.00 | 0.00 | C |
| ATOM | 50 | C | PRO | A | 436 | −16.648 | −19.252 | −17.638 | 1.00 | 0.00 | C |
| ATOM | 51 | O | PRO | A | 436 | −17.524 | −19.331 | −16.780 | 1.00 | 0.00 | O |
| ATOM | 52 | CB | PRO | A | 436 | −14.397 | −20.205 | −16.998 | 1.00 | 0.00 | C |
| ATOM | 53 | CG | PRO | A | 436 | −13.421 | −19.823 | −15.888 | 1.00 | 0.00 | C |
| ATOM | 54 | CD | PRO | A | 436 | −14.246 | −18.853 | −15.048 | 1.00 | 0.00 | C |
| ATOM | 55 | N | ASP | A | 437 | −16.879 | −19.548 | −18.926 | 1.00 | 0.00 | N |
| ATOM | 56 | CA | ASP | A | 437 | −18.074 | −20.269 | −19.395 | 1.00 | 0.00 | C |
| ATOM | 57 | C | ASP | A | 437 | −17.714 | −20.894 | −20.767 | 1.00 | 0.00 | C |
| ATOM | 58 | O | ASP | A | 437 | −16.603 | −20.727 | −21.279 | 1.00 | 0.00 | O |
| ATOM | 59 | CB | ASP | A | 437 | −19.286 | −19.311 | −19.524 | 1.00 | 0.00 | C |
| ATOM | 60 | CG | ASP | A | 437 | −19.432 | −18.576 | −20.863 | 1.00 | 0.00 | C |
| ATOM | 61 | OD1 | ASP | A | 437 | −18.401 | −18.234 | −21.488 | 1.00 | 0.00 | O |
| ATOM | 62 | OD2 | ASP | A | 437 | −20.588 | −18.413 | −21.310 | 1.00 | 0.00 | O1− |
| ATOM | 63 | N | GLN | A | 438 | −18.686 | −21.579 | −21.382 | 1.00 | 0.00 | N |
| ATOM | 64 | CA | GLN | A | 438 | −18.583 | −22.193 | −22.711 | 1.00 | 0.00 | C |
| ATOM | 65 | C | GLN | A | 438 | −18.781 | −21.192 | −23.881 | 1.00 | 0.00 | C |
| ATOM | 66 | O | GLN | A | 438 | −19.164 | −21.586 | −24.984 | 1.00 | 0.00 | O |
| ATOM | 67 | CG | GLN | A | 438 | −19.526 | −23.419 | −22.730 | 1.00 | 0.00 | C |
| ATOM | 68 | CG | GLN | A | 438 | −19.521 | −24.327 | −23.975 | 1.00 | 0.00 | C |
| ATOM | 69 | CD | GLN | A | 438 | −18.125 | −24.629 | −24.516 | 1.00 | 0.00 | C |
| ATOM | 70 | OE1 | GLN | A | 438 | −17.430 | −25.498 | −24.019 | 1.00 | 0.00 | O |
| ATOM | 71 | NE2 | GLN | A | 438 | −17.579 | −23.786 | −25.373 | 1.00 | 0.00 | N |
| ATOM | 72 | N | LYS | A | 439 | −18.502 | −19.896 | −23.680 | 1.00 | 0.00 | N |
| ATOM | 73 | CA | LYS | A | 439 | −18.755 | −18.810 | −24.640 | 1.00 | 0.00 | C |
| ATOM | 74 | C | LYS | A | 439 | −20.241 | −18.676 | 25.008 | 1.00 | 0.00 | C |
| ATOM | 75 | O | LYS | A | 439 | −20.605 | −18.657 | −26.184 | 1.00 | 0.00 | O |
| ATOM | 76 | CB | LYS | A | 439 | −17.825 | −18.889 | −25.866 | 1.00 | 0.00 | C |
| ATOM | 77 | CG | LYS | A | 439 | −16.326 | −13.959 | −25.515 | 1.00 | 0.00 | C |
| ATOM | 78 | CD | LYS | A | 439 | −15.445 | −18.780 | −26.762 | 1.00 | 0.00 | C |
| ATOM | 79 | CE | LYS | A | 439 | −15.501 | −17.327 | −27.246 | 1.00 | 0.00 | C |
| ATOM | 80 | NZ | LYS | A | 439 | −15.069 | −17.170 | −28.649 | 1.00 | 0.00 | N1+ |
| ATOM | 81 | N | PRO | A | 441 | −22.183 | −15.978 | −24.229 | 1.00 | 0.00 | N |
| ATOM | 82 | CA | PRO | A | 441 | −21.821 | −14.882 | −25.118 | 1.00 | 0.00 | C |
| ATOM | 83 | C | PRO | A | 441 | −20.373 | −15.066 | −25.610 | 1.00 | 0.00 | C |
| ATOM | 84 | O | PRO | A | 441 | −19.578 | −15.780 | −24.992 | 1.00 | 0.00 | O |
| ATOM | 85 | CB | PRO | A | 441 | −21.974 | −13.635 | −24.246 | 1.00 | 0.00 | C |
| ATOM | 86 | CG | PRO | A | 441 | −21.471 | −14.126 | −22.886 | 1.00 | 0.00 | C |
| ATOM | 87 | CD | PRO | A | 441 | −21.854 | −15.610 | −22.858 | 1.00 | 0.00 | C |
| ATOM | 88 | N | ASP | A | 442 | −19.957 | −14.316 | −26.638 | 1.00 | 0.00 | N |
| ATOM | 89 | CA | ASP | A | 442 | −18.521 | −14.184 | −26.893 | 1.00 | 0.00 | C |
| ATOM | 90 | O | ASP | A | 442 | −17.822 | −13.329 | −25.814 | 1.00 | 0.00 | C |
| ATOM | 91 | O | ASP | A | 442 | −18.372 | −12.363 | −25.290 | 1.00 | 0.00 | O |
| ATOM | 92 | CB | ASP | A | 442 | −18.174 | −13.795 | −28.337 | 1.00 | 0.00 | C |
| ATOM | 93 | CG | ASP | A | 442 | −16.653 | −13.681 | −28.492 | 1.00 | 0.00 | C |
| ATOM | 94 | OD1 | ASP | A | 442 | −16.016 | −14.720 | −28.781 | 1.00 | 0.00 | O |
| ATOM | 95 | OD2 | ASP | A | 442 | −16.108 | −12.615 | −28.149 | 1.00 | 0.00 | O1− |
| ATOM | 96 | N | ARG | A | 443 | −16.615 | −13.760 | −25.448 | 1.00 | 0.00 | N |
| ATOM | 97 | CA | ARG | A | 443 | −15.816 | −13.316 | −24.288 | 1.00 | 0.00 | C |
| ATOM | 98 | C | ARG | A | 443 | −14.659 | −12.398 | −24.648 | 1.00 | 0.00 | C |
| ATOM | 99 | O | ARG | A | 443 | −13.894 | −11.983 | −23.764 | 1.00 | 0.00 | O |
| ATOM | 100 | CB | ARG | A | 443 | −15.275 | −14.535 | −23.532 | 1.00 | 0.00 | C |
| ATOM | 101 | CG | ARG | A | 443 | −16.339 | −15.613 | −23.233 | 1.00 | 0.00 | C |
| ATOM | 102 | CD | ARG | A | 443 | −17.527 | −15.092 | −22.405 | 1.00 | 0.00 | C |
| ATOM | 103 | NE | ARG | A | 443 | −17.127 | −14.544 | −21.110 | 1.00 | 0.00 | N |
| ATOM | 104 | C2 | ARG | A | 443 | −16.920 | −15.260 | −20.003 | 1.00 | 0.00 | C |
| ATOM | 105 | NH1 | ARG | A | 443 | −17.056 | −16.582 | −19.981 | 1.00 | 0.00 | N |
| ATOM | 106 | NH2 | ARG | A | 443 | −16.565 | −14.635 | −18.893 | 1.00 | 0.00 | N1+ |
| ATOM | 107 | N | PRO | A | 445 | −14.752 | −9.166 | −26.209 | 1.00 | 0.00 | N |
| ATOM | 108 | CA | PRO | A | 445 | −14.802 | −7.706 | −26.056 | 1.00 | 0.00 | C |
| ATOM | 109 | C | PRO | A | 445 | −14.157 | −7.076 | −24.798 | 1.00 | 0.00 | C |
| ATOM | 110 | O | PRO | A | 445 | −13.461 | −6.063 | −24.938 | 1.00 | 0.00 | O |
| ATOM | 111 | CB | PRO | A | 445 | −16.295 | −7.397 | −26.143 | 1.00 | 0.00 | C |
| ATOM | 112 | CG | PRO | A | 445 | −16.858 | −8.524 | −27.020 | 1.00 | 0.00 | C |
| ATOM | 113 | CD | PRO | A | 445 | −16.083 | −9.722 | −26.560 | 1.00 | 0.00 | C |
| ATOM | 114 | N | PRO | A | 446 | −14.371 | −7.648 | −23.592 | 1.00 | 0.00 | N |
| ATOM | 115 | CA | PRO | A | 446 | −13.713 | −7.052 | −22.414 | 1.00 | 0.00 | C |
| ATOM | 116 | C | PRO | A | 446 | −12.191 | −7.021 | −22.488 | 1.00 | 0.00 | C |
| ATOM | 117 | O | PRO | A | 446 | −11.585 | −6.044 | −22.030 | 1.00 | 0.00 | O |
| ATOM | 118 | CB | PRO | A | 446 | −14.154 | −7.962 | −21.270 | 1.00 | 0.00 | C |
| ATOM | 119 | CG | PRO | A | 446 | −15.496 | −8.476 | −21.731 | 1.00 | 0.00 | C |
| ATOM | 120 | CD | PRO | A | 446 | −15.255 | −8.755 | −23.190 | 1.00 | 0.00 | C |
| ATOM | 121 | N | LEU | A | 447 | −11.588 | −8.082 | −23.017 | 1.00 | 0.00 | N |
| ATOM | 122 | CA | LEU | A | 447 | −10.120 | −8.109 | −23.165 | 1.00 | 0.00 | C |
| ATOM | 123 | C | LEU | A | 447 | −9.656 | −7.046 | −24.133 | 1.00 | 0.00 | C |
| ATOM | 124 | O | LEU | A | 447 | −8.672 | −6.351 | −23.873 | 1.00 | 0.00 | O |
| ATOM | 125 | CB | LEU | A | 447 | −9.608 | −9.471 | −23.616 | 1.00 | 0.00 | C |
| ATOM | 126 | CG | LEU | A | 447 | −8.078 | −9.566 | −23.658 | 1.00 | 0.00 | C |
| ATOM | 127 | CD1 | LEU | A | 447 | −7.516 | −9.382 | −22.266 | 1.00 | 0.00 | C |
| ATOM | 128 | CD2 | LEU | A | 447 | −7.559 | −10.896 | −24.269 | 1.00 | 0.00 | C |
| ATOM | 129 | N | ARG | A | 478 | −2.065 | −15.925 | −19.842 | 1.00 | 0.00 | N |
| ATOM | 130 | CA | ARG | A | 478 | −2.905 | −14.760 | −19.971 | 1.00 | 0.00 | C |
| ATOM | 131 | C | ARG | A | 478 | −2.032 | −13.521 | −20.234 | 1.00 | 0.00 | C |
| ATOM | 132 | O | ARG | A | 478 | −2.380 | −12.658 | −21.022 | 1.00 | 0.00 | O |
| ATOM | 133 | CB | ARG | A | 478 | −3.713 | −14.552 | −18.711 | 1.00 | 0.00 | C |
| ATOM | 134 | CG | ARG | A | 478 | −4.575 | −15.745 | −18.341 | 1.00 | 0.00 | C |
| ATOM | 135 | CD | ARG | A | 478 | −5.350 | −15.459 | −17.103 | 1.00 | 0.00 | C |
| ATOM | 136 | NE | ARG | A | 478 | −6.041 | −16.660 | −16.674 | 1.00 | 0.00 | N |
| ATOM | 137 | CZ | ARG | A | 478 | −6.846 | −16.715 | −15.625 | l.00 | 0.00 | C |
| ATOM | 138 | NH1 | ARG | A | 478 | −7.089 | −15.621 | −14.896 | 1.00 | 0.00 | N |
| ATOM | 139 | NH2 | ARG | A | 478 | −7.429 | −17.876 | −15.331 | 1.00 | 0.00 | N1+ |
| ATOM | 140 | N | CYS | A | 481 | −1.179 | −13.480 | −23.821 | 1.00 | 0.00 | N |
| ATOM | 141 | CA | CYS | A | 481 | −2.377 | −13.111 | −24.590 | 1.00 | 0.00 | C |
| ATOM | 142 | C | CYS | A | 481 | −2.539 | −11.600 | −24.600 | 1.00 | 0.00 | C |
| ATOM | 143 | O | CVS | A | 481 | −2.787 | −10.996 | −25.646 | 1.00 | 0.00 | O |
| ATOM | 144 | CB | CYS | A | 481 | −3.622 | −13.732 | −24.015 | 1.00 | 0.00 | C |
| ATOM | 145 | SG | CYS | A | 481 | −3.709 | −15.489 | −24.307 | 1.00 | 0.00 | S |
| ATOM | 146 | N | PHE | A | 482 | −2.404 | −10.996 | −23.428 | 1.00 | 0.00 | N |
| ATOM | 147 | CA | PHE | A | 482 | −2.586 | −9.566 | −23.308 | 1.00 | 0.00 | C |
| ATOM | 148 | C | PHE | A | 482 | −1.532 | −8.807 | −24.125 | 1.00 | 0.00 | C |
| ATOM | 149 | O | PHE | A | 482 | −1.842 | −7.835 | −24.839 | 1.00 | 0.00 | O |
| ATOM | 150 | CB | PHE | A | 482 | −2.510 | −9.116 | −21.854 | 1.00 | 0.00 | C |
| ATOM | 151 | CG | PHE | A | 482 | −2.599 | −7.627 | −21.706 | 1.00 | 0.00 | C |
| ATOM | 152 | CD1 | PHE | A | 482 | −3.824 | −6.999 | −21.653 | 1.00 | 0.00 | C |
| ATOM | 153 | CD2 | PHE | A | 482 | −1.452 | −6.848 | −21.733 | 1.00 | 0.00 | C |
| ATOM | 154 | CE1 | PHE | A | 482 | −3.892 | −5.612 | −21.587 | 1.00 | 0.00 | C |
| ATOM | 155 | CE2 | PHE | A | 482 | −1.509 | −5.459 | −21.653 | 1.00 | 0.00 | C |
| ATOM | 156 | CZ | PHE | A | 482 | −2.726 | −4.849 | −21.584 | 1.00 | 0.00 | C |
| ATOM | 157 | N | THR | A | 517 | −1.365 | −21.939 | −23.929 | 1.00 | 0.00 | N |
| ATOM | 158 | CA | THR | A | 517 | −2.292 | −22.951 | −24.438 | 1.00 | 0.00 | C |
| ATOM | 159 | C | THR | A | 517 | −2.810 | −22.581 | −25.817 | 1.00 | 0.00 | C |
| ATOM | 160 | O | THR | A | 517 | −2.842 | −21.395 | −26.193 | 1.00 | 0.00 | O |
| ATOM | 161 | CB | THR | A | 517 | −3.507 | −23.142 | −23.506 | 1.00 | 0.00 | C |
| ATOM | 162 | OG1 | THR | A | 517 | −4.328 | −21.979 | −23.541 | 1.00 | 0.00 | O |
| ATOM | 163 | CG2 | THR | A | 517 | −3.054 | −23.348 | −22.088 | 1.00 | 0.00 | C |
| ATOM | 164 | N | GLU | A | 519 | −5.906 | −22.842 | −26.661 | 1.00 | 0.00 | N |
| ATOM | 165 | CA | GLU | A | 519 | −7.130 | −22.072 | −26.421 | 1.00 | 0.00 | C |
| ATOM | 166 | C | GLU | A | 519 | −6.798 | −20.592 | −26.288 | 1.00 | 0.00 | C |
| ATOM | 167 | O | GLU | A | 519 | −7.487 | −19.727 | −26.837 | 1.00 | 0.00 | O |
| ATOM | 168 | CB | GLU | A | 519 | −7.818 | −22.565 | −25.150 | 1.00 | 0.00 | C |
| ATOM | 169 | CG | GLU | A | 519 | −8.389 | −23.950 | −25.275 | 1.00 | 0.00 | C |
| ATOM | 170 | CD | GLU | A | 519 | −7.400 | −25.100 | −25.037 | 1.00 | 0.00 | C |
| ATOM | 171 | OE1 | GLU | A | 519 | −8.201 | −24.901 | −24.710 | 1.00 | 0.00 | O |
| ATOM | 172 | OE2 | GLU | A | 519 | −7.878 | −26.243 | −25.154 | 1.00 | 0.00 | O1− |
| ATOM | 173 | N | GLN | A | 520 | −5.730 | −20.280 | −25.556 | 1.00 | 0.00 | N |
| ATOM | 174 | CA | GLN | A | 520 | −5.272 | −18.904 | −25.480 | 1.00 | 0.00 | C |
| ATOM | 175 | C | GLN | A | 520 | −4.918 | −18.337 | −26.868 | 1.00 | 0.00 | C |
| ATOM | 176 | O | GLN | A | 520 | −5.288 | −17.211 | −27.204 | 1.00 | 0.00 | O |
| ATOM | 177 | CB | GLN | A | 520 | −4.061 | −18.797 | −24.549 | 1.00 | 0.00 | C |
| ATOM | 178 | CG | GLN | A | 520 | −4.496 | −18.884 | −23.058 | 1.00 | 0.00 | C |
| ATOM | 179 | CD | GLN | A | 520 | −3.347 | −19.190 | −22.148 | 1.00 | 0.00 | C |
| ATOM | 180 | OE1 | GLN | A | 520 | −2.203 | −19.282 | −22.594 | 1.00 | 0.00 | O |
| ATOM | 181 | NE2 | GLN | A | 520 | −3.641 | −19.354 | −20.840 | 1.00 | 0.00 | N |
| ATOM | 182 | N | GLN | A | 522 | −5.901 | −19.293 | −29.818 | 1.00 | 0.00 | N |
| ATOM | 183 | CA | GLN | A | 522 | −7.161 | −19.058 | −30.518 | 1.00 | 0.00 | C |
| ATOM | 184 | C | GLN | A | 522 | −7.886 | −17.803 | −30.023 | 1.00 | 0.00 | C |
| ATOM | 185 | O | GLN | A | 522 | −8.414 | −17.033 | −30.826 | 1.00 | 0.00 | O |
| ATOM | 186 | CB | GLN | A | 522 | −8.048 | −20.269 | −30.403 | 1.00 | 0.00 | C |
| ATOM | 187 | CG | GLN | A | 522 | −9.385 | −20.074 | −31.158 | 1.00 | 0.00 | C |
| ATOM | 188 | CD | GLN | A | 522 | −10.126 | −21.352 | −31.430 | 1.00 | 0.00 | C |
| ATOM | 189 | OE1 | GLN | A | 522 | −9.993 | −22.331 | −30.693 | 1.00 | 0.00 | O |
| ATOM | 190 | NE2 | GLN | A | 522 | −10.960 | −21.340 | −32.478 | 1.00 | 0.00 | N |
| ATOM | 191 | N | PHE | A | 523 | −7.892 | −17.585 | −28.716 | 1.00 | 0.00 | N |
| ATOM | 192 | CA | PHE | A | 523 | −8.558 | −16.402 | −28.141 | 1.00 | 0.00 | C |
| ATOM | 193 | C | PHE | A | 523 | −7.970 | −15.104 | −28.678 | 1.00 | 0.00 | C |
| ATOM | 194 | O | PHE | A | 523 | −8.702 | −14.168 | −29.012 | 1.00 | 0.00 | O |
| ATOM | 195 | CB | PHE | A | 523 | −8.447 | −l6.445 | −26.600 | 1.00 | 0.00 | C |
| ATOM | 196 | CG | PHE | A | 523 | −9.373 | −15.501 | −25.890 | 1.00 | 0.00 | C |
| ATOM | 197 | CD1 | PHE | A | 523 | −10.697 | −15.401 | −26.244 | 1.00 | 0.00 | C |
| ATOM | 198 | CD2 | PHE | A | 523 | −8.929 | −14.785 | −24.794 | 1.00 | 0.00 | C |
| ATOM | 199 | CE1 | PHE | A | 523 | −11.558 | −14.553 | −25.580 | 1.00 | 0.00 | C |
| ATOM | 200 | CE2 | PHE | A | 523 | −9.766 | −13.941 | −24.117 | 1.00 | 0.00 | C |
| ATOM | 201 | CZ | PHE | A | 523 | −11.101 | −13.827 | −24.497 | 1.00 | 0.00 | C |
| ATOM | 202 | N | HIS | A | 526 | −9.505 | −14.581 | −31.938 | 1.00 | 0.00 | N |
| ATOM | 203 | CA | HIS | A | 526 | −10.890 | −14.077 | −31.780 | 1.00 | 0.00 | C |
| ATOM | 204 | C | HIS | A | 526 | −10.902 | −12.563 | −31.506 | 1.00 | 0.00 | C |
| ATOM | 205 | O | HIS | A | 526 | −11.708 | −11.829 | −32.070 | 1.00 | 0.00 | O |
| ATOM | 206 | CB | HIS | A | 526 | −11.640 | −14.804 | −30.658 | 1.00 | 0.00 | C |
| ATOM | 207 | CG | HIS | A | 526 | −11.995 | −16.236 | −30.946 | 1.00 | 0.00 | C |
| ATOM | 208 | ND1 | HIS | A | 526 | −12.434 | −16.668 | −32.175 | 1.00 | 0.00 | N |
| ATOM | 209 | CD2 | HIS | A | 526 | −12.043 | −17.320 | −30.131 | 1.00 | 0.00 | C |
| ATOM | 210 | CE1 | HIS | A | 526 | −12.730 | −17.957 | −32.111 | 1.00 | 0.00 | C |
| ATOM | 211 | NE2 | HIS | A | 526 | −12.516 | −18.374 | −30.876 | 1.00 | 0.00 | N |
| TER | 212 | | HIS | A | 526 | | | | | | |
| END | | | | | | | | | | | |

TABLE 7
Coordinates of the Enrichment Model Example 4: LYP (PTPN22, PEP,
PTPN8

| | | | | | | | | | | | |
|---|---|---|---|---|---|---|---|---|---|---|---|
| ATOM | 9 | N | TYR | B | 190 | 8.029 | 22.003 | 74.928 | 1.00 | 0.00 | N1+ |
| ATOM | 10 | CA | TYR | B | 190 | 8.411 | 23.235 | 74.250 | 1.00 | 0.00 | C |
| ATOM | 11 | C | TYR | B | 190 | 8.179 | 24.521 | 75.049 | 1.00 | 0.00 | C |
| ATOM | 12 | O | TYR | B | 190 | 7.124 | 24.651 | 75.679 | 1.00 | 0.00 | O |
| ATOM | 13 | CB | TYR | B | 190 | 7.856 | 23.196 | 72.805 | 1.00 | 0.00 | C |
| ATOM | 14 | CG | TYR | B | 190 | 7.781 | 24.513 | 72.064 | 1.00 | 0.00 | C |
| ATOM | 15 | CD1 | TYR | B | 190 | 6.502 | 25.006 | 72.734 | 1.00 | 0.00 | C |
| ATOM | 16 | CD2 | TYR | B | 190 | 8.937 | 25.177 | 71.605 | 1.00 | 0.00 | C |
| ATOM | 17 | CE1 | TYR | B | 190 | 6.378 | 26.147 | 70.923 | 1.00 | 0.00 | C |
| ATOM | 18 | CE2 | TYR | B | 190 | 8.816 | 26.330 | 70.807 | 1.00 | 0.00 | C |
| ATOM | 19 | CZ | TYR | B | 190 | 7.533 | 26.807 | 70.452 | 1.00 | 0.00 | C |
| ATOM | 20 | OH | TYR | B | 190 | 7.397 | 27.905 | 69.655 | 1.00 | 0.00 | O |
| ATOM | 21 | N | LYS | B | 191 | 9.155 | 25.450 | 75.041 | 1.00 | 0.00 | N |
| ATOM | 22 | CA | LYS | B | 191 | 8.968 | 26.748 | 75.708 | 1.00 | 0.00 | C |
| ATOM | 23 | C | LYS | B | 191 | 10.226 | 27.424 | 76.297 | 1.00 | 0.00 | C |
| ATOM | 24 | O | LYS | B | 191 | 10.056 | 28.243 | 77.192 | 1.00 | 0.00 | O |
| ATOM | 25 | CB | LYS | B | 191 | 8.222 | 27.692 | 74.736 | 1.00 | 0.00 | C |
| ATOM | 26 | CG | LYS | B | 191 | 7.500 | 28.831 | 75.482 | 1.00 | 0.00 | C |
| ATOM | 27 | CD | LYS | B | 191 | 6.787 | 29.707 | 74.440 | 1.00 | 0.00 | C |
| ATOM | 28 | CE | LYS | B | 191 | 6.099 | 30.848 | 75.210 | 1.00 | 0.00 | C |
| ATOM | 29 | NZ | LYS | B | 191 | 4.708 | 30.445 | 75.497 | 1.00 | 0.00 | N1+ |
| ATOM | 30 | N | TRP | B | 193 | 12.474 | 29.560 | 74.449 | 1.00 | 0.00 | N1+ |
| ATOM | 31 | CA | TRP | B | 193 | 12.246 | 30.771 | 73.651 | 1.00 | 0.00 | C |
| ATOM | 32 | C | TRP | B | 193 | 13.353 | 31.853 | 73.760 | 1.00 | 0.00 | C |
| ATOM | 33 | O | TRP | B | 193 | 13.837 | 32.153 | 74.852 | 1.00 | 0.00 | O |
| ATOM | 34 | CB | TRP | B | 193 | 10.823 | 31.290 | 73.923 | 1.00 | 0.00 | C |
| ATOM | 35 | CG | TRR | B | 193 | 10.247 | 32.257 | 72.930 | 1.00 | 0.00 | C |
| ATOM | 36 | CD1 | TRP | B | 193 | 10.211 | 33.607 | 73.028 | 1.00 | 0.00 | C |
| ATOM | 37 | CD2 | TRP | B | 193 | 9.822 | 31.962 | 71.664 | 1.00 | 0.00 | C |
| ATOM | 38 | NE1 | TRP | B | 193 | 9.630 | 34.146 | 71.893 | 1.00 | 0.00 | N |
| ATOM | 39 | CE2 | TRP | B | 193 | 9.401 | 33.175 | 70.941 | 1.00 | 0.00 | C |
| ATOM | 40 | CE3 | TRP | B | 193 | 9.791 | 30.786 | 70.780 | 1.00 | 0.00 | C |
| ATOM | 41 | CZ2 | TRP | B | 193 | 8.915 | 33.207 | 69.625 | 1.00 | 0.00 | C |
| ATOM | 42 | CZ3 | TRP | B | 193 | 9.362 | 30.722 | 69.440 | 1.00 | 0.00 | C |
| ATOM | 43 | CH2 | TRP | B | 193 | 8.906 | 32.024 | 68.871 | 1.00 | 0.00 | C |
| ATOM | 44 | N | ASP | B | 197 | 12.207 | 37.873 | 69.131 | 1.00 | 0.00 | N1+ |
| ATOM | 45 | CA | ASP | B | 197 | 11.151 | 37.247 | 68.316 | 1.00 | 0.00 | C |
| ATOM | 46 | C | ASP | B | 197 | 10.747 | 37.900 | 66.972 | 1.00 | 0.00 | C |
| ATOM | 47 | O | ASP | B | 197 | 9.576 | 38.014 | 66.606 | 1.00 | 0.00 | O |
| ATOM | 48 | CB | ASP | B | 197 | 10.035 | 36.614 | 69.171 | 1.00 | 0.00 | C |
| ATOM | 49 | CG | ASP | B | 197 | 9.573 | 37.418 | 70.389 | 1.00 | 0.00 | C |
| ATOM | 50 | OD1 | ASP | B | 197 | 9.839 | 38.640 | 70.426 | 1.00 | 0.00 | O |
| ATOM | 51 | OD2 | ASP | B | 197 | 8.990 | 36.770 | 71.291 | 1.00 | 0.00 | O1− |
| ATOM | 52 | N | VAL | B | 198 | 11.771 | 33.243 | 66.187 | 1.00 | 0.00 | N |
| ATOM | 53 | CA | VAL | B | 198 | 11.721 | 38.522 | 64.744 | 1.00 | 0.00 | C |
| ATOM | 54 | C | VAL | B | 198 | 11.291 | 37.278 | 63.915 | 1.00 | 0.00 | C |
| ATOM | 55 | O | VAL | B | 198 | 11.155 | 36.196 | 64.497 | 1.00 | 0.00 | O |
| ATOM | 56 | CB | VAL | B | 198 | 13.036 | 39.229 | 64.303 | 1.00 | 0.00 | C |
| ATOM | 57 | CG1 | VAL | B | 198 | 13.610 | 40.196 | 65.350 | 1.00 | 0.00 | C |
| ATOM | 58 | CG2 | VAL | B | 198 | 14.141 | 38.229 | 63.919 | 1.00 | 0.00 | C |
| ATOM | 59 | N | PRO | B | 199 | 11.053 | 37.346 | .62.581 | 1.00 | 0.00 | N |
| ATOM | 60 | CA | PRO | B | 199 | 10.413 | 36.234 | 61.866 | 1.00 | 0.00 | C |
| ATOM | 61 | C | PRO | B | 199 | 11.240 | 34.938 | 61.832 | 1.00 | 0.00 | C |
| ATOM | 62 | O | PRO | B | 199 | 10.633 | 33.874 | 61.951 | 1.00 | 0.00 | O |
| ATOM | 63 | CB | PRO | B | 199 | 10.066 | 36.756 | 60.469 | 1.00 | 0.00 | C |
| ATOM | 64 | CG | PRO | B | 199 | 11.038 | 37.912 | 60.264 | 1.00 | 0.00 | C |
| ATOM | 65 | CD | PRO | B | 199 | 11.225 | 38.473 | 61.673 | 1.00 | 0.00 | C |
| ATOM | 66 | N | SER | B | 201 | 12.343 | 33.701 | 64.381 | 1.00 | 0.00 | N1+ |
| ATOM | 67 | CA | SER | B | 201 | 11 966 | 33.002 | 65.614 | 1.00 | 0.00 | C |
| ATOM | 68 | C | SER | B | 201 | 10.844 | 31.976 | 65.378 | 1.00 | 0.00 | C |
| ATOM | 69 | O | SER | B | 201 | 10.533 | 31.244 | 66.262 | 1.00 | 0.00 | O |
| ATOM | 70 | CB | SER | B | 201 | 11.526 | 33.966 | 66.724 | 1.00 | 0.00 | C |
| ATOM | 71 | OG | SER | B | 201 | 10.179 | 34.397 | 66.537 | 1.00 | 0.00 | O |
| ATOM | 72 | N | ILE | B | 202 | 10.221 | 31.951 | 64.199 | 1.00 | 0.00 | N |
| ATOM | 73 | CA | ILE | B | 202 | 9.269 | 30.907 | 63.870 | 1.00 | 0.00 | C |
| ATOM | 74 | C | ILE | B | 202 | 9.969 | 29.581 | 63.622 | 1.00 | 0.00 | C |
| ATOM | 75 | O | ILE | B | 202 | 9.374 | 28.533 | 63.788 | 1.00 | 0.00 | O |
| ATOM | 76 | CB | ILE | B | 202 | 8.414 | 31.303 | 62.643 | 1.00 | 0.00 | C |
| ATOM | 77 | CG1 | ILE | B | 202 | 7.421 | 32.395 | 63.036 | 1.00 | 0.00 | C |
| ATOM | 78 | CG2 | ILE | B | 202 | 7.641 | 30.106 | 62.111 | 1.00 | 0.00 | C |
| ATOM | 79 | CD1 | ILE | B | 202 | 6.820 | 33.155 | 61.857 | 1.00 | 0.00 | C |
| ATOM | 80 | N | ILE | B | 205 | 9.514 | 26.378 | 66.014 | 1.00 | 0.00 | N1+ |
| ATOM | 81 | CA | ILE | B | 205 | 8.236 | 25.691 | 66.107 | 1.00 | 0.00 | C |
| ATOM | 82 | C | ILE | B | 205 | 8.072 | 24.743 | 64.932 | 1.00 | 0.00 | C |
| ATOM | 83 | O | ILE | B | 205 | 7.473 | 23.655 | 65.057 | 1.00 | 0.00 | O |
| ATOM | 84 | CB | ILE | B | 205 | 7.039 | 26.637 | 66.242 | 1.00 | 0.00 | C |
| ATOM | 85 | CG1 | ILE | B | 205 | 5.886 | 25.943 | 66.974 | 1.00 | 0.00 | C |
| ATOM | 86 | CG2 | ILE | B | 205 | 6.602 | 27.218 | 64.905 | 1.00 | 0.00 | C |
| ATOM | 87 | CD1 | ILE | B | 205 | 4.698 | 26.857 | 67.185 | 1.00 | 0.00 | C |
| ATOM | 115 | N | GLY | B | 232 | −4.912 | 28.335 | 72.425 | 1.00 | 0.00 | N |
| ATOM | 116 | CA | GLY | B | 232 | −3.954 | 29.411 | 72.082 | 1.00 | 0.00 | C |
| ATOM | 117 | C | GLY | B | 232 | −2.795 | 28.674 | 71.437 | 1.00 | 0.00 | C |
| ATOM | 118 | O | GLY | B | 232 | −2.848 | 28.312 | 70.278 | 1.00 | 0.00 | O |
| ATOM | 119 | N | ARG | B | 233 | −1.761 | 28.435 | 72.217 | 1.00 | 0.00 | N |
| ATOM | 120 | CA | ARG | B | 233 | −0.585 | 27.749 | 71.748 | 1.00 | 0.00 | C |
| ATOM | 121 | C | ARG | B | 233 | −0.944 | 26.369 | 71.196 | 1.00 | 0.00 | C |
| ATOM | 122 | O | ARG | B | 233 | −0.472 | 25.987 | 70.141 | 1.00 | 0.00 | O |
| ATOM | 123 | CB | ARG | B | 233 | 0.448 | 27.685 | 72.899 | 1.00 | 0.00 | C |
| ATOM | 124 | CG | ARG | B | 233 | 1.115 | 29.044 | 73.165 | 1.00 | 0.00 | C |
| ATOM | 125 | CD | ARG | B | 233 | 1.902 | 28.965 | 74.427 | 1.00 | 0.00 | C |
| ATOM | 126 | NE | ARG | B | 233 | 1.014 | 28.920 | 75.594 | 1.00 | 0.00 | N |
| ATOM | 127 | CZ | ARG | B | 233 | 1.417 | 29.016 | 76.861 | 1.00 | 0.00 | C |
| ATOM | 128 | NH1 | ARG | B | 233 | 2.704 | 29.124 | 77.123 | 1.00 | 0.00 | N |
| ATOM | 129 | NH2 | ARG | B | 233 | 0.538 | 28.963 | 77.870 | 1.00 | 0.00 | N1+ |
| ATOM | 137 | N | VAL | B | 236 | −2.395 | 26.812 | 67.753 | 1.00 | 0.00 | N1+ |
| ATOM | 138 | CA | VAL | B | 236 | −1.384 | 27.262 | 66.795 | 1.00 | 0.00 | C |
| ATOM | 139 | C | VAL | B | 236 | −0.665 | 26.026 | 66.268 | 1.00 | 0.00 | C |
| ATOM | 140 | O | VAL | B | 236 | −0.430 | 25.865 | 65.050 | 1.00 | 0.00 | O |
| ATOM | 141 | CB | VAL | B | 236 | −0.366 | 28.205 | 67.422 | 1.00 | 0.00 | C |
| ATOM | 142 | CG1 | VAL | B | 236 | 0.836 | 28.420 | 66.513 | 1.00 | 0.00 | C |
| ATOM | 143 | CG2 | VAL | B | 236 | −1.012 | 29.603 | 67.769 | 1.00 | 0.00 | C |
| ATOM | 144 | N | ILE | B | 237 | −0.303 | 25.161 | 67.195 | 1.00 | 0.00 | N |
| ATOM | 145 | CA | ILE | B | 237 | 0.397 | 23.904 | 66.787 | 1.00 | 0.00 | C |
| ATOM | 146 | C | ILE | B | 237 | −0.462 | 23.027 | 65.882 | 1.00 | 0.00 | C |
| ATOM | 147 | O | ILE | B | 237 | 0.001 | 22.580 | 64.797 | 1.00 | 0.00 | O |
| ATOM | 148 | CB | ILE | B | 237 | 0.935 | 23.137 | 67.990 | 1.00 | 0.00 | C |
| ATOM | 149 | CG1 | ILE | B | 237 | 2.091 | 23.959 | 68.619 | 1.00 | 0.00 | C |
| ATOM | 150 | CG2 | ILE | B | 237 | 1.422 | 21.741 | 67.526 | 1.00 | 0.00 | C |
| ATOM | 151 | CD1 | ILE | B | 237 | 2.405 | 23.647 | 70.075 | 1.00 | 0.00 | C |
| ATOM | 161 | N | THR | B | 275 | −5.746 | 34.419 | 70.381 | 1.00 | 0.00 | N |
| ATOM | 162 | CA | THR | B | 275 | −5.351 | 35.833 | 70.217 | 1.00 | 0.00 | C |
| ATOM | 163 | C | THR | B | 275 | −5.000 | 36.188 | 68.762 | 1.00 | 0.00 | C |
| ATOM | 164 | O | THR | B | 275 | −4.703 | 35.303 | 67.970 | 1.00 | 0.00 | O |
| ATOM | 165 | CB | THR | B | 275 | −4.173 | 36.185 | 71.131 | 1.00 | 0.00 | C |
| ATOM | 166 | OG1 | THR | B | 275 | −2.963 | 35.592 | 70.633 | 1.00 | 0.00 | O |
| ATOM | 167 | CG2 | THR | B | 275 | −4.413 | 35.676 | 72.566 | 1.00 | 0.00 | C |
| ATOM | 168 | N | GLU | B | 277 | −2.395 | 37.699 | 67.900 | 1.00 | 0.00 | N1+ |
| ATOM | 169 | CA | GLU | B | 277 | −0.968 | 37.453 | 67.753 | 1.00 | 0.00 | C |
| ATOM | 170 | C | GLU | B | 277 | −0.707 | 35.998 | 67.344 | 1.00 | 0.00 | C |
| ATOM | 171 | O | GLU | B | 277 | 0.124 | 35.719 | 66.476 | 1.00 | 0.00 | O |
| ATOM | 172 | CB | GLU | B | 27 | −0.236 | 37.828 | 69.036 | 1.00 | 0.00 | C |
| ATOM | 173 | CG | GLU | B | 277 | −0.270 | 39.363 | 69.366 | 1.00 | 0.00 | C |
| ATOM | 174 | CD | GLU | B | 277 | −1.660 | 40.006 | 69.731 | 1.00 | 0.00 | C |
| ATOM | 175 | OE1 | GLU | B | 277 | −2.566 | 39.337 | 70.319 | 1.00 | 0.00 | O |
| ATOM | 176 | OE2 | GLU | B | 277 | −1.830 | 41.230 | 69.418 | 1.00 | 0.00 | O1− |
| ATOM | 177 | N | GLN | B | 278 | −1.426 | 35.057 | 67.967 | 1.00 | 0.00 | N |
| ATOM | 178 | CA | GLN | B | 278 | −1.313 | 33.651 | 67.559 | 1.00 | 0.00 | C |
| ATOM | 179 | C | GLN | B | 278 | −1.849 | 33.418 | 66.140 | 1.00 | 0.00 | C |
| ATOM | 180 | O | GLN | B | 278 | −1.326 | 32.576 | 65.379 | 1.00 | 0.00 | O |
| ATOM | 181 | CB | GLN | B | 278 | −2.100 | 32.799 | 68.600 | 1.00 | 0.00 | C |
| ATOM | 182 | CG | GLN | B | 278 | −1.531 | 32.802 | 70.015 | 1.00 | 0.00 | C |
| ATOM | 183 | CD | GLN | B | 278 | −2.579 | 32.381 | 71.077 | 1.00 | 0.00 | C |
| ATOM | 184 | OE1 | GLN | B | 278 | −3.747 | 32.120 | 70.762 | 1.00 | 0.00 | O |
| ATOM | 185 | NE2 | GLN | B | 278 | −2.146 | 32.318 | 72.343 | 1.00 | 0.00 | N |
| ATOM | 186 | N | GLU | B | 280 | −1.719 | 35.621 | 63.699 | 1.00 | 0.00 | N1+ |
| ATOM | 187 | CA | GLU | B | 280 | −0.615 | 36.043 | 62.841 | 1.00 | 0.00 | C |
| ATOM | 188 | C | GLU | B | 280 | 0.501 | 35.022 | 62.757 | 1.00 | 0.00 | C |
| ATOM | 189 | O | GLU | B | 280 | 1.092 | 34.832 | 61.675 | 1.00 | 0.00 | O |
| ATOM | 190 | CB | GLU | B | 280 | −0.043 | 37.408 | 63.291 | 1.00 | 0.00 | C |
| ATOM | 191 | CG | GLU | B | 280 | −1.094 | 38.473 | 63.286 | 1.00 | 0.00 | C |
| ATOM | 192 | CD | GLU | B | 280 | −0.600 | 39.853 | 63.600 | 1.00 | 0.00 | C |
| ATOM | 193 | OE1 | GLU | B | 280 | 0.476 | 40.201 | 63.093 | 1.00 | 0.00 | O |
| ATOM | 194 | OE2 | GLU | B | 280 | −1.329 | 40.591 | 64.307 | 1.00 | 0.00 | O1− |
| ATOM | 195 | N | LEU | B | 281 | 0.834 | 34.402 | 63.879 | 1.00 | 0.00 | N |
| ATOM | 196 | CA | LEU | B | 281 | 1.890 | 33.402 | 63.891 | 1.00 | 0.00 | C |
| ATOM | 197 | C | LEU | B | 281 | 1.549 | 32.233 | 62.973 | 1.00 | 0.00 | C |
| ATOM | 198 | O | LEU | B | 281 | 2.403 | 31.765 | 62.226 | 1.00 | 0.00 | O |
| ATOM | 199 | CB | LEU | B | 281 | 2.141 | 32.878 | 65.307 | 1.00 | 0.00 | C |
| ATOM | 200 | CG | LEU | B | 281 | 3.187 | 31.767 | 65.510 | 1.00 | 0.00 | C |
| ATOM | 201 | CD1 | LEU | B | 281 | 4.493 | 32.098 | 64.812 | 1.00 | 0.00 | C |
| ATOM | 202 | CD2 | LEU | B | 281 | 3.428 | 31.484 | 67.074 | 1.00 | 0.00 | C |
| ATOM | 203 | N | ASN | B | 284 | 1.988 | 33.440 | 59.363 | 1.00 | 0.00 | N1+ |
| ATOM | 204 | CA | ASN | B | 284 | 3.390 | 33.634 | 58.990 | 1.00 | 0.00 | C |
| ATOM | 205 | C | ASN | B | 284 | 4.118 | 32.317 | 58.835 | 1.00 | 0.00 | C |
| ATOM | 206 | O | ASN | B | 284 | 4.941 | 32.144 | 57.923 | 1.00 | 0.00 | O |
| ATOM | 207 | CB | ASN | B | 284 | 4.072 | 34.510 | 60.034 | 1.00 | 0.00 | C |
| ATOM | 208 | CG | ASN | B | 284 | 3.681 | 35.963 | 59.908 | 1.00 | 0.00 | C |
| ATOM | 209 | OD1 | ASN | B | 284 | 3.384 | 36.437 | 58.814 | 1.00 | 0.00 | O |
| ATOM | 210 | ND2 | ASN | B | 234 | 3.714 | 36.682 | 61.020 | 1.00 | 0.00 | N |
| TER | 211 | | ASN | B | 284 | | | | | | |
| END | | | | | | | | | | | |

Methods for Comparison of Phosphatase Enrichment Models

Preparation of the Enrichment Model for Comparison

Enrichment Models for the modulation of SHP-2, PTP-PEST (PTPN12, PTPG1), LYP (PTPN22, PEP, PTPN8), PTP1B and STEP were constructed by the preparation of the 3-dimensional representation of the proteins based on but not limited to the crystallographic structure of the SHP-2 protein and the application of computer algorithms to modify regions important for phosphatase function as explained in methods.

The Selection of the SHP-2 Enrichment Model Residues

Selection of SHP-2 Enrichment Model 1 Residues

To select the residues for SHP-2 Enrichment Model 1 missing loops and side-chains were constructed for the SHP-2 structure (PDB access code: 2SHP) using homology modeling with the available full sequence (UnitProtKB entry Q06124) from the SWISSPROT data base. Once these were added to the SHP-2 structure it was fully relaxed in the presence of solvent to relieve bad crystallographic contacts or other geometry issues. Missing data was replaced and corrected before using the structure for Enrichment Model residue selection. Enrichment Model 1 residues are T⁵⁹, G⁶⁰, D⁶¹, Y⁶², E³⁶¹, R³⁶², K³⁶⁴, K³⁶⁶, W⁴²³, P⁴²⁴, D⁴²⁵, H⁴²⁶, G⁴²⁷, V⁴²⁸, G⁴⁶⁴, R⁴⁶⁵, Q⁵¹⁰.

Selection of SHP-2 Enrichment Model 2 and 3 Residues

The SHP-2 structure (PDB access code: 4DGP) last resolved residue is Glu528 out of 533 residues in the construct, while the full sequence has 597 residues. The last 67 residues correspond to the C-terminus region which has been implicated in the SHP-2 phosphatase function. This region undergoes phosphorylation by PDGFR at residues 546 and 584 and then interacts with the N-SH2 domain removing it from the PTP domain and activating SHP-2. This selection of residues for use in this Enrichment Method requires the use of C-terminus of SHP-2 which is further expected to be located close to the αF helix (residues 437-451) which is connected to the WPD loop. Modulators of SHP-2 identified in this enrichment method are expected to bind and modulate the movement of the WPD-loop which is essential for activation of SHP-2.

To select the residues for the SHP-2 Enrichment Models 2 and 3, missing loops and side-chains were constructed using the SHP-2 structure (PDB access code: 4DGP) using homology modeling with the available full sequence (UnitProtKB entry Q06124) from the SWISSPROT data base, excluding the C-terminus. The backbone and side chains were completed and errors corrected. Hydrogen atoms were included and partial charges calculated. Once these were added to the SHP-2 structures the protein models were fully relaxed in the presence of solvent to avoid clashes using the standard Molecular Mechanics force field to relieve bad crystallographic contacts or other geometry issues. A C-terminus short peptide was further included in the Enrichment Models 2 and 3. To select the residues for Enrichment Model 2 a homology model of the catalytic domain of SHP-2 was built employing the structure of PTP1B phosphatase (PDB access code 2NT7) which includes the C-terminus α7 helix (S285-D298). Then the short C-terminus peptide was saved as a chain and then connected to the SHP-2 structure. To select the residues for Enrichment Model 3 the C-terminus α7 helix (S285-D298) of PTP1B phosphatase (PDB access code 2NT7) was employed as the short peptide with direct grafting of the α7 helix from the homology model on to the SHP-2 structure using a Protein Editor.

Selection of Residues of SHP-2 for Enrichment Model 2

A homology model of the catalytic domain of SHP-2 was built employing the structure of PTP1B phosphatase (PDB access code 2NT7) which includes the C-terminus α7 helix (S285-D298). Then the short C-terminus peptide was manually grafted onto the SHP-2 structure.

The last 14 residues (S285-D298) of the α7 helix of the catalytic domain of PTP1B (PDB access code 2NT7) were grafted to the prepared SHP-2 structure of the General Method.

To avoid clashes with residues from the SHP-2 beta strands βJ-βK only the last eight SHP-2 residues I⁵³³EEEQKSK⁵⁴⁰ were retained.

Enrichment Model 2 residues are G⁴³⁷, L⁴⁴⁰, D⁴⁴¹, E⁴⁴⁴, E⁴⁴⁵, H⁴⁴⁸, H⁵²⁴, Y⁵²⁵, E⁵²⁷, T⁵²⁸, R⁵³¹, R⁵³², I⁵³³, E⁵³⁴, E⁵³⁵, E⁵³⁶, K⁵⁴⁰.

Selection of Residues of SHP-2 for Enrichment Model 3

The PTP1B (S285-D298) α7 helix was grafted directly to the full length of SHP-2 prepared in the general method using the Protein Editor. The helix did not overlay with the PTP1B template structure. In this case the application placed the short peptide avoiding clashes with SHP-2 beta strands βJ-βK which are placed differently in the PTP1B structure.

Enrichment Model 3 residues are P³¹², E³¹³, F³¹⁴, E³¹⁵, K³²², P³²³, K³²⁴, K³²⁵, S³²⁶, Y³²⁷, H⁴⁴⁷, Q⁴⁵⁰, E⁴⁵¹, I⁴⁵³, M⁴⁵⁴, A⁴⁵⁶, G⁴⁵⁷, P⁴⁵⁸, V⁴⁵⁹, D⁴⁷⁷, I⁴⁷⁸, D⁴⁸¹, I⁴⁸², R⁴⁸⁴, E⁴⁸⁵, K⁴⁸⁶, E⁵³⁴, E⁵³⁵, E⁵³⁶, Q⁵³⁷, K⁵³⁸, S⁵³⁹, K⁵⁴⁰, R⁵⁴¹, K⁵⁴², G⁵⁴³, H⁵⁴⁴, E⁵⁴⁵, Y⁵⁴⁶, T⁵⁴⁷.

Selection of SHP-2 Enrichment Model 4 Residues

Enrichment Model 4 Collection and Their Use

The SHP-2 residues selected from this method are utilized in a process to identify SHP-2, PTP-PEST (PTPN12, PTPG1), LYP (PTPN22, PEP, PTPN8), PTP1B and STEP modulators by utilization of the movement of the WPD-loop and the connecting αF helix (SHP-2 residues 437-451). Multiple conformations of the WPD loop are expected to provide multiple Enrichment Models, which vary in electrostatic and steric properties as the WPD-loop changes its orientation. The process employed provides multiple Enrichment Models which are hereto collected and described as the Enrichment Model Collection 4. Collectively or singularly the use of these models will identify Candidate Modulators of SHP-2. The SHP-2 structure (PDB access code: 4DGP) was employed for the selection of residues for the Enrichment Model 4 Collection.

General Method Description:

To construct the Enrichment Model 4 Collection different conformations of the WPD-loop and the αF helix were generated by Conformational Search. In order to provide the SHP-2 residues for construction of the Enrichment Model 4 Collection two approaches were used to select residues for the conformational search. In the first case residues within 4.5 Å sphere from Leu440 in the αF-helix were selected and in the second case WPD-loop residues Phe424 to Gly433 were selected.

Enrichment Model 4 Example 1 (EM4.1) contains residues: Y³²⁷, V³⁵⁴, D³⁹⁵, F⁴²⁴, T⁴²⁶, W⁴²⁷, P⁴³³, D⁴³⁵, P⁴³⁶, G⁴³⁷, G⁴³⁸, V⁴³⁹, L⁴⁴⁰, D⁴⁴¹, F⁴⁴², L⁴⁴³, E⁴⁴⁴, V⁴⁴⁶, V⁴⁵⁹, V⁴⁶¹, F⁴⁷³, I⁴⁷⁴, I⁴⁷⁶, D⁴⁷⁷, I⁴⁸⁰, F⁵¹⁷, A⁵²¹, V⁵²², H⁵²⁴, Y⁵²⁵, T⁵²⁸, R⁵³².

Enrichment Model 4 Example 2 (EM4.2) contains residues: H³⁹⁴, D³⁹⁵, F⁴²⁴, T⁴²⁶, W⁴²⁷, P⁴²⁸, V⁴³², P⁴³³, S⁴³⁴, D⁴³⁵, P⁴³⁶, G⁴³⁷, G⁴³⁸, V⁴³⁹, R⁴⁶⁹, T⁴⁷², F⁴⁷³, Q⁵¹⁴, F⁵¹⁷.

Selection of the Residue for the Enrichment Model 4 Collection

Enrichment Model 4 Example 1 contains selected residues within 4.5 Å sphere from L440 in the αF-helix.

For Enrichment Model 4 example 2 the WPD loop residues Phe424 to Gly433 were selected.

Preparation of the Enrichment Models for Comparison of SHP-2 with PTP-PEST (PTPN12, PTPG1), LYP (PTPN22, PEP, PTPN8), PTP1B and STEP

Models were built for SHP-2, PTP-PEST (PTPN12, PTPG1), LYP (PTPN22, PEP, PTPN8), PTP1B and STEP. For SHP-2 the sequence from the available crystal structures, and the others used the complete and/or canonical sequences. Side-chain positions remained unchanged. Protein sequences used in this comparison are listed below with the corresponding UniProtKB (see, web site at uniprot.org) descriptor:
SHP-2=“Q06124-2” [canonical isoform_1, this is isoform_2/crystal structure seq.]
LYP (PTPN22, PEP, PTPN8)=“Q9Y2R2-1” [canonical is isoform_1, this is isoform_1],
PTP1B=“P18031” [complete],
STEP=“P54829” [complete],
PTP-PEST (PTP12, PTPG1)=“Q05209” [complete].

Method 1

Description of Comparison Method 1

To provide this level of utility assessment of the Enrichment Models for SHP-2, PTP-PEST (PTPN12, PTPG1), LYP (PTPN22, PEP, PTPN8), PTP1B and STEP and by extension to other phosphatase derived Enrichment Models, Method 1 employs a weighting system which is applied for the comparison of amino acid residues included in the Enrichment Models.

Two penalty levels are assigned (one severe (−2), one moderate (−1)) to residues which contribute negatively to the similarity assessment relative to SHP-2.

In one embodiment of the invention, residues are assigned the following weights as set forth in Table 1. The weight factors selected provide a dynamic range of 4 as they range from 2 to −2. A weight of 2 indicates an identical residue whereas a weight of −2 indicates a change in amino acid charge. Determination of the similarity assessment provides a critical first analysis of the Enrichment Model selectivity assessment.

TABLE 1
Weighting factors for residues in the Model 1 Enrichment Model
Comparison.

2: For residues which are identical to the SHP-2 model
1: For residues of the same grouping (hydrophobic, hydrophylic, acidic
or basic)
−1: For residues which are of different grouping but do not represent a
polarity change
−2: For resides which represent a change in polarity i.e. from acidic to
basic, or conversely basic to acid

In one instance the sum of the weighting factors is indicative of the degree of similarity to SHP-2. Those phosphatases scoring similarly to SHP-2 would be expected to generate modulators with a high degree of similarity leading to non-selectivity.

In a second instance inspection of those residues with higher penalty levels provides a further degree of selectivity assessment. Large changes in polarity in comparison to SHP-2 are expected to provide more structural diversity and hence lead to improved selectivity relative to SHP-2.

Furthermore by inspection of the individual amino acids which are most similar or dissimilar between the phosphatases being compared it will be the case that the difference between modulators binding at the respective Enrichment Models will be determined.

The following illustrative examples demonstrate the application and utility of Comparison Method 1 when applied to the following phosphatases: PTP-PEST, LYP, PTP1B, and STEP. Enrichment Model 1 for SHP-2 contains residues located within the SH-domain of SHP-2 in addition to others from different locations within SHP-2. The residues of SHP-2/Enrichment Model 1 are listed in the first column of Table 2. Columns 3, 5, 7, and 9 list the amino acid residues of Enrichment Models for the phosphatases PTP1B, STEP, LYP, and PTP-PEST, respectively. The word “none” is used to indicate where such a corresponding residue is missing. Thus, by employing Method 1, it is clear that PTP1B, STEP, LYP, and PTP-PEST lack four critical residues of the enrichment model, and that any putative binding site models and/or pharmacophore models for the identification of modulators of the protein's function will be significantly different at the location of the missing residues.

Results of the Method 1 assessment of the Enrichment Model EM Tables. 1-4.2

TABLE 2
Amino acid weighting comparison of SHP-2 Enrichment Model 1
Enrichment Model 1

| SHP2 | wt | PTP1B | wt | STEP | wt | LYP | wt | PEST | wt |
|---|---|---|---|---|---|---|---|---|---|
| THR_59 | 2 | none | −1 | none | −1 | none | −1 | none | −1 |
| GLY_60 | 2 | none | −1 | none | −1 | none | −1 | none | −1 |
| ASP_61 | 2 | none | −1 | none | −1 | none | −1 | none | −1 |
| TYR_62 | 2 | none | −1 | none | −1 | none | −1 | none | −1 |
| GLU_361 | 2 | GLU_115 | 2 | GLU_403 | 2 | GLU_133 | 2 | GLU_137 | 2 |
| ARG_362 | 2 | LYS_116 | 1 | MET_404 | −1 | MET_134 | −1 | MET_138 | −1 |
| LYS_364 | 2 | SER_118 | −1 | ASN_405 | −1 | LYS_136 | 2 | ARG_140 | 1 |
| LYS_366 | 2 | LYS_120 | 2 | LYS_407 | 2 | LYS_138 | 2 | LYS_142 | 2 |
| TRP_423 | 2 | TRP_179 | 2 | TRP_459 | 2 | TRP_193 | 2 | TRP_197 | 2 |
| PRO_424 | 2 | PRO_180 | 2 | PRO_460 | 2 | PRO_194 | 2 | PRO_198 | 2 |
| ASP_425 | 2 | ASP_181 | 2 | ASP_461 | 2 | ASP_195 | 2 | ASP_199 | 2 |
| HIS_426 | 2 | PHE_182 | −1 | GLN_462 | −1 | HIS_196 | 2 | HIS_200 | 2 |
| GLY_427 | 2 | GLY_183 | 2 | LYS_463 | −1 | ASP_197 | −1 | ASP_201 | −1 |
| VAL_428 | 2 | VAL_184 | 2 | TYR_464 | −1 | VAL_198 | 2 | VAL_202 | 2 |
| GLY_464 | 2 | GLY_220 | 2 | GLY_501 | 2 | GLY_232 | 2 | GLY_236 | 2 |
| ARG_465 | 2 | ARG_221 | 2 | ARG_502 | 2 | ARG_233 | 2 | ARG_237 | 2 |
| GLN_510 | 2 | GLN_266 | 2 | GLN_544 | 2 | GLN_278 | 2 | GLN_282 | 2 |
| Total | 34 | | 15 | | 7 | | 16 | | 15 |

TABLE 3
Amino acid weighting comparison of SHP-2 Enrichment Model 2
Enrichment Model 2

| SHP2 | wt | PTP1B | wt | STEP | wt | LYP | wt | PEST | wt |
|---|---|---|---|---|---|---|---|---|---|
| GLY_437 | 2 | ALA_189 | 1 | PRO_469 | −1 | ASP_203 | −2 | ASP_207 | −1 |
| LEU_440 | 2 | LEU_192 | 2 | LEU_472 | 2 | LEU_206 | 2 | LEU_210 | 2 |
| ASP_441 | 2 | ASN_192 | −2 | HIS_473 | 1 | GLU_207 | 1 | ASP_211 | 2 |
| GLU_444 | 2 | PHE_196 | −1 | ARG_476 | −2 | TRP_210 | −1 | SER_214 | −1 |
| GLU_445 | 2 | LYS_197 | −2 | GLU_477 | 2 | ASP_211 | 1 | LEU_215 | −1 |
| HIS_448 | 2 | GLU_200 | −2 | GLU_480 | −2 | CYS_214 | −1 | LYS_218 | 1 |
| HIS_524 | 2 | GLU_276 | −2 | LEU_554 | −1 | GLU_228 | −2 | GLN_292 | −1 |
| TYR_525 | 2 | GLY_277 | −1 | TYR_555 | 2 | LEU_289 | 1 | LEU_293 | 1 |
| GLU_527 | 2 | LYS_279 | −2 | LYS_557 | −2 | LYS_291 | −2 | GLU_295 | 2 |
| THR_528 | 2 | PHE_280 | 1 | GLN_558 | −1 | ARG_292 | −2 | LYS_296 | −1 |
| ARG_531 | 2 | GLY_283 | −1 | HIS_561 | 1 | ASP_295 | −2 | GLN_299 | −1 |
| ARG_532 | 2 | ASP_284 | 2 | GLN_562 | −1 | VAL_296 | −1 | LEU_300 | −1 |
| ILE_533 | 2 | SER_285 | 1 | SER_563 | 1 | ILE_297 | 2 | TYR_301 | −1 |
| GLU_534 | 2 | SER_286 | −1 | PRO_564 | −1 | ARG_298 | −2 | GLU_302 | 2 |
| GLU_535 | 2 | VAL_287 | −1 | GLU_565 | 2 | ASP_299 | 1 | ILE_303 | −1 |
| GLU_536 | 2 | GLU_288 | 2 | none | −1 | LYS_300 | −2 | HIS_304 | −2 |
| LYS_540 | 2 | LYS_292 | 2 | none | −1 | ASN_304 | −1 | LYS_308 | 2 |
| Totals | 34 | | −8 | | −2 | | −10 | | 1 |

TABLE 4
Amino acid weighting comparison of SHP-2 Enrichment Model 3
Enrichment Mode 3

| SHP2 | wt | PTP1B | wt | STEP | wt | LYP | wt | PEST | wt |
|---|---|---|---|---|---|---|---|---|---|
| PRO_312 | 2 | MET_74 | −1 | none | −1 | GLY_92 | 1 | GLY_96 | 1 |
| GLU_313 | 2 | GLU_75 | 2 | none | −1 | none | −1 | VAL_97 | −1 |
| LYS_324 | 2 | GLN_78 | −1 | none | −1 | PRO_96 | −1 | PRO_100 | −1 |
| LYS_325 | 2 | ARG_79 | 1 | none | −1 | LYS_97 | 2 | LYS_101 | 2 |
| SER_326 | 2 | SER_80 | 2 | VAL_368 | −1 | ALA_98 | −1 | ALA_102 | −1 |
| TYR_327 | 2 | TYR_81 | 2 | TYR_369 | 2 | TYR_99 | 2 | TYR_103 | 2 |
| HIS_447 | 2 | ARG_199 | 1 | GLU_479 | −2 | ARG_213 | 1 | ARG_217 | 1 |
| GLU_451 | 2 | LEU_204 | −1 | GLN_483 | −1 | GLU_217 | 2 | GLU_221 | 2 |
| ASP_481 | 2 | LEU_233 | −1 | GLN_514 | −1 | MET_245 | −1 | ASN_249 | −1 |
| ARG_484 | 2 | ASP_236 | −2 | ARG_517 | 2 | LYS_248 | 1 | LYS_252 | 1 |
| LYS_485 | 2 | LYS_237 | −2 | GLN_518 | −1 | ASP_249 | 1 | ALA_253 | 1 |
| LYS_538 | 2 | GLN_290 | −1 | none | −1 | SER_302 | −1 | ALA_306 | −1 |
| SER_539 | 2 | TRP_291 | −1 | none | −1 | GLY_303 | −1 | GLN_307 | 1 |
| LYS_542 | 2 | LEU_294 | −1 | none | −1 | SER_306 | −1 | ALA_310 | −1 |
| GLY_543 | 2 | SER_295 | −1 | none | −1 | GLN_307 | −1 | ASP_311 | −1 |
| HIS_544 | 2 | HIS_296 | 2 | none | −1 | ALA_308 | −1 | GLY_312 | −1 |
| GLU_545 | 2 | GLU_297 | 2 | none | −1 | LYS_309 | −2 | VAL_313 | −1 |
| TYR_546 | 2 | ASP_298 | −1 | none | −1 | HIS_310 | −1 | ASN_314 | 1 |
| THR_547 | 2 | LEU_298 | −1 | none | −1 | CYS_311 | 1 | GLU_315 | −1 |
| Totals | 38 | | −2 | | −14 | | −1 | | 0 |

TABLE 5
Amino acid weighting comparison of SHP-2 Enrichment Model 4.1
Enrichment Mode 4.1

| SHP2 | wt | PTP1B | wt | STEP | wt | LYP | wt | PEST | wt |
|---|---|---|---|---|---|---|---|---|---|
| TYR_327 | 2 | TYR_81 | 2 | TYR_369 | 2 | TYR_99 | 2 | TYR_103 | 2 |
| VAL_354 | 2 | VAL_108 | 2 | VAL_396 | 2 | VAL_126 | 2 | VAL_130 | 2 |
| ASP_365 | 2 | TYR_152 | −1 | ASP_434 | 2 | ASP_168 | 2 | ASP_172 | 2 |
| PHE_424 | 2 | TYR_176 | −1 | PHE_456 | 2 | TYR_190 | −1 | TYR_194 | −1 |
| THR_426 | 2 | THR_178 | 2 | SER_458 | 1 | ASN_192 | 1 | ASN_106 | 1 |
| TRP_427 | 2 | TRP_197 | 2 | TRP_459 | 2 | TRP_193 | 2 | TRP_197 | 2 |
| PRO_433 | 2 | PRO_185 | 2 | PRO_465 | 2 | PRO_199 | 2 | PRO_203 | 2 |
| ASP_435 | 2 | SER_187 | −1 | ARG_467 | −2 | SER_201 | −1 | SER_205 | −1 |
| PRO_436 | 2 | PRO_188 | 2 | ALA_468 | 1 | ILE_202 | 1 | PHE_206 | 1 |
| GLY_437 | 2 | ALA_189 | 1 | PRO_469 | 1 | ASP_203 | −1 | ASP_207 | −1 |
| GLY_438 | 2 | SER_190 | −1 | PRO_470 | 1 | PRO_204 | 1 | SER_208 | −1 |
| VAL_439 | 2 | PHE_191 | 1 | LEU_471 | 1 | ILE_205 | 1 | ILE_209 | 1 |
| LEU_440 | 2 | LEU_192 | 2 | LEU_472 | 2 | LEU_206 | 2 | LEU_210 | 2 |
| ASP_441 | 2 | ASN_193 | 1 | HIS_ 473 | −2 | GLU_207 | 1 | ASP_211 | 2 |
| PHE_442 | 2 | PHE_194 | 2 | LEU_474 | 1 | LEU_208 | 1 | MET_212 | 1 |
| LEU_443 | 2 | LEU_195 | 2 | VAL_475 | 1 | ILE_209 | 1 | ILE_213 | 1 |
| GLU_444 | 2 | PHE_196 | −1 | ARG_476 | −2 | TYP_210 | −1 | SER_214 | −1 |
| VAL_446 | 2 | VAL_198 | 2 | VAL_478 | 2 | VAL_212 | 2 | MET_216 | 1 |
| VAL_459 | 2 | VAL_211 | 2 | ILE_492 | 1 | ILE_223 | 1 | ILE_227 | 1 |
| VAL_461 | 2 | VAL_213 | 2 | VAL_494 | 2 | ILE_225 | 1 | ILE_229 | 1 |
| PHE_473 | 2 | PHE_225 | 2 | PHE_506 | 1 | ILE_237 | 21 | ILE_241 | 1 |
| ILE_474 | 2 | CYS_226 | −1 | ILE_507 | 2 | CYS_238 | −1 | CYS_242 | −1 |
| ILE_476 | 2 | ALA_228 | 1 | THR_509 | −1 | ILE_240 | 2 | ILE_244 | 2 |
| ASP_477 | 2 | ASP_229 | 2 | SER_510 | −1 | ASP_241 | 2 | ASP_245 | 2 |
| ILE_480 | 2 | LEU_232 | 1 | CYS_513 | −1 | TRP_244 | 1 | TRP_248 | 1 |
| PHE_517 | 2 | PHE_264 | 2 | PHE_547 | 2 | LEU_281 | 1 | LEU_285 | 1 |
| ALA_521 | 2 | ALA_273 | 2 | VAL_551 | 1 | ALA_285 | 2 | ALA_289 | 2 |
| VAL_522 | 2 | VAL_274 | 2 | MET_552 | 1 | VAL_286 | 1 | ILE_290 | 1 |
| HIS_524 | 2 | GLU_276 | −2 | LEU_554 | −1 | GLU_288 | −2 | GLN_292 | −1 |
| TYR_525 | 2 | GLY_277 | −1 | TYR_555 | 2 | LEU_289 | −1 | LEU_293 | −1 |
| THR_528 | 2 | PHE_280 | −1 | GLN_558 | −1 | ARG_292 | −1 | LYS_296 | −1 |
| ARG_532 | 2 | ASP_284 | −2 | GLN_562 | −1 | VAL_296 | −1 | LEU_300 | −1 |
| Total | 64 | | 25 | | 23 | | 23 | | 22 |

TABLE 6
Amino acid weighting comparison of SHP-2 Enrichment Model 4.2
Enrichment Model 4.2

| SHP2 | wt | PTP1B | wt | STEP | wt | LYP | wt | PEST | wt |
|---|---|---|---|---|---|---|---|---|---|
| HIS_394 | 2 | SER_151 | −1 | GLU_433 | −2 | SER_167 | −1 | THR_171 | −1 |
| ASP_395 | 2 | TYR_152 | −1 | ASP_434 | 2 | ASP_168 | 2 | ASP_172 | 2 |
| PHE_424 | 2 | TYR_176 | 1 | PHE_456 | 2 | TYR_190 | −1 | TYR_194 | −1 |
| THR_426 | 2 | THR_178 | 2 | SER_458 | 1 | ASN_192 | −1 | ASN_196 | 1 |
| TRP_477 | 2 | TRP_179 | 2 | TRP_459 | 2 | TRP_193 | 2 | TRP_197 | 2 |
| PRO_428 | 2 | PRO_180 | 2 | PRO_460 | 2 | PRO_194 | 2 | PRO_198 | 2 |
| VAL_432 | 2 | VAL_184 | 2 | THR_464 | 1 | VAL_198 | 2 | VAL_202 | 2 |
| PRO_433 | 2 | PRO_185 | 2 | PRO_465 | 2 | PRO_199 | 2 | PRO_203 | 2 |
| SER_434 | 2 | GLU_186 | −1 | ASP_466 | −1 | SER_200 | 2 | SER_204 | 2 |
| ASP_435 | 2 | SER_187 | −1 | ARG_467 | −2 | SER_201 | −1 | SER_205 | −1 |
| PRO_436 | 2 | PRO_188 | 2 | ALA_468 | 1 | ILE_202 | 1 | PHE_206 | 1 |
| GLY_437 | 2 | ALA_189 | 1 | PRO_469 | 1 | ASP_203 | −1 | ASP_207 | −1 |
| GLY_438 | 2 | SER_190 | 1 | PRO_470 | 1 | PRO_204 | 1 | SER_208 | −1 |
| VAL_439 | 2 | PHE_191 | 1 | LEU_471 | 1 | ILE_205 | 1 | ILE_209 | 1 |
| ARG_469 | 2 | ARG_221 | 2 | ARG_502 | 2 | ARG_233 | 2 | ARG_237 | 2 |
| THR_472 | 2 | THR_224 | 2 | CYS_505 | 1 | VAL_236 | −1 | ALA_240 | −1 |
| PHE_473 | 2 | PHE_225 | 2 | PHE_506 | 2 | ILE_237 | 1 | ILE_241 | 1 |
| GLN_514 | 2 | GLN_266 | 2 | GLN_544 | 2 | GLN_278 | 2 | GLN_282 | 2 |
| PHE_517 | 2 | PHE_269 | 2 | PHE_547 | 2 | LEU_281 | 1 | LEU_285 | 1 |
| Total | 38 | | 20 | | 20 | | 15 | | 15 |

Description of Enrichment Model Comparison Method 2

Visualization of the Enrichment Model can be achieved by a method such as via a Chime plugin (http://www.umass.edu/microbio/chime/abtchime.htm) embedded in HTML pages. The backbone overlay models were created using those of the residues corresponding to SHP-2 positions from each of the Enrichment Models.

Enrichment Model comparison tables of the residues were constructed using Comparison Method 2 described below:

All protein atoms in the original (non-overlay) builds were set as van der Waals radii with a 1.4A solvent-accessible surface applied over the spheres. Each residue in the Enrichment Model was assessed to determine the degree of solvent exposure per atom following the methodology presented below:

Exposure ranking values: “full” = 1.00, “moderate” = 0.50,
“minimal” = 0.25
Hydrophobicty (“HP”): (each C and Met S atom) × (exposure
value) = residue's HP value
Hydrophilicity: (each N and O atom) × (exposure value) = residue's
polarity value
Charge: (each standard + labile atom) × (exposure value) = residue's
charge
H-bond donation: (each H-bond donating atom) × (exposure value) =
residue's H-bond donation potential
H-bond acceptance: (each H-bond accepting atom) × (exposure
value) = residue's H-bond acceptance potential

Values used for H-bonding potentials are listed below in Table 7. Three amino acids (Arg, Lys and Trp) have hydrogen bond donor atoms in their side chains, two amino acids (Asp and Glu) have hydrogen acceptor atoms in their side chains and six amino acids (Asn, Gln, His, Ser, Thr, and Tyr) have both hydrogen donor and acceptor atoms in their side chains. The remaining amino acids have no donor or acceptor atoms in their side chains and therefore are not included in Table 7.

Table 7 also sets forth the number of sp hydrogens that can donate or accept hydrogen bonds. These values are recorded as numbers within parentheses in each column (McDonald and Thornton, J. Mol. Biol., 1994, 233:777-793 and Thornton et. al., Phil. Trans. R. Soc. Lond. A., 1993, 345:113-129, and presented on the internet at the web site http://www.imgt.org/IMGTeducation/Aide-memoire/UK/aminoacids/charge/).

TABLE 7
Values used for H-bonding potentials:

| Amino acids | H-Bond Donors | H-Bond Acceptors |
|---|---|---|
| Arg, R | NE(1), NH1(2), NH2(2) | |
| Asn, N | ND2(2) | OD1(2) |
| Asp, D | | OD1(2), OD2(2) |
| Gln, Q | NE2(2) | OE1(2) |
| Glu, E | | OE1(2), OE2(2) |
| His, H | ND1(1), NE2(1) | ND1(1), NE2(1) |
| Lys, K | NZ(3) | |
| Ser, S | OG(1) | OG(2) |
| Thr, T | OG1(1) | OG1(2) |
| Trp, W | NE1(1) | |
| Tyr, Y | OH(1) | OH(1) |

Results for Comparison Method 2 Assessment of Enrichment Model 1-4.2

TABLE 8
Scoring of Enrichment Model 1 for SHP-2

| EM-1 | | | | | | | |
|---|---|---|---|---|---|---|---|
| | full access | half access | min access | (hp) (polar) | (−1) (0) (+1) | (total SC) | (total SC) |
| SHP2 | 1.00 | 0.50 | 0.25 | polarity | charge | donate HBs | accept HBs |
| T59 | OG1, CG1 | O, CA, CB | N, C | \|2.25\| \|1.75\| | \|0.00\| \|0.00\| | \|1.25\| | \|1.50\| |
| G60 | | | CA | \|0.25\| \|0.00\| | \|0.00\| \|0.00\| | \|0.00\| | \|0.00\| |
| D61 | OD2 | | N, CB, | \|0.50\| \|1.25\| | \|−0.50\| \|0.00\| | \|0.25\| | \|2.00\| |
| | | | CG | | | | |
| Y62 | OH, CE1 | CZ | N, O | \|1.50\| \|1.50\| | \|0.00\| \|0.00\| | \|1.25\| | \|1.25\| |
| E361 | | | C, CA, | \|1.00\| \|0.00\| | \|0.00\| \|0.00\| | \|0.00\| | \|0.00\| |
| | | | CB, CG | | | | |
| R362 | O, C, CA, | N, CG | NE | \|5.50\| \|3.75\| | \|0.00\| \|1.00\| | \|3.25\| | \|0.00\| |
| | CB, CD, | | | | | | |
| | CZ, NH1, | | | | | | |
| | NH2 | | | | | | |
| K364 | CD, CE | NZ | C, CA, | \|3.00\| \|0.50\| | \|0.00\| \|0.50\| | \|0.50\| | \|0.00\| |
| | | | CB, CG | | | | |
| K366 | | NZ | CD, CZ | \|0.50\| \|0.50\| | \|0.00\| \|0.50\| | \|0.50\| | \|0.00\| |
| W423 | | O | C | \|0.25\| \|0.50\| | \|0.00\| \|0.00\| | \|0.00\| | \|0.00\| |
| P424 | CB | O | C, CA, | \|2.00\| \|0.50\| | \|0.00\| \|0.00\| | \|0.00\| | \|0.50\| |
| | | | CD, CG | | | | |
| D424 | OD2 | OD1, CG | CB | \|0.75\| \|1.50\| | \|−0.75\| \|0.00\| | \|3.00\| | \|3.00\| |
| H426 | CB, CG, | O, C | CA | \|4.75\| \|2.50\| | \|0.00\| \|0.00\| | \|2.00\| | \|2.00\| |
| | CD1, ND1, | | | | | | |
| | CE1, NE2 | | | | | | |
| G427 | | CA | N | \|0.50\| \|0.25\| | \|0.00\| \|0.00\| | \|0.25\| | \|0.00\| |
| V428 | O, CG2 | N | | \|1.00\| \|1.50\| | \|0.00\| \|0.00\| | \|0.50\| | \|1.50\| |
| G464 | | | | \|0.00\| \|0.00\| | \|0.00\| \|0.00\| | \|0.00\| | \|0.00\| |
| R465 | | NE, NH2 | | \|0.00\| \|1.00\| | \|0.00\| \|0.50\| | \|1.00\| | \|0.00\| |
| Q510 | | | NE2, CG, | \|0.50\| \|0.25\| | \|0.00\| \|0.00\| | \|0.50\| | \|0.00\| |
| | | | CD | | | | |

| | | | | |
|---|---|---|---|---|
| TOTALS | \|19.75\| \|12.75\| | \|−0.75\| \|2.50\| | \|11.50\| | \|7.00\| |

TABLE 9
Scoring of Enrichment Model 1 for PTP1B

| EM1 | | | | | | | |
|---|---|---|---|---|---|---|---|
| | full access | half access | min access | (hp) (polar) | (−1) (0) (+1) | (total SC) | (total SC) |
| PTP1B | 1.00 | 0.50 | 0.25 | polarity | charge | donate HBs | accept HBs |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| E115 | | CB, OE2 | O, C, CD | \|1.00\| \|0.75\| | \|−0.50\| \|0.00\| | \|0.00\| | \|1.00\| |
| K116 | NZ, CE | O, C, CA, | | \|3.50\| \|1.50\| | \|0.00\| \|1.00\| | \|3.00\| | \|0.00\| |
| | | CB, CG, CD | | | | | |
| S118 | OG | CB | CA | \|0.75\| \|1.00\| | \|0.00\| \|0.00\| | \|0.00\| | \|0.00\| |
| K120 | NZ | CE | CG, CD | \|1.00\| \|1.00\| | \|0.00\| \|1.00\| | \|3.00\| | \|0.00\| |
| W179 | | O | C | \|0.25\| \|0.25\| | \|0.00\| \|0.00\| | \|0.00\| | \|0.00\| |
| P180 | CB | CA | CG, CD | \|2.00\| \|0.00\| | \|0.00\| \|0.00\| | \|0.00\| | \|0.00\| |
| D181 | OD2, CG | N, CB | OD1, | \|1.50\| \|1.75\| | \|−0.75\| \|0.00\| | \|0.00\| | \|2.50\| |
| F182 | CB, CG, | O | N, C, CA | \|7.50\| \|0.75\| | \|0.00\| \|0.00\| | \|0.00\| | \|0.00\| |
| | CD1, | | | | | | |
| | CD2, CE1, | | | | | | |
| | CE2, CZ | | | | | | |
| G183 | | N, CA | | \|0.50\| \|0.50\| | \|0.00\| \|0.00\| | \|0.00\| | \|0.00\| |
| V184 | | O, CG2 | | \|0.50\| \|0.50\| | \|0.00\| \|0.00\| | \|0.00\| | \|0.00\| |
| G220 | | | | \|0.00\| \|0.00\| | \|0.00\| \|0.00\| | \|0.00\| | \|0.00\| |
| R221 | | NH2 | CZ | \|0.25\| \|0.50\| | \|0.00\| \|0.50\| | \|1.00\| | \|0.00\| |
| Q266 | | NE2 | | \|0.00\| \|0.50\| | \|0.00\| \|0.00\| | \|1.00\| | \|0.00\| |

| | | | | |
|---|---|---|---|---|
| TOTALS | \|18.75\| \|9.00\| | \|−1.25\| \|2.50\| | \|8.00\| | \|3.50\| |

TABLE 10
Scoring of Enrichment Model 1 for STEP

| EM-1 | | | | | | | |
|---|---|---|---|---|---|---|---|
| | full access | half access | min access | (hp) | (−1) (0) (+1) | (total SC) | (total SC) |
| STEP | 1.00 | 0.50 | 0.25 | polarity | charge | donate HBs | accept HBs |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| E403 | | O, CA, OE1 | C, CB, CG | \|1.75\| \|1.00\| | \|−0.50\| \|0.00\| | \|0.00\| | \|1.00\| |
| M404 | CG, CE, SD | O, CB | C, CA | \|4.00\| \|0.50\| | \|0.00\| \|0.00\| | \|0.00\| | \|0.00\| |
| N405 | ND2, OD1 | CA, CB, CG | C | \|1.75\| \|2.00\| | \|0.00\| \|0.00\| | \|4.00\| | \|0.00\| |
| K407 | | CE, NZ | CA, CG, N | \|1.00\| \|0.75\| | \|0.00\| \|0.50\| | \|1.50\| | \|0.00\| |
| W459 | | | O, C, CB, | \|0.50\| \|0.25\| | \|0.00\| \|0.00\| | \|0.00\| | \|0.00\| |
| P460 | | CA, CB | CG, CD | \|1.50\| \|0.00\| | \|0.00\| \|0.00\| | \|0.00\| | \|0.00\| |
| D461 | OD2 | O, N, CB, | C, CB | \|1.50\| \|2.50\| | \|0.00\| \|0.00\| | \|0.00\| | \|3.00\| |
| | | CG, OD1 | | | | | |
| Q462 | OE1, NE2 | O, C, CA, | N | \|2.50\| \|3.75\| | \|0.00\| \|0.00\| | \|2.00\| | \|2.00\| |
| | | CB, CG, CD | | | | | |
| K463 | CD, CE, NZ | CB, CG | C, CA | \|3.50\| \|1.00\| | \|0.00\| \|1.00\| | \|3.00\| | \|0.00\| |
| T464 | | O, OG1 | CG2 | \|0.25\| \|1.00\| | \|0.00\| \|0.00\| | \|0.50\| | \|1.00\| |
| G501 | | CA | N | \|0.50\| \|0.25\| | \|0.00\| \|0.00\| | \|0.00\| | \|0.00\| |
| R502 | | CG, CD, | N | \|1.50\| \|1.25\| | \|0.00\| \|0.75\| | \|1.50\| | \|0.00\| |
| | | CZ, NE, | | | | | |
| | | NH2 | | | | | |
| Q544 | | CG, NE2 | | \|0.50\| \|0.50\| | \|0.00\| \|0.00\| | \|1.00\| | \|0.00\| |

| | | | | |
|---|---|---|---|---|
| TOTALS | \|20.75\| \|14.25\| | \|−0.50\| \|2.25\| | \|13.50\| | \|7.00\| |

TABLE 11
Scoring of Enrichment Model 1 for LYP (PTPN22, PEP, PTPN8)

| EM-1 | | | | | | | |
|---|---|---|---|---|---|---|---|
| | full access | half access | min access | (hp) (polar) | (−1) (0) (+1) | (total SC) | (total SC) |
| LYP | 1.00 | 0.50 | 0.25 | polarity | charge | donate HBs | accept HBs |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| E133 | | CA, CG, CD | C, CB, | \|2.00\| \|0.25\| | \|−0.50\| \|0.00\| | \|0.00\| | \|0.50\| |
| | | | OE2 | | | | |
| M134 | C, O, CA, | N, CB, CG | | \|5.00\| \|1.50\| | \|0.00\| \|0.00\| | \|0.00\| | \|0.00\| |
| | CE, SD | | | | | | |
| K136 | CG, CD, | CA, CB | C | \|4.25\| \|1.00\| | \|0.00\| \|1.00\| | \|3.00\| | \|0.00\| |
| | CE, NZ | | | | | | |
| K138 | NZ | CE | CG, CD | \|1.00\| \|1.00\| | \|0.00\| \|1.00\| | \|3.00\| | \|0.00\| |
| W193 | | O | C | \|0.25\| \|0.50\| | \|0.00\| \|0.00\| | \|0.00\| | \|0.00\| |
| P194 | CB, CG | CA | | \|2.50\| \|0.00\| | \|0.00\| \|0.00\| | \|0.00\| | \|0.00\| |
| D195 | OD2 | N, CB, CG, | | \|1.00\| \|2.00\| | \|−0.75\| \|0.00\| | \|0.00\| | \|3.00\| |
| | | OD1 | | | | | |
| H196 | CD2, NE2, | CA, CB, CG, | N, O | \|3.50\| \|2.00\| | \|0.00\| \|0.00\| | \|1.50\| | \|1.50\| |
| | CE1 | ND1 | | | | | |
| D197 | CG, OD1 | N, O, C | | \|2.50\| \|2.50\| | \|−0.75\| \|0.00\| | \|0.00\| | \|3.00\| |
| | | CA, CB, | | | | | |
| | | OD2 | | | | | |
| V198 | | O, CG2 | | \|0.50\| \|0.50\| | \|0.00\| \|0.00\| | \|0.00\| | \|0.00\| |
| G232 | | N, CA | | \|0.50\| \|0.50\| | \|0.00\| \|0.00\| | \|0.00\| | \|0.00\| |
| R233 | | CG, NE, | N, CZ | \|0.75\| \|1.25\| | \|−0.75\| \|0.00\| | \|1.50\| | \|0.00\| |
| | | NH2 | | | | | |
| Q278 | | C, OE1 | | \|0.50\| \|0.50\| | \|0.00\| \|0.00\| | \|0.00\| | \|1.00\| |

| | | | | |
|---|---|---|---|---|
| TOTALS | \|24.25\| \|13.50\| | \|−2.75\| \|2.00\| | \|9.00\| | \|9.00\| |

TABLE 12
Scoring of Enrichment Model 1 for PTP-PEST (PTPN12, PTPG1)

| EM-1 | | | | | | | |
|---|---|---|---|---|---|---|---|
| | full access | half access | min access | (hp) (polar) | (−1) (0) (+1) | (total SC) | (total SC) |
| PTP-PEST | 1.00 | 0.50 | 0.25 | polarity | charge | donate HBs | accept HBs |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| N/A | N/A | N/A | N/A | N/A | N/A | N/A | N/A |
| E137 | | | C, CA, | \|0.75\| \|0.00\| | \|0.00\| \|0.00\| | \|0.00\| | \|0.00\| |
| | | | CD | | | | |
| M138 | CE, SD | O, C, CA, | N, CG | \|3.75\| \|0.75\| | \|0.00\| \|0.00\| | \|0.00\| | \|0.00\| |
| | | CB | | | | | |
| R140 | NH1, NH2 | CG, CD, | C, CA, | \|2.25\| \|2.25\| | \|0.00\| \|0.75\| | \|4.00\| | \|0.00\| |
| | | CZ | CB, NE | | | | |
| K142 | | NZ | CD, CE | \|0.50\| \|0.50\| | \|0.00\| \|0.50\| | \|1.50\| | \|0.00\| |
| W197 | | | O, C, | \|0.50\| \|0.50\| | \|0.00\| \|0.00\| | \|0.25\| | \|0.00\| |
| | | | NE1, CD1 | | | | |
| P198 | | CA, CB | CG, CD | \|1.50\| \|0.00\| | \|0.00\| \|0.00\| | \|0.00\| | \|0.00\| |
| D199 | OD2 | CB | N, O, CA, | \|2.00\| \|0.75\| | \|0.75\| \|0.00\| | \|0.00\| | \|2.50\| |
| | | | CG, OD1 | | | | |
| H200 | CE1 | O, NE2 | N, CA, | \|1.50\| \|1.75\| | \|0.00\| \|0.00\| | \|0.50\| | \|0.00\| |
| | | | CB, ND1 | | | | |
| D201 | | CB, CG, | | \|0.50\| \|0.25\| | \|0.25\| \|0.00\| | \|0.00\| | \|0.50\| |
| | | OD2 | | | | | |
| V202 | | O | CG2 | \|0.25\| \|0.50\| | \|0.00\| \|0.00\| | \|0.00\| | \|0.00\| |
| G236 | | | CA, N | \|0.25\| \|0.25\| | \|0.00\| \|0.00\| | \|0.00\| | \|0.00\| |
| R237 | | | N, CG, | \|0.50\| \|0.75\| | \|0.00\| 0.25\| | \|0.50\| | \|0.00\| |
| | | | CZ, | | | | |
| | | | NE, NH2 | | | | |
| Q282 | | | NE2, CG | \|0.25\| \|0.25\| | \|0.00\| \|0.00\| | \|0.50\| | \|0.00\| |

| | | | | |
|---|---|---|---|---|
| TOTALS | \|14.50\|8.50\| | \|−1.00\| \|1.50\| | \|7.25\| | \|3.00\| |

TABLE 13
Comparison of the scoring for Enrichment Model 1

| EM1 | | | Charge | | H-Bonds | |
|---|---|---|---|---|---|---|
| | hydrophobicity | polarity | negative | positive | donate | accept |
| SHP2 | 19.75 | 12.75 | −0.75 | 2.5 | 11.5 | 7 |
| PTP1B | 18.75 | 9 | −1.25 | 2.5 | 8 | 3.5 |
| STEP | 20.75 | 14.25 | −0.5 | 2.25 | 13.5 | 7 |
| LYP | 24.25 | 13.5 | −2.75 | 2 | 9 | 9 |
| PEST | 14.5 | 85.5 | −1 | 1.5 | 7.25 | 3 |

TABLE 14
Difference results for the Enrichment Model 1 compared to SHP-2

| EM1 | | | Charge | | H-Bonds | |
|---|---|---|---|---|---|---|
| | hydrophobicity | polarity | negative | positive | donate | accept |
| SHP2 | 1 | 1 | 1 | 1 | 1 | 1 |
| PTP1B | 0.95 | 0.71 | 1.67 | 1 | 0.7 | 0.5 |
| STEP | 1.05 | 1.12 | 0.67 | 0.9 | 1.17 | 1 |
| LYP | 1.23 | 1.06 | 3.67 | 0.8 | 0.78 | 1.29 |
| PEST | 0.73 | 0.67 | 1.33 | 0.6 | 0.63 | 0.43 |

Utilization of the Assessment Factors (AF)

To provide a numerical Comparison Value (CV) for each of the Assessment Factors (AF) the absolute value of each AF was recorded in Table 13. To utilize the SHP-2 model as a comparator each AF was divided by the corresponding AF for SHP-2. Table 14 sets out these values as compared to SHP-2, which was set to 1 to provide normalization of the results.

TABLE 15
Scoring of Enrichment Model 2 for SHP-2

| EM-2 | | | | | | (only SC) | (only SC) |
|---|---|---|---|---|---|---|---|
| | full access | half access | min access | (hp) (polar) | (−1) (0) (+1) | donate | accept |
| SHP2 | 1.00 | 0.50 | 0.25 | polarity | charge | HBs | HBs |
| G437 | | CA | O, N, C | \|0.75\| \|0.50\| | \|0.00\| \|0.00\| | \|0.00\| | \|0.00\| |
| L440 | | | C, CB, CD1 | \|0.00\| \|0.75\| | \|0.00\| \|0.00\| | \|0.00\| | \|0.00\| |
| D441 | | OD2 | N, CG, OD1 | \|0.25\| \|1.00\| | \|−0.50\| \|0.00\| | \|0.00\| | \|1.50\| |
| E444 | | CG, CD, | CB | \|1.25\| \|0.50\| | \|−0.25\| \|0.00\| | \|0.00\| | \|1.00\| |
| | | OE1 | | | | | |
| E445 | | | CB, CD, OE1 | \|0.50\| \|0.25\| | \|−0.25\| \|0.00\| | \|0.00\| | \|0.50\| |
| H448 | | | C, CB, CG, | \|1.00\| \|0.00\| | \|0.00\| \|0.00\| | \|0.00\| | \|0.00\| |
| | | | CD2 | | | | |
| H524 | CD2, NE2 | CE1 | CB | \|1.75\| \|0.00\| | \|0.00\| \|0.00\| | \|1.00\| | \|1.00\| |
| Y525 | | | CD1, CE1, | \|1.00\| \|0.25\| | \|0.00\| \|0.00\| | \|0.25\| | \|0.25\| |
| | | | CE2, CZ, OH | | | | |
| E527 | OE2 | O, CA, CB, | C, CG | \|2.00\| \|2.00\| | \|−0.75\| \|0.00\| | \|0.00\| | \|3.00\| |
| | | CD, OE1 | | | | | |
| T528 | CG2 | CB | O, C, CA, | \|2.00\| \|0.50\| | \|0.00\| \|0.00\| | \|0.25\| | \|0.50\| |
| | | | OG1 | | | | |
| R531 | CD, NH1, | O, CA, CB, | N, C | \|3.25\| \|2.75\| | \|0.00\| \|1.00\| | \|4.50\| | \|0.00\| |
| | NH2 | CG, CZ, NE | | | | | |
| R532 | NH1, NH2 | CG, CD, | CA, CB | \|2.00\| \|2.50\| | \|0.00\| \|1.00\| | \|4.50\| | \|0.00\| |
| | | CZ, NE | | | | | |
| I533 | | CB, CG1, | N | \|1.50\| \|0.25\| | \|0.00\| \|0.00\| | \|0.00\| | \|0.00\| |
| | | CG2 | | | | | |
| E534 | OE2 | CG, CD | CB, OE1 | \|1.25\| \|1.25\| | \|−0.75\| \|0.00\| | \|0.00\| | \|2.50\| |
| E535 | CD, OE2 | CB, CG, | O, C, CA | \|2.50\| \|1.75\| | \|−0.75\| \|0.00\| | \|0.00\| | \|3.00\| |
| | | OE1 | | | | | |
| E536 | CG, CD, | CB, OE1 | O, C | \|2.75\| \|1.75\| | \|−0.75\| \|0.00\| | \|0.00\| | \|3.00\| |
| | OE2 | | | | | | |
| K540 | NZ | CA, CB, | | \|2.50\| \|1.00\| | \|0.00\| \|1.00\| | \|3.00\| | \|0.00\| |
| | | CG, CD, CE | | | | | |

| | | | | |
|---|---|---|---|---|
| TOTALS | \|26.25\| \|17.00\| | \|−4.00\| \|3.00\| | \|13.50\| | \|16.25\| |

TABLE 16
Scoring of Enrichment Model 2 for PTP1B

| EM-2 | | | | | | (only SC) | (only SC) |
|---|---|---|---|---|---|---|---|
| | full access | half access | min access | (hp) (polar) | (−1) (0) (+1) | donate | accept |
| PTP1B | 1.00 | 0.50 | 0.25 | polarity | charge | HBs | HBs |
| A189 | | | CB, N | \|0.25\| \|0.25\| | \|0.00\| \|0.00\| | \|0.00\| | \|0.00\| |
| L192 | | | | \|0.00\| \|0.00\| | \|0.00\| \|0.00\| | \|0.00\| | \|0.00\| |
| N193 | | | | \|0.00\| \|0.00\| | \|0.00\| \|0.00\| | \|0.00\| | \|0.00\| |
| F196 | | | CD2, CE2 | \|0.50\| \|0.00\| | \|0.00\| \|0.00\| | \|0.00\| | \|0.00\| |
| K197 | | | CA, CB, CD, | \|1.00\| \|0.25\| | \|0.00\| \|0.25\| | \|0.75\| | \|0.00\| |
| | | | CE, NZ | | | | |
| E200 | | | C, CA, CB, | \|1.25\| \|0.50\| | \|−0.25\| \|0.00\| | \|0.00\| | \|1.00\| |
| | | | CG, CD, | | | | |
| | | | OE1, OE2 | | | | |
| E276 | | OE1 | CA, CB, CD, | \|0.75\| \|0.75\| | \|−0.50\| \|0.00\| | \|0.00\| | \|1.50\| |
| | | | OE2 | | | | |
| G277 | | | | \|0.00\| \|0.00\| | \|0.00\| \|0.00\| | \|0.00\| | \|0.00\| |
| K279 | | | | \|0.00\| \|0.00\| | \|0.00\| \|0.00\| | \|0.00\| | \|0.00\| |
| [symbol] 280 | | | | \|0.00\| \|0.00\| | \|0.00\| \|0.00\| | \|0.00\| | \|0.00\| |
| G283 | CA | O, C | | \|1.50\| \|0.50\| | \|0.00\| \|0.00\| | \|0.00\| | \|0.00\| |
| D284 | CB, CG, | CA, OD1 | N | \|2.50\| \|1.75\| | \|−0.75\| \|0.00\| | \|0.00\| | \|3.00\| |
| | OD2 | | | | | | |
| S285 | OG | CB | O, N, CA | \|0.75\| \|1.50\| | \|0.00\| \|0.00\| | \|1.00\| | \|2.00\| |
| S286 | CB | OG | O, N, C | \|1.25\| \|1.00\| | \|0.00\| \|0.00\| | \|0.50\| | \|1.00\| |
| V287 | | | CA, CB, CG2 | \|0.75\| \|0.00\| | \|0.00\| \|0.00\| | \|0.00\| | \|0.00\| |
| Q288 | | | C, CB, CG, | \|0.75\| \|0.25\| | \|0.00\| \|0.00\| | \|0.50\| | \|0.00\| |
| | | | NE2 | | | | |
| K292 | | | CE, NZ | \|0.25\| \|0.25\| | \|0.00\| \|0.25\| | \|0.75\| | \|0.00\| |

| | | | | |
|---|---|---|---|---|
| TOTALS | \|11.50\| \|7.00\| | \|−1.50\| \|0.50\| | \|3.50\| | \|8.50\| |

[symbol]

indicates data missing or illegible when filed

TABLE 17
Scoring of Enrichment Model 2 for STEP

| EM-2 | | | | | | (only SC) | (only SC) |
|---|---|---|---|---|---|---|---|
| | full access | half access | min access | (hp) (polar) | (−1) (0) (+1) | donate | accept |
| STEP | 1.00 | 0.50 | 0.25 | polarity | charge | HBs | HBs |
| P469 | | CB, CG | CA, CD | \|1.50\| \|0.00\| | \|0.00\| \|0.00\| | \|0.00\| | \|0.00\| |
| L472 | | CD1 | CB, CG | \|1.00\| \|0.00\| | \|0.00\| \|0.00\| | \|0.00\| | \|0.00\| |
| H473 | CE1, ND1, | CB, CG, | O, CA CD2 | \|3.00\| \|2.25\| | \|0.00\| \|0.00\| | \|2.00\| | \|2.00\| |
| | NE2 | CD2 | | | | | |
| R476 | CZ, NH1 | CG, CD, | O, CA, CB, | \|2.50\| \|2.00\| | \|0.00\| \|0.75\| | \|3.25\| | \|0.00\| |
| | | NH2 | N [symbol] | | | | |
| E477 | OE2 | CG, CD, | O | \|1.00\| \|1.75\| | \|−0.75\| \|0.00\| | \|0.00\| | \|3.00\| |
| | | OE1 | | | | | |
| E480 | OE2 | CG, CD, | CA, CB | \|1.50\| \|1.50\| | \|−0.75\| \|0.00\| | \|0.00\| | \|3.00\| |
| | | OE1 | | | | | |
| L554 | CD2 | CD1 | CA, CB | \|2.00\| \|0.00\| | \|0.00\| \|0.00\| | \|0.00\| | \|0.00\| |
| Y555 | | CE1, OH | CZ | \|0.75\| \|0.50\| | \|0.00\| \|0.00\| | \|0.50\| | \|0.50\| |
| K557 | CD, CE, | O, CG | C, CA, CB | \|3.25\| \|1.50\| | \|0.00\| \|1.00\| | \|3.00\| | \|0.00\| |
| | NZ | | | | | | |
| Q558 | O, NE2 | CA, CB | C, CG, CD, | \|1.75\| \|2.25\| | \|0.00\| \|0.00\| | \|2.00\| | \|0.50\| |
| | | | OE1 | | | | |
| H561 | CD2, CE1, | O, CB, CG, | N, CA | \|3.25\| \|2.25\| | \|0.00\| \|0.00\| | \|1.50\| | \|1.50\| |
| | NE2 | ND1 | | | | | |
| Q562 | | O, N, C, | CA, OE1 | \|2.25\| \|1.75\| | \|0.00\| \|0.00\| | \|1.00\| | \|0.50\| |
| | | CB, CG, CD, | | | | | |
| | | NE2 | | | | | |
| S563 | O, CB, OG | | N, C, CA | \|1.50\| \|2.25\| | \|0.00\| \|0.00\| | \|1.00\| | \|2.00\| |
| P564 | CG, CD | O, CB | CA | \|2.75\| \|0.50\| | \|0.00\| \|0.00\| | \|0.00\| | \|0.00\| |
| E565 | | OE2 | CG, CD, OE1 | \|0.50\| \|0.75\| | \|−0.50\| \|0.00\| | \|0.00\| | \|1.50\| |
| * | | | | \|0.00\| \|0.00\| | \|0.00\| \|0.00\| | \|0.00\| | \|0.00\| |
| * | | | | \|0.00\| \|0.00\| | \|0.00\| \|0.00\| | \|0.00\| | \|0.00\| |

| | | | | |
|---|---|---|---|---|
| TOTALS | \|28.50\| \|19.50\| | \|−2.00\| \|1.75\| | \|14.25\| | \|14.50\| |

[symbol]

indicates data missing or illegible when filed

TABLE 18
Scoring of Enrichment Model 2 for LYP (PTPN22, PEP, PTPN8)

| EM-2 | | | | | | (only SC) | (only SC) |
|---|---|---|---|---|---|---|---|
| | full access | half access | min access | (hp) (polar) | (−1) (0) (+1) | donate | accept |
| LYP | 1.00 | 0.50 | 0.25 | polarity | charge | HBs | HBs |
| D203 | CB, OD2 | CG | O, C, CA, | \|2.00\| \|1.50\| | \|−0.75\| \|0.00\| | \|0.00\| | \|2.50\| |
| | | | OD1 | | | | |
| L206 | | | CB, O | \|0.25\| \|0.25\| | \|0.00\| \|0.00\| | \|0.00\| | \|0.00\| |
| [symbol] 207 | OE1, OE2 | CB, CG, CD | CA | \|1.75\| \|2.00\| | \|−1.00\| \|0.00\| | \|0.00\| | \|4.00\| |
| W210 | CH2, CZ2, | NE1 | CB, CG, | \|4.50\| \|0.50\| | \|0.00\| \|0.00\| | \|0.50\| | \|0.00\| |
| | CZ3 | | CD1, CD2, | | | | |
| | | | CE2, CE3 | | | | |
| D211 | | CG, OD1, | CA | \|0.75\| \|1.00\| | \|−0.50\| \|0.00\| | \|0.00\| | \|2.00\| |
| | | OD2 | | | | | |
| C214 | | CB, SG | O, C, CA | \|1.50\| \|0.25\| | \|0.00\| \|0.00\| | \|0.00\| | \|0.00\| |
| E288 | | CD, OE1, | CA, CB | \|1.00\| \|1.00\| | \|−0.50\| \|0.00\| | \|0.00\| | \|2.00\| |
| | | OE2 | | | | | |
| L289 | | | O, C, CB, | \|0.75\| \|0.25\| | \|0.00\| \|0.00\| | \|0.00\| | \|0.00\| |
| | | | CD2 | | | | |
| K291 | CD, CE, | | O, CB, CG | \|2.50\| \|1.25\| | \|0.00\| \|1.00\| | \|3.00\| | \|0.00\| |
| | NZ | | | | | | |
| R292 | CD, NH2 | CZ, NE | C, CA, CB, | \|2.25\| \|1.75\| | \|0.00\| \|0.75\| | \|3.00\| | \|0.00\| |
| | | | NH1 | | | | |
| D295 | OD1, OD2 | CG | O, CA, CB | \|1.00\| \|2.25\| | \|−1.00\| \|0.00\| | \|0.00\| | \|4.00\| |
| V296 | O, CG1 | C, CA, CB, | | \|3.00\| \|1.00\| | \|0.00\| \|0.00\| | \|0.00\| | \|0.00\| |
| | | CG2 | | | | | |
| I297 | | O, CA, CG1, | CB, CG2 | \|2.50\| \|0.50\| | \|0.00\| \|0.00\| | \|0.00\| | \|0.00\| |
| | | CG2, CD1 | | | | | |
| R298 | NH2 | CZ, NH1 | O, C, CA, | \|1.25\| \|2.00\| | \|0.00\| \|0.75\| | \|3.25\| | \|0.00\| |
| | | | CG, NE | | | | |
| D299 | O, CB, | CG, OD1 | C, CA, N | \|2.00\| \|2.75\| | \|−0.75\| \|0.00\| | \|0.00\| | \|3.00\| |
| | OD2 | | | | | | |
| K300 | CE, NZ | O, CA, CB, | | \|2.50\| \|1.50\| | \|0.00\| \|1.00\| | \|3.00\| | \|0.00\| |
| | | CG | | | | | |
| T304 | | CG2 | O, CA, CB | \|1.00\| \|0.25\| | \|0.00\| \|0.00\| | \|0.00\| | \|0.00\| |

| | | | | |
|---|---|---|---|---|
| TOTALS | \|30.50\| \|20.00\| | \|−4.50\| \|3.50\| | \|12.75\| | \|17.50\| |

[symbol]

indicates data missing or illegible when filed

TABLE 19
Scoring of Enrichment Model 2 for PTP-PEST (PTPN12, PTPG1)

| EM-2 | | | | | | (only SC) | (only SC) |
|---|---|---|---|---|---|---|---|
| | full access | half access | min access | (hp) (polar) | (−1) (0) (+1) | donate | accept |
| PEST | 1.00 | 0.50 | 0.25 | polarity | charge | HBs | HBs |
| D207 | CG, OD2 | CB, OD1 | N | \|1.50\| \|1.75\| | \|−0.75\| \|0.00\| | \|0.00\| | \|3.00\| |
| L210 | | | CB, CD2 | \|0.50\| \|0.00\| | \|0.00\| \|0.00\| | \|0.00\| | \|0.00\| |
| D211 | CB, OD2 | | CA, CG | \|1.50\| \|1.00\| | \|−0.50\| \|0.00\| | \|0.00\| | \|2.00\| |
| S214 | | CB, OG | | \|1.00\| \|0.00\| | \|0.00\| \|0.00\| | \|0.50\| | \|1.00\| |
| L215 | CD2 | CD1 | CG | \|1.75\| \|0.00\| | \|0.00\| \|0.00\| | \|0.00\| | \|0.00\| |
| K218 | CE | CB, CG, | O, C | \|2.75\| \|0.75\| | \|0.00\| \|0.50\| | \|1.50\| | \|0.00\| |
| | | CD, NZ | | | | | |
| Q292 | OE1 | CA, CB, CD | CG, NE2 | \|1.75\| \|1.25\| | \|0.00\| \|0.00\| | \|0.50\| | \|2.00\| |
| L293 | | | CG, CD1, | \|0.75\| \|0.00\| | \|0.00\| \|0.00\| | \|0.00\| | \|0.00\| |
| | | | CD2 | | | | |
| E295 | CG, CD, | CA, CB, | O | \|3.00\| \|1.75\| | \|−0.75\| \|0.00\| | \|0.00\| | \|3.00\| |
| | OE2 | OE1 | | | | | |
| K296 | | CG, CD, | O, C, | \|2.25\| \|0.75\| | \|0.00\| \|0.50\| | \|1.50\| | \|0.00\| |
| | | CE, NZ | CA, CB | | | | |
| Q299 | NE2, OE1 | CB, CD | O, CA, | \|1.50\| \|2.25\| | \|0.00\| \|0.00\| | \|2.00\| | \|2.00\| |
| | | | CG | | | | |
| L300 | CD1, CD2 | | CA, CB, CG | \|2.75\| \|0.00\| | \|0.00\| \|0.00\| | \|0.00\| | \|0.00\| |
| Y301 | CD2, CE2 | O, CB, CE [symbol] | N, CA, CG, | \|4.50\| \|1.00\| | \|0.00\| \|0.00\| | \|0.00\| | \|0.00\| |
| | | CD1, CZ | OH | | | | |
| E302 | OE1, OE2 | O, CB, CG, | C | \|1.75\| \|2.50\| | \|−1.00\| \|0.00\| | \|0.00\| | \|4.00\| |
| | | CD | | | | | |
| I303 | CB | O, CA | C, CB | \|2.00\| \|0.50\| | \|0.00\| \|0.00\| | \|0.00\| | \|0.00\| |
| H304 | CB | O, CA | N, CG, CD2, | \|2.25\| \|0.75\| | \|0.00\| \|0.00\| | \|0.00\| | \|0.00\| |
| | | | CE1 | | | | |
| K308 | CE, NZ | CD | CA, CB, CG | \|2.25\| \|1.00\| | \|0.00\| \|1.00\| | \|3.00\| | \|0.00\| |

| | | | | |
|---|---|---|---|---|
| TOTALS | \|33.75\| \|15.25\| | \|−3.00\| \|2.00\| | \|9.00\| | \|17.00\| |

[symbol]

indicates data missing or illegible when filed

TABLE 20
Comparison of the scoring for Enrichment Model 2

| EM2 | | | Charge | | H-Bonds | |
|---|---|---|---|---|---|---|
| | hydrophobicity | polarity | negative | positive | donate | accept |
| SHP2 | 26.25 | 17 | −4 | 3 | 13.5 | 16.25 |
| PTP1B | 11.5 | 7 | −1.5 | 0.5 | 3.5 | 8.5 |
| STEP | 28.5 | 19.5 | −2 | 1.75 | 14.25 | 14.5 |
| LYP | 30.5 | 20 | −4.5 | 3.5 | 12.75 | 17.5 |
| PEST | 33.75 | 15.25 | −5 | 2 | 9 | 17 |

TABLE 21
Difference results for the Enrichment Model 2 compared to SHP-2

| EM2 | | | Charge | | H-bonds | |
|---|---|---|---|---|---|---|
| | hydrophobicity | polarity | negative | positive | donate | accept |
| SHP2 | 1 | 1 | 1 | 1 | 1 | 1 |
| PTP1B | 0.44 | 0.41 | 0.38 | 0.17 | 0.26 | 0.52 |
| STEP | 1.09 | 1.15 | 0.5 | 0.58 | 1.06 | 0.89 |
| LYP | 1.16 | 1.18 | 1.13 | 1.17 | 0.94 | 1.08 |
| PEST | 1.29 | 0.9 | 0.75 | 0.67 | 0.67 | 1.05 |

Utilization of the Assessment Factors (AF)

To provide a numerical Comparison Value (CV) for each of the Assessment Factors (AF) the absolute value of each AF was recorded in Table 20. To utilize the SHP-2 model as a comparator each AF was divided by the corresponding AF for SHP-2. Table 21 sets out these values as compared to SHP-2, which was set to 1 to provide normalization of the results

TABLE 22
Scoring of Enrichment Model 3 for SHP-2

| EM-3 | | | | | | (only SC) | (only SC) |
|---|---|---|---|---|---|---|---|
| | full access | half access | min access | (hp) (polar) | (−1) (0) (+1) | donate | accept |
| SHP2 | 1.00 | 0.50 | 0.25 | polarity | charge | HBs | HBs |
| P312 | | | O, CB, CG | \|0.50\| \|0.25\| | \|0.00\| \|0.00\| | \|0.00\| | \|0.00\| |
| [symbol] 313 | | OE1 | CD, OE2 | \|0.25\| \|0.75\| | \|−0.50\| \|0.00\| | \|0.00\| | \|1.50\| |
| K324 | | CE | O, C, CA, | \|1.25\| \|0.50\| | \|0.00\| \|0.25\| | \|0.75\| | \|0.00\| |
| | | | CD, NZ | | | | |
| K325 | | | N, CA, CA, | \|1.25\| \|0.50\| | \|0.00\| \|0.25\| | \|0.75\| | \|0.00\| |
| | | | CB, CG, | | | | |
| | | | CE, NZ | | | | |
| S326 | | OG | O, N, CB | \|0.25\| \|1.00\| | \|0.00\| \|0.00\| | \|0.50\| | \|1.00\| |
| Y327 | | | OH | \|0.00\| \|0.25\| | \|0.00\| \|0.00\| | \|0.25\| | \|0.25\| |
| H447 | | | CE1, O | \|0.25\| \|0.25\| | \|0.00\| \|0.00\| | \|0.00\| | \|0.00\| |
| E451 | | | O, C, CA, | \|1.25\| \|0.50\| | \|−0.25\| \|0.00\| | \|0.00\| | \|0.50\| |
| | | | CB, CG, | | | | |
| | | | CD, OE2 | | | | |
| D481 | | | O, C, CB, | \|0.75\| \|0.25\| | \|0.00\| \|0.00\| | \|0.00\| | \|0.00\| |
| | | | CG | | | | |
| R484 | | | O, CA, | \|0.75\| \|0.50\| | \|0.00\| \|0.25\| | \|0.50\| | \|0.00\| |
| | | | CG, CD, | | | | |
| | | | NH1 | | | | |
| E485 | | O, CG, | CA, CB, | \|1.50\| \|1.25\| | \|−0.50\| \|0.00\| | \|0.00\| | \|1.50\| |
| | | CD, OE2 | OE1 | | | | |
| K538 | | CB, CD, | CA, CG | \|2.00\| \|0.50\| | \|0.00\| \|0.50\| | \|1.50\| | \|0.00\| |
| | | CE, NZ | | | | | |
| S539 | CB | OG | O, N, C, | \|1.50\| \|1.00\| | \|0.00\| \|0.00\| | \|0.50\| | \|1.00\| |
| | | | CA | | | | |
| K542 | | CE | O, C, CG, | \|1.25\| \|0.50\| | \|0.00\| \|0.25\| | \|0.75\| | \|0.00\| |
| | | | CD, NZ | | | | |
| G543 | | CA, C | O | \|1.00\| \|0.25\| | \|0.00\| \|0.00\| | \|0.00\| | \|0.00\| |
| H544 | CE1 | CD2, ND1, | CA, CB, | \|2.25\| \|1.00\| | \|0.00\| \|0.00\| | \|0.50\| | \|0.50\| |
| | | NE2 | CG | \|0.75\| \|0.75\| | \|−0.25\| \|0.00\| | \|0.00\| | \|1.00\| |
| E545 | | | O, CA, | | | | |
| | | | CG, CD, | | | | |
| | | | OE1, OE2 | | | | |
| Y546 | | | O, CB, | \|1.25\| \|0.50\| | \|0.00\| \|0.00\| | \|0.25\| | \|0.25\| |
| | | | CG, CD1, | | | | |
| | | | CD2, CE1, | | | | |
| | | | OH | | | | |
| T547 | | CG2, CB | CA | \|1.25\| \|0.00\| | \|0.00\| \|0.00\| | \|0.00\| | \|0.00\| |

| | | | | |
|---|---|---|---|---|
| TOTALS | \|19.25\| \|10.50\| | \|−1.50\| \|1.50\| | \|6.25\| | \|7.50\| |

[symbol]

indicates data missing or illegible when filed

TABLE 23
Scoring of Enrichment Model 3 for PTP1B

| EM-3 | | | | | | (only SC) | (only SC) |
|---|---|---|---|---|---|---|---|
| | full access | half access | min access | (hp) (polar) | (−1) (0) (+1) | donate | accept |
| PTP1B | 1.00 | 0.50 | 0.25 | polarity | charge | HBs | HBs |
| M74 | | | O, C, CA, | \|1.25\| \|0.25\| | \|0.00\| \|0.00\| | \|0.00\| | \|0.00\| |
| | | | CB, SD, CE | | | | |
| E75 | OE1, OE2 | CB, CG, | O, N, CA | \|1.75\| \|2.50\| | \|−1.00\| \|0.00\| | \|0.00\| | \|4.00\| |
| | | CD | | | | | |
| Q78 | CG, CD, | O, C, CA, | | \|3.50\| \|2.50\| | \|0.00\| \|0.00\| | \|2.00\| | \|2.00\| |
| | OE1, NE2 | CB | | | | | |
| R79 | | | C, CA, CD, | \|1.00\| \|0.50\| | \|0.00\| \|0.25\| | \|0.75\| | \|0.00\| |
| | | | CZ, NE, | | | | |
| | | | NH2 | | | | |
| S80 | | CB, OG | O, N | \|0.50\| \|1.00\| | \|0.00\| \|0.00\| | \|0.50\| | \|1.00\| |
| Y81 | | | | \|0.00\| \|0.00\| | \|0.00\| \|0.00\| | \|0.00\| | \|0.00\| |
| R199 | | CB | O, C, CA, | \|1.75\| \|0.50\| | \|0.00\| \|0.25\| | \|0.50\| | \|0.00\| |
| | | | CG, CD, | | | | |
| | | | CZ, NH1 | | | | |
| S203 | | | | \|0.00\| \|0.00\| | \|0.00\| \|0.00\| | \|0.00\| | \|0.00\| |
| L233 | | CD1 | CA, CD2 | \|1.00\| \|0.00\| | \|0.00\| \|0.00\| | \|0.00\| | \|0.00\| |
| D236 | | | O, C, CA, | \|1.00\| \|0.75\| | \|−0.25\| \|0.00\| | \|0.00\| | \|1.00\| |
| | | | CB, CG, | | | | |
| | | | OD1, OD2 | | | | |
| K237 | O | CA, CB, | C, CD | \|2.50\| \|1.50\| | \|0.00\| \|0.50\| | \|1.50\| | \|0.00\| |
| | | CG, CE, NZ | | | | | |
| Q290 | NE2 | CG, CD, | O, CA, CB | \|1.50\| \|1.75\| | \|0.00\| \|0.00\| | \|2.00\| | \|1.00\| |
| | | OE1 | | | | | |
| W291 | | | CD1 | \|0.00\| \|0.25\| | \|0.00\| \|0.00\| | \|0.00\| | \|0.00\| |
| L294 | CD2 | CG | O, C, CA, | \|2.50\| \|0.25\| | \|0.00\| \|0.00\| | \|0.00\| | \|0.00\| |
| | | | CB, CD1 | | | | |
| S295 | | | O, C | \|0.25\| \|0.25\| | \|0.00\| \|0.00\| | \|0.00\| | \|0.00\| |
| H296 | CE1 | CB, CG, | O | \|2.50\| \|1.25\| | \|0.00\| \|0.00\| | \|1.00\| | \|1.00\| |
| | | CD2, ND1, | | | | | |
| | | NE2 | | | | | |
| E297 | | | | \|0.00\| \|0.00\| | \|0.00\| \|0.00\| | \|0.00\| | \|0.00\| |
| D298 | | O | CA, CB, | \|0.75\| \|0.50\| | \|0.00\| \|0.00\| | \|0.00\| | \|0.00\| |
| | | | CG | | | | |
| L299 | CD1, CD2 | O, CG | C, CB | \|3.00\| \|0.50\| | \|0.00\| \|0.00\| | \|0.00\| | \|0.00\| |

| | | | | |
|---|---|---|---|---|
| TOTALS | \|24.75\| \|14.25\| | \|−1.25\| \|1.00\| | \|8.25\| | \|10.00\| |

TABLE 24
Scoring of Enrichment Model 3 for STEP

| EM-3 | | | | | | (only SC) | (only SC) |
|---|---|---|---|---|---|---|---|
| | full access | half access | min access | (hp) (polar) | (−1) (0) (+1) | donate | accept |
| STEP | 1.00 | 0.50 | 0.25 | polarity | charge | HBs | HBs |
| G361 | | O | N, C, CA | \|0.50\| \|0.75\| | \|0.00\| \|0.00\| | \|0.00\| | \|0.00\| |
| Y362 | | CE2 | O, N, C, | \|2.25\| \|0.50\| | \|0.00\| \|0.00\| | \|0.25\| | \|0.25\| |
| | | | CA, CB, | | | | |
| | | | CD1, CD2, | | | | |
| | | | CE1, CZ, | | | | |
| | | | OH | | | | |
| E366 | OE1, OE2 | CG, CD | O, N, C, | \|1.75\| \|2.50\| | \|−1.00\| \|0.00\| | \|0.00\| | \|4.00\| |
| | | | CA, CB | | | | |
| K367 | | CD | O, N, C, | \|1.25\| \|0.75\| | \|0.00\| \|0.25\| | \|0.75\| | \|0.00\| |
| | | | CB, CE, | | | | |
| | | | NZ | | | | |
| V368 | | CB, CG [symbol] 0, | C, CA | \|2.00\| \|0.00\| | \|0.00\| \|0.00\| | \|0.00\| | \|0.00\| |
| | | CG2 | | | | | |
| Y369 | | | CD1, CE1, | \|0.75\| \|0.25\| | \|0.00\| \|0.00\| | \|0.25\| | \|0.25\| |
| | | | CZ, OH | | | | |
| E479 | | CD, OE1 | O, C, CA, | \|1.50\| \|1.00\| | \|−0.50\| \|0.00\| | \|0.00\| | \|1.50\| |
| | | | CB, CG, | | | | |
| | | | OE2 | | | | |
| Q483 | O, OE1, | CB, CD | C, CA, CG | \|1.75\| \|3.00\| | \|0.00\| \|0.00\| | \|2.00\| | \|2.00\| |
| | NE2 | | | | | | |
| Q514 | | CG, NE2 | C, CA, CB, | \|1.50\| \|0.75\| | \|0.00\| \|0.00\| | \|1.00\| | \|0.50\| |
| | | | CD, OE1 | | | | |
| R517 | CZ, NH1, | O, CB, | C, CA, CG | \|2.75\| \|3.00\| | \|−1.00\| \|0.00\| | \|4.50\| | \|0.00\| |
| | NE2 | CD, NE | | | | | |
| Q518 | NE2 | O, CD, | C, CA, CB, | \|1.50\| \|2.00\| | \|0.00\| \|0.00\| | \|2.00\| | \|1.00\| |
| | | OE1 | CG | | | | |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |
| none | | | | \|0.00\| \|0.00\| | \|0.00\| \|0.00\| | \|0.00\| | \|0.00\| |

| | | | | |
|---|---|---|---|---|
| TOTALS | \|17.50\| \|14.50\| | \|−2.50\| \|0.25\| | \|10.75\| | \|9.50\| |

[symbol]

indicates data missing or illegible when filed

TABLE 5
Scoring of Enrichment Model 3 for LYP (PTPN22, PEP, PTPN8)

| EM-3 | | | | | | (only SC) | (only SC) |
|---|---|---|---|---|---|---|---|
| | full access | half access | min access | (hp) (polar) | (−1) (0) (+1) | donate | accept |
| LYP | 1.00 | 0.50 | 0.25 | polarity | charge | HBs | HBs |
| G92 | | | O, C | \|0.25\| \|0.25\| | \|0.00\| \|0.00\| | \|0.00\| | \|0.00\| |
| V93 | | CG1 | O, CA, | \|1.25\| \|0.25\| | \|0.00\| \|0.00\| | \|0.00\| | \|0.00\| |
| | | | CB, CG2 | | | | |
| P96 | | CB, CG, | O, C, CA | \|2.00\| \|0.25\| | \|0.00\| \|0.00\| | \|0.00\| | \|0.00\| |
| | | CD | | | | | |
| K97 | | | N, CA, | \|1.00\| \|0.25\| | \|0.00\| \|0.00\| | \|0.00\| | \|0.00\| |
| | | | CB, CG, | | | | |
| | | | CE | | | | |
| A98 | | CB | N | \|0.50\| \|0.25\| | \|0.00\| \|0.00\| | \|0.00\| | \|0.00\| |
| Y99 | | | | \|0.00\| \|0.00\| | \|0.00\| \|0.00\| | \|0.00\| | \|0.00\| |
| R213 | | | C, CB, CZ, | \|0.75\| \|0.25\| | \|0.00\| \|0.25\| | \|0.50\| | \|0.00\| |
| | | | NH2 | | | | |
| E217 | O, CD, | CB, CG, | C, CA | \|2.50\| \|2.50\| | \|−0.75\| \|0.00\| | \|0.00\| | \|3.00\| |
| | OE1 | OE2 | | | | | |
| M245 | | CE | SD | \|0.75\| \|0.00\| | \|0.00\| \|0.00\| | \|0.00\| | \|0.00\| |
| K248 | NZ | CE | O, C, CA, | \|1.75\| \|1.25\| | \|0.00\| \|1.00\| | \|3.00\| | \|0.00\| |
| | | | CB, CG, | | | | |
| | | | CD | | | | |
| D249 | O | CA, CB, | C | \|1.75\| \|2.00\| | \|−0.50\| \|0.00\| | \|0.00\| | \|2.00\| |
| | | CG, OD1, | | | | | |
| | | OD2 | | | | | |
| S302 | CB, OG | O, C, CA | | \|2.00\| \|1.50\| | \|0.00\| \|0.00\| | \|1.00\| | \|2.00\| |
| G303 | | CA | O, N, C | \|0.75\| \|0.50\| | \|0.00\| \|0.00\| | \|0.00\| | \|0.00\| |
| S306 | CB | O, OG | N, C, CA | \|1.50\| \|1.25\| | \|0.00\| \|0.00\| | \|0.50\| | \|1.00\| |
| Q307 | NE2 | O, CA, | C, CG, | \|2.00\| \|1.75\| | \|0.00\| \|0.00\| | \|2.00\| | \|0.25\| |
| | | CB, CD | OE1 | | | | |
| A308 | CB | CA | O, C | \|1.75\| \|0.25\| | \|0.00\| \|0.00\| | \|0.00\| | \|0.00\| |
| K309 | CD, CE, | O, CB, CG | N, C, CA | \|3.50\| \|1.75\| | \|0.00\| \|1.00\| | \|3.00\| | \|0.00\| |
| | NZ | | | | | | |
| H310 | CE1, NE2 | O, CD2, | N, C, CA, | \|2.50\| \|1.75\| | \|0.00\| \|0.00\| | \|1.50\| | \|1.50\| |
| | | ND1 | CB, CG | | | | |
| C311 | S [symbol] , CB | O | C, CA | \|2.50\| \|0.50\| | \|0.00\| \|0.00\| | \|0.00\| | \|0.00\| |

| | | | | |
|---|---|---|---|---|
| TOTALS | \|29.00\| \|16.50\| | −\|1.25\| \|2.25\| | \|11.50\| | \|9.75\| |

[symbol]

indicates data missing or illegible when filed

TABLE 26
Scoring of Enrichment Model 3 for PTP-PEST (PTPN12, PTPG1)

| EM-3 | | | | | | (only SC) | (only SC) |
|---|---|---|---|---|---|---|---|
| PTP- | full access | half access | min access | (hp) (polar) | (−1) (0) (+1) | donate | accept |
| PEST | 1.00 | 0.50 | 0.25 | polarity | charge | HBs | HBs |
| G96 | | | O, C | \|0.25\| \|0.25\| | \|0.00\| \|0.00\| | \|0.00\| | \|0.00\| |
| V97 | CG1, CG2 | CA, CB | N | \|3.00\| \|0.25\| | \|0.00\| \|0.00\| | \|0.00\| | \|0.00\| |
| P100 | CG, CD | O, CA, CB | C | \|3.25\| \|0.50\| | \|0.00\| \|0.00\| | \|0.00\| | \|0.00\| |
| K101 | | CE | N, O, CA | \|1.25\| \|0.50\| | \|0.00\| \|0.00\| | \|0.00\| | \|0.00\| |
| A102 | | CB | N, O | \|0.50\| \|0.50\| | \|0.00\| \|0.00\| | \|0.00\| | \|0.00\| |
| Y103 | | | OH | \|0.00\| \|0.25\| | \|0.00\| \|0.00\| | \|0.25\| | \|0.25\| |
| R217 | | NH1, NH2 | CD, CZ | \|0.50\| \|1.00\| | \|0.00\| \|0.50\| | \|2.00\| | \|0.00\| |
| E221 | CG | O, CB, | C, OE1 | \|2.25\| \|1.25\| | \|−0.50\| \|0.00\| | \|0.00\| | \|1.50\| |
| | | CD, OE2 | | | | | |
| N249 | | ND2 | O, CG, | \|0.25\| \|1.00\| | \|0.00\| \|0.00\| | \|1.00\| | \|0.50\| |
| | | | OD1 | | | | |
| K252 | | CE, NZ | O, CG, CD | \|1.00\| \|0.75\| | \|0.00\| \|0.50\| | \|1.50\| | \|0.00\| |
| A253 | CB | O, CA | C | \|1.75\| \|0.50\| | \|0.00\| \|0.00\| | \|0.00\| | \|0.00\| |
| A306 | CB | CA | N, O, C | \|1.75\| \|0.50\| | \|0.00\| \|0.00\| | \|0.00\| | \|0.00\| |
| Q307 | CB, CG, | O, CA, | C | \|3.75\| \|2.00\| | \|0.00\| \|0.00\| | \|2.00\| | \|1.00\| |
| | CD, NE2 | OE1 | | | | | |
| A310 | CB | | O, C, CA | \|1.50\| \|0.25\| | \|0.00\| \|0.00\| | \|0.00\| | \|0.00\| |
| D311 | CG, OD2 | CA, CB, | N, O | \|2.00\| \|2.00\| | \|−0.75\| \|0.00\| | \|0.00\| | \|3.00\| |
| | | OD1 | | | | | |
| G312 | | O, CA | C | \|0.75\| \|0.50\| | \|0.00\| \|0.00\| | \|0.00\| | \|0.00\| |
| V313 | CG1, CG2 | O | CA, CB | \|2.50\| \|0.50\| | \|0.00\| \|0.00\| | \|0.00\| | \|0.00\| |
| N314 | ND2 | CG, OD1 | O, C, CA, | \|1.25\| \|1.75\| | \|0.00\| \|0.00\| | \|2.00\| | \|1.00\| |
| | | | CB | | | | |
| E315 | CG, CD, | N, O, CB | CA | \|2.75\| \|3.00\| | \|−1.00\| \|0.00\| | \|0.00\| | \|4.00\| |
| | OE1, OE2 | | | | | | |

| | | | | |
|---|---|---|---|---|
| TOTALS | \|30.25\| \|17.25\| | \|−2.25\| \|1.00\| | \|8.75\| | \|11.25\| |

TABLE 27
Comparison of the scoring for Enrichment Model 3

| EM3 | | | Charge | | H-Bonds | |
|---|---|---|---|---|---|---|
| | hydrophobicity | polarity | negative | positive | donate | accept |
| SHP2 | 19.25 | 10.5 | −1.5 | 1.5 | 6.25 | 7.5 |
| PTP1B | 24.75 | 14.25 | −1.25 | 1 | 8.25 | 10 |
| STEP | 17.5 | 14.5 | −2.5 | 0.25 | 10.75 | 9.5 |
| LYP | 29 | 16.5 | −1.25 | 2.25 | 11.5 | 9.75 |
| PEST | 30.25 | 17.25 | −2.25 | 1 | 8.75 | 11.25 |

TABLE 28
Difference results for Enrichment Model 3 Compared to SHP-2

| EM3 | | | Charge | | H-bonds | |
|---|---|---|---|---|---|---|
| | hydrophobicity | polarity | negative | positive | donate | accept |
| SHP2 | 1 | 1 | 1 | 1 | 1 | 1 |
| PTP1B | 1.29 | 1.36 | 0.83 | 0.67 | 1.32 | 1.33 |
| STEP | 0.91 | 1.38 | 1.67 | 0.17 | 1.72 | 1.27 |
| LYP | 1.51 | 1.57 | 0.83 | 1.5 | 1.84 | 1.3 |
| PEST | 1.57 | 1.64 | 1.5 | 0.67 | 1.4 | 1.5 |

Utilization of the Assessment Factors (AF)

To provide a numerical Comparison Value (CV) for each of the Assessment Factors (AF) the absolute value of each AF was recorded in Table 27. To utilize the SHP-2 model as a comparator each AF was divided by the corresponding AF for SHP-2. Table 28 sets out these values as compared to SHP-2, which was set to 1 to provide normalization of the results.

TABLE 29
Scoring of Enrichment Model 4.1 for SHP-2

| EM-4.1 | | | | | (−1) | | |
|---|---|---|---|---|---|---|---|
| | full | half | min | (hp) | (0) | (only SC) | (only SC) |
| | access | access | access | (polar) | (+1) | donate | accept |
| SHP2 | 1.00 | 0.50 | 0.25 | polarity | charge | HBs | HBs |
| Y327 | | | OH | \|0.00\| \|0.25\| | \|0.00\| \|0.00\| | \|0.25\| | \|0.25\| |
| V354 | | | | \|0.00\| \|0.00\| | \|0.00\| \|0.00\| | \|0.00\| | \|0.00\| |
| D395 | | OD1, | N, CB, | \|0.50\| \|1.25\| | \|−0.50\| \|0.00\| | \|0.00\| | \|2.00\| |
| | | OD2 | CG | | | | |
| F424 | | | CE1, CZ | \|0.50\| \|0.00\| | \|0.00\| \|0.00\| | \|0.00\| | \|0.00\| |
| T426 | CB, | O, CA | C, OG1 | \|2.75\| \|0.75\| | \|0.00\| \|0.00\| | \|0.25\| | \|0.50\| |
| | CG2 | | | | | | |
| W427 | | O | C, CB, | \|1.25\| \|0.75\| | \|0.00\| \|0.00\| | \|0.25\| | \|0.00\| |
| | | | CE2, CD1, | | | | |
| | | | CZ2, NE1 | | | | |
| P433 | | CA, CB | C, O | \|1.25\| \|0.25\| | \|0.00\| \|0.00\| | \|0.00\| | \|0.00\| |
| P435 | CB, | CA | O, N | \|2.50\| \|2.50\| | \|−1.00\| \|0.00\| | \|0.00\| | \|4.00\| |
| | CG, | | | | | | |
| | OD1, | | | | | | |
| | OD2 | | | | | | |
| P436 | | CG, CD | | \|1.00\| \|0.00\| | \|0.00\| \|0.00\| | \|0.00\| | \|0.00\| |
| G437 | | C, CA | O, N | \|1.00\| \|0.50\| | \|0.00\| \|0.00\| | \|0.00\| | \|0.00\| |
| G438 | | CA | C, N | \|0.75\| \|0.25\| | \|0.00\| \|0.00\| | \|0.00\| | \|0.00\| |
| V439 | | | N, CG1, | \|0.50\| \|0.25\| | \|0.00\| \|0.00\| | \|0.00\| | \|0.00\| |
| | | | CG2 | | | | |
| L440 | | CB | C, CD1 | \|1.00\| \|0.00\| | \|0.00\| \|0.00\| | \|0.00\| | \|0.00\| |
| D441 | | CD2 | N, CA, | \|1.00\| \|0.50\| | \|−0.25\| \|0.00\| | \|0.00\| | \|0.25\| |
| | | | CG, OD1 | | | | |
| F442 | | | CA | \|0.25\| \|0.00\| | \|0.00\| \|0.00\| | \|0.00\| | \|0.00\| |
| L443 | | | | \|0.00\| \|0.00\| | \|0.00\| \|0.00\| | \|0.00\| | \|0.00\| |
| E444 | | CG | C, CB, | \|1.25\| \|0.25\| | \|−0.25\| \|0.00\| | \|0.00\| | \|0.50\| |
| | | | CD, OE1 | | | | |
| V446 | | | | \|0.00\| \|0.00\| | \|0.00\| \|0.00\| | \|0.00\| | \|0.00\| |
| V459 | | | | \|0.00\| \|0.00\| | \|0.00\| \|0.00\| | \|0.00\| | \|0.00\| |
| V461 | | | | \|0.00\| \|0.00\| | \|0.00\| \|0.00\| | \|0.00\| | \|0.00\| |
| V473 | | | | \|0.00\| \|0.00\| | \|0.00\| \|0.00\| | \|0.00\| | \|0.00\| |
| I474 | | | | \|0.00\| \|0.00\| | \|0.00\| \|0.00\| | \|0.00\| | \|0.00\| |
| I476 | | | | \|0.00\| \|0.00\| | \|0.00\| \|0.00\| | \|0.00\| | \|0.00\| |
| D477 | | | | \|0.00\| \|0.00\| | \|0.00\| \|0.00\| | \|0.00\| | \|0.00\| |
| I480 | | | | \|0.00\| \|0.00\| | \|0.00\| \|0.00\| | \|0.00\| | \|0.00\| |
| F517 | | CD1 | CA, CE1 | \|1.00\| \|0.00\| | \|0.00\| \|0.00\| | \|0.00\| | \|0.00\| |
| A521 | | | CA, CB | \|0.50\| \|0.00\| | \|0.00\| \|0.00\| | \|0.00\| | \|0.00\| |
| V522 | | | | \|0.00\| \|0.00\| | \|0.00\| \|0.00\| | \|0.00\| | \|0.00\| |
| H524 | CD2, | CE1 | CB | \|1.75\| \|1.00\| | \|0.00\| \|0.00\| | \|1.00\| | \|1.00\| |
| | NF2 | | | | | | |
| Y525 | | CE1 | CE2, CD1, | \|1.25\| \|0.25\| | \|0.00\| \|0.00\| | \|0.25\| | \|0.25\| |
| | | | CZ, OH | | | | |
| T528 | CB, | C, CA | O, OG1 | \|3.00\| \|0.25\| | \|0.00\| \|0.00\| | \|0.25\| | \|0.50\| |
| | CG2 | | | | | | |
| R532 | CD, CZ | CG | CA, CB | \|3.00\| \|2.00\| | \|0.00\| \|1.00\| | \|4.00\| | \|0.00\| |
| | NH1, NH2 | | | | | | |

| | | | | |
|---|---|---|---|---|
| TOTALS | \|26.00\| \|11.00\| | \|−2.00\| \|1.00\| | \|6.25\| | \|9.25\| |

TABLE 30
Scoring of Enrichment Model 4.1 for PTP1B

| EM-4.1 | | | | | (−1) | | |
|---|---|---|---|---|---|---|---|
| | full | half | min | (hp) | (0) | (only SC) | (only SC) |
| | access | access | access | (polar) | (+1) | donate | accept |
| PTP1B | 1.00 | 0.50 | 0.25 | polarity | charge | HBs | HBs |
| Y81 | | | | \|0.00\| \|0.00\| | \|0.00\| \|0.00\| | \|0.00\| | \|0.00\| |
| V108 | | | | \|0.00\| \|0.00\| | \|0.00\| \|0.00\| | \|0.00\| | \|0.00\| |
| Y152 | | CB | CA, CG, | \|1.25\| \|0.00\| | \|0.00\| \|0.00\| | \|0.00\| | \|0.00\| |
| | | | CD1 | | | | |
| Y176 | | | O | \|0.00\| \|0.25\| | \|0.00\| \|0.00\| | \|0.00\| | \|0.00\| |
| T178 | OG1 | CB, CG2 | O, C, CA | \|1.50\| \|1.25\| | \|0.00\| \|0.00\| | \|1.00\| | \|2.00\| |
| W179 | | | O, N, C | \|0.25\| \|0.50\| | \|0.00\| \|0.00\| | \|0.00\| | \|0.00\| |
| P185 | | | O, C | \|0.25\| \|0.25\| | \|0.00\| \|0.00\| | \|0.00\| | \|0.00\| |
| S187 | | CB, OG | CA | \|0.75\| \|0.50\| | \|0.00\| \|0.00\| | \|0.50\| | \|1.00\| |
| P188 | | CG, CD | CB | \|0.75\| \|0.00\| | \|0.00\| \|0.00\| | \|0.00\| | \|0.00\| |
| A189 | | CB | N, CA | \|0.75\| \|0.25\| | \|0.00\| \|0.00\| | \|0.00\| | \|0.00\| |
| S190 | | | | \|0.00\| \|0.00\| | \|0.00\| \|0.00\| | \|0.00\| | \|0.00\| |
| F191 | | | | \|0.00\| \|0.00\| | \|0.00\| \|0.00\| | \|0.00\| | \|0.00\| |
| L192 | | | | \|0.00\| \|0.00\| | \|0.00\| \|0.00\| | \|0.00\| | \|0.00\| |
| N193 | | | | \|0.00\| \|0.00\| | \|0.00\| \|0.00\| | \|0.00\| | \|0.00\| |
| F194 | | | | \|0.00\| \|0.00\| | \|0.00\| \|0.00\| | \|0.00\| | \|0.00\| |
| L195 | | | | \|0.00\| \|0.00\| | \|0.00\| \|0.00\| | \|0.00\| | \|0.00\| |
| F196 | | | CD2, CE2 | \|0.00\| \|0.50\| | \|0.00\| \|0.00\| | \|0.00\| | \|0.00\| |
| V198 | | | | \|0.00\| \|0.00\| | \|0.00\| \|0.00\| | \|0.00\| | \|0.00\| |
| V211 | | | N, CB, | \|0.50\| \|0.25\| | \|0.00\| \|0.00\| | \|0.00\| | \|0.00\| |
| | | | CG1 | | | | |
| F225 | | | | \|0.00\| \|0.00\| | \|0.00\| \|0.00\| | \|0.00\| | \|0.00\| |
| C226 | | | | \|0.00\| \|0.00\| | \|0.00\| \|0.00\| | \|0.00\| | \|0.00\| |
| A228 | | | | \|0.00\| \|0.00\| | \|0.00\| \|0.00\| | \|0.00\| | \|0.00\| |
| D229 | | | | \|0.00\| \|0.00\| | \|0.00\| \|0.00\| | \|0.00\| | \|0.00\| |
| L232 | | | | \|0.00\| \|0.00\| | \|0.00\| \|0.00\| | \|0.00\| | \|0.00\| |
| F269 | | CD1 | CA, CB, | \|1.25\| \|0.00\| | \|0.00\| \|0.00\| | \|0.00\| | \|0.00\| |
| | | | CE1 | | | | |
| A273 | | | | \|0.00\| \|0.00\| | \|0.00\| \|0.00\| | \|0.00\| | \|0.00\| |
| V274 | | | | \|0.00\| \|0.00\| | \|0.00\| \|0.00\| | \|0.00\| | \|0.00\| |
| E276 | OE1 | CD | CA, CB | \|1.00\| \|1.50\| | \|−0.75\| \|0.00\| | \|0.00\| | \|3.00\| |
| G277 | | | | \|0.00\| \|0.00\| | \|0.00\| \|0.00\| | \|0.00\| | \|0.00\| |
| F280 | | CE2, CZ | O, CD1, | \|1.50\| \|0.25\| | \|0.00\| \|0.00\| | \|0.00\| | \|0.00\| |
| | | | CE1 | | | | |
| D284 | CB, CG, | CA, OD1 | N | \|2.50\| \|1.75\| | \|−0.75\| \|0.00\| | \|0.00\| | \|3.00\| |
| | OD2 | | | | | | |

| | | | | |
|---|---|---|---|---|
| TOTALS | \|12.25\| \|7.25\| | \|−1.50\| \|0.00\| | \|1.50\| | \|9.00\| |

TABLE 31
Scoring of Enrichment Model 4.1 for STEP

| EM-4.1 | | | | | (−1) | | |
|---|---|---|---|---|---|---|---|
| | full | half | min | (hp) | (0) | (only SC) | (only SC) |
| | access | access | access | (polar) | (+1) | donate | accept |
| STEP | 1.00 | 0.50 | 0.25 | polarity | charge | HBs | HBs |
| Y369 | | CE1 | CD1, CZ, | \|1.00\| \|0.25\| | \|0.00\| \|0.00\| | \|0.25\| | \|0.25\| |
| | | | OH | | | | |
| V396 | | | | \|0.00\| \|0.00\| | \|0.00\| \|0.00\| | \|0.00\| | \|0.00\| |
| D434 | | CB | N | \|0.50\| \|0.25\| | \|0.00\| \|0.00\| | \|0.00\| | \|0.00\| |
| F456 | | | CG1, CZ | \|0.50\| \|0.00\| | \|0.00\| \|0.00\| | \|0.00\| | \|0.00\| |
| S458 | | O, CA, | C | \|1.25\| \|1.00\| | \|0.00\| \|0.00\| | \|0.50\| | \|1.00\| |
| | | CB, OG | | | | | |
| W459 | | | O, C, | \|0.75\| \|0.50\| | \|0.00\| \|0.00\| | \|0.25\| | \|0.00\| |
| | | | CE2, CZ2, | | | | |
| | | | NE1 | | | | |
| P465 | | O, CB | C, CA, | \|1.25\| \|0.50\| | \|0.00\| \|0.00\| | \|0.00\| | \|0.00\| |
| | | | CG | | | | |
| R467 | CD, CZ, | CB, OG | O, N, | \|3.50\| \|2.50\| | \|0.00\| \|0.75\| | \|4.00\| | \|0.00\| |
| | NH1, | | CA, CB | | | | |
| | NH2 | | | | | | |
| A468 | | | N, CB | \|0.25\| \|0.25\| | \|0.00\| \|0.00\| | \|0.00\| | \|0.00\| |
| P469 | | CB, CG | CA, CD | \|1.50\| \|0.00\| | \|0.00\| \|0.00\| | \|0.00\| | \|0.00\| |
| P470 | | CG1 | CA, CB, | \|1.25\| \|0.00\| | \|0.00\| \|0.00\| | \|0.00\| | \|0.00\| |
| | | | CD | | | | |
| L471 | | | N, CB, | \|0.50\| \|0.25\| | \|0.00\| \|0.00\| | \|0.00\| | \|0.00\| |
| | | | CD1 | | | | |
| L472 | | CD1 | CB, CG | \|1.00\| \|0.00\| | \|0.00\| \|0.00\| | \|0.00\| | \|0.00\| |
| H473 | CE1, | CG, | O, CA, | \|2.50\| \|2.25\| | \|0.00\| \|0.00\| | \|2.00\| | \|2.00\| |
| | ND1, | CD2 | CB | | | | |
| | NE2 | | | | | | |
| L474 | | | | \|0.00\| \|0.00\| | \|0.00\| \|0.00\| | \|0.00\| | \|0.00\| |
| V475 | | | CG1 | \|0.25\| \|0.00\| | \|0.00\| \|0.00\| | \|0.00\| | \|0.00\| |
| R476 | CZ, | CG, | O, CA, | \|2.50\| \|2.25\| | \|0.00\| \|0.75\| | \|4.50\| | \|0.00\| |
| | NH1, | CD, | CB | | | | |
| | NH2 | NE | | | | | |
| V478 | | | | \|0.00\| \|0.00\| | \|0.00\| \|0.00\| | \|0.00\| | \|0.00\| |
| I492 | | | CD1 | \|0.25\| \|0.00\| | \|0.00\| \|0.00\| | \|0.00\| | \|0.00\| |
| V494 | | | | \|0.00\| \|0.00\| | \|0.00\| \|0.00\| | \|0.00\| | \|0.00\| |
| F506 | | | | \|0.00\| \|0.00\| | \|0.00\| \|0.00\| | \|0.00\| | \|0.00\| |
| I507 | | | | \|0.00\| \|0.00\| | \|0.00\| \|0.00\| | \|0.00\| | \|0.00\| |
| T509 | | | | \|0.00\| \|0.00\| | \|0.00\| \|0.00\| | \|0.00\| | \|0.00\| |
| S510 | | | O, C, | \|0.75\| \|0.25\| | \|0.00\| \|0.00\| | \|0.00\| | \|0.00\| |
| | | | CA, CB | | | | |
| C513 | | CB | C, SG | \|1.00\| \|0.00\| | \|0.00\| \|0.00\| | \|0.00\| | \|0.00\| |
| F547 | | | CA, CB, | \|1.00\| \|0.00\| | \|0.00\| \|0.00\| | \|0.00\| | \|0.00\| |
| | | | CD1, CE1 | | | | |
| V551 | | | | \|0.00\| \|0.00\| | \|0.00\| \|0.00\| | \|0.00\| | \|0.00\| |
| M552 | | | | \|0.00\| \|0.00\| | \|0.00\| \|0.00\| | \|0.00\| | \|0.00\| |
| L554 | CD2 | CD1 | N, CA, | \|2.00\| \|0.25\| | \|0.00\| \|0.00\| | \|0.00\| | \|0.00\| |
| | | | CB | | | | |
| Y555 | | CE1, OH | CZ | \|0.75\| \|0.50\| | \|0.00\| \|0.00\| | \|0.50\| | \|0.50\| |
| Q558 | O, | CA, CB | C, CG, | \|1.75\| \|2.25\| | \|0.00\| \|0.00\| | \|2.00\| | \|0.50\| |
| | NE2 | | CD, OE1 | | | | |
| Q562 | | O, N, | C, CA, | \|1.50\| \|1.50\| | \|0.00\| \|0.00\| | \|0.50\| | \|0.50\| |
| | | CG | CB, CD, | | | | |
| | | | OE1, NE2 | | | | |

| | | | | |
|---|---|---|---|---|
| TOTALS | \|27.50\| \|14.75\| | \|−0.00\| \|1.50\| | \|14.50\| | \|4.75\| |

TABLE 32
Scoring of Enrichment Model 4.1 for LYP (PTPN22, PEP, PTPN8)

| EM-4.1 | | | | | (−1) | | |
|---|---|---|---|---|---|---|---|
| | full | half | min | (hp) | (0) | (only SC) | (only SC) |
| | access | access | access | (polar) | (+1) | donate | accept |
| LYP | 1.00 | 0.50 | 0.25 | polarity | charge | HBs | HBs |
| Y99 | | | | \|0.00\| \|0.00\| | \|0.00\| \|0.00\| | \|0.00\| | \|0.00\| |
| V126 | | | | \|0.00\| \|0.00\| | \|0.00\| \|0.00\| | \|0.00\| | \|0.00\| |
| D168 | | OD1 | C, CA, | \|0.75\| \|0.50\| | \|−0.25\| \|0.00\| | \|0.00\| | \|1.00\| |
| | | | CB | | | | |
| Y190 | | | OH | \|0.00\| \|0.25\| | \|0.00\| \|0.00\| | \|0.25\| | \|0.25\| |
| N192 | ND2 | O, CA, | C | \|1.25\| \|2.00\| | \|0.00\| \|0.00\| | \|2.00\| | \|1.00\| |
| | | CG, OD1 | | | | | |
| W193 | | O | C, CB | \|0.50\| \|0.50\| | \|0.00\| \|0.00\| | \|0.00\| | \|0.00\| |
| P199 | | | O, CA, | \|0.50\| \|0.25\| | \|0.00\| \|0.00\| | \|0.00\| | \|0.00\| |
| | | | CB | | | | |
| S201 | CB | OG | O, N, | \|1.25\| \|1.00\| | \|0.00\| \|0.00\| | \|0.50\| | \|1.00\| |
| | | | CA | | | | |
| I202 | CD1 | | CG2 | \|1.25\| \|0.00\| | \|0.00\| \|0.00\| | \|0.00\| | \|0.00\| |
| D203 | OD2 | CB | O, C, | \|1.25\| \|1.50\| | \|−0.75\| \|0.00\| | \|0.00\| | \|2.50\| |
| | | | CA, CG, | | | | |
| | | | OD1 | | | | |
| P204 | | CG | CA, CB, | \|1.25\| \|0.00\| | \|0.00\| \|0.00\| | \|0.00\| | \|0.00\| |
| | | | CD | | | | |
| I205 | | | | \|0.00\| \|0.00\| | \|0.00\| \|0.00\| | \|0.00\| | \|0.00\| |
| L206 | | | O, C, | \|0.75\| \|0.25\| | \|0.00\| \|0.00\| | \|0.00\| | \|0.00\| |
| | | | CB, CD2 | | | | |
| E207 | OE1, | CD | N, CA, | \|1.25\| \|2.25\| | \|−1.00\| \|0.00\| | \|0.00\| | \|4.00\| |
| | OE2 | | CB, CG | | | | |
| L208 | | | | \|0.00\| \|0.00\| | \|0.00\| \|0.00\| | \|0.00\| | \|0.00\| |
| I209 | | | CB, CG2, | \|0.75\| \|0.00\| | \|0.00\| \|0.00\| | \|0.00\| | \|0.00\| |
| | | | CD1 | | | | |
| W210 | CZ2, | CD1, | CB, CG | \|5.50\| \|0.50\| | \|0.00\| \|0.00\| | \|0.50\| | \|0.00\| |
| | CZ3, | CD2, | | | | | |
| | CH2 | CE2, | | | | | |
| | | CE3, | | | | | |
| | | NE1 | | | | | |
| V212 | | | | \|0.00\| \|0.00\| | \|0.00\| \|0.00\| | \|0.00\| | \|0.00\| |
| I223 | | | CD1 | \|0.25\| \|0.00\| | \|0.00\| \|0.00\| | \|0.00\| | \|0.00\| |
| I225 | | | | \|0.00\| \|0.00\| | \|0.00\| \|0.00\| | \|0.00\| | \|0.00\| |
| I237 | | | | \|0.00\| \|0.00\| | \|0.00\| \|0.00\| | \|0.00\| | \|0.00\| |
| C238 | | | | \|0.00\| \|0.00\| | \|0.00\| \|0.00\| | \|0.00\| | \|0.00\| |
| I240 | | | | \|0.00\| \|0.00\| | \|0.00\| \|0.00\| | \|0.00\| | \|0.00\| |
| D241 | | | | \|0.00\| \|0.00\| | \|0.00\| \|0.00\| | \|0.00\| | \|0.00\| |
| W244 | | CZ2 | CG, CD1, | \|1.75\| \|0.25\| | \|0.00\| \|0.00\| | \|0.25\| | \|0.00\| |
| | | | CD2, CE2, | | | | |
| | | | NE1, CH2 | | | | |
| L281 | | CD1 | CA, CB | \|1.00\| \|0.00\| | \|0.00\| \|0.00\| | \|0.00\| | \|0.00\| |
| A285 | | | | \|0.00\| \|0.00\| | \|0.00\| \|0.00\| | \|0.00\| | \|0.00\| |
| V286 | | | | \|0.00\| \|0.00\| | \|0.00\| \|0.00\| | \|0.00\| | \|0.00\| |
| E288 | | CA, CD, | N, CB | \|1.25\| \|1.25\| | \|−0.50\| \|0.00\| | \|0.00\| | \|2.00\| |
| | | OE1, OE2 | | | | | |
| L289 | | | O, CB, | \|0.50\| \|0.25\| | \|0.00\| \|0.00\| | \|0.00\| | \|0.00\| |
| | | | CD2 | | | | |
| R292 | NH2 | CD, CZ, | C, CA, | \|1.75\| \|1.75\| | \|0.00\| \|0.50\| | \|3.00\| | \|0.00\| |
| | | NE | CB, NH1 | | | | |
| V296 | O, | CB, CG2 | C, CA | \|2.50\| \|1.00\| | \|0.00\| \|0.50\| | \|3.00\| | \|0.00\| |
| | CG1 | | | | | | |

| | | | | |
|---|---|---|---|---|
| TOTALS | \|25.25\| \|13.50\| | \|−2.50\| \|0.50\| | \|6.50\| | \|11.75\| |

TABLE 33
Scoring of Enrichment Model 4.1 for PTP-PEST (PTPN12, PTPG1)

| EM-4.1 | | | | | (−1) | | |
|---|---|---|---|---|---|---|---|
| | full | half | min | (hp) | (0) | (only SC) | (only SC) |
| | access | access | access | (polar) | (+1) | donate | accept |
| PEST | 1.00 | 0.50 | 0.25 | polarity | charge | HBs | HBs |
| Y103 | | | CE1, CD1, | \|0.50\| \|0.25\| | \|0.00\| \|0.00\| | \|0.25\| | \|0.25\| |
| | | | OH | | | | |
| V130 | | | | \|0.00\| \|0.00\| | \|0.00\| \|0.00\| | \|0.00\| | \|0.00\| |
| D172 | | OD1, OD2 | CB, CG | \|0.50\| \|2.00\| | \|−0.50\| \|0.00\| | \|0.00\| | \|2.00\| |
| Y194 | | | OH | \|0.00\| \|0.25\| | \|0.00\| \|0.00\| | \|0.25\| | \|0.25\| |
| N196 | ND2, | O, CG | C, CA, CB | \|1.25\| \|2.50\| | \|0.00\| \|0.00\| | \|2.00\| | \|2.00\| |
| | OD1 | | | | | | |
| W197 | | O | C, CD1, | \|0.50\| \|0.75\| | \|0.00\| \|0.00\| | \|0.25\| | \|0.00\| |
| | | | NE1 | | | | |
| P203 | | CB | O, C, CA | \|1.00\| \|0.25\| | \|0.00\| \|0.00\| | \|0.50\| | \|1.00\| |
| S205 | OG | CB | N, O, CA | \|0.75\| \|1.50\| | \|0.00\| \|0.00\| | \|1.00\| | \|2.00\| |
| F206 | | CE1, CD1 | N, CZ | \|1.25\| \|0.25\| | \|0.00\| \|0.00\| | \|0.00\| | \|0.00\| |
| D207 | CB, | | N | \|2.00\| \|2.25\| | \|−1.00\| \|0.00\| | \|0.00\| | \|4.00\| |
| | CG, | | | | | | |
| | OD1, | | | | | | |
| | OD2 | | | | | | |
| S208 | | OG | CA, CB | \|0.50\| \|0.50\| | \|0.00\| \|0.00\| | \|0.50\| | \|1.00\| |
| I209 | | | | \|0.00\| \|0.00\| | \|0.00\| \|0.00\| | \|0.00\| | \|0.00\| |
| L210 | | | CB, CD2 | \|0.50\| \|0.00\| | \|0.00\| \|0.00\| | \|0.00\| | \|0.00\| |
| D211 | CB, | | CA, CG | \|1.50\| \|1.25\| | \|−0.75\| \|0.00\| | \|0.00\| | \|2.50\| |
| | OD2 | | OD1 | | | | |
| M212 | | | | \|0.00\| \|0.00\| | \|0.00\| \|0.00\| | \|0.00\| | \|0.00\| |
| I213 | | | CB, CG2, | \|0.00\| \|0.75\| | \|0.00\| \|0.00\| | \|0.00\| | \|0.00\| |
| | | | CD1 | | | | |
| S214 | | CB, OG | | \|0.50\| \|0.50\| | \|0.00\| \|0.00\| | \|0.50\| | \|1.00\| |
| M216 | | | | \|0.00\| \|0.00\| | \|0.00\| \|0.00\| | \|0.00\| | \|0.00\| |
| I227 | | | CG1 | \|0.00\| \|0.25\| | \|0.00\| \|0.00\| | \|0.00\| | \|0.00\| |
| I229 | | | | \|0.00\| \|0.00\| | \|0.00\| \|0.00\| | \|0.00\| | \|0.00\| |
| I241 | | | | \|0.00\| \|0.00\| | \|0.00\| \|0.00\| | \|0.00\| | \|0.00\| |
| C242 | | | | \|0.00\| \|0.00\| | \|0.00\| \|0.00\| | \|0.00\| | \|0.00\| |
| I244 | | | | \|0.00\| \|0.00\| | \|0.00\| \|0.00\| | \|0.00\| | \|0.00\| |
| D245 | | | | \|0.00\| \|0.00\| | \|0.00\| \|0.00\| | \|0.00\| | \|0.00\| |
| W248 | | CZ2 | CE2, CE3, | \|1.75\| \|0.25\| | \|0.00\| \|0.00\| | \|0.25\| | \|0.00\| |
| | | | CD2, CZ3, | | | | |
| | | | CH2, NE1 | | | | |
| L285 | | | CA, CD1 | \|0.50\| \|0.00\| | \|0.00\| \|0.00\| | \|0.00\| | \|0.00\| |
| A289 | | | | \|0.00\| \|0.00\| | \|0.00\| \|0.00\| | \|0.00\| | \|0.00\| |
| I290 | | | | \|0.00\| \|0.00\| | \|0.00\| \|0.00\| | \|0.00\| | \|0.00\| |
| Q292 | OE1 | CB, CD, | N, CA, CG | \|1.50\| \|1.75\| | \|0.00\| \|0.00\| | \|1.00\| | \|2.00\| |
| | | NE2 | | | | | |
| L593 | | CG, CD1, | | \|1.50\| \|0.00\| | \|0.00\| \|0.00\| | \|0.00\| | \|0.00\| |
| | | CD2 | | | | | |
| K296 | | CG, CD, | O, C, | \|2.25\| \|0.75\| | \|0.00\| \|0.50\| | \|1.50\| | \|0.00\| |
| | | CE, NZ | CA, CB | | | | |
| L300 | CD1, | | CA, CB, | \|2.75\| \|0.00\| | \|0.00\| \|0.00\| | \|0.00\| | \|0.00\| |
| | CD2 | | CG | | | | |

| | | | | |
|---|---|---|---|---|
| TOTALS | \|21.00\| \|16.00\| | \|−2.25\| \|0.50\| | \|7.50\| | \|17.00\| |

TABLE 34
Comparison of the scoring for Enrichment Model 4.1

| EM4.1 | | | | | | |
|---|---|---|---|---|---|---|
| | hydro- | | Charge | | H-Bonds | |
| | phobicity | polarity | negative | positive | donate | accept |
| SHP2 | 26 | 11 | −2 | 1 | 6.25 | 9.25 |
| PTP1B | 12.25 | 7.25 | −1.5 | 0 | 1.5 | 9 |
| STEP | 27.5 | 14.75 | 0 | 1.5 | 14.5 | 4.75 |
| LYP | 25.25 | 13.5 | −2.5 | 0.5 | 6 | 11.75 |
| PEST | 21 | 16 | −2.25 | 0.5 | 7.5 | 17 |

TABLE 35
Difference results for Enrichment Model 4.1 compared to SHP-2

| EM4.1 | | | | | | |
|---|---|---|---|---|---|---|
| | hydro- | | Charge | | H-Bond | |
| | phobicity | polarity | negative | positve | donate | accept |
| SHP2 | 1 | 1 | 1 | 1 | 1 | 1 |
| PTP1B | 0.47 | 0.66 | 0.75 | 0 | 0.24 | 0.97 |
| STEP | 1.06 | 1.34 | 0 | 1.5 | 2.32 | 0.51 |
| LYP | 0.97 | 1.23 | 1.25 | 0.5 | 1.04 | 1.27 |
| PEST | 0.81 | 1.45 | 1.13 | 0.5 | 1.2 | 1.84 |

Utilization of the Assessment Factors (AF)

To provide a numerical Comparison Value (CV) for each of the Assessment Factors (AF) the absolute value of each AF was recorded in Table 34. To utilize the SHP-2 model as a comparator each AF was divided by the corresponding AF for SHP-2. Table 35 sets out these values as compared to SHP-2, which was set to 1 to provide normalization of the results.

TABLE 36
Scoring of Enrichment Model 4.2 for SHP-2

| EM-4.2 | | | | | (−1) | | |
|---|---|---|---|---|---|---|---|
| | full | half | min | | (0) | (only SC) | (only SC) |
| | access | access | access | (hp) | (+1) | donate | accept |
| SHP2 | 1.00 | 0.50 | 0.25 | polarity | charge | HBs | HBs |
| H394 | CB, CE1, | O, N, CG, | CA, OD1 | \|3.25\| | \|0.00\| \|0.00\| | \|1.50\| | \|1.50\| |
| | ND1 | CD2, NE2 | | \|2.75\| | | | |
| D395 | | OD2 | N, CB, | \|0.50\| | \|−0.75\| \|0.00\| | \|0.00\| | \|1.50\| |
| | | | CG, OD1 | \|1.00\| | | | |
| F424 | | | CE1, CZ | \|0.50\| | \|0.00\| \|0.00\| | \|0.00\| | \|0.00\| |
| | | | | \|0.00\| | | | |
| T426 | CB, CG2 | O | C, CA, | \|2.50\| | \|0.00\| \|0.00\| | \|0.25\| | \|0.50\| |
| | | | OG1 | \|0.75\| | | | |
| W427 | | O | C, CB, N | \|0.50\| | \|0.00\| \|0.00\| | \|0.00\| | \|0.00\| |
| | | | | \|0.75\| | | | |
| P428 | CB | CA, CG, | O, C | \|2.75\| | \|0.00\| \|0.00\| | \|0.00\| | \|0.00\| |
| | | CD | | \|0.25\| | | | |
| V432 | | O, CG2 | N | \|0.50\| | \|0.00\| \|0.00\| | \|0.00\| | \|0.00\| |
| | | | | \|0.75\| | | | |
| P433 | | | O, C, | \|0.75\| | \|0.00\| \|0.00\| | \|0.00\| | \|0.00\| |
| | | | CA, CB | \|0.25\| | | | |
| S434 | O, CB, | CA | C, N | \|1.75\| | \|0.00\| \|0.00\| | \|1.00\| | \|2.00\| |
| | OG | | | \|2.25\| | | | |
| D435 | CG, OD1, | CB | O, N, | \|1.75\| | \|−1.00\| \|0.00\| | \|0.00\| | \|4.00\| |
| | OD2 | | CA | \|2.50\| | | | |
| P436 | | C, CA | CB | \|1.25\| | \|0.00\| \|0.00\| | \|0.00\| | \|0.00\| |
| | | | | \|0.00\| | | | |
| G437 | | C, CA | O, N | \|1.00\| | \|0.00\| \|0.00\| | \|0.00\| | \|0.00\| |
| | | | | \|0.50\| | | | |
| G438 | | CA | N, C | \|0.75\| | \|0.00\| \|0.00\| | \|0.00\| | \|0.00\| |
| | | | | \|0.25\| | | | |
| V439 | | | N, CG2 | \|0.25\| | \|0.00\| \|0.00\| | \|0.00\| | \|0.00\| |
| | | | | \|0.25\| | | | |
| R469 | | | CG, CZ, | \|0.50\| | \|0.00\| \|0.25\| | \|0.75\| | \|0.00\| |
| | | | NE, NH2 | \|0.50\| | | | |
| T472 | | | | \|0.00\| | \|0.00\| \|0.00\| | \|0.00\| | \|0.00\| |
| | | | | \|0.00\| | | | |
| F473 | | | | \|0.00\| | \|0.00\| \|0.00\| | \|0.00\| | \|0.00\| |
| | | | | \|0.00\| | | | |
| Q514 | | | CG, CD, | \|0.50\| | \|0.00\| \|0.00\| | \|0.50\| | \|0.00\| |
| | | | NE2 | \|0.25\| | | | |
| F517 | | | CA, CD1, | \|1.00\| | \|0.00\| \|0.00\| | \|0.00\| | \|0.00\| |
| | | | CE1, CZ | \|0.00\| | | | |

| | | | | |
|---|---|---|---|---|
| TOTALS | \|20.00\| | \|−1.75\| \|0.25\| | \|4.00\| | \|9.50\| |

TABLE 37
Scoring of Enrichment Model 4.2 for PTP1B

| EM-4.2 | | | | | (−1) | | |
|---|---|---|---|---|---|---|---|
| | full | half | min | (hp) | (0) | (only SC) | (only SC) |
| | access | access | access | (polar) | (+1) | donate | accept |
| PTP1B | 1.00 | 0.50 | 0.25 | polarity | charge | HBs | HBs |
| S151 | | O | C, CA, | \|0.75\| | \|0.00\| \|0.00\| | \|0.25\| | \|0.50\| |
| | | | CB, OG | \|0.75\| | | | |
| Y152 | | CB | C, CA | \|1.50\| | \|0.00\| \|0.00\| | \|0.00\| | \|0.00\| |
| | | | CG, CD1 | \|0.00\| | | | |
| Y176 | | | O | \|0.00\| | \|0.00\| \|0.00\| | \|0.00\| | \|0.00\| |
| | | | | \|0.25\| | | | |
| T178 | OG1 | CA, CB | O, N, | \|1.50\| | \|0.00\| \|0.00\| | \|1.00\| | \|2.00\| |
| | | | CG2 | \|1.50\| | | | |
| W179 | | | O, N, C | \|0.25\| | \|0.00\| \|0.00\| | \|0.00\| | \|0.00\| |
| | | | | \|0.50\| | | | |
| P180 | CB, CG | CA, CD | | \|3.00\| | \|0.00\| \|0.00\| | \|0.00\| | \|0.00\| |
| | | | | \|0.00\| | | | |
| V184 | | CG2 | O, N | \|0.50\| | \|0.00\| \|0.00\| | \|0.00\| | \|0.00\| |
| | | | | \|0.50\| | | | |
| P185 | | | O, C | \|0.25\| | \|0.00\| \|0.00\| | \|0.00\| | \|0.00\| |
| | | | | \|0.25\| | | | |
| E186 | O, CB, | CA, CD | N, CG, | \|2.25\| | \|−0.75\| \|0.00\| | \|1.00\| | \|2.50\| |
| | OE1 | | OE2 | \|2.50\| | | | |
| S187 | | CB, OG | CA | \|0.75\| | \|0.00\| \|0.00\| | \|0.50\| | \|1.00\| |
| | | | | \|0.50\| | | | |
| P188 | CD | CG | CB | \|1.75\| | \|0.00\| \|0.00\| | \|0.00\| | \|0.00\| |
| | | | | \|0.00\| | | | |
| A189 | | | N, CA, | \|0.50\| | \|0.00\| \|0.00\| | \|0.00\| | \|0.00\| |
| | | | CB | \|0.25\| | | | |
| S190 | | | | \|0.00\| | \|0.00\| \|0.00\| | \|0.00\| | \|0.00\| |
| | | | | \|0.00\| | | | |
| F191 | | | | \|0.00\| | \|0.00\| \|0.00\| | \|0.00\| | \|0.00\| |
| | | | | \|0.00\| | | | |
| R221 | | | N, CG, | \|0.25\| | \|0.00\| \|0.25\| | \|0.75\| | \|0.00\| |
| | | | NE, NH2 | \|0.75\| | | | |
| T224 | | | | \|0.00\| | \|0.00\| \|0.00\| | \|0.00\| | \|0.00\| |
| | | | | \|0.00\| | | | |
| F224 | | | | \|0.00\| | \|0.00\| \|0.00\| | \|0.00\| | \|0.00\| |
| | | | | \|0.00\| | | | |
| Q266 | | | | \|0.00\| | \|0.00\| \|0.00\| | \|0.00\| | \|0.00\| |
| | | | | \|0.00\| | | | |
| F269 | | CD1 | CA, CB, | \|1.25\| | \|0.00\| \|0.00\| | \|0.00\| | \|0.00\| |
| | | | CE1 | \|0.00\| | | | |

| | | | | |
|---|---|---|---|---|
| TOTALS | \|14.50\| | \|−0.75\| \|0.25\| | \|2.50\| | \|6.00\| |
| | \|7.75\| | | | |

TABLE 38
Scoring of Enrichment Model 4.2 for STEP

| EM-4.2 | | | | | (−1) | | |
|---|---|---|---|---|---|---|---|
| | full | half | min | (hp) | (0) | (only SC) | (only SC) |
| | access | access | access | (polar) | (+1) | donate | accept |
| STEP | 1.00 | 0.50 | 0.25 | polarity | charge | HBs | HBs |
| E433 | CB, CG, | O, CD, | N, CA | \|2.75\| | \|−1.00\| \|0.00\| | \|0.00\| | \|3.00\| |
| | OE2 | OE1 | | \|2.25\| | | | |
| D434 | | CB | N | \|0.50\| | \|0.00\| \|0.00\| | \|0.00\| | \|0.00\| |
| | | | | \|0.25\| | | | |
| F456 | | | CZ | \|0.25\| | \|0.00\| \|0.00\| | \|0.00\| | \|0.00\| |
| | | | | \|0.00\| | | | |
| S458 | CB | O, CA, | C | \|1.75\| | \|0.00\| \|0.00\| | \|0.50\| | \|1.00\| |
| | | OG | | \|1.00\| | | | |
| W459 | | | O, C, | \|0.75\| | \|0.00\| \|0.00\| | \|0.25\| | \|0.00\| |
| | | | CE2, CZ2, | \|0.50\| | | | |
| | | | NE1 | | | | |
| P460 | CB | | CA, CG, | \|1.75\| | \|0.00\| \|0.00\| | \|0.00\| | \|0.00\| |
| | | | CD | \|0.00\| | | | |
| T464 | | OG1 | O, N, | \|0.25\| | \|0.00\| \|0.00\| | \|0.50\| | \|1.00\| |
| | | | CG1 | \|1.00\| | | | |
| P465 | | CB | O, C, | \|1.25\| | \|0.00\| \|0.00\| | \|0.00\| | \|0.00\| |
| | | | CA, CG | \|0.25\| | | | |
| D466 | O, CB, | | N, C, | \|2.50\| | \|−1.00\| \|0.00\| | \|0.00\| | \|4.00\| |
| | CG, OD1, | | CA | \|3.25\| | | | |
| | OD2 | | | | | | |
| R467 | NH1, | CB, CD, | O, CA, | \|2.00\| | \|0.00\| \|0.75\| | \|4.25\| | \|0.00\| |
| | NH2 | CZ | CG, NE | \|2.50\| | | | |
| A468 | | | N, CB | \|0.25\| | \|0.00\| \|0.00\| | \|0.00\| | \|0.00\| |
| | | | | \|0.25\| | | | |
| P469 | | CB, CG | C, CA, | \|1.75\| | \|0.00\| \|0.00\| | \|0.00\| | \|0.00\| |
| | | | CD | \|0.00\| | | | |
| P470 | | CG | CA, CB, | \|1.25\| | \|0.00\| \|0.00\| | \|0.00\| | \|0.00\| |
| | | | CD | \|0.00\| | | | |
| L471 | | | N, CB, | \|0.50\| | \|0.00\| \|0.00\| | \|0.00\| | \|0.00\| |
| | | | CD1 | \|0.25\| | | | |
| R502 | | CG, CD, | N, NH1 | \|1.50\| | \|0.00\| \|0.50\| | \|2.00\| | \|0.00\| |
| | | CZ, NE, | | \|1.50\| | | | |
| | | NH2 | | | | | |
| C505 | | | | \|0.00\| | \|0.00\| \|0.00\| | \|0.00\| | \|0.00\| |
| | | | | \|0.00\| | | | |
| F506 | | | CE2, CZ | \|0.50\| | \|0.00\| \|0.00\| | \|0.00\| | \|0.00\| |
| | | | | \|0.00\| | | | |
| Q544 | | NE2 | CG | \|0.25\| | \|0.00\| \|0.00\| | \|0.00\| | \|0.00\| |
| | | | | \|0.25\| | | | |
| F547 | | | CA, CB, | \|1.00\| | \|0.00\| \|0.00\| | \|0.00\| | \|0.00\| |
| | | | CD1, CE1 | \|0.00\| | | | |

| | | | | |
|---|---|---|---|---|
| TOTALS | \|20.75\| | \|−2.00\| \|1.25\| | \|7.50\| | \|9.00\| |
| | \|13.25\| | | | |

TABLE 39
Scoring of Enrichment Model 4.2 for LYP (PTPN22, PEP, PTPN8)

| EM-4.2 | | | | | (−1) | | |
|---|---|---|---|---|---|---|---|
| | full | half | min | (hp) | (0) | (only SC) | (only SC) |
| | access | access | access | (polar) | (+1) | donate | accept |
| LYP | 1.00 | 0.50 | 0.25 | polarity | charge | HBs | HBs |
| S167 | CB, OG | O, CA | N, C | \|1.75\| | \|0.00\| \|0.00\| | \|1.00\| | \|2.00\| |
| | | | | \|1.75\| | | | |
| D168 | | OD1 | C, CA, | \|0.75\| | \|−0.25\| \|0.00\| | \|0.00\| | \|1.00\| |
| | | | CB | \|0.50\| | | | |
| Y190 | | | OH | \|0.00\| | \|0.00\| \|0.00\| | \|0.25\| | \|0.25\| |
| | | | | \|0.25\| | | | |
| N192 | ND2 | O, CA, | C, CB | \|1.50\| | \|0.00\| \|0.00\| | \|2.00\| | \|1.00\| |
| | | CG, | | \|2.00\| | | | |
| | | OD1 | | | | | |
| W193 | | O | C, CB | \|0.50\| | \|0.00\| \|0.00\| | \|0.00\| | \|0.00\| |
| | | | | \|0.50\| | | | |
| P194 | CB, CG | CA | | \|2.50\| | \|0.00\| \|0.00\| | \|0.00\| | \|0.00\| |
| | | | | \|0.00\| | | | |
| V198 | | CG2 | O, CG1 | \|0.75\| | \|0.00\| \|0.00\| | \|0.00\| | \|0.00\| |
| | | | | \|0.25\| | | | |
| P199 | | | O, C, | \|0.75\| | \|0.00\| \|0.00\| | \|0.00\| | \|0.00\| |
| | | | CA, CB | \|0.25\| | | | |
| S200 | O, CB, | CA | C, N | \|2.75\| | \|0.00\| \|0.00\| | \|0.00\| | \|0.00\| |
| | CG | | | \|1.25\| | | | |
| S201 | CB | OG | O, N, | \|1.25\| | \|0.00\| \|0.00\| | \|0.50\| | \|1.00\| |
| | | | CA | \|1.00\| | | | |
| I202 | CD1 | | CG2 | \|1.25\| | \|0.00\| \|0.00\| | \|0.00\| | \|0.00\| |
| | | | | \|0.00\| | | | |
| D203 | OD2 | CB | O, C, | \|1.00\| | \|−0.75\| \|0.00\| | \|0.00\| | \|2.50\| |
| | | | CG, OD1 | \|1.50\| | | | |
| P204 | CG | | CA, CB, | \|1.75\| | \|0.00\| \|0.00\| | \|0.00\| | \|0.00\| |
| | | | CD | \|0.00\| | | | |
| I205 | | | | \|0.00\| | \|0.00\| \|0.00\| | \|0.00\| | \|0.00\| |
| | | | | \|0.00\| | | | |
| R233 | | NE, NH2 | N, CG, | \|0.50\| | \|0.00\| \|0.50\| | \|0.00\| | \|1.50\| |
| | | | CZ | \|1.25\| | | | |
| V236 | | | | \|0.00\| | \|0.00\| \|0.00\| | \|0.00\| | \|0.00\| |
| | | | | \|0.00\| | | | |
| I237 | | | | \|0.00\| | \|0.00\| \|0.00\| | \|0.00\| | \|0.00\| |
| | | | | \|0.00\| | | | |
| Q278 | | | CG, CD, | \|0.50\| | \|0.00\| \|0.00\| | \|0.00\| | \|0.00\| |
| | | | OE1 | \|0.25\| | | | |
| L281 | | CA, CD1 | N | \|1.00\| | \|0.00\| \|0.00\| | \|0.00\| | \|0.00\| |
| | | | | \|0.25\| | | | |

| | | | | |
|---|---|---|---|---|
| TOTALS | \|18.50\| | \|−1.00\| \|0.50\| | \|3.75\| | \|9.25\| |
| | \|11.00\| | | | |

TABLE 40
Scoring of Enrichment Model 4.2 for PTP-PEST (PTPN12, PTPG1)

| EM-4.2 | | | | | (−1) | | |
|---|---|---|---|---|---|---|---|
| | full | half | min | (hp) | (0) | (only SC) | (only SC) |
| | access | access | access | (polar) | (+1) | donate | accept |
| PEST | 1.00 | 0.50 | 0.25 | polarity | charge | HBs | HBs |
| T171 | CG2 | O, CA, | N, C | \|2.25\| | \|0.00\| \|0.00\| | \|0.50\| | \|1.00\| |
| | | CB, OG1 | | \|1.25\| | | | |
| D172 | | OD2 | CB, CG, | \|0.50\| | \|−0.50\| \|0.00\| | \|0.00\| | \|1.50\| |
| | | | OD1 | \|0.75\| | | | |
| Y194 | | | OH | \|0.00\| | \|0.00\| \|0.00\| | \|0.25\| | \|0.25\| |
| | | | | \|0.25\| | | | |
| N196 | CG, ND2 | O, CB | C, CA | \|2.00\| | \|0.00\| \|0.00\| | \|2.00\| | \|2.00\| |
| | OD1 | | | \|2.50\| | | | |
| W197 | | | O, C | \|0.25\| | \|0.00\| \|0.00\| | \|0.00\| | \|0.00\| |
| | | | | \|0.25\| | | | |
| P198 | CB | CA | CG, CD | \|2.00\| | \|0.00\| \|0.00\| | \|0.00\| | \|0.00\| |
| | | | | \|0.00\| | | | |
| V202 | | O, CG2 | CG1 | \|0.75\| | \|0.00\| \|0.00\| | \|0.00\| | \|0.00\| |
| | | | | \|0.50\| | | | |
| P203 | | CA, CB | O, C | \|1.25\| | \|0.00\| \|0.00\| | \|0.00\| | \|0.00\| |
| | | | | \|0.25\| | | | |
| S204 | O, CB, OG | CA, | N, C | \|1.75\| | \|0.00\| \|0.00\| | \|1.00\| | \|2.00\| |
| | | | | \|2.25\| | | | |
| S205 | CB, OG | | N, O, CA | \|1.25\| | \|0.00\| \|0.00\| | \|1.00\| | \|2.00\| |
| | | | | \|1.50\| | | | |
| F206 | | CD1, | N, CZ | \|1.25\| | \|0.00\| \|0.00\| | \|0.00\| | \|0.00\| |
| | | CE1 | | \|0.25\| | | | |
| D207 | CG, OD2 | CB, OD1 | N | \|1.50\| | \|−1.00\| \|0.00\| | \|0.00\| | \|3.00\| |
| | | | | \|1.75\| | | | |
| S208 | | OG | CA, CB | \|0.50\| | \|0.00\| \|0.00\| | \|0.50\| | \|1.00\| |
| | | | | \|0.50\| | | | |
| I209 | | | | \|0.00\| | \|0.00\| \|0.00\| | \|0.00\| | \|0.00\| |
| | | | | \|0.00\| | | | |
| R237 | | CG, CZ, | N, CD | \|1.25\| | \|0.00\| \|0.50\| | \|2.00\| | \|0.00\| |
| | | NE, NH2 | | \|1.25\| | | | |
| A240 | | | | \|0.00\| | \|0.00\| \|0.00\| | \|0.00\| | \|0.00\| |
| | | | | \|0.00\| | | | |
| I241 | | | | \|0.00\| | \|0.00\| \|0.00\| | \|0.00\| | \|0.00\| |
| | | | | \|0.00\| | | | |
| Q282 | | | CG, | \|0.25\| | \|0.00\| \|0.00\| | \|0.50\| | \|0.00\| |
| | | | NE2 | \|0.25\| | | | |
| L285 | | | CA, CD1 | \|0.50\| | \|0.00\| \|0.00\| | \|0.00\| | \|0.00\| |
| | | | | \|0.00\| | | | |

| | | | | |
|---|---|---|---|---|
| TOTALS | \|17.25\| | \|−1.50\| \|0.50\| | \|7.75\| | \|12.75\| |
| | \|13.50\| | | | |

TABLE 41
Comparison of the scoring for Enrichment Model 4.1

| EM4.2 | | | | | | |
|---|---|---|---|---|---|---|
| | hydro- | | Charge | | H-Bonds | |
| | phobicity | polarity | negative | positive | donate | accept |
| SHP2 | 20 | 13 | −1.75 | 0.25 | 4 | 9.5 |
| PTP1B | 14.5 | 7.75 | −0.75 | 0.25 | 2.5 | 6 |
| STEP | 20.75 | 13.25 | −2 | 1.25 | 7.5 | 9 |
| LYP | 18.5 | 11 | −1 | 0.5 | 3.75 | 9.25 |
| PEST | 17.25 | 13.5 | −1.5 | 0.5 | 7.75 | 12.75 |

TABLE 42
Difference results for Enrichment Model 4.2 compared to SHP-2

| EM4.2 | | | | | | |
|---|---|---|---|---|---|---|
| | hydro- | | Charge | | H-Bonds | |
| | phobicity | polarity | negative | positive | donate | accept |
| SHP2 | 1 | 1 | 1 | 1 | 1 | 1 |
| PTP1B | 0.73 | 0.6 | 0.43 | 1 | 0.63 | 0.63 |
| STEP | 1.04 | 1.02 | 1.14 | 5 | 1.88 | 0.95 |
| LYP | 0.93 | 0.85 | 0.57 | 2 | 0.94 | 0.97 |
| PEST | 0.86 | 1.04 | 0.86 | 2 | 1.94 | 1.34 |

Utilization of the Assessment Factors (AF)

To provide a numerical Comparison Value (CV) for each of the Assessment Factors (AF) the absolute value of each AF in was recorded in Table 41. To utilize the SHP-2 model as a comparator each AF was divided by the corresponding AF for SHP-2. Table 42 sets out these values as compared to SHP-2, which was set to 1 to provide normalization of the results. SHP1 has 4 isoforms, isoform 1 is the canonical sequence:

sp|P29350|PTN6
HUMAN Tyrosine-protein phosphatase non-receptor
type 6 OS = Homo sapiens
GN = PTPN6 PE =1 SV = 1
MVRWFHRDLSGLDAETLLKGRGVHGSFLARPSRKNQGDFSLSVRVGDQV
THIRIQNSGDFQDLYGGEKFATLTELVEYYTQQQGVLQDRDGTHHLKYP
LNCSDPTSERWYHGHMSGGQAETLLQAKGEPWTFLVRESLSQPGDFVLS
VLSDQPKAGPGSPLRVTHIKVMCEGGRYTVGGLETFDSLTDLVEHFKKT
GIEEASGAFVYLRQPYYATRVNAADIENRVLELNKKQESEDTAKAGFWE
EFESLQKQEVKNLHQRLEGQRPENKGKNRYKNILPFDHSRVILQGRDSN
IPGSDYINANYIKNQLLGPDENAKTYIASQGCLEATVNDFWQMAWQENS
RVIVMTTREVEKGRNKCVPYWPEVGMQRAYGPYSVTNCGEHDTTEYKLR
TLQVSPLDNGDLIREIWHYQYLSWPDHGVPSEPGGVLSFLDQINQRQES
LPHAGPIIVHCSAGIGRTGTHVIDMLMENISTKGLDCDIDIQKTIQMVR
AQRSGMVQTEAQYKFIYVAIAQFIETTKKKLEVLQSQKGQESEYGNITY
PPAMKNAHAKASRTSSKHKEDVYENLHTKNKREEKVKKQRSADKEKSKG
SLKRK

SHP2 has 3 isoforms, isoform 1 is the canonical sequence

sp|Q06124|PTN11
HUMAN Tyrosine-protein phosphatase non-receptor
type 11 OS = Homo sapiens
GN = PTPN11 PE = 1 SV = 2
MTSRRWFHPNITGVEAENLLLTRGVDGSFLARPSKSNPGDFTLSVRRNG
AVTHIKIQNTGDYYDLYGGEKFATLAELVQYYMEHHGQLKEKNGDVIEL
KYPLNCADPTSERWFHGHLSGKEAEKLLTEKGKHGSFLVRESQSHPGDF
VLSVRTGDDKGESNDGKSKVTHVMIRCQELKYDVGGGERFDSLTDLVEH
YKKNPMVETLGTVLQLKQPLNTTRINAAEIESRVRELSKLAETTDKVKQ
GFWEEFETLQQQECKLLYSRKEGQRQENKNKNRYKNILPFDHTRVVLHD
GDPNEPVSDYINANIIMPEFETKCNNSKPKKSYIATQGCLQNTVNDFWR
MVFQENSRVIVMTTKEVERGKSKCVKYWPDEYALKEYGVMRVRNVKESA
AHDYTLRELKLSKVGQALLQGNTERTVWQYHFRTWPDHGVPSDPGGVLD
FLEEVHHKQESIMDAGPVVVHCSAGIGRTGTFIVIDILIDIIREKGVDC
DIDVPKTIQMVRSQRSGMVQTEAQRYFIYMAVQHYIETLQRRIEEEQKS
KRKGHEYTNIKYSLADQTSGDQSPLPPCTPTPPCAEMREDSARVYENVG
LMQQQKSFR

Isoform 2 is the most common biological sequence and is the present in the crystal structures in the RCSB

>sp|Q06124-2|PTN11
HUMAN Isoform 2 of Tyrosine-protein phosphatase
non-receptor type 11 OS = Homo sapiens
GN = PTPN11
MTSRRWFHPNITGVEAENLLLTRGVDGSFLARPSKSNPGDFTLSVRRNG
AVTHIKIQNTGDYYDLYGGEKFATLAELVQYYMEHHGQLKEKNGDVIKL
KYPLNCADPTSERWFHGHLSGKEAEKLLTEKGKHGSFLVRESQSHPGDF
VLSVRTGDDKGESNDGKSKVTHVMIRCQELKYDVGGGERFDSLTDLVEH
YKKNPMVETLGTVLQLKQPLNTTRINAAEIESRVRELSKLAETTDKVKQ
GFWEEFETLQQQECKLLYSRKEGQRQENKNKNRYKNILPFDHTRVVLHD
GDPNEPVSDYINANIIMPEFETKCNNSKPKKSYIATQGCLQNTVNDFWR
MVFQENSRVIVMTTKEVERGKSKCVKYWPDEYALKEYGVMRVRNVKESA
AHDYTLRELKLSKVGQGNTERTVWQYHFRTWPDHGVPSDPGGVLDFLEE
VHHKQESIMDAGPVVVHCSAGIGRTGTFIVIDILIFIIREKGVDCDIDV
PKTIQMVRSQRSGMVQTEAQYRFIYMAVQHYIETLQRRIEEEQKSKRKG
HEYTNIKYSLADQTSGDQSPLPPCTPTPPCAEMREDSARVYENVGLMQQ
QKSFR

The sequence of this isoform differs from the canonical sequence as follows: 408-411: Missing

What is claimed is:

1. A method for making, a 3-dimensional enrichment model for a phosphatase enzyme comprising the steps of
constructing missing loops and side chains using homology modeling with a target peptide sequence;
add missing components to target peptide;
check completed peptide for errors:
relax completed peptide in a solvent;
search peptide for presence of molecular features suitable for binding:
measure size and polarity of suspected binding sites; and
identify structural features of peptide capable of binding.

2-3. (canceled)

4. The method of claim 1, wherein said phosphatase enzyme is a tyrosine phosphatase.

5. The method of claim 4, wherein said tyrosine phosphatase is SHP1 or SHP2.

6. (canceled)

7. A method to enrich a chemical library for a modulator of a tyrosine phosphatase enzyme comprising the following steps:
using a computer algorithm to generate a binding model;
preparing a 3-dimensional conformation database of candidate modulators;
preparing a 3-dimensional representation of the target enzyme;
generating 3-dimensional representations of modulation sites;
determining the structure coordinates of the amino acid residues that constitute the binding sites;
comparing said binding model to a candidate molecule;
estimating the attraction, repulsion and steric hindrance of a potential ligand to the enrichment model; and
selecting molecules that are compatible with the enrichment model.

8-10. (canceled)

11. The method of claim 7, wherein said tyrosine phosphatase enzyme is SHP1 or SHP2.

12-30. (canceled)

31. A method of using a SHP1 or SHP2 enrichment model to identify ligands that bind to SHP1 or SHP2 and modulate its function, comprising the steps of
generating one or more electronic representations of a compound or fragment;
assembling an electronic representation or representations in an electronic database;
positioning selected chemical entities in a variety of orientations inside an enrichment model;
using selected chemical entities to perform a manual or computer-assisted fitting of said electronic representations and an enrichment model;
analyzing the results of said fitting operation to quantify the association between said chemical entities and said enrichment model;
evaluating the quality of the fitting of said chemical entities to said enrichment mode using a scoring function, shape complementarity, interaction energy estimate and visual inspection followed by energy minimization and molecular dynamics; and
identifying suitable chemical entities and connecting same into a single compound in relation to said enrichment model.

32-39. (canceled)

40. A virtual 3-dimensional SHP2 enrichment model according to claim 5, comprising
amino acid residues G⁶⁰, D⁶¹, Y⁶², E³⁶¹, R³⁶², K³⁶⁴, K³⁶⁶, W⁴²³, P⁴²⁴, D⁴²⁵, H⁴²⁶, G⁴²⁷, V⁴²⁸, G⁴⁶⁴, R⁴⁶⁵, Q⁵¹⁰;
amino acid residues G⁴³⁷, L⁴⁴⁰, D⁴⁴¹, E⁴⁴⁴, E⁴⁴⁵, H⁴⁴⁸, H⁵²⁴, Y⁵²⁵, E⁵²⁷, T⁵²⁸, R⁵³¹, R⁵³², I⁵³³, E⁵³⁴, E⁵³⁵, E⁵³⁶, K⁵⁴⁰;
amino acid residues P³¹², E³¹³, F³¹⁴, E³¹⁵, K³²², P³²³, K³²⁴, K³²⁵, S³²⁶, Y³²⁷, H⁴⁴⁷, Q⁴⁵⁰, E⁴⁵¹, I⁴⁵³, M⁴⁵⁴, A⁴⁵⁶, G⁴⁵⁷, P⁴⁵⁸, V⁴⁵⁹, D⁴⁷⁷, I⁴⁷⁸, D⁴⁸¹, I⁴⁸², R⁴⁸⁴, E⁴⁸⁵, K⁴⁸⁶, E⁵³⁴, E⁵³⁵, E⁵³⁶, Q⁵³⁷, K⁵³⁸, S⁵³⁹, K⁵⁴⁰, R⁵⁴¹, K⁵⁴², G⁵⁴³, H⁵⁴⁴, E⁵⁴⁵, Y⁵⁴⁶, T⁵⁴⁷;
amino acid residues Y³²⁷, V³⁵⁴, D³⁹⁵, F⁴²⁴, T⁴²⁶, W⁴²⁷, P⁴³³, D⁴³⁵, P⁴³⁶, G⁴³⁷, G⁴³⁸, V⁴³⁹, L⁴⁴⁰, D⁴⁴¹, F⁴⁴², L⁴⁴³, E⁴⁴⁴, V⁴⁴⁶, V⁴⁵⁹, V⁴⁶¹, F⁴⁷³, I⁴⁷⁴, I⁴⁷⁶, D⁴⁷⁷, I⁴⁸⁰, F⁵¹⁷, A⁵²¹, V⁵²², H⁵²⁴, Y⁵²⁵, T⁵²⁸, R⁵³²; or
amino acid residues H³⁹⁴, D³⁹⁵, F⁴²⁴, T⁴²⁶, W⁴²⁷, P⁴²⁸, V⁴³², B⁴³³, S⁴³⁴, D⁴³⁵, P⁴³⁶, G⁴³⁷, G⁴³⁸, V⁴³⁹, R⁴⁶⁹, T⁴⁷², F⁴⁷³, Q⁵¹⁴, F⁵¹⁷.

41-42. (canceled)

43. A compound selected from an enriched chemical library according to claim 7 that modulates protein tyrosine phosphatase activity wherein said compound inhibits tumor cell growth.

44. The compound of claim 43, wherein said compound is a small molecule.

45. The compound of claim 43, wherein said compound inhibits the activity of SHP1 or SHP2.

46-48. (canceled)

49. A modulator of protein tyrosine phosphatase discovered using an enrichment model according to claim 4, wherein said protein tyrosine phosphatase is selected from the group consisting of PTB1B, PTP-PEST, LYP and striatal-enriched phosphatase (STEP) wherein said modulator is used to inhibit the activity of said protein tyrosine phosphatase.

50-52. (canceled)

53. The modulator according to claim 49, wherein said modulator is used to prevent the negative regulation of B and T cell signaling; treat Alzheimer's disease, schizophrenia, fragile X syndrome, epileptogenesis and alcohol-induced memory loss; or to treat autoimmune disorders selected from the group consisting of rheumatoid arthritis, systemic lupus, erythematosus, vitiligo and Graves' Disease.

54-58. (canceled)

59. A 3-dimensional enrichment model for PTP-PEST, LYP, PTP1B and STEP made from the method according to claim 1.

60. A chemical library for PTP-PEST, LYP, PTP1B, STEP, SHP1 and SHP2 using the enrichment models according to claim 7.

61. (canceled)

62. A method of using an enrichment model of claim 59 to determine the degree of similarity between different enrichment models derived from different proteins to enrich a chemical library, wherein said comparison identifies modulators with similar or dissimilar structural features.

63-64. (canceled)

65. The library according to claim 62, wherein said protein tyrosine phosphatase is selected from the group consisting of PTP-PEST, LYP, PTP1B and STEP.

66. An enrichment model for de-phosphorylation enzymes according to claim 1 selected from the group consisting of a PTP-PEST (PTPN12, PTPG1) enrichment model containing the residues: A₁₃₂, Y₁₉₄, N₁₉₆, W₁₉₇, H₂₀₀, D₂₀₁, V₂₀₂, S₂₀₅, F₂₀₆, S₂₀₈, I₂₀₉, G₂₃₆, R₂₃₇, A₂₄₀, I₂₄₁, E281, Q₂₈₂, E284, L₂₈₅, R₂₈₈ used to prevent the negative regulation of B and T cell signaling; a STEP Enrichment model containing the residues: I₃₇₄, N₃₇₆, F₄₃₂, S₄₃₄, W₄₃₅, P₄₃₆, D₄₃₇, Q₄₃₈, K₄₃₉, P₄₄₁, D₄₄₂, R₄₄₃, P₄₄₅, P₄₄₆, L₄₄₇, R₄₇₈, C₄₈₁, F₄₈₂, T₅₁₇, E₅₁₉, Q₅₂₀, Q₅₂₂, F₅₂₃, H₅₂₆ used to treat Alzheimer's disease, schizophrenia, fragile X syndrome, epileptogenesis and alcohol-induced memory loss; and a LYP (PTPN22, PEP, PTPN8) Enrichment model containing the residues: Y₁₉₀, K₁₉₁, W₁₉₃, D₁₉₇, V₁₉₈, P₁₉₉, S₂₀₁, I₂₀₂, I₂₀₅, G₂₃₂, R₂₃₃, V₂₃₆, I₂₃₇, T₂₇₅, E₂₇₇, Q₂₇₈, E₂₈₀, L₂₈₁, N₂₈₄ used to treat to treat rheumatoid arthritis, systemic lupus, erythematosus, vitiligo or Graves' Disease.

67-78. (canceled)
